SEQUENCE LISTING (567 sequences)

SEQ ID NO: 1 - protein, 439 aa, synthetic construct
  REGION 1..439: note = Engineered antibody sequence
QSLEESGGRLVTPGTPLTLTCTVSGLDLSSYYMQWVRQAPGKGLEWIGVIGINDNTYYASWAKGRFTISRASSTTVDLKMTSLTTEDTATYFCARGDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 109 aa, synthetic construct
  REGION 1..109: note = Engineered antibody sequence
QSLEESGGRLVTPGTPLTLTCTVSGLDLSSYYMQWVRQAPGKGLEWIGVIGINDNTYYASWAKGRFTISRASSTTVDLKMTSLTTEDTATYFCARGDIWGPGTLVTVSS

SEQ ID NO: 3 - protein, 29 aa, synthetic construct
  REGION 1..29: note = Engineered antibody sequence
QSLEESGGRLVTPGTPLTLTCTVSGLDLS

SEQ ID NO: 4 - protein, 5 aa, synthetic construct
  REGION 1..5: note = Engineered antibody sequence
SYYMQ

SEQ ID NO: 5 - protein, 14 aa, synthetic construct
  REGION 1..14: note = Engineered antibody sequence
WVRQAPGKGLEWIG

SEQ ID NO: 6 - protein, 16 aa, synthetic construct
  REGION 1..16: note = Engineered antibody sequence
VIGINDNTYYASWAKG

SEQ ID NO: 7 - protein, 31 aa, synthetic construct
  REGION 1..31: note = Engineered antibody sequence
RFTISRASSTTVDLKMTSLTTEDTATYFCAR

SEQ ID NO: 8 - skipped

SEQ ID NO: 9 - protein, 11 aa, synthetic construct
  REGION 1..11: note = Engineered antibody sequence
WGPGTLVTVSS

SEQ ID NO: 10 - protein, 330 aa, synthetic construct
  REGION 1..330: note = Engineered antibody sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1320 bp, synthetic construct
  misc_feature 1..1320: note = Engineered antibody sequence
  source 1..1320: mol_type = other DNA
cagtcgctggaggagtccgggggtcgcctggtcacgcctgggacacccctgacactcacctgcacagtctctggactcgacctcagtagctactacatgcaatgggtccgccaggctccagggaaggggctggaatggatcggagtcattggtattaatgataacacatactacgcgagctgggcgaaaggccgattcaccatctccagagcctcgtcgaccacggtggatctgaaaatgaccagtctgacaaccgaggacacggccacctatttctgtgccagaggggacatctggggcccaggcaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - DNA, 327 bp, synthetic construct
  misc_feature 1..327: note = Engineered antibody sequence
  source 1..327: mol_type = other DNA
cagtcgctggaggagtccgggggtcgcctggtcacgcctgggacacccctgacactcacctgcacagtctctggactcgacctcagtagctactacatgcaatgggtccgccaggctccagggaaggggctggaatggatcggagtcattggtattaatgataacacatactacgcgagctgggcgaaaggccgattcaccatctccagagcctcgtcgaccacggtggatctgaaaatgaccagtctgacaaccgaggacacggccacctatttctgtgccagaggggacatctggggcccaggcaccctcgtcaccgtctcgagc

SEQ ID NO: 13 - DNA, 87 bp, synthetic construct
  misc_feature 1..87: note = Engineered antibody sequence
  source 1..87: mol_type = other DNA
cagtcgctggaggagtccgggggtcgcctggtcacgcctgggacacccctgacactcacctgcacagtctctggactcgacctcagt

SEQ ID NO: 14 - DNA, 15 bp, synthetic construct
  misc_feature 1..15: note = Engineered antibody sequence
  source 1..15: mol_type = other DNA
agctactacatgcaa

SEQ ID NO: 15 - DNA, 42 bp, synthetic construct
  misc_feature 1..42: note = Engineered antibody sequence
  source 1..42: mol_type = other DNA
tgggtccgccaggctccagggaaggggctggaatggatcgga

SEQ ID NO: 16 - DNA, 48 bp, synthetic construct
  misc_feature 1..48: note = Engineered antibody sequence
  source 1..48: mol_type = other DNA
gtcattggtattaatgataacacatactacgcgagctgggcgaaaggc

SEQ ID NO: 17 - DNA, 93 bp, synthetic construct
  misc_feature 1..93: note = Engineered antibody sequence
  source 1..93: mol_type = other DNA
cgattcaccatctccagagcctcgtcgaccacggtggatctgaaaatgaccagtctgacaaccgaggacacggccacctatttctgtgccaga

SEQ ID NO: 18 - skipped

SEQ ID NO: 19 - DNA, 33 bp, synthetic construct
  misc_feature 1..33: note = Engineered antibody sequence
  source 1..33: mol_type = other DNA
tggggcccaggcaccctcgtcaccgtctcgagc

SEQ ID NO: 20 - DNA, 993 bp, synthetic construct
  misc_feature 1..993: note = Engineered antibody sequence
  source 1..993: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 21 - protein, 219 aa, synthetic construct
  REGION 1..219: note = Engineered antibody sequence
QVLTQTASPVSAAVGSTVTINCQASQSVYDNNYLAWYQQKPGQPPKQLIYSTSTLASGVSSRFKGSGSGTQFTLTISDLECADAATYYCLGSYDCSSGDCFVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 113 aa, synthetic construct
  REGION 1..113: note = Engineered antibody sequence
QVLTQTASPVSAAVGSTVTINCQASQSVYDNNYLAWYQQKPGQPPKQLIYSTSTLASGVSSRFKGSGSGTQFTLTISDLECADAATYYCLGSYDCSSGDCFVFGGGTEVVVKR

SEQ ID NO: 23 - protein, 22 aa, synthetic construct
  REGION 1..22: note = Engineered antibody sequence
QVLTQTASPVSAAVGSTVTINC

SEQ ID NO: 24 - protein, 13 aa, synthetic construct
  REGION 1..13: note = Engineered antibody sequence
QASQSVYDNNYLA

SEQ ID NO: 25 - protein, 15 aa, synthetic construct
  REGION 1..15: note = Engineered antibody sequence
WYQQKPGQPPKQLIY

SEQ ID NO: 26 - protein, 7 aa, synthetic construct
  REGION 1..7: note = Engineered antibody sequence
STSTLAS

SEQ ID NO: 27 - protein, 32 aa, synthetic construct
  REGION 1..32: note = Engineered antibody sequence
GVSSRFKGSGSGTQFTLTISDLECADAATYYC

SEQ ID NO: 28 - protein, 13 aa, synthetic construct
  REGION 1..13: note = Engineered antibody sequence
LGSYDCSSGDCFV

SEQ ID NO: 29 - protein, 11 aa, synthetic construct
  REGION 1..11: note = Engineered antibody sequence
FGGGTEVVVKR

SEQ ID NO: 30 - protein, 106 aa, synthetic construct
  REGION 1..106: note = Engineered antibody sequence
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 660 bp, synthetic construct
  misc_feature 1..660: note = Engineered antibody sequence
  source 1..660: mol_type = other DNA
caagtgctgacccagactgcatcccccgtgtctgcagctgtgggaagcacagtcaccatcaattgccaggccagtcagagtgtttatgataacaactacctagcctggtatcagcagaaaccagggcagcctcccaagcaactgatctattctacatccactctggcatctggggtctcatcgcggttcaaaggcagtggatctgggacacagttcactctcaccatcagcgacctggagtgtgccgatgctgccacttactactgtctaggcagttatgattgtagtagtggtgattgttttgttttcggcggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 32 - DNA, 339 bp, synthetic construct
  misc_feature 1..339: note = Engineered antibody sequence
  source 1..339: mol_type = other DNA
caagtgctgacccagactgcatcccccgtgtctgcagctgtgggaagcacagtcaccatcaattgccaggccagtcagagtgtttatgataacaactacctagcctggtatcagcagaaaccagggcagcctcccaagcaactgatctattctacatccactctggcatctggggtctcatcgcggttcaaaggcagtggatctgggacacagttcactctcaccatcagcgacctggagtgtgccgatgctgccacttactactgtctaggcagttatgattgtagtagtggtgattgttttgttttcggcggagggaccgaggtggtggtcaaacgt

SEQ ID NO: 33 - DNA, 66 bp, synthetic construct
  misc_feature 1..66: note = Engineered antibody sequence
  source 1..66: mol_type = other DNA
caagtgctgacccagactgcatcccccgtgtctgcagctgtgggaagcacagtcaccatcaattgc

SEQ ID NO: 34 - DNA, 39 bp, synthetic construct
  misc_feature 1..39: note = Engineered antibody sequence
  source 1..39: mol_type = other DNA
caggccagtcagagtgtttatgataacaactacctagcc

SEQ ID NO: 35 - DNA, 45 bp, synthetic construct
  misc_feature 1..45: note = Engineered antibody sequence
  source 1..45: mol_type = other DNA
tggtatcagcagaaaccagggcagcctcccaagcaactgatctat

SEQ ID NO: 36 - DNA, 21 bp, synthetic construct
  misc_feature 1..21: note = Engineered antibody sequence
  source 1..21: mol_type = other DNA
tctacatccactctggcatct

SEQ ID NO: 37 - DNA, 96 bp, synthetic construct
  misc_feature 1..96: note = Engineered antibody sequence
  source 1..96: mol_type = other DNA
ggggtctcatcgcggttcaaaggcagtggatctgggacacagttcactctcaccatcagcgacctggagtgtgccgatgctgccacttactactgt

SEQ ID NO: 38 - DNA, 39 bp, synthetic construct
  misc_feature 1..39: note = Engineered antibody sequence
  source 1..39: mol_type = other DNA
ctaggcagttatgattgtagtagtggtgattgttttgtt

SEQ ID NO: 39 - DNA, 33 bp, synthetic construct
  misc_feature 1..33: note = Engineered antibody sequence
  source 1..33: mol_type = other DNA
ttcggcggagggaccgaggtggtggtcaaacgt

SEQ ID NO: 40 - DNA, 321 bp, synthetic construct
  misc_feature 1..321: note = Engineered antibody sequence
  source 1..321: mol_type = other DNA
acggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 41 - protein, 441 aa, synthetic construct
  REGION 1..441: note = Engineered antibody sequence
EVQLVESGGGLVQPGGSLRLSCAVSGLDLSSYYMQWVRQAPGKGLEWVGVIGINDNTYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 111 aa, synthetic construct
  REGION 1..111: note = Engineered antibody sequence
EVQLVESGGGLVQPGGSLRLSCAVSGLDLSSYYMQWVRQAPGKGLEWVGVIGINDNTYYASWAKGRFTISRDNSKTTVYLQMNSLRAEDTAVYFCARGDIWGQGTLVTVSS

SEQ ID NO: 43 - protein, 30 aa, synthetic construct
  REGION 1..30: note = Engineered antibody sequence
EVQLVESGGGLVQPGGSLRLSCAVSGLDLS

SEQ ID NO: 44 - protein, 5 aa, synthetic construct
  REGION 1..5: note = Engineered antibody sequence
SYYMQ

SEQ ID NO: 45 - protein, 14 aa, synthetic construct
  REGION 1..14: note = Engineered antibody sequence
WVRQAPGKGLEWVG

SEQ ID NO: 46 - protein, 16 aa, synthetic construct
  REGION 1..16: note = Engineered antibody sequence
VIGINDNTYYASWAKG

SEQ ID NO: 47 - protein, 32 aa, synthetic construct
  REGION 1..32: note = Engineered antibody sequence
RFTISRDNSKTTVYLQMNSLRAEDTAVYFCAR

SEQ ID NO: 48 - skipped

SEQ ID NO: 49 - protein, 11 aa, synthetic construct
  REGION 1..11: note = Engineered antibody sequence
WGQGTLVTVSS

SEQ ID NO: 50 - protein, 330 aa, synthetic construct; identical to SEQ ID NO: 10
  REGION 1..330: note = Engineered antibody sequence

SEQ ID NO: 51 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggactcgacctcagtagctactacatgcaatgggtccgtcaggctccagggaaggggctggagtgggtcggagtcattggtatcaatgataacacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 52 - DNA, 333 bp, synthetic construct
  misc_feature 1..333: note = Engineered antibody sequence
  source 1..333: mol_type = other DNA
gaggtgcagcttgtggagtctgggggaggcttggtccagcctggggggtccctgagactctcctgtgcagtctctggactcgacctcagtagctactacatgcaatgggtccgtcaggctccagggaaggggctggagtgggtcggagtcattggtatcaatgataacacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagc

SEQ ID NO: 53 - DNA, 90 bp, synthetic construct
  misc_feature 1..90: note = Engineered antibody sequence
  source 1..90: mol_type = other DNA
gaggtgcagcttgtggagtctgggggaggcttggtccagcctggggggtccctgagactctcctgtgcagtctctggactcgacctcagt

SEQ ID NO: 54 - DNA, 15 bp, synthetic construct
  misc_feature 1..15: note = Engineered antibody sequence
  source 1..15: mol_type = other DNA
agctactacatgcaa

SEQ ID NO: 55 - DNA, 42 bp, synthetic construct
  misc_feature 1..42: note = Engineered antibody sequence
  source 1..42: mol_type = other DNA
tgggtccgtcaggctccagggaaggggctggagtgggtcgga

SEQ ID NO: 56 - DNA, 48 bp, synthetic construct
  misc_feature 1..48: note = Engineered antibody sequence
  source 1..48: mol_type = other DNA
gtcattggtatcaatgataacacatactacgcgagctgggcgaaaggc

SEQ ID NO: 57 - DNA, 96 bp, synthetic construct
  misc_feature 1..96: note = Engineered antibody sequence
  source 1..96: mol_type = other DNA
cgattcaccatctccagagacaattccaagaccacggtgtatcttcaaatgaacagcctgagagctgaggacactgctgtgtatttctgtgctaga

SEQ ID NO: 58 - skipped

SEQ ID NO: 59 - DNA, 33 bp, synthetic construct
  misc_feature 1..33: note = Engineered antibody sequence
  source 1..33: mol_type = other DNA
tggggccaagggaccctcgtcaccgtctcgagc

SEQ ID NO: 60 - DNA, 993 bp, synthetic construct; identical to SEQ ID NO: 20
  misc_feature 1..993: note = Engineered antibody sequence
  source 1..993: mol_type = other DNA

SEQ ID NO: 61 - protein, 219 aa, synthetic construct
  REGION 1..219: note = Engineered antibody sequence
QVLTQSPSSLSASVGDRVTINCQASQSVYDNNYLAWYQQKPGKVPKQLIYSTSTLASGVPSRFSGSGSGTDFTLTISSLQPEDVATYYCLGSYDCSSGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 113 aa, synthetic construct
  REGION 1..113: note = Engineered antibody sequence
QVLTQSPSSLSASVGDRVTINCQASQSVYDNNYLAWYQQKPGKVPKQLIYSTSTLASGVPSRFSGSGSGTDFTLTISSLQPEDVATYYCLGSYDCSSGDCFVFGGGTKVEIKR

SEQ ID NO: 63 - protein, 22 aa, synthetic construct
  REGION 1..22: note = Engineered antibody sequence
QVLTQSPSSLSASVGDRVTINC

SEQ ID NO: 64 - protein, 13 aa, synthetic construct
  REGION 1..13: note = Engineered antibody sequence
QASQSVYDNNYLA

SEQ ID NO: 65 - protein, 15 aa, synthetic construct
  REGION 1..15: note = Engineered antibody sequence
WYQQKPGKVPKQLIY

SEQ ID NO: 66 - protein, 7 aa, synthetic construct
  REGION 1..7: note = Engineered antibody sequence
STSTLAS

SEQ ID NO: 67 - protein, 32 aa, synthetic construct
  REGION 1..32: note = Engineered antibody sequence
GVPSRFSGSGSGTDFTLTISSLQPEDVATYYC

SEQ ID NO: 68 - protein, 13 aa, synthetic construct
  REGION 1..13: note = Engineered antibody sequence
LGSYDCSSGDCFV

SEQ ID NO: 69 - protein, 11 aa, synthetic construct
  REGION 1..11: note = Engineered antibody sequence
FGGGTKVEIKR

SEQ ID NO: 70 - protein, 106 aa, synthetic construct; identical to SEQ ID NO: 30
  REGION 1..106: note = Engineered antibody sequence

SEQ ID NO: 71 - DNA, 660 bp, synthetic construct
  misc_feature 1..660: note = Engineered antibody sequence
  source 1..660: mol_type = other DNA
caagtgctgacccagtctccatcctccctgtctgcatctgtaggagacagagtcaccatcaattgccaggccagtcagagtgtttatgataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctaggcagttatgattgtagtagtggtgattgttttgttttcggcggaggaaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 72 - DNA, 339 bp, synthetic construct
  misc_feature 1..339: note = Engineered antibody sequence
  source 1..339: mol_type = other DNA
caagtgctgacccagtctccatcctccctgtctgcatctgtaggagacagagtcaccatcaattgccaggccagtcagagtgtttatgataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctaggcagttatgattgtagtagtggtgattgttttgttttcggcggaggaaccaaggtggaaatcaaacgt

SEQ ID NO: 73 - DNA, 66 bp, synthetic construct
  misc_feature 1..66: note = Engineered antibody sequence
  source 1..66: mol_type = other DNA
caagtgctgacccagtctccatcctccctgtctgcatctgtaggagacagagtcaccatcaattgc

SEQ ID NO: 74 - DNA, 39 bp, synthetic construct; identical to SEQ ID NO: 34
  misc_feature 1..39: note = Engineered antibody sequence
  source 1..39: mol_type = other DNA

SEQ ID NO: 75 - DNA, 45 bp, synthetic construct
  misc_feature 1..45: note = Engineered antibody sequence
  source 1..45: mol_type = other DNA
tggtatcagcagaaaccagggaaagttcctaagcaactgatctat

SEQ ID NO: 76 - DNA, 21 bp, synthetic construct
  misc_feature 1..21: note = Engineered antibody sequence
  source 1..21: mol_type = other DNA
tctacatccactctggcatct

SEQ ID NO: 77 - DNA, 96 bp, synthetic construct
  misc_feature 1..96: note = Engineered antibody sequence
  source 1..96: mol_type = other DNA
ggggtcccatctcgtttcagtggcagtggatctgggacagatttcactctcaccatcagcagcctgcagcctgaagatgttgcaacttattactgt

SEQ ID NO: 78 - DNA, 39 bp, synthetic construct; identical to SEQ ID NO: 38
  misc_feature 1..39: note = Engineered antibody sequence
  source 1..39: mol_type = other DNA

SEQ ID NO: 79 - DNA, 33 bp, synthetic construct
  misc_feature 1..33: note = Engineered antibody sequence
  source 1..33: mol_type = other DNA
ttcggcggaggaaccaaggtggaaatcaaacgt

SEQ ID NO: 80 - DNA, 321 bp, synthetic construct; identical to SEQ ID NO: 40
  misc_feature 1..321: note = Engineered antibody sequence
  source 1..321: mol_type = other DNA

SEQ ID NO: 81 - protein, 441 aa, synthetic construct
  REGION 1..441: note = Engineered antibody sequence
EVQLVESGGGLVQPGGSLRLSCAVSGLDLSSYYMQWVRQAPGKGLEWVGVIGINDNTYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 111 aa, synthetic construct; identical to SEQ ID NO: 42
  REGION 1..111: note = Engineered antibody sequence

SEQ ID NO: 83 - protein, 30 aa, synthetic construct; identical to SEQ ID NO: 43
  REGION 1..30: note = Engineered antibody sequence

SEQ ID NO: 84 - protein, 5 aa, synthetic construct
  REGION 1..5: note = Engineered antibody sequence
SYYMQ

SEQ ID NO: 85 - protein, 14 aa, synthetic construct
  REGION 1..14: note = Engineered antibody sequence
WVRQAPGKGLEWVG

SEQ ID NO: 86 - protein, 16 aa, synthetic construct
  REGION 1..16: note = Engineered antibody sequence
VIGINDNTYYASWAKG

SEQ ID NO: 87 - protein, 32 aa, synthetic construct; identical to SEQ ID NO: 47
  REGION 1..32: note = Engineered antibody sequence

SEQ ID NO: 88 - skipped

SEQ ID NO: 89 - protein, 11 aa, synthetic construct
  REGION 1..11: note = Engineered antibody sequence
WGQGTLVTVSS

SEQ ID NO: 90 - protein, 330 aa, synthetic construct
  REGION 1..330: note = Engineered antibody sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggactcgacctcagtagctactacatgcaatgggtccgtcaggctccagggaaggggctggagtgggtcggagtcattggtatcaatgataacacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacgcgagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 92 - DNA, 333 bp, synthetic construct; identical to SEQ ID NO: 52
  misc_feature 1..333: note = Engineered antibody sequence
  source 1..333: mol_type = other DNA

SEQ ID NO: 93 - DNA, 90 bp, synthetic construct; identical to SEQ ID NO: 53
  misc_feature 1..90: note = Engineered antibody sequence
  source 1..90: mol_type = other DNA

SEQ ID NO: 94 - DNA, 15 bp, synthetic construct
  misc_feature 1..15: note = Engineered antibody sequence
  source 1..15: mol_type = other DNA
agctactacatgcaa

SEQ ID NO: 95 - DNA, 42 bp, synthetic construct; identical to SEQ ID NO: 55
  misc_feature 1..42: note = Engineered antibody sequence
  source 1..42: mol_type = other DNA

SEQ ID NO: 96 - DNA, 48 bp, synthetic construct; identical to SEQ ID NO: 56
  misc_feature 1..48: note = Engineered antibody sequence
  source 1..48: mol_type = other DNA

SEQ ID NO: 97 - DNA, 96 bp, synthetic construct; identical to SEQ ID NO: 57
  misc_feature 1..96: note = Engineered antibody sequence
  source 1..96: mol_type = other DNA

SEQ ID NO: 98 - skipped

SEQ ID NO: 99 - DNA, 33 bp, synthetic construct; identical to SEQ ID NO: 59
  misc_feature 1..33: note = Engineered antibody sequence
  source 1..33: mol_type = other DNA

SEQ ID NO: 100 - DNA, 993 bp, synthetic construct
  misc_feature 1..993: note = Engineered antibody sequence
  source 1..993: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacgcgagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 101 - protein, 219 aa, synthetic construct; identical to SEQ ID NO: 61
  REGION 1..219: note = Engineered antibody sequence

SEQ ID NO: 102 - protein, 113 aa, synthetic construct; identical to SEQ ID NO: 62
  REGION 1..113: note = Engineered antibody sequence

SEQ ID NO: 103 - protein, 22 aa, synthetic construct
  REGION 1..22: note = Engineered antibody sequence
QVLTQSPSSLSASVGDRVTINC

SEQ ID NO: 104 - protein, 13 aa, synthetic construct
  REGION 1..13: note = Engineered antibody sequence
QASQSVYDNNYLA

SEQ ID NO: 105 - protein, 15 aa, synthetic construct
  REGION 1..15: note = Engineered antibody sequence
WYQQKPGKVPKQLIY

SEQ ID NO: 106 - protein, 7 aa, synthetic construct
  REGION 1..7: note = Engineered antibody sequence
STSTLAS

SEQ ID NO: 107 - protein, 32 aa, synthetic construct; identical to SEQ ID NO: 67
  REGION 1..32: note = Engineered antibody sequence

SEQ ID NO: 108 - protein, 13 aa, synthetic construct
  REGION 1..13: note = Engineered antibody sequence
LGSYDCSSGDCFV

SEQ ID NO: 109 - protein, 11 aa, synthetic construct
  REGION 1..11: note = Engineered antibody sequence
FGGGTKVEIKR

SEQ ID NO: 110 - protein, 106 aa, synthetic construct; identical to SEQ ID NO: 30
  REGION 1..106: note = Engineered antibody sequence

SEQ ID NO: 111 - DNA, 660 bp, synthetic construct; identical to SEQ ID NO: 71
  misc_feature 1..660: note = Engineered antibody sequence
  source 1..660: mol_type = other DNA

SEQ ID NO: 112 - DNA, 339 bp, synthetic construct; identical to SEQ ID NO: 72
  misc_feature 1..339: note = Engineered antibody sequence
  source 1..339: mol_type = other DNA

SEQ ID NO: 113 - DNA, 66 bp, synthetic construct; identical to SEQ ID NO: 73
  misc_feature 1..66: note = Engineered antibody sequence
  source 1..66: mol_type = other DNA

SEQ ID NO: 114 - DNA, 39 bp, synthetic construct; identical to SEQ ID NO: 34
  misc_feature 1..39: note = Engineered antibody sequence
  source 1..39: mol_type = other DNA

SEQ ID NO: 115 - DNA, 45 bp, synthetic construct; identical to SEQ ID NO: 75
  misc_feature 1..45: note = Engineered antibody sequence
  source 1..45: mol_type = other DNA

SEQ ID NO: 116 - DNA, 21 bp, synthetic construct
  misc_feature 1..21: note = Engineered antibody sequence
  source 1..21: mol_type = other DNA
tctacatccactctggcatct

SEQ ID NO: 117 - DNA, 96 bp, synthetic construct; identical to SEQ ID NO: 77
  misc_feature 1..96: note = Engineered antibody sequence
  source 1..96: mol_type = other DNA

SEQ ID NO: 118 - DNA, 39 bp, synthetic construct; identical to SEQ ID NO: 38
  misc_feature 1..39: note = Engineered antibody sequence
  source 1..39: mol_type = other DNA

SEQ ID NO: 119 - DNA, 33 bp, synthetic construct; identical to SEQ ID NO: 79
  misc_feature 1..33: note = Engineered antibody sequence
  source 1..33: mol_type = other DNA

SEQ ID NO: 120 - DNA, 321 bp, synthetic construct; identical to SEQ ID NO: 40
  misc_feature 1..321: note = Engineered antibody sequence
  source 1..321: mol_type = other DNA

SEQ ID NO: 121 - protein, 439 aa, synthetic construct
  REGION 1..439: note = Engineered antibody sequence
QSLEESGGRLVTPGTPLTLTCSVSGIDLSGYYMNWVRQAPGKGLEWIGVIGINGATYYASWAKGRFTISKTSSTTVDLKMTSLTTEDTATYFCARGDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 109 aa, synthetic construct
  REGION 1..109: note = Engineered antibody sequence
QSLEESGGRLVTPGTPLTLTCSVSGIDLSGYYMNWVRQAPGKGLEWIGVIGINGATYYASWAKGRFTISKTSSTTVDLKMTSLTTEDTATYFCARGDIWGPGTLVTVSS

SEQ ID NO: 123 - protein, 29 aa, synthetic construct
  REGION 1..29: note = Engineered antibody sequence
QSLEESGGRLVTPGTPLTLTCSVSGIDLS

SEQ ID NO: 124 - protein, 5 aa, synthetic construct
  REGION 1..5: note = Engineered antibody sequence
GYYMN

SEQ ID NO: 125 - protein, 14 aa, synthetic construct
  REGION 1..14: note = Engineered antibody sequence
WVRQAPGKGLEWIG

SEQ ID NO: 126 - protein, 16 aa, synthetic construct
  REGION 1..16: note = Engineered antibody sequence
VIGINGATYYASWAKG

SEQ ID NO: 127 - protein, 31 aa, synthetic construct
  REGION 1..31: note = Engineered antibody sequence
RFTISKTSSTTVDLKMTSLTTEDTATYFCAR

SEQ ID NO: 128 - skipped

SEQ ID NO: 129 - protein, 11 aa, synthetic construct
  REGION 1..11: note = Engineered antibody sequence
WGPGTLVTVSS

SEQ ID NO: 130 - protein, 330 aa, synthetic construct; identical to SEQ ID NO: 10
  REGION 1..330: note = Engineered antibody sequence

SEQ ID NO: 131 - DNA, 1320 bp, synthetic construct
  misc_feature 1..1320: note = Engineered antibody sequence
  source 1..1320: mol_type = other DNA
cagtcgctggaggagtccgggggtcgcctggtcacgcctgggacacccctgacactcacctgttccgtctctggcatcgacctcagtggctactacatgaactgggtccgccaggctccagggaaggggctggaatggatcggagtcattggtattaatggtgccacatactacgcgagctgggcgaaaggccgattcaccatctccaaaacctcgtcgaccacggtggatctgaaaatgaccagtctgacaaccgaggacacggccacctatttctgtgccagaggggacatctggggcccgggcaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 132 - DNA, 327 bp, synthetic construct
  misc_feature 1..327: note = Engineered antibody sequence
  source 1..327: mol_type = other DNA
cagtcgctggaggagtccgggggtcgcctggtcacgcctgggacacccctgacactcacctgttccgtctctggcatcgacctcagtggctactacatgaactgggtccgccaggctccagggaaggggctggaatggatcggagtcattggtattaatggtgccacatactacgcgagctgggcgaaaggccgattcaccatctccaaaacctcgtcgaccacggtggatctgaaaatgaccagtctgacaaccgaggacacggccacctatttctgtgccagaggggacatctggggcccgggcaccctcgtcaccgtctcgagc

SEQ ID NO: 133 - DNA, 87 bp, synthetic construct
  misc_feature 1..87: note = Engineered antibody sequence
  source 1..87: mol_type = other DNA
cagtcgctggaggagtccgggggtcgcctggtcacgcctgggacacccctgacactcacctgttccgtctctggcatcgacctcagt

SEQ ID NO: 134 - DNA, 15 bp, synthetic construct
  misc_feature 1..15: note = Engineered antibody sequence
  source 1..15: mol_type = other DNA
ggctactacatgaac

SEQ ID NO: 135 - DNA, 42 bp, synthetic construct; identical to SEQ ID NO: 15
  misc_feature 1..42: note = Engineered antibody sequence
  source 1..42: mol_type = other DNA

SEQ ID NO: 136 - DNA, 48 bp, synthetic construct
  misc_feature 1..48: note = Engineered antibody sequence
  source 1..48: mol_type = other DNA
gtcattggtattaatggtgccacatactacgcgagctgggcgaaaggc

SEQ ID NO: 137 - DNA, 93 bp, synthetic construct
  misc_feature 1..93: note = Engineered antibody sequence
  source 1..93: mol_type = other DNA
cgattcaccatctccaaaacctcgtcgaccacggtggatctgaaaatgaccagtctgacaaccgaggacacggccacctatttctgtgccaga

SEQ ID NO: 138 - skipped

SEQ ID NO: 139 - DNA, 33 bp, synthetic construct
  misc_feature 1..33: note = Engineered antibody sequence
  source 1..33: mol_type = other DNA
tggggcccgggcaccctcgtcaccgtctcgagc

SEQ ID NO: 140 - DNA, 993 bp, synthetic construct; identical to SEQ ID NO: 20
  misc_feature 1..993: note = Engineered antibody sequence
  source 1..993: mol_type = other DNA

SEQ ID NO: 141 - protein, 219 aa, synthetic construct
  REGION 1..219: note = Engineered antibody sequence
QVLTQTPSPVSAAVGSTVTINCQASQSVYHNTYLAWYQQKPGQPPKQLIYDASTLASGVPSRFSGSGSGTQFTLTISGVQCNDAAAYYCLGSYDCTNGDCFVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 113 aa, synthetic construct
  REGION 1..113: note = Engineered antibody sequence
QVLTQTPSPVSAAVGSTVTINCQASQSVYHNTYLAWYQQKPGQPPKQLIYDASTLASGVPSRFSGSGSGTQFTLTISGVQCNDAAAYYCLGSYDCTNGDCFVFGGGTEVVVKR

SEQ ID NO: 143 - protein, 22 aa, synthetic construct
  REGION 1..22: note = Engineered antibody sequence
QVLTQTPSPVSAAVGSTVTINC

SEQ ID NO: 144 - protein, 13 aa, synthetic construct
  REGION 1..13: note = Engineered antibody sequence
QASQSVYHNTYLA

SEQ ID NO: 145 - protein, 15 aa, synthetic construct
  REGION 1..15: note = Engineered antibody sequence
WYQQKPGQPPKQLIY

SEQ ID NO: 146 - protein, 7 aa, synthetic construct
  REGION 1..7: note = Engineered antibody sequence
DASTLAS

SEQ ID NO: 147 - protein, 32 aa, synthetic construct
  REGION 1..32: note = Engineered antibody sequence
GVPSRFSGSGSGTQFTLTISGVQCNDAAAYYC

SEQ ID NO: 148 - protein, 13 aa, synthetic construct
  REGION 1..13: note = Engineered antibody sequence
LGSYDCTNGDCFV

SEQ ID NO: 149 - protein, 11 aa, synthetic construct
  REGION 1..11: note = Engineered antibody sequence
FGGGTEVVVKR

SEQ ID NO: 150 - protein, 106 aa, synthetic construct; identical to SEQ ID NO: 30
  REGION 1..106: note = Engineered antibody sequence

SEQ ID NO: 151 - DNA, 660 bp, synthetic construct
  misc_feature 1..660: note = Engineered antibody sequence
  source 1..660: mol_type = other DNA
caagtgctgacccagactccatcccccgtgtctgcagctgtgggaagcacagtcaccatcaattgccaggccagtcagagtgtttatcataacacctacctggcctggtatcagcagaaaccagggcagcctcccaaacaactgatctatgatgcatccactctggcgtctggggtcccatcgcggttcagcggcagtggatctgggacacagttcactctcaccatcagcggcgtgcagtgtaacgatgctgccgcttactactgtctgggcagttatgattgtactaatggtgattgttttgttttcggcggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 152 - DNA, 339 bp, synthetic construct
  misc_feature 1..339: note = Engineered antibody sequence
  source 1..339: mol_type = other DNA
caagtgctgacccagactccatcccccgtgtctgcagctgtgggaagcacagtcaccatcaattgccaggccagtcagagtgtttatcataacacctacctggcctggtatcagcagaaaccagggcagcctcccaaacaactgatctatgatgcatccactctggcgtctggggtcccatcgcggttcagcggcagtggatctgggacacagttcactctcaccatcagcggcgtgcagtgtaacgatgctgccgcttactactgtctgggcagttatgattgtactaatggtgattgttttgttttcggcggagggaccgaggtggtggtcaaacgt

SEQ ID NO: 153 - DNA, 66 bp, synthetic construct
  misc_feature 1..66: note = Engineered antibody sequence
  source 1..66: mol_type = other DNA
caagtgctgacccagactccatcccccgtgtctgcagctgtgggaagcacagtcaccatcaattgc

SEQ ID NO: 154 - DNA, 39 bp, synthetic construct
  misc_feature 1..39: note = Engineered antibody sequence
  source 1..39: mol_type = other DNA
caggccagtcagagtgtttatcataacacctacctggcc

SEQ ID NO: 155 - DNA, 45 bp, synthetic construct
  misc_feature 1..45: note = Engineered antibody sequence
  source 1..45: mol_type = other DNA
tggtatcagcagaaaccagggcagcctcccaaacaactgatctat

SEQ ID NO: 156 - DNA, 21 bp, synthetic construct
  misc_feature 1..21: note = Engineered antibody sequence
  source 1..21: mol_type = other DNA
gatgcatccactctggcgtct

SEQ ID NO: 157 - DNA, 96 bp, synthetic construct
  misc_feature 1..96: note = Engineered antibody sequence
  source 1..96: mol_type = other DNA
ggggtcccatcgcggttcagcggcagtggatctgggacacagttcactctcaccatcagcggcgtgcagtgtaacgatgctgccgcttactactgt

SEQ ID NO: 158 - DNA, 39 bp, synthetic construct
  misc_feature 1..39: note = Engineered antibody sequence
  source 1..39: mol_type = other DNA
ctgggcagttatgattgtactaatggtgattgttttgtt

SEQ ID NO: 159 - DNA, 33 bp, synthetic construct; identical to SEQ ID NO: 39
  misc_feature 1..33: note = Engineered antibody sequence
  source 1..33: mol_type = other DNA

SEQ ID NO: 160 - DNA, 321 bp, synthetic construct; identical to SEQ ID NO: 40
  misc_feature 1..321: note = Engineered antibody sequence
  source 1..321: mol_type = other DNA

SEQ ID NO: 161 - protein, 441 aa, synthetic construct
  REGION 1..441: note = Engineered antibody sequence
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 111 aa, synthetic construct
  REGION 1..111: note = Engineered antibody sequence
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSS

SEQ ID NO: 163 - protein, 30 aa, synthetic construct
  REGION 1..30: note = Engineered antibody sequence
EVQLVESGGGLVQPGGSLRLSCAVSGIDLS

SEQ ID NO: 164 - protein, 5 aa, synthetic construct
  REGION 1..5: note = Engineered antibody sequence
GYYMN

SEQ ID NO: 165 - protein, 14 aa, synthetic construct
  REGION 1..14: note = Engineered antibody sequence
WVRQAPGKGLEWVG

SEQ ID NO: 166 - protein, 16 aa, synthetic construct
  REGION 1..16: note = Engineered antibody sequence
VIGINGATYYASWAKG

SEQ ID NO: 167 - protein, 32 aa, synthetic construct; identical to SEQ ID NO: 47
  REGION 1..32: note = Engineered antibody sequence

SEQ ID NO: 168 - skipped

SEQ ID NO: 169 - protein, 11 aa, synthetic construct
  REGION 1..11: note = Engineered antibody sequence
WGQGTLVTVSS

SEQ ID NO: 170 - protein, 330 aa, synthetic construct; identical to SEQ ID NO: 10
  REGION 1..330: note = Engineered antibody sequence

SEQ ID NO: 171 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcgacctcagtggctactacatgaactgggtccgtcaggctccagggaaggggctggagtgggtcggagtcattggtattaatggtgccacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 172 - DNA, 333 bp, synthetic construct
  misc_feature 1..333: note = Engineered antibody sequence
  source 1..333: mol_type = other DNA
gaggtgcagcttgtggagtctgggggaggcttggtccagcctggggggtccctgagactctcctgtgcagtctctggaatcgacctcagtggctactacatgaactgggtccgtcaggctccagggaaggggctggagtgggtcggagtcattggtattaatggtgccacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagc

SEQ ID NO: 173 - DNA, 90 bp, synthetic construct
  misc_feature 1..90: note = Engineered antibody sequence
  source 1..90: mol_type = other DNA
gaggtgcagcttgtggagtctgggggaggcttggtccagcctggggggtccctgagactctcctgtgcagtctctggaatcgacctcagt

SEQ ID NO: 174 - DNA, 15 bp, synthetic construct
  misc_feature 1..15: note = Engineered antibody sequence
  source 1..15: mol_type = other DNA
ggctactacatgaac

SEQ ID NO: 175 - DNA, 42 bp, synthetic construct; identical to SEQ ID NO: 55
  misc_feature 1..42: note = Engineered antibody sequence
  source 1..42: mol_type = other DNA

SEQ ID NO: 176 - DNA, 48 bp, synthetic construct; identical to SEQ ID NO: 136
  misc_feature 1..48: note = Engineered antibody sequence
  source 1..48: mol_type = other DNA

SEQ ID NO: 177 - DNA, 96 bp, synthetic construct; identical to SEQ ID NO: 57
  misc_feature 1..96: note = Engineered antibody sequence
  source 1..96: mol_type = other DNA

SEQ ID NO: 178 - skipped

SEQ ID NO: 179 - DNA, 33 bp, synthetic construct; identical to SEQ ID NO: 59
  misc_feature 1..33: note = Engineered antibody sequence
  source 1..33: mol_type = other DNA

SEQ ID NO: 180 - DNA, 993 bp, synthetic construct; identical to SEQ ID NO: 20
  misc_feature 1..993: note = Engineered antibody sequence
  source 1..993: mol_type = other DNA

SEQ ID NO: 181 - protein, 219 aa, synthetic construct
  REGION 1..219: note = Engineered antibody sequence
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 113 aa, synthetic construct
  REGION 1..113: note = Engineered antibody sequence
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKR

SEQ ID NO: 183 - protein, 22 aa, synthetic construct
  REGION 1..22: note = Engineered antibody sequence
QVLTQSPSSLSASVGDRVTINC

SEQ ID NO: 184 - protein, 13 aa, synthetic construct
  REGION 1..13: note = Engineered antibody sequence
QASQSVYHNTYLA

SEQ ID NO: 185 - protein, 15 aa, synthetic construct
  REGION 1..15: note = Engineered antibody sequence
WYQQKPGKVPKQLIY

SEQ ID NO: 186 - protein, 7 aa, synthetic construct
  REGION 1..7: note = Engineered antibody sequence
DASTLAS

SEQ ID NO: 187 - protein, 32 aa, synthetic construct; identical to SEQ ID NO: 67
  REGION 1..32: note = Engineered antibody sequence

SEQ ID NO: 188 - protein, 13 aa, synthetic construct
  REGION 1..13: note = Engineered antibody sequence
LGSYDCTNGDCFV

SEQ ID NO: 189 - protein, 11 aa, synthetic construct
  REGION 1..11: note = Engineered antibody sequence
FGGGTKVEIKR

SEQ ID NO: 190 - protein, 106 aa, synthetic construct; identical to SEQ ID NO: 30
  REGION 1..106: note = Engineered antibody sequence

SEQ ID NO: 191 - DNA, 660 bp, synthetic construct
  misc_feature 1..660: note = Engineered antibody sequence
  source 1..660: mol_type = other DNA
caagtgctgacccagtctccatcctccctgtctgcatctgtaggagacagagtcaccatcaattgccaggccagtcagagtgtttatcataacacctacctggcctggtatcagcagaaaccagggaaagttcctaagcaactgatctatgatgcatccactctggcatctggggtcccatctcgtttcagtggcagtggatctgggacagatttcactctcaccatcagcagcctgcagcctgaagatgttgcaacttattactgtctgggcagttatgattgtactaatggtgattgttttgttttcggcggaggaaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 192 - DNA, 339 bp, synthetic construct
  misc_feature 1..339: note = Engineered antibody sequence
  source 1..339: mol_type = other DNA
caagtgctgacccagtctccatcctccctgtctgcatctgtaggagacagagtcaccatcaattgccaggccagtcagagtgtttatcataacacctacctggcctggtatcagcagaaaccagggaaagttcctaagcaactgatctatgatgcatccactctggcatctggggtcccatctcgtttcagtggcagtggatctgggacagatttcactctcaccatcagcagcctgcagcctgaagatgttgcaacttattactgtctgggcagttatgattgtactaatggtgattgttttgttttcggcggaggaaccaaggtggaaatcaaacgt

SEQ ID NO: 193 - DNA, 66 bp, synthetic construct; identical to SEQ ID NO: 73
  misc_feature 1..66: note = Engineered antibody sequence
  source 1..66: mol_type = other DNA

SEQ ID NO: 194 - DNA, 39 bp, synthetic construct; identical to SEQ ID NO: 154
  misc_feature 1..39: note = Engineered antibody sequence
  source 1..39: mol_type = other DNA

SEQ ID NO: 195 - DNA, 45 bp, synthetic construct; identical to SEQ ID NO: 75
  misc_feature 1..45: note = Engineered antibody sequence
  source 1..45: mol_type = other DNA

SEQ ID NO: 196 - DNA, 21 bp, synthetic construct
  misc_feature 1..21: note = Engineered antibody sequence
  source 1..21: mol_type = other DNA
gatgcatccactctggcatct

SEQ ID NO: 197 - DNA, 96 bp, synthetic construct; identical to SEQ ID NO: 77
  misc_feature 1..96: note = Engineered antibody sequence
  source 1..96: mol_type = other DNA

SEQ ID NO: 198 - DNA, 39 bp, synthetic construct; identical to SEQ ID NO: 158
  misc_feature 1..39: note = Engineered antibody sequence
  source 1..39: mol_type = other DNA

SEQ ID NO: 199 - DNA, 33 bp, synthetic construct; identical to SEQ ID NO: 79
  misc_feature 1..33: note = Engineered antibody sequence
  source 1..33: mol_type = other DNA

SEQ ID NO: 200 - DNA, 321 bp, synthetic construct; identical to SEQ ID NO: 40
  misc_feature 1..321: note = Engineered antibody sequence
  source 1..321: mol_type = other DNA

SEQ ID NO: 201 - protein, 441 aa, synthetic construct
  REGION 1..441: note = Engineered antibody sequence
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 111 aa, synthetic construct; identical to SEQ ID NO: 162
  REGION 1..111: note = Engineered antibody sequence

SEQ ID NO: 203 - protein, 30 aa, synthetic construct; identical to SEQ ID NO: 163
  REGION 1..30: note = Engineered antibody sequence

SEQ ID NO: 204 - protein, 5 aa, synthetic construct
  REGION 1..5: note = Engineered antibody sequence
GYYMN

SEQ ID NO: 205 - protein, 14 aa, synthetic construct
  REGION 1..14: note = Engineered antibody sequence
WVRQAPGKGLEWVG

SEQ ID NO: 206 - protein, 16 aa, synthetic construct
  REGION 1..16: note = Engineered antibody sequence
VIGINGATYYASWAKG

SEQ ID NO: 207 - protein, 32 aa, synthetic construct; identical to SEQ ID NO: 47
  REGION 1..32: note = Engineered antibody sequence

SEQ ID NO: 208 - skipped

SEQ ID NO: 209 - protein, 11 aa, synthetic construct
  REGION 1..11: note = Engineered antibody sequence
WGQGTLVTVSS

SEQ ID NO: 210 - protein, 330 aa, synthetic construct; identical to SEQ ID NO: 90
  REGION 1..330: note = Engineered antibody sequence

SEQ ID NO: 211 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcgacctcagtggctactacatgaactgggtccgtcaggctccagggaaggggctggagtgggtcggagtcattggtattaatggtgccacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacgcgagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 212 - DNA, 333 bp, synthetic construct; identical to SEQ ID NO: 172
  misc_feature 1..333: note = Engineered antibody sequence
  source 1..333: mol_type = other DNA

SEQ ID NO: 213 - DNA, 90 bp, synthetic construct; identical to SEQ ID NO: 173
  misc_feature 1..90: note = Engineered antibody sequence
  source 1..90: mol_type = other DNA

SEQ ID NO: 214 - DNA, 15 bp, synthetic construct
  misc_feature 1..15: note = Engineered antibody sequence
  source 1..15: mol_type = other DNA
ggctactacatgaac

SEQ ID NO: 215 - DNA, 42 bp, synthetic construct; identical to SEQ ID NO: 55
  misc_feature 1..42: note = Engineered antibody sequence
  source 1..42: mol_type = other DNA

SEQ ID NO: 216 - DNA, 48 bp, synthetic construct; identical to SEQ ID NO: 136
  misc_feature 1..48: note = Engineered antibody sequence
  source 1..48: mol_type = other DNA

SEQ ID NO: 217 - DNA, 96 bp, synthetic construct; identical to SEQ ID NO: 57
  misc_feature 1..96: note = Engineered antibody sequence
  source 1..96: mol_type = other DNA

SEQ ID NO: 218 - skipped

SEQ ID NO: 219 - DNA, 33 bp, synthetic construct; identical to SEQ ID NO: 59
  misc_feature 1..33: note = Engineered antibody sequence
  source 1..33: mol_type = other DNA

SEQ ID NO: 220 - DNA, 993 bp, synthetic construct; identical to SEQ ID NO: 100
  misc_feature 1..993: note = Engineered antibody sequence
  source 1..993: mol_type = other DNA

SEQ ID NO: 221 - protein, 219 aa, synthetic construct; identical to SEQ ID NO: 181
  REGION 1..219: note = Engineered antibody sequence

SEQ ID NO: 222 - protein, 113 aa, synthetic construct; identical to SEQ ID NO: 182
  REGION 1..113: note = Engineered antibody sequence

SEQ ID NO: 223 - protein, 22 aa, synthetic construct
  REGION 1..22: note = Engineered antibody sequence
QVLTQSPSSLSASVGDRVTINC

SEQ ID NO: 224 - protein, 13 aa, synthetic construct
  REGION 1..13: note = Engineered antibody sequence
QASQSVYHNTYLA

SEQ ID NO: 225 - protein, 15 aa, synthetic construct
  REGION 1..15: note = Engineered antibody sequence
WYQQKPGKVPKQLIY

SEQ ID NO: 226 - protein, 7 aa, synthetic construct
  REGION 1..7: note = Engineered antibody sequence
DASTLAS

SEQ ID NO: 227 - protein, 32 aa, synthetic construct; identical to SEQ ID NO: 67
  REGION 1..32: note = Engineered antibody sequence

SEQ ID NO: 228 - protein, 13 aa, synthetic construct
  REGION 1..13: note = Engineered antibody sequence
LGSYDCTNGDCFV

SEQ ID NO: 229 - protein, 11 aa, synthetic construct
  REGION 1..11: note = Engineered antibody sequence
FGGGTKVEIKR

SEQ ID NO: 230 - protein, 106 aa, synthetic construct; identical to SEQ ID NO: 30
  REGION 1..106: note = Engineered antibody sequence

SEQ ID NO: 231 - DNA, 660 bp, synthetic construct; identical to SEQ ID NO: 191
  misc_feature 1..660: note = Engineered antibody sequence
  source 1..660: mol_type = other DNA

SEQ ID NO: 232 - DNA, 339 bp, synthetic construct; identical to SEQ ID NO: 192
  misc_feature 1..339: note = Engineered antibody sequence
  source 1..339: mol_type = other DNA

SEQ ID NO: 233 - DNA, 66 bp, synthetic construct; identical to SEQ ID NO: 73
  misc_feature 1..66: note = Engineered antibody sequence
  source 1..66: mol_type = other DNA

SEQ ID NO: 234 - DNA, 39 bp, synthetic construct; identical to SEQ ID NO: 154
  misc_feature 1..39: note = Engineered antibody sequence
  source 1..39: mol_type = other DNA

SEQ ID NO: 235 - DNA, 45 bp, synthetic construct; identical to SEQ ID NO: 75
  misc_feature 1..45: note = Engineered antibody sequence
  source 1..45: mol_type = other DNA

SEQ ID NO: 236 - DNA, 21 bp, synthetic construct
  misc_feature 1..21: note = Engineered antibody sequence
  source 1..21: mol_type = other DNA
gatgcatccactctggcatct

SEQ ID NO: 237 - DNA, 96 bp, synthetic construct; identical to SEQ ID NO: 77
  misc_feature 1..96: note = Engineered antibody sequence
  source 1..96: mol_type = other DNA

SEQ ID NO: 238 - DNA, 39 bp, synthetic construct; identical to SEQ ID NO: 158
  misc_feature 1..39: note = Engineered antibody sequence
  source 1..39: mol_type = other DNA

SEQ ID NO: 239 - DNA, 33 bp, synthetic construct; identical to SEQ ID NO: 79
  misc_feature 1..33: note = Engineered antibody sequence
  source 1..33: mol_type = other DNA

SEQ ID NO: 240 - DNA, 321 bp, synthetic construct; identical to SEQ ID NO: 40
  misc_feature 1..321: note = Engineered antibody sequence
  source 1..321: mol_type = other DNA

SEQ ID NO: 241 - protein, 440 aa, synthetic construct
  REGION 1..440: note = Engineered antibody sequence
QEQLKESGGRLVTPGTSLTLTCTVSGIDLSNHYMQWVRQAPGKGLEWIGVVGINGRTYYASWAKGRFTISRTSSTTVDLKMTRLTTEDTATYFCARGDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 110 aa, synthetic construct
  REGION 1..110: note = Engineered antibody sequence
QEQLKESGGRLVTPGTSLTLTCTVSGIDLSNHYMQWVRQAPGKGLEWIGVVGINGRTYYASWAKGRFTISRTSSTTVDLKMTRLTTEDTATYFCARGDIWGPGTLVTVSS

SEQ ID NO: 243 - protein, 30 aa, synthetic construct
  REGION 1..30: note = Engineered antibody sequence
QEQLKESGGRLVTPGTSLTLTCTVSGIDLS

SEQ ID NO: 244 - protein, 5 aa, synthetic construct
  REGION 1..5: note = Engineered antibody sequence
NHYMQ

SEQ ID NO: 245 - protein, 14 aa, synthetic construct
  REGION 1..14: note = Engineered antibody sequence
WVRQAPGKGLEWIG

SEQ ID NO: 246 - protein, 16 aa, synthetic construct
  REGION 1..16: note = Engineered antibody sequence
VVGINGRTYYASWAKG

SEQ ID NO: 247 - protein, 31 aa, synthetic construct
  REGION 1..31: note = Engineered antibody sequence
RFTISRTSSTTVDLKMTRLTTEDTATYFCAR

SEQ ID NO: 248 - skipped

SEQ ID NO: 249 - protein, 11 aa, synthetic construct
  REGION 1..11: note = Engineered antibody sequence
WGPGTLVTVSS

SEQ ID NO: 250 - protein, 330 aa, synthetic construct; identical to SEQ ID NO: 10
  REGION 1..330: note = Engineered antibody sequence

SEQ ID NO: 251 - DNA, 1323 bp, synthetic construct
  misc_feature 1..1323: note = Engineered antibody sequence
  source 1..1323: mol_type = other DNA
caggagcagctgaaggagtccgggggtcgcctggtcacgcctgggacatccctgacactcacctgcaccgtctctggaatcgacctcagtaaccactacatgcaatgggtccgccaggctccagggaaggggctggagtggatcggagtcgttggtattaatggtcgcacatactacgcgagctgggcgaaaggccgattcaccatctccagaacctcgtcgaccacggtggatctgaaaatgaccaggctgacaaccgaggacacggccacctatttctgtgccagaggggacatctggggcccaggcaccctggtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 252 - DNA, 330 bp, synthetic construct
  misc_feature 1..330: note = Engineered antibody sequence
  source 1..330: mol_type = other DNA
caggagcagctgaaggagtccgggggtcgcctggtcacgcctgggacatccctgacactcacctgcaccgtctctggaatcgacctcagtaaccactacatgcaatgggtccgccaggctccagggaaggggctggagtggatcggagtcgttggtattaatggtcgcacatactacgcgagctgggcgaaaggccgattcaccatctccagaacctcgtcgaccacggtggatctgaaaatgaccaggctgacaaccgaggacacggccacctatttctgtgccagaggggacatctggggcccaggcaccctggtcaccgtctcgagc

SEQ ID NO: 253 - DNA, 90 bp, synthetic construct
  misc_feature 1..90: note = Engineered antibody sequence
  source 1..90: mol_type = other DNA
caggagcagctgaaggagtccgggggtcgcctggtcacgcctgggacatccctgacactcacctgcaccgtctctggaatcgacctcagt

SEQ ID NO: 254 - DNA, 15 bp, synthetic construct
  misc_feature 1..15: note = Engineered antibody sequence
  source 1..15: mol_type = other DNA
aaccactacatgcaa

SEQ ID NO: 255 - DNA, 42 bp, synthetic construct
  misc_feature 1..42: note = Engineered antibody sequence
  source 1..42: mol_type = other DNA
tgggtccgccaggctccagggaaggggctggagtggatcgga

SEQ ID NO: 256 - DNA, 48 bp, synthetic construct
  misc_feature 1..48: note = Engineered antibody sequence
  source 1..48: mol_type = other DNA
gtcgttggtattaatggtcgcacatactacgcgagctgggcgaaaggc

SEQ ID NO: 257 - DNA, 93 bp, synthetic construct
  misc_feature 1..93: note = Engineered antibody sequence
  source 1..93: mol_type = other DNA
cgattcaccatctccagaacctcgtcgaccacggtggatctgaaaatgaccaggctgacaaccgaggacacggccacctatttctgtgccaga

SEQ ID NO: 258 - skipped

SEQ ID NO: 259 - DNA, 33 bp, synthetic construct
  misc_feature 1..33: note = Engineered antibody sequence
  source 1..33: mol_type = other DNA
tggggcccaggcaccctggtcaccgtctcgagc

SEQ ID NO: 260 - DNA, 993 bp, synthetic construct; identical to SEQ ID NO: 20
  misc_feature 1..993: note = Engineered antibody sequence
  source 1..993: mol_type = other DNA

SEQ ID NO: 261 - protein, 219 aa, synthetic construct
  REGION 1..219: note = Engineered antibody sequence
QVLTQTASPVSAAVGSTVTINCQASQSVYNYNYLAWYQQKPGQPPKQLIYSTSTLASGVSSRFKGSGSGTQFTLTISDVQCDDAATYYCLGSYDCSTGDCFVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 262 - protein, 113 aa, synthetic construct
  REGION 1..113: note = Engineered antibody sequence
QVLTQTASPVSAAVGSTVTINCQASQSVYNYNYLAWYQQKPGQPPKQLIYSTSTLASGVSSRFKGSGSGTQFTLTISDVQCDDAATYYCLGSYDCSTGDCFVFGGGTEVVVKR

SEQ ID NO: 263 - protein, 22 aa, synthetic construct
  REGION 1..22: note = Engineered antibody sequence
QVLTQTASPVSAAVGSTVTINC

SEQ ID NO: 264 - protein, 13 aa, synthetic construct
  REGION 1..13: note = Engineered antibody sequence
QASQSVYNYNYLA

SEQ ID NO: 265 - protein, 15 aa, synthetic construct
  REGION 1..15: note = Engineered antibody sequence
WYQQKPGQPPKQLIY

SEQ ID NO: 266 - protein, 7 aa, synthetic construct
  REGION 1..7: note = Engineered antibody sequence
STSTLAS

SEQ ID NO: 267 - protein, 32 aa, synthetic construct
  REGION 1..32: note = Engineered antibody sequence
GVSSRFKGSGSGTQFTLTISDVQCDDAATYYC

SEQ ID NO: 268 - protein, 13 aa, synthetic construct
  REGION 1..13: note = Engineered antibody sequence
LGSYDCSTGDCFV

SEQ ID NO: 269 - protein, 11 aa, synthetic construct
  REGION 1..11: note = Engineered antibody sequence
FGGGTEVVVKR

SEQ ID NO: 270 - protein, 106 aa, synthetic construct; identical to SEQ ID NO: 30
  REGION 1..106: note = Engineered antibody sequence

SEQ ID NO: 271 - DNA, 660 bp, synthetic construct
  misc_feature 1..660: note = Engineered antibody sequence
  source 1..660: mol_type = other DNA
caagtgctgacccagactgcatcccccgtgtctgcagctgtgggaagcacagtcaccatcaattgccaggccagtcagagtgtttataattacaactaccttgcctggtatcagcagaaaccagggcagcctcccaagcaactgatctattctacatccactctggcatctggggtctcatcgcgattcaaaggcagtggatctgggacacagttcactctcaccatcagcgacgtgcagtgtgacgatgctgccacttactactgtctaggcagttatgactgtagtactggtgattgttttgttttcggcggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 272 - DNA, 339 bp, synthetic construct
  misc_feature 1..339: note = Engineered antibody sequence
  source 1..339: mol_type = other DNA
caagtgctgacccagactgcatcccccgtgtctgcagctgtgggaagcacagtcaccatcaattgccaggccagtcagagtgtttataattacaactaccttgcctggtatcagcagaaaccagggcagcctcccaagcaactgatctattctacatccactctggcatctggggtctcatcgcgattcaaaggcagtggatctgggacacagttcactctcaccatcagcgacgtgcagtgtgacgatgctgccacttactactgtctaggcagttatgactgtagtactggtgattgttttgttttcggcggagggaccgaggtggtggtcaaacgt

SEQ ID NO: 273 - DNA, 66 bp, synthetic construct; identical to SEQ ID NO: 33
  misc_feature 1..66: note = Engineered antibody sequence
  source 1..66: mol_type = other DNA

SEQ ID NO: 274 - DNA, 39 bp, synthetic construct
  misc_feature 1..39: note = Engineered antibody sequence
  source 1..39: mol_type = other DNA
caggccagtcagagtgtttataattacaactaccttgcc

SEQ ID NO: 275 - DNA, 45 bp, synthetic construct; identical to SEQ ID NO: 35
  misc_feature 1..45: note = Engineered antibody sequence
  source 1..45: mol_type = other DNA

SEQ ID NO: 276 - DNA, 21 bp, synthetic construct
  misc_feature 1..21: note = Engineered antibody sequence
  source 1..21: mol_type = other DNA
tctacatccactctggcatct

SEQ ID NO: 277 - DNA, 96 bp, synthetic construct
  misc_feature 1..96: note = Engineered antibody sequence
  source 1..96: mol_type = other DNA
ggggtctcatcgcgattcaaaggcagtggatctgggacacagttcactctcaccatcagcgacgtgcagtgtgacgatgctgccacttactactgt

SEQ ID NO: 278 - DNA, 39 bp, synthetic construct
  misc_feature 1..39: note = Engineered antibody sequence
  source 1..39: mol_type = other DNA
ctaggcagttatgactgtagtactggtgattgttttgtt

SEQ ID NO: 279 - DNA, 33 bp, synthetic construct; identical to SEQ ID NO: 39
  misc_feature 1..33: note = Engineered antibody sequence
  source 1..33: mol_type = other DNA

SEQ ID NO: 280 - DNA, 321 bp, synthetic construct; identical to SEQ ID NO: 40
  misc_feature 1..321: note = Engineered antibody sequence
  source 1..321: mol_type = other DNA

SEQ ID NO: 281 - protein, 441 aa, synthetic construct
  REGION 1..441: note = Engineered antibody sequence
EVQLVESGGGLVQPGGSLRLSCAVSGIDLSNHYMQWVRQAPGKGLEWVGVVGINGRTYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 111 aa, synthetic construct
  REGION 1..111: note = Engineered antibody sequence
EVQLVESGGGLVQPGGSLRLSCAVSGIDLSNHYMQWVRQAPGKGLEWVGVVGINGRTYYASWAKGRFTISRDNSKTTVYLQMNSLRAEDTAVYFCARGDIWGQGTLVTVSS

SEQ ID NO: 283 - protein, 30 aa, synthetic construct; identical to SEQ ID NO: 163
  REGION 1..30: note = Engineered antibody sequence

SEQ ID NO: 284 - protein, 5 aa, synthetic construct
  REGION 1..5: note = Engineered antibody sequence
NHYMQ

SEQ ID NO: 285 - protein, 14 aa, synthetic construct
  REGION 1..14: note = Engineered antibody sequence
WVRQAPGKGLEWVG

SEQ ID NO: 286 - protein, 16 aa, synthetic construct
  REGION 1..16: note = Engineered antibody sequence
VVGINGRTYYASWAKG

SEQ ID NO: 287 - protein, 32 aa, synthetic construct; identical to SEQ ID NO: 47
  REGION 1..32: note = Engineered antibody sequence

SEQ ID NO: 288 - skipped

SEQ ID NO: 289 - protein, 11 aa, synthetic construct
  REGION 1..11: note = Engineered antibody sequence
WGQGTLVTVSS

SEQ ID NO: 290 - protein, 330 aa, synthetic construct; identical to SEQ ID NO: 10
  REGION 1..330: note = Engineered antibody sequence

SEQ ID NO: 291 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcgacctcagtaaccactacatgcaatgggtccgtcaggctccagggaaggggctggagtgggtcggagtcgttggtatcaatggtcgcacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 292 - DNA, 333 bp, synthetic construct
  misc_feature 1..333: note = Engineered antibody sequence
  source 1..333: mol_type = other DNA
gaggtgcagcttgtggagtctgggggaggcttggtccagcctggggggtccctgagactctcctgtgcagtctctggaatcgacctcagtaaccactacatgcaatgggtccgtcaggctccagggaaggggctggagtgggtcggagtcgttggtatcaatggtcgcacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagc

SEQ ID NO: 293 - DNA, 90 bp, synthetic construct; identical to SEQ ID NO: 173
  misc_feature 1..90: note = Engineered antibody sequence
  source 1..90: mol_type = other DNA

SEQ ID NO: 294 - DNA, 15 bp, synthetic construct
  misc_feature 1..15: note = Engineered antibody sequence
  source 1..15: mol_type = other DNA
aaccactacatgcaa

SEQ ID NO: 295 - DNA, 42 bp, synthetic construct; identical to SEQ ID NO: 55
  misc_feature 1..42: note = Engineered antibody sequence
  source 1..42: mol_type = other DNA

SEQ ID NO: 296 - DNA, 48 bp, synthetic construct
  misc_feature 1..48: note = Engineered antibody sequence
  source 1..48: mol_type = other DNA
gtcgttggtatcaatggtcgcacatactacgcgagctgggcgaaaggc

SEQ ID NO: 297 - DNA, 96 bp, synthetic construct; identical to SEQ ID NO: 57
  misc_feature 1..96: note = Engineered antibody sequence
  source 1..96: mol_type = other DNA

SEQ ID NO: 298 - skipped

SEQ ID NO: 299 - DNA, 33 bp, synthetic construct; identical to SEQ ID NO: 59
  misc_feature 1..33: note = Engineered antibody sequence
  source 1..33: mol_type = other DNA

SEQ ID NO: 300 - DNA, 993 bp, synthetic construct; identical to SEQ ID NO: 20
  misc_feature 1..993: note = Engineered antibody sequence
  source 1..993: mol_type = other DNA

SEQ ID NO: 301 - protein, 219 aa, synthetic construct
  REGION 1..219: note = Engineered antibody sequence
QVLTQSPSSLSASVGDRVTINCQASQSVYNYNYLAWYQQKPGKVPKQLIYSTSTLASGVPSRFSGSGSGTDFTLTISSLQPEDVATYYCLGSYDCSTGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 302 - protein, 113 aa, synthetic construct
  REGION 1..113: note = Engineered antibody sequence
QVLTQSPSSLSASVGDRVTINCQASQSVYNYNYLAWYQQKPGKVPKQLIYSTSTLASGVPSRFSGSGSGTDFTLTISSLQPEDVATYYCLGSYDCSTGDCFVFGGGTKVEIKR

SEQ ID NO: 303 - protein, 22 aa, synthetic construct
  REGION 1..22: note = Engineered antibody sequence
QVLTQSPSSLSASVGDRVTINC

SEQ ID NO: 304 - protein, 13 aa, synthetic construct
  REGION 1..13: note = Engineered antibody sequence
QASQSVYNYNYLA

SEQ ID NO: 305 - protein, 15 aa, synthetic construct
  REGION 1..15: note = Engineered antibody sequence
WYQQKPGKVPKQLIY

SEQ ID NO: 306 - protein, 7 aa, synthetic construct
  REGION 1..7: note = Engineered antibody sequence
STSTLAS

SEQ ID NO: 307 - protein, 32 aa, synthetic construct; identical to SEQ ID NO: 67
  REGION 1..32: note = Engineered antibody sequence

SEQ ID NO: 308 - protein, 13 aa, synthetic construct
  REGION 1..13: note = Engineered antibody sequence
LGSYDCSTGDCFV

SEQ ID NO: 309 - protein, 11 aa, synthetic construct
  REGION 1..11: note = Engineered antibody sequence
FGGGTKVEIKR

SEQ ID NO: 310 - protein, 106 aa, synthetic construct; identical to SEQ ID NO: 30
  REGION 1..106: note = Engineered antibody sequence

SEQ ID NO: 311 - DNA, 660 bp, synthetic construct
  misc_feature 1..660: note = Engineered antibody sequence
  source 1..660: mol_type = other DNA
caagtgctgacccagtctccatcctccctgtctgcatctgtaggagacagagtcaccatcaattgccaggccagtcagagtgtttacaattacaactaccttgcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtactggtgattgttttgttttcggcggaggaaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 312 - DNA, 339 bp, synthetic construct
  misc_feature 1..339: note = Engineered antibody sequence
  source 1..339: mol_type = other DNA
caagtgctgacccagtctccatcctccctgtctgcatctgtaggagacagagtcaccatcaattgccaggccagtcagagtgtttacaattacaactaccttgcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtactggtgattgttttgttttcggcggaggaaccaaggtggaaatcaaacgt

SEQ ID NO: 313 - DNA, 66 bp, synthetic construct; identical to SEQ ID NO: 73
  misc_feature 1..66: note = Engineered antibody sequence
  source 1..66: mol_type = other DNA

SEQ ID NO: 314 - DNA, 39 bp, synthetic construct
  misc_feature 1..39: note = Engineered antibody sequence
  source 1..39: mol_type = other DNA
caggccagtcagagtgtttacaattacaactaccttgcc

SEQ ID NO: 315 - DNA, 45 bp, synthetic construct; identical to SEQ ID NO: 75
  misc_feature 1..45: note = Engineered antibody sequence
  source 1..45: mol_type = other DNA

SEQ ID NO: 316 - DNA, 21 bp, synthetic construct
  misc_feature 1..21: note = Engineered antibody sequence
  source 1..21: mol_type = other DNA
tctacatccactctggcatct

SEQ ID NO: 317 - DNA, 96 bp, synthetic construct; identical to SEQ ID NO: 77
  misc_feature 1..96: note = Engineered antibody sequence
  source 1..96: mol_type = other DNA

SEQ ID NO: 318 - DNA, 39 bp, synthetic construct
  misc_feature 1..39: note = Engineered antibody sequence
  source 1..39: mol_type = other DNA
ctgggcagttatgattgtagtactggtgattgttttgtt

SEQ ID NO: 319 - DNA, 33 bp, synthetic construct; identical to SEQ ID NO: 79
  misc_feature 1..33: note = Engineered antibody sequence
  source 1..33: mol_type = other DNA

SEQ ID NO: 320 - DNA, 321 bp, synthetic construct; identical to SEQ ID NO: 40
  misc_feature 1..321: note = Engineered antibody sequence
  source 1..321: mol_type = other DNA

SEQ ID NO: 321 - protein, 439 aa, synthetic construct
  REGION 1..439: note = Engineered antibody sequence
QSLEESGGRLVTPGTPLTLTCTVSGIGLSSYYMQWVRQSPGRGLEWIGVIGSDGKTYYATWAKGRFTISKTSSTTVDLRMASLTTEDTATYFCTRGDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 109 aa, synthetic construct
  REGION 1..109: note = Engineered antibody sequence
QSLEESGGRLVTPGTPLTLTCTVSGIGLSSYYMQWVRQSPGRGLEWIGVIGSDGKTYYATWAKGRFTISKTSSTTVDLRMASLTTEDTATYFCTRGDIWGPGTLVTVSS

SEQ ID NO: 323 - protein, 29 aa, synthetic construct
  REGION 1..29: note = Engineered antibody sequence
QSLEESGGRLVTPGTPLTLTCTVSGIGLS

SEQ ID NO: 324 - protein, 5 aa, synthetic construct
  REGION 1..5: note = Engineered antibody sequence
SYYMQ

SEQ ID NO: 325 - protein, 14 aa, synthetic construct
  REGION 1..14: note = Engineered antibody sequence
WVRQSPGRGLEWIG

SEQ ID NO: 326 - protein, 16 aa, synthetic construct
  REGION 1..16: note = Engineered antibody sequence
VIGSDGKTYYATWAKG

SEQ ID NO: 327 - protein, 31 aa, synthetic construct
  REGION 1..31: note = Engineered antibody sequence
RFTISKTSSTTVDLRMASLTTEDTATYFCTR

SEQ ID NO: 328 - skipped

SEQ ID NO: 329 - protein, 11 aa, synthetic construct
  REGION 1..11: note = Engineered antibody sequence
WGPGTLVTVSS

SEQ ID NO: 330 - protein, 330 aa, synthetic construct; identical to SEQ ID NO: 10
  REGION 1..330: note = Engineered antibody sequence

SEQ ID NO: 331 - DNA, 1320 bp, synthetic construct
  misc_feature 1..1320: note = Engineered antibody sequence
  source 1..1320: mol_type = other DNA
cagtcgctggaggagtccgggggtcgcctggtcacgcctgggacacccctgacactcacctgcacagtctctggaatcggcctcagtagctactacatgcagtgggtccgccagtctccagggagggggctggaatggatcggagtcattggtagtgatggtaagacatactacgcgacctgggcgaaaggccgattcaccatctccaagacctcgtcgaccacggtggatctgagaatggccagtctgacaaccgaggacacggccacctatttctgtaccagaggggacatctggggcccggggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 332 - DNA, 327 bp, synthetic construct
  misc_feature 1..327: note = Engineered antibody sequence
  source 1..327: mol_type = other DNA
cagtcgctggaggagtccgggggtcgcctggtcacgcctgggacacccctgacactcacctgcacagtctctggaatcggcctcagtagctactacatgcagtgggtccgccagtctccagggagggggctggaatggatcggagtcattggtagtgatggtaagacatactacgcgacctgggcgaaaggccgattcaccatctccaagacctcgtcgaccacggtggatctgagaatggccagtctgacaaccgaggacacggccacctatttctgtaccagaggggacatctggggcccggggaccctcgtcaccgtctcgagc

SEQ ID NO: 333 - DNA, 87 bp, synthetic construct
  misc_feature 1..87: note = Engineered antibody sequence
  source 1..87: mol_type = other DNA
cagtcgctggaggagtccgggggtcgcctggtcacgcctgggacacccctgacactcacctgcacagtctctggaatcggcctcagt

SEQ ID NO: 334 - DNA, 15 bp, synthetic construct
  misc_feature 1..15: note = Engineered antibody sequence
  source 1..15: mol_type = other DNA
agctactacatgcag

SEQ ID NO: 335 - DNA, 42 bp, synthetic construct
  misc_feature 1..42: note = Engineered antibody sequence
  source 1..42: mol_type = other DNA
tgggtccgccagtctccagggagggggctggaatggatcgga

SEQ ID NO: 336 - DNA, 48 bp, synthetic construct
  misc_feature 1..48: note = Engineered antibody sequence
  source 1..48: mol_type = other DNA
gtcattggtagtgatggtaagacatactacgcgacctgggcgaaaggc

SEQ ID NO: 337 - DNA, 93 bp, synthetic construct
  misc_feature 1..93: note = Engineered antibody sequence
  source 1..93: mol_type = other DNA
cgattcaccatctccaagacctcgtcgaccacggtggatctgagaatggccagtctgacaaccgaggacacggccacctatttctgtaccaga

SEQ ID NO: 338 - skipped

SEQ ID NO: 339 - DNA, 33 bp, synthetic construct
  misc_feature 1..33: note = Engineered antibody sequence
  source 1..33: mol_type = other DNA
tggggcccggggaccctcgtcaccgtctcgagc

SEQ ID NO: 340 - DNA, 993 bp, synthetic construct; identical to SEQ ID NO: 20
  misc_feature 1..993: note = Engineered antibody sequence
  source 1..993: mol_type = other DNA

SEQ ID NO: 341 - protein, 219 aa, synthetic construct
  REGION 1..219: note = Engineered antibody sequence
QVLTQTPSPVSAAVGSTVTINCQASQNVYNNNYLAWYQQKPGQPPKQLIYSTSTLASGVSSRFRGSGSGTQFTLTISDVQCDDAATYYCLGSYDCSRGDCFVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342 - protein, 113 aa, synthetic construct
  REGION 1..113: note = Engineered antibody sequence
QVLTQTPSPVSAAVGSTVTINCQASQNVYNNNYLAWYQQKPGQPPKQLIYSTSTLASGVSSRFRGSGSGTQFTLTISDVQCDDAATYYCLGSYDCSRGDCFVFGGGTEVVVKR

SEQ ID NO: 343 - protein, 22 aa, synthetic construct
  REGION 1..22: note = Engineered antibody sequence
QVLTQTPSPVSAAVGSTVTINC

SEQ ID NO: 344 - protein, 13 aa, synthetic construct
  REGION 1..13: note = Engineered antibody sequence
QASQNVYNNNYLA

SEQ ID NO: 345 - protein, 15 aa, synthetic construct
  REGION 1..15: note = Engineered antibody sequence
WYQQKPGQPPKQLIY

SEQ ID NO: 346 - protein, 7 aa, synthetic construct
  REGION 1..7: note = Engineered antibody sequence
STSTLAS

SEQ ID NO: 347 - protein, 32 aa, synthetic construct
  REGION 1..32: note = Engineered antibody sequence
GVSSRFRGSGSGTQFTLTISDVQCDDAATYYC

SEQ ID NO: 348 - protein, 13 aa, synthetic construct
  REGION 1..13: note = Engineered antibody sequence
LGSYDCSRGDCFV

SEQ ID NO: 349 - protein, 11 aa, synthetic construct
  REGION 1..11: note = Engineered antibody sequence
FGGGTEVVVKR

SEQ ID NO: 350 - protein, 106 aa, synthetic construct; identical to SEQ ID NO: 30
  REGION 1..106: note = Engineered antibody sequence

SEQ ID NO: 351 - DNA, 660 bp, synthetic construct
  misc_feature 1..660: note = Engineered antibody sequence
  source 1..660: mol_type = other DNA
caagtgctgacccagactccatcccccgtgtctgcagctgtgggaagcacagtcaccatcaattgccaggccagtcagaatgtttataataacaactacctagcctggtatcagcagaaaccagggcagcctcccaagcaactgatctattctacgtccactctggcatctggggtctcatcgcgattcagaggcagtggatctgggacacagttcactctcaccatcagcgacgtgcagtgtgacgatgctgccacttactactgtctaggcagttatgattgtagtcgtggtgattgttttgttttcggcggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 352 - DNA, 339 bp, synthetic construct
  misc_feature 1..339: note = Engineered antibody sequence
  source 1..339: mol_type = other DNA
caagtgctgacccagactccatcccccgtgtctgcagctgtgggaagcacagtcaccatcaattgccaggccagtcagaatgtttataataacaactacctagcctggtatcagcagaaaccagggcagcctcccaagcaactgatctattctacgtccactctggcatctggggtctcatcgcgattcagaggcagtggatctgggacacagttcactctcaccatcagcgacgtgcagtgtgacgatgctgccacttactactgtctaggcagttatgattgtagtcgtggtgattgttttgttttcggcggagggaccgaggtggtggtcaaacgt

SEQ ID NO: 353 - DNA, 66 bp, synthetic construct; identical to SEQ ID NO: 153
  misc_feature 1..66: note = Engineered antibody sequence
  source 1..66: mol_type = other DNA

SEQ ID NO: 354 - DNA, 39 bp, synthetic construct
  misc_feature 1..39: note = Engineered antibody sequence
  source 1..39: mol_type = other DNA
caggccagtcagaatgtttataataacaactacctagcc

SEQ ID NO: 355 - DNA, 45 bp, synthetic construct; identical to SEQ ID NO: 35
  misc_feature 1..45: note = Engineered antibody sequence
  source 1..45: mol_type = other DNA

SEQ ID NO: 356 - DNA, 21 bp, synthetic construct
  misc_feature 1..21: note = Engineered antibody sequence
  source 1..21: mol_type = other DNA
tctacgtccactctggcatct

SEQ ID NO: 357 - DNA, 96 bp, synthetic construct
  misc_feature 1..96: note = Engineered antibody sequence
  source 1..96: mol_type = other DNA
ggggtctcatcgcgattcagaggcagtggatctgggacacagttcactctcaccatcagcgacgtgcagtgtgacgatgctgccacttactactgt

SEQ ID NO: 358 - DNA, 39 bp, synthetic construct
  misc_feature 1..39: note = Engineered antibody sequence
  source 1..39: mol_type = other DNA
ctaggcagttatgattgtagtcgtggtgattgttttgtt

SEQ ID NO: 359 - DNA, 33 bp, synthetic construct; identical to SEQ ID NO: 39
  misc_feature 1..33: note = Engineered antibody sequence
  source 1..33: mol_type = other DNA

SEQ ID NO: 360 - DNA, 321 bp, synthetic construct; identical to SEQ ID NO: 40
  misc_feature 1..321: note = Engineered antibody sequence
  source 1..321: mol_type = other DNA

SEQ ID NO: 361 - protein, 441 aa, synthetic construct
  REGION 1..441: note = Engineered antibody sequence
EVQLVESGGGLVQPGGSLRLSCAVSGIGLSSYYMQWVRQAPGKGLEWVGVIGSDGKTYYATWAKGRFTISRDNSKTTVYLQMNSLRAEDTAVYFCTRGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 111 aa, synthetic construct
  REGION 1..111: note = Engineered antibody sequence
EVQLVESGGGLVQPGGSLRLSCAVSGIGLSSYYMQWVRQAPGKGLEWVGVIGSDGKTYYATWAKGRFTISRDNSKTTVYLQMNSLRAEDTAVYFCTRGDIWGQGTLVTVSS

SEQ ID NO: 363 - protein, 30 aa, synthetic construct
  REGION 1..30: note = Engineered antibody sequence
EVQLVESGGGLVQPGGSLRLSCAVSGIGLS

SEQ ID NO: 364 - protein, 5 aa, synthetic construct
  REGION 1..5: note = Engineered antibody sequence
SYYMQ

SEQ ID NO: 365 - protein, 14 aa, synthetic construct
  REGION 1..14: note = Engineered antibody sequence
WVRQAPGKGLEWVG

SEQ ID NO: 366 - protein, 16 aa, synthetic construct
  REGION 1..16: note = Engineered antibody sequence
VIGSDGKTYYATWAKG

SEQ ID NO: 367 - protein, 32 aa, synthetic construct
  REGION 1..32: note = Engineered antibody sequence
RFTISRDNSKTTVYLQMNSLRAEDTAVYFCTR

SEQ ID NO: 368 - skipped

SEQ ID NO: 369 - protein, 11 aa, synthetic construct
  REGION 1..11: note = Engineered antibody sequence
WGQGTLVTVSS

SEQ ID NO: 370 - protein, 330 aa, synthetic construct; identical to SEQ ID NO: 10
  REGION 1..330: note = Engineered antibody sequence

SEQ ID NO: 371 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcggcctcagtagctactacatgcaatgggtccgtcaggctccagggaaggggctggagtgggtcggagtcattggtagtgatggtaagacatactacgcgacctgggcgaaaggccgattcaccatctccagagacaattccaagaccacggtgtatcttcaaatgaacagcctgagagctgaggacactgctgtgtatttctgtaccagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 372 - DNA, 333 bp, synthetic construct
  misc_feature 1..333: note = Engineered antibody sequence
  source 1..333: mol_type = other DNA
gaggtgcagcttgtggagtctgggggaggcttggtccagcctggggggtccctgagactctcctgtgcagtctctggaatcggcctcagtagctactacatgcaatgggtccgtcaggctccagggaaggggctggagtgggtcggagtcattggtagtgatggtaagacatactacgcgacctgggcgaaaggccgattcaccatctccagagacaattccaagaccacggtgtatcttcaaatgaacagcctgagagctgaggacactgctgtgtatttctgtaccagaggggacatctggggccaagggaccctcgtcaccgtctcgagc

SEQ ID NO: 373 - DNA, 90 bp, synthetic construct
  misc_feature 1..90: note = Engineered antibody sequence
  source 1..90: mol_type = other DNA
gaggtgcagcttgtggagtctgggggaggcttggtccagcctggggggtccctgagactctcctgtgcagtctctggaatcggcctcagt

SEQ ID NO: 374 - DNA, 15 bp, synthetic construct
  misc_feature 1..15: note = Engineered antibody sequence
  source 1..15: mol_type = other DNA
agctactacatgcaa

SEQ ID NO: 375 - DNA, 42 bp, synthetic construct; identical to SEQ ID NO: 55
  misc_feature 1..42: note = Engineered antibody sequence
  source 1..42: mol_type = other DNA

SEQ ID NO: 376 - DNA, 48 bp, synthetic construct; identical to SEQ ID NO: 336
  misc_feature 1..48: note = Engineered antibody sequence
  source 1..48: mol_type = other DNA

SEQ ID NO: 377 - DNA, 96 bp, synthetic construct
  misc_feature 1..96: note = Engineered antibody sequence
  source 1..96: mol_type = other DNA
cgattcaccatctccagagacaattccaagaccacggtgtatcttcaaatgaacagcctgagagctgaggacactgctgtgtatttctgtaccaga

SEQ ID NO: 378 - skipped

SEQ ID NO: 379 - DNA, 33 bp, synthetic construct; identical to SEQ ID NO: 59
  misc_feature 1..33: note = Engineered antibody sequence
  source 1..33: mol_type = other DNA

SEQ ID NO: 380 - DNA, 993 bp, synthetic construct; identical to SEQ ID NO: 20
  misc_feature 1..993: note = Engineered antibody sequence
  source 1..993: mol_type = other DNA

SEQ ID NO: 381 - protein, 219 aa, synthetic construct
  REGION 1..219: note = Engineered antibody sequence
QVLTQSPSSLSASVGDRVTINCQASQNVYNNNYLAWYQQKPGKVPKQLIYSTSTLASGVPSRFSGSGSGTDFTLTISSLQPEDVATYYCLGSYDCSRGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 382 - protein, 113 aa, synthetic construct
  REGION 1..113: note = Engineered antibody sequence
QVLTQSPSSLSASVGDRVTINCQASQNVYNNNYLAWYQQKPGKVPKQLIYSTSTLASGVPSRFSGSGSGTDFTLTISSLQPEDVATYYCLGSYDCSRGDCFVFGGGTKVEIKR

SEQ ID NO: 383 - protein, 22 aa, synthetic construct
  REGION 1..22: note = Engineered antibody sequence
QVLTQSPSSLSASVGDRVTINC

SEQ ID NO: 384 - protein, 13 aa, synthetic construct
  REGION 1..13: note = Engineered antibody sequence
QASQNVYNNNYLA

SEQ ID NO: 385 - protein, 15 aa, synthetic construct
  REGION 1..15: note = Engineered antibody sequence
WYQQKPGKVPKQLIY

SEQ ID NO: 386 - protein, 7 aa, synthetic construct
  REGION 1..7: note = Engineered antibody sequence
STSTLAS

SEQ ID NO: 387 - protein, 32 aa, synthetic construct; identical to SEQ ID NO: 67
  REGION 1..32: note = Engineered antibody sequence

SEQ ID NO: 388 - protein, 13 aa, synthetic construct
  REGION 1..13: note = Engineered antibody sequence
LGSYDCSRGDCFV

SEQ ID NO: 389 - protein, 11 aa, synthetic construct
  REGION 1..11: note = Engineered antibody sequence
FGGGTKVEIKR

SEQ ID NO: 390 - protein, 106 aa, synthetic construct; identical to SEQ ID NO: 30
  REGION 1..106: note = Engineered antibody sequence

SEQ ID NO: 391 - DNA, 660 bp, synthetic construct
  misc_feature 1..660: note = Engineered antibody sequence
  source 1..660: mol_type = other DNA
caagtgctgacccagtctccatcctccctgtctgcatctgtaggagacagagtcaccatcaattgccaggccagtcagaatgtttacaataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtcgtggtgattgttttgttttcggcggaggaaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 392 - DNA, 339 bp, synthetic construct
  misc_feature 1..339: note = Engineered antibody sequence
  source 1..339: mol_type = other DNA
caagtgctgacccagtctccatcctccctgtctgcatctgtaggagacagagtcaccatcaattgccaggccagtcagaatgtttacaataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtcgtggtgattgttttgttttcggcggaggaaccaaggtggaaatcaaacgt

SEQ ID NO: 393 - DNA, 66 bp, synthetic construct; identical to SEQ ID NO: 73
  misc_feature 1..66: note = Engineered antibody sequence
  source 1..66: mol_type = other DNA

SEQ ID NO: 394 - DNA, 39 bp, synthetic construct
  misc_feature 1..39: note = Engineered antibody sequence
  source 1..39: mol_type = other DNA
caggccagtcagaatgtttacaataacaactacctagcc

SEQ ID NO: 395 - DNA, 45 bp, synthetic construct; identical to SEQ ID NO: 75
  misc_feature 1..45: note = Engineered antibody sequence
  source 1..45: mol_type = other DNA

SEQ ID NO: 396 - DNA, 21 bp, synthetic construct
  misc_feature 1..21: note = Engineered antibody sequence
  source 1..21: mol_type = other DNA
tctacatccactctggcatct

SEQ ID NO: 397 - DNA, 96 bp, synthetic construct; identical to SEQ ID NO: 77
  misc_feature 1..96: note = Engineered antibody sequence
  source 1..96: mol_type = other DNA

SEQ ID NO: 398 - DNA, 39 bp, synthetic construct
  misc_feature 1..39: note = Engineered antibody sequence
  source 1..39: mol_type = other DNA
ctgggcagttatgattgtagtcgtggtgattgttttgtt

SEQ ID NO: 399 - DNA, 33 bp, synthetic construct; identical to SEQ ID NO: 79
  misc_feature 1..33: note = Engineered antibody sequence
  source 1..33: mol_type = other DNA

SEQ ID NO: 400 - DNA, 321 bp, synthetic construct; identical to SEQ ID NO: 40
  misc_feature 1..321: note = Engineered antibody sequence
  source 1..321: mol_type = other DNA

SEQ ID NO: 401 - protein, 439 aa, synthetic construct
  REGION 1..439: note = Engineered antibody sequence
QSLEESGGRLVTPGGSLTLTCTVSGIDVTNYYMQWVRQAPGKGLEWIGVIGVNGKRYYASWAKGRFTISKTSSTTVDLKMTSLTTEDTATYFCARGDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 402 - protein, 109 aa, synthetic construct
  REGION 1..109: note = Engineered antibody sequence
QSLEESGGRLVTPGGSLTLTCTVSGIDVTNYYMQWVRQAPGKGLEWIGVIGVNGKRYYASWAKGRFTISKTSSTTVDLKMTSLTTEDTATYFCARGDIWGPGTLVTVSS

SEQ ID NO: 403 - protein, 29 aa, synthetic construct
  REGION 1..29: note = Engineered antibody sequence
QSLEESGGRLVTPGGSLTLTCTVSGIDVT

SEQ ID NO: 404 - protein, 5 aa, synthetic construct
  REGION 1..5: note = Engineered antibody sequence
NYYMQ

SEQ ID NO: 405 - protein, 14 aa, synthetic construct
  REGION 1..14: note = Engineered antibody sequence
WVRQAPGKGLEWIG

SEQ ID NO: 406 - protein, 16 aa, synthetic construct
  REGION 1..16: note = Engineered antibody sequence
VIGVNGKRYYASWAKG

SEQ ID NO: 407 - protein, 31 aa, synthetic construct; identical to SEQ ID NO: 127
  REGION 1..31: note = Engineered antibody sequence

SEQ ID NO: 408 - skipped

SEQ ID NO: 409 - protein, 11 aa, synthetic construct
  REGION 1..11: note = Engineered antibody sequence
WGPGTLVTVSS

SEQ ID NO: 410 - protein, 330 aa, synthetic construct; identical to SEQ ID NO: 10
  REGION 1..330: note = Engineered antibody sequence

SEQ ID NO: 411 - DNA, 1320 bp, synthetic construct
  misc_feature 1..1320: note = Engineered antibody sequence
  source 1..1320: mol_type = other DNA
cagtcgctggaggagtccgggggtcgcctggtcacgcctggaggatccctgacactcacctgcacagtctctggaatcgacgtcactaactactatatgcaatgggtccgccaggctccagggaaggggctggaatggatcggagtcattggtgtgaatggtaagagatactacgcgagctgggcgaaaggccgattcaccatctccaaaacctcgtcgaccacggtggatctgaaaatgaccagtctgacaaccgaggacacggccacctatttctgtgccagaggcgacatctggggcccggggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 412 - DNA, 327 bp, synthetic construct
  misc_feature 1..327: note = Engineered antibody sequence
  source 1..327: mol_type = other DNA
cagtcgctggaggagtccgggggtcgcctggtcacgcctggaggatccctgacactcacctgcacagtctctggaatcgacgtcactaactactatatgcaatgggtccgccaggctccagggaaggggctggaatggatcggagtcattggtgtgaatggtaagagatactacgcgagctgggcgaaaggccgattcaccatctccaaaacctcgtcgaccacggtggatctgaaaatgaccagtctgacaaccgaggacacggccacctatttctgtgccagaggcgacatctggggcccggggaccctcgtcaccgtctcgagc

SEQ ID NO: 413 - DNA, 87 bp, synthetic construct
  misc_feature 1..87: note = Engineered antibody sequence
  source 1..87: mol_type = other DNA
cagtcgctggaggagtccgggggtcgcctggtcacgcctggaggatccctgacactcacctgcacagtctctggaatcgacgtcact

SEQ ID NO: 414 - DNA, 15 bp, synthetic construct
  misc_feature 1..15: note = Engineered antibody sequence
  source 1..15: mol_type = other DNA
aactactatatgcaa

SEQ ID NO: 415 - DNA, 42 bp, synthetic construct; identical to SEQ ID NO: 15
  misc_feature 1..42: note = Engineered antibody sequence
  source 1..42: mol_type = other DNA

SEQ ID NO: 416 - DNA, 48 bp, synthetic construct
  misc_feature 1..48: note = Engineered antibody sequence
  source 1..48: mol_type = other DNA
gtcattggtgtgaatggtaagagatactacgcgagctgggcgaaaggc

SEQ ID NO: 417 - DNA, 93 bp, synthetic construct; identical to SEQ ID NO: 137
  misc_feature 1..93: note = Engineered antibody sequence
  source 1..93: mol_type = other DNA

SEQ ID NO: 418 - skipped

SEQ ID NO: 419 - DNA, 33 bp, synthetic construct; identical to SEQ ID NO: 339
  misc_feature 1..33: note = Engineered antibody sequence
  source 1..33: mol_type = other DNA

SEQ ID NO: 420 - DNA, 993 bp, synthetic construct; identical to SEQ ID NO: 20
  misc_feature 1..993: note = Engineered antibody sequence
  source 1..993: mol_type = other DNA

SEQ ID NO: 421 - protein, 219 aa, synthetic construct
  REGION 1..219: note = Engineered antibody sequence
QVLTQTASPVSPAVGSTVTINCRASQSVYYNNYLAWYQQKPGQPPKQLIYSTSTLASGVSSRFKGSGSGTQFTLTISDVQCDDAATYYCLGSYDCSNGDCFVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 422 - protein, 113 aa, synthetic construct
  REGION 1..113: note = Engineered antibody sequence
QVLTQTASPVSPAVGSTVTINCRASQSVYYNNYLAWYQQKPGQPPKQLIYSTSTLASGVSSRFKGSGSGTQFTLTISDVQCDDAATYYCLGSYDCSNGDCFVFGGGTEVVVKR

SEQ ID NO: 423 - protein, 22 aa, synthetic construct
  REGION 1..22: note = Engineered antibody sequence
QVLTQTASPVSPAVGSTVTINC

SEQ ID NO: 424 - protein, 13 aa, synthetic construct
  REGION 1..13: note = Engineered antibody sequence
RASQSVYYNNYLA

SEQ ID NO: 425 - protein, 15 aa, synthetic construct
  REGION 1..15: note = Engineered antibody sequence
WYQQKPGQPPKQLIY

SEQ ID NO: 426 - protein, 7 aa, synthetic construct
  REGION 1..7: note = Engineered antibody sequence
STSTLAS

SEQ ID NO: 427 - protein, 32 aa, synthetic construct; identical to SEQ ID NO: 267
  REGION 1..32: note = Engineered antibody sequence

SEQ ID NO: 428 - protein, 13 aa, synthetic construct
  REGION 1..13: note = Engineered antibody sequence
LGSYDCSNGDCFV

SEQ ID NO: 429 - protein, 11 aa, synthetic construct
  REGION 1..11: note = Engineered antibody sequence
FGGGTEVVVKR

SEQ ID NO: 430 - protein, 106 aa, synthetic construct; identical to SEQ ID NO: 30
  REGION 1..106: note = Engineered antibody sequence

SEQ ID NO: 431 - DNA, 660 bp, synthetic construct
  misc_feature 1..660: note = Engineered antibody sequence
  source 1..660: mol_type = other DNA
caggtgctgacccagactgcatcccccgtgtctccagctgtgggaagcacagtcaccatcaattgccgggccagtcagagtgtttattataacaactacctagcctggtatcagcagaaaccagggcagcctcccaagcaactgatctattctacatccactctggcatctggggtctcatcgcggttcaaaggcagtggatctgggacacagttcactctcaccatcagcgacgtgcagtgtgacgatgctgccacttactactgtctaggcagttatgattgtagtaatggtgattgttttgttttcggcggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 432 - DNA, 339 bp, synthetic construct
  misc_feature 1..339: note = Engineered antibody sequence
  source 1..339: mol_type = other DNA
caggtgctgacccagactgcatcccccgtgtctccagctgtgggaagcacagtcaccatcaattgccgggccagtcagagtgtttattataacaactacctagcctggtatcagcagaaaccagggcagcctcccaagcaactgatctattctacatccactctggcatctggggtctcatcgcggttcaaaggcagtggatctgggacacagttcactctcaccatcagcgacgtgcagtgtgacgatgctgccacttactactgtctaggcagttatgattgtagtaatggtgattgttttgttttcggcggagggaccgaggtggtggtcaaacgt

SEQ ID NO: 433 - DNA, 66 bp, synthetic construct
  misc_feature 1..66: note = Engineered antibody sequence
  source 1..66: mol_type = other DNA
caggtgctgacccagactgcatcccccgtgtctccagctgtgggaagcacagtcaccatcaattgc

SEQ ID NO: 434 - DNA, 39 bp, synthetic construct
  misc_feature 1..39: note = Engineered antibody sequence
  source 1..39: mol_type = other DNA
cgggccagtcagagtgtttattataacaactacctagcc

SEQ ID NO: 435 - DNA, 45 bp, synthetic construct; identical to SEQ ID NO: 35
  misc_feature 1..45: note = Engineered antibody sequence
  source 1..45: mol_type = other DNA

SEQ ID NO: 436 - DNA, 21 bp, synthetic construct
  misc_feature 1..21: note = Engineered antibody sequence
  source 1..21: mol_type = other DNA
tctacatccactctggcatct

SEQ ID NO: 437 - DNA, 96 bp, synthetic construct
  misc_feature 1..96: note = Engineered antibody sequence
  source 1..96: mol_type = other DNA
ggggtctcatcgcggttcaaaggcagtggatctgggacacagttcactctcaccatcagcgacgtgcagtgtgacgatgctgccacttactactgt

SEQ ID NO: 438 - DNA, 39 bp, synthetic construct
  misc_feature 1..39: note = Engineered antibody sequence
  source 1..39: mol_type = other DNA
ctaggcagttatgattgtagtaatggtgattgttttgtt

SEQ ID NO: 439 - DNA, 33 bp, synthetic construct; identical to SEQ ID NO: 39
  misc_feature 1..33: note = Engineered antibody sequence
  source 1..33: mol_type = other DNA

SEQ ID NO: 440 - DNA, 321 bp, synthetic construct; identical to SEQ ID NO: 40
  misc_feature 1..321: note = Engineered antibody sequence
  source 1..321: mol_type = other DNA

SEQ ID NO: 441 - protein, 441 aa, synthetic construct
  REGION 1..441: note = Engineered antibody sequence
EVQLVESGGGLVQPGGSLRLSCAVSGIDVTNYYMQWVRQAPGKGLEWVGVIGVNGKRYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 442 - protein, 111 aa, synthetic construct
  REGION 1..111: note = Engineered antibody sequence
EVQLVESGGGLVQPGGSLRLSCAVSGIDVTNYYMQWVRQAPGKGLEWVGVIGVNGKRYYASWAKGRFTISRDNSKTTVYLQMNSLRAEDTAVYFCARGDIWGQGTLVTVSS

SEQ ID NO: 443 - protein, 30 aa, synthetic construct
  REGION 1..30: note = Engineered antibody sequence
EVQLVESGGGLVQPGGSLRLSCAVSGIDVT

SEQ ID NO: 444 - protein, 5 aa, synthetic construct
  REGION 1..5: note = Engineered antibody sequence
NYYMQ

SEQ ID NO: 445 - protein, 14 aa, synthetic construct
  REGION 1..14: note = Engineered antibody sequence
WVRQAPGKGLEWVG

SEQ ID NO: 446 - protein, 16 aa, synthetic construct
  REGION 1..16: note = Engineered antibody sequence
VIGVNGKRYYASWAKG

SEQ ID NO: 447 - protein, 32 aa, synthetic construct; identical to SEQ ID NO: 47
  REGION 1..32: note = Engineered antibody sequence

SEQ ID NO: 448 - skipped

SEQ ID NO: 449 - protein, 11 aa, synthetic construct
  REGION 1..11: note = Engineered antibody sequence
WGQGTLVTVSS

SEQ ID NO: 450 - protein, 330 aa, synthetic construct; identical to SEQ ID NO: 10
  REGION 1..330: note = Engineered antibody sequence

SEQ ID NO: 451 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcgacgtcactaactactacatgcaatgggtccgtcaggctccagggaaggggctggagtgggtcggagtcattggtgtgaatggtaagagatactacgcgagctgggcgaaaggccgattcaccatctccagagacaattccaagaccacggtgtatcttcaaatgaacagcctgagagctgaggacactgctgtgtatttctgtgccagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 452 - DNA, 333 bp, synthetic construct
  misc_feature 1..333: note = Engineered antibody sequence
  source 1..333: mol_type = other DNA
gaggtgcagcttgtggagtctgggggaggcttggtccagcctggggggtccctgagactctcctgtgcagtctctggaatcgacgtcactaactactacatgcaatgggtccgtcaggctccagggaaggggctggagtgggtcggagtcattggtgtgaatggtaagagatactacgcgagctgggcgaaaggccgattcaccatctccagagacaattccaagaccacggtgtatcttcaaatgaacagcctgagagctgaggacactgctgtgtatttctgtgccagaggggacatctggggccaagggaccctcgtcaccgtctcgagc

SEQ ID NO: 453 - DNA, 90 bp, synthetic construct
  misc_feature 1..90: note = Engineered antibody sequence
  source 1..90: mol_type = other DNA
gaggtgcagcttgtggagtctgggggaggcttggtccagcctggggggtccctgagactctcctgtgcagtctctggaatcgacgtcact

SEQ ID NO: 454 - DNA, 15 bp, synthetic construct
  misc_feature 1..15: note = Engineered antibody sequence
  source 1..15: mol_type = other DNA
aactactacatgcaa

SEQ ID NO: 455 - DNA, 42 bp, synthetic construct; identical to SEQ ID NO: 55
  misc_feature 1..42: note = Engineered antibody sequence
  source 1..42: mol_type = other DNA

SEQ ID NO: 456 - DNA, 48 bp, synthetic construct; identical to SEQ ID NO: 416
  misc_feature 1..48: note = Engineered antibody sequence
  source 1..48: mol_type = other DNA

SEQ ID NO: 457 - DNA, 96 bp, synthetic construct
  misc_feature 1..96: note = Engineered antibody sequence
  source 1..96: mol_type = other DNA
cgattcaccatctccagagacaattccaagaccacggtgtatcttcaaatgaacagcctgagagctgaggacactgctgtgtatttctgtgccaga

SEQ ID NO: 458 - skipped

SEQ ID NO: 459 - DNA, 33 bp, synthetic construct; identical to SEQ ID NO: 59
  misc_feature 1..33: note = Engineered antibody sequence
  source 1..33: mol_type = other DNA

SEQ ID NO: 460 - DNA, 993 bp, synthetic construct; identical to SEQ ID NO: 20
  misc_feature 1..993: note = Engineered antibody sequence
  source 1..993: mol_type = other DNA

SEQ ID NO: 461 - protein, 219 aa, synthetic construct
  REGION 1..219: note = Engineered antibody sequence
QVLTQSPSSLSASVGDRVTINCRASQSVYYNNYLAWYQQKPGKVPKQLIYSTSTLASGVPSRFSGSGSGTDFTLTISSLQPEDVATYYCLGSYDCSNGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 462 - protein, 113 aa, synthetic construct
  REGION 1..113: note = Engineered antibody sequence
QVLTQSPSSLSASVGDRVTINCRASQSVYYNNYLAWYQQKPGKVPKQLIYSTSTLASGVPSRFSGSGSGTDFTLTISSLQPEDVATYYCLGSYDCSNGDCFVFGGGTKVEIKR

SEQ ID NO: 463 - protein, 22 aa, synthetic construct
  REGION 1..22: note = Engineered antibody sequence
QVLTQSPSSLSASVGDRVTINC

SEQ ID NO: 464 - protein, 13 aa, synthetic construct
  REGION 1..13: note = Engineered antibody sequence
RASQSVYYNNYLA

SEQ ID NO: 465 - protein, 15 aa, synthetic construct
  REGION 1..15: note = Engineered antibody sequence
WYQQKPGKVPKQLIY

SEQ ID NO: 466 - protein, 7 aa, synthetic construct
  REGION 1..7: note = Engineered antibody sequence
STSTLAS

SEQ ID NO: 467 - protein, 32 aa, synthetic construct; identical to SEQ ID NO: 67
  REGION 1..32: note = Engineered antibody sequence

SEQ ID NO: 468 - protein, 13 aa, synthetic construct
  REGION 1..13: note = Engineered antibody sequence
LGSYDCSNGDCFV

SEQ ID NO: 469 - protein, 11 aa, synthetic construct
  REGION 1..11: note = Engineered antibody sequence
FGGGTKVEIKR

SEQ ID NO: 470 - protein, 106 aa, synthetic construct; identical to SEQ ID NO: 30
  REGION 1..106: note = Engineered antibody sequence

SEQ ID NO: 471 - DNA, 660 bp, synthetic construct
  misc_feature 1..660: note = Engineered antibody sequence
  source 1..660: mol_type = other DNA
caagtgctgacccagtctccatcctccctgtctgcatctgtaggagacagagtcaccatcaattgccgggccagtcagagtgtttactataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtaatggtgattgttttgttttcggcggaggaaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 472 - DNA, 339 bp, synthetic construct
  misc_feature 1..339: note = Engineered antibody sequence
  source 1..339: mol_type = other DNA
caagtgctgacccagtctccatcctccctgtctgcatctgtaggagacagagtcaccatcaattgccgggccagtcagagtgtttactataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtaatggtgattgttttgttttcggcggaggaaccaaggtggaaatcaaacgt

SEQ ID NO: 473 - DNA, 66 bp, synthetic construct; identical to SEQ ID NO: 73
  misc_feature 1..66: note = Engineered antibody sequence
  source 1..66: mol_type = other DNA

SEQ ID NO: 474 - DNA, 39 bp, synthetic construct
  misc_feature 1..39: note = Engineered antibody sequence
  source 1..39: mol_type = other DNA
cgggccagtcagagtgtttactataacaactacctagcc

SEQ ID NO: 475 - DNA, 45 bp, synthetic construct; identical to SEQ ID NO: 75
  misc_feature 1..45: note = Engineered antibody sequence
  source 1..45: mol_type = other DNA

SEQ ID NO: 476 - DNA, 21 bp, synthetic construct
  misc_feature 1..21: note = Engineered antibody sequence
  source 1..21: mol_type = other DNA
tctacatccactctggcatct

SEQ ID NO: 477 - DNA, 96 bp, synthetic construct; identical to SEQ ID NO: 77
  misc_feature 1..96: note = Engineered antibody sequence
  source 1..96: mol_type = other DNA

SEQ ID NO: 478 - DNA, 39 bp, synthetic construct
  misc_feature 1..39: note = Engineered antibody sequence
  source 1..39: mol_type = other DNA
ctgggcagttatgattgtagtaatggtgattgttttgtt

SEQ ID NO: 479 - DNA, 33 bp, synthetic construct; identical to SEQ ID NO: 79
  misc_feature 1..33: note = Engineered antibody sequence
  source 1..33: mol_type = other DNA

SEQ ID NO: 480 - DNA, 321 bp, synthetic construct; identical to SEQ ID NO: 40
  misc_feature 1..321: note = Engineered antibody sequence
  source 1..321: mol_type = other DNA

SEQ ID NO: 481 - protein, 441 aa, synthetic construct
  REGION 1..441: note = Engineered antibody sequence
QSVEESGGGLVQPEGSLTLTCTASGFDFSSNAMWWVRQAPGKGLEWIGCIYNGDGSTYYASWVNGRFSISKTSSTTVTLQLNSLTVADTATYYCARDLDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 482 - protein, 111 aa, synthetic construct
  REGION 1..111: note = Engineered antibody sequence
QSVEESGGGLVQPEGSLTLTCTASGFDFSSNAMWWVRQAPGKGLEWIGCIYNGDGSTYYASWVNGRFSISKTSSTTVTLQLNSLTVADTATYYCARDLDLWGPGTLVTVSS

SEQ ID NO: 483 - protein, 29 aa, synthetic construct
  REGION 1..29: note = Engineered antibody sequence
QSVEESGGGLVQPEGSLTLTCTASGFDFS

SEQ ID NO: 484 - protein, 5 aa, synthetic construct
  REGION 1..5: note = Engineered antibody sequence
SNAMW

SEQ ID NO: 485 - protein, 14 aa, synthetic construct
  REGION 1..14: note = Engineered antibody sequence
WVRQAPGKGLEWIG

SEQ ID NO: 486 - protein, 17 aa, synthetic construct
  REGION 1..17: note = Engineered antibody sequence
CIYNGDGSTYYASWVNG

SEQ ID NO: 487 - protein, 31 aa, synthetic construct
  REGION 1..31: note = Engineered antibody sequence
RFSISKTSSTTVTLQLNSLTVADTATYYCAR

SEQ ID NO: 488 - protein, 4 aa, synthetic construct
  REGION 1..4: note = Engineered antibody sequence
DLDL

SEQ ID NO: 489 - protein, 11 aa, synthetic construct
  REGION 1..11: note = Engineered antibody sequence
WGPGTLVTVSS

SEQ ID NO: 490 - protein, 330 aa, synthetic construct; identical to SEQ ID NO: 10
  REGION 1..330: note = Engineered antibody sequence

SEQ ID NO: 491 - DNA, 1326 bp, synthetic construct
  misc_feature 1..1326: note = Engineered antibody sequence
  source 1..1326: mol_type = other DNA
cagtcggtggaggagtccgggggaggcctggtccagcctgagggatccctgacactcacctgcacagcctctggattcgacttcagtagcaatgcaatgtggtgggtccgccaggctccagggaaggggctggagtggatcggatgcatttacaatggtgatggcagcacatactacgcgagctgggtgaatggccgattctccatctccaaaacctcgtcgaccacggtgactctgcaactgaatagtctgacagtcgcggacacggccacgtattattgtgcgagagatcttgacttgtggggcccgggcaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 492 - DNA, 333 bp, synthetic construct
  misc_feature 1..333: note = Engineered antibody sequence
  source 1..333: mol_type = other DNA
cagtcggtggaggagtccgggggaggcctggtccagcctgagggatccctgacactcacctgcacagcctctggattcgacttcagtagcaatgcaatgtggtgggtccgccaggctccagggaaggggctggagtggatcggatgcatttacaatggtgatggcagcacatactacgcgagctgggtgaatggccgattctccatctccaaaacctcgtcgaccacggtgactctgcaactgaatagtctgacagtcgcggacacggccacgtattattgtgcgagagatcttgacttgtggggcccgggcaccctcgtcaccgtctcgagc

SEQ ID NO: 493 - DNA, 87 bp, synthetic construct
  misc_feature 1..87: note = Engineered antibody sequence
  source 1..87: mol_type = other DNA
cagtcggtggaggagtccgggggaggcctggtccagcctgagggatccctgacactcacctgcacagcctctggattcgacttcagt

SEQ ID NO: 494 - DNA, 15 bp, synthetic construct
  misc_feature 1..15: note = Engineered antibody sequence
  source 1..15: mol_type = other DNA
agcaatgcaatgtgg

SEQ ID NO: 495 - DNA, 42 bp, synthetic construct; identical to SEQ ID NO: 255
  misc_feature 1..42: note = Engineered antibody sequence
  source 1..42: mol_type = other DNA

SEQ ID NO: 496 - DNA, 51 bp, synthetic construct
  misc_feature 1..51: note = Engineered antibody sequence
  source 1..51: mol_type = other DNA
tgcatttacaatggtgatggcagcacatactacgcgagctgggtgaatggc

SEQ ID NO: 497 - DNA, 93 bp, synthetic construct
  misc_feature 1..93: note = Engineered antibody sequence
  source 1..93: mol_type = other DNA
cgattctccatctccaaaacctcgtcgaccacggtgactctgcaactgaatagtctgacagtcgcggacacggccacgtattattgtgcgaga

SEQ ID NO: 498 - DNA, 12 bp, synthetic construct
  misc_feature 1..12: note = Engineered antibody sequence
  source 1..12: mol_type = other DNA
gatcttgacttg

SEQ ID NO: 499 - DNA, 33 bp, synthetic construct; identical to SEQ ID NO: 139
  misc_feature 1..33: note = Engineered antibody sequence
  source 1..33: mol_type = other DNA

SEQ ID NO: 500 - DNA, 993 bp, synthetic construct; identical to SEQ ID NO: 20
  misc_feature 1..993: note = Engineered antibody sequence
  source 1..993: mol_type = other DNA

SEQ ID NO: 501 - protein, 219 aa, synthetic construct
  REGION 1..219: note = Engineered antibody sequence
AIVMTQTPSSKSVPVGDTVTINCQASESLYNNNALAWFQQKPGQPPKRLIYDASKLASGVPSRFSGGGSGTQFTLTISGVQCDDAATYYCGGYRSDSVDGVAFAGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 502 - protein, 113 aa, synthetic construct
  REGION 1..113: note = Engineered antibody sequence
AIVMTQTPSSKSVPVGDTVTINCQASESLYNNNALAWFQQKPGQPPKRLIYDASKLASGVPSRFSGGGSGTQFTLTISGVQCDDAATYYCGGYRSDSVDGVAFAGGTEVVVKR

SEQ ID NO: 503 - protein, 23 aa, synthetic construct
  REGION 1..23: note = Engineered antibody sequence
AIVMTQTPSSKSVPVGDTVTINC

SEQ ID NO: 504 - protein, 13 aa, synthetic construct
  REGION 1..13: note = Engineered antibody sequence
QASESLYNNNALA

SEQ ID NO: 505 - protein, 15 aa, synthetic construct
  REGION 1..15: note = Engineered antibody sequence
WFQQKPGQPPKRLIY

SEQ ID NO: 506 - protein, 7 aa, synthetic construct
  REGION 1..7: note = Engineered antibody sequence
DASKLAS

SEQ ID NO: 507 - protein, 32 aa, synthetic construct
  REGION 1..32: note = Engineered antibody sequence
GVPSRFSGGGSGTQFTLTISGVQCDDAATYYC

SEQ ID NO: 508 - protein, 12 aa, synthetic construct
  REGION 1..12: note = Engineered antibody sequence
GGYRSDSVDGVA

SEQ ID NO: 509 - protein, 11 aa, synthetic construct
  REGION 1..11: note = Engineered antibody sequence
FAGGTEVVVKR

SEQ ID NO: 510 - protein, 106 aa, synthetic construct; identical to SEQ ID NO: 30
  REGION 1..106: note = Engineered antibody sequence

SEQ ID NO: 511 - DNA, 660 bp, synthetic construct
  misc_feature 1..660: note = Engineered antibody sequence
  source 1..660: mol_type = other DNA
gccatcgtgatgacccagactccatcttccaagtctgtccctgtgggagacacagtcaccatcaattgccaggccagtgagagtctttataataacaacgccttggcctggtttcagcagaaaccagggcagcctcccaagcgcctgatctatgatgcatccaaactggcatctggggtcccatcgcggttcagtggcggtgggtctgggacacagttcactctcaccatcagtggcgtgcagtgtgacgatgctgccacttactactgtggaggctacagaagtgatagtgttgatggtgttgctttcgccggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 512 - DNA, 339 bp, synthetic construct
  misc_feature 1..339: note = Engineered antibody sequence
  source 1..339: mol_type = other DNA
gccatcgtgatgacccagactccatcttccaagtctgtccctgtgggagacacagtcaccatcaattgccaggccagtgagagtctttataataacaacgccttggcctggtttcagcagaaaccagggcagcctcccaagcgcctgatctatgatgcatccaaactggcatctggggtcccatcgcggttcagtggcggtgggtctgggacacagttcactctcaccatcagtggcgtgcagtgtgacgatgctgccacttactactgtggaggctacagaagtgatagtgttgatggtgttgctttcgccggagggaccgaggtggtggtcaaacgt

SEQ ID NO: 513 - DNA, 69 bp, synthetic construct
  misc_feature 1..69: note = Engineered antibody sequence
  source 1..69: mol_type = other DNA
gccatcgtgatgacccagactccatcttccaagtctgtccctgtgggagacacagtcaccatcaattgc

SEQ ID NO: 514 - DNA, 39 bp, synthetic construct
  misc_feature 1..39: note = Engineered antibody sequence
  source 1..39: mol_type = other DNA
caggccagtgagagtctttataataacaacgccttggcc

SEQ ID NO: 515 - DNA, 45 bp, synthetic construct
  misc_feature 1..45: note = Engineered antibody sequence
  source 1..45: mol_type = other DNA
tggtttcagcagaaaccagggcagcctcccaagcgcctgatctat

SEQ ID NO: 516 - DNA, 21 bp, synthetic construct
  misc_feature 1..21: note = Engineered antibody sequence
  source 1..21: mol_type = other DNA
gatgcatccaaactggcatct

SEQ ID NO: 517 - DNA, 96 bp, synthetic construct
  misc_feature 1..96: note = Engineered antibody sequence
  source 1..96: mol_type = other DNA
ggggtcccatcgcggttcagtggcggtgggtctgggacacagttcactctcaccatcagtggcgtgcagtgtgacgatgctgccacttactactgt

SEQ ID NO: 518 - DNA, 36 bp, synthetic construct
  misc_feature 1..36: note = Engineered antibody sequence
  source 1..36: mol_type = other DNA
ggaggctacagaagtgatagtgttgatggtgttgct

SEQ ID NO: 519 - DNA, 33 bp, synthetic construct
  misc_feature 1..33: note = Engineered antibody sequence
  source 1..33: mol_type = other DNA
ttcgccggagggaccgaggtggtggtcaaacgt

SEQ ID NO: 520 - DNA, 321 bp, synthetic construct; identical to SEQ ID NO: 40
  misc_feature 1..321: note = Engineered antibody sequence
  source 1..321: mol_type = other DNA

SEQ ID NO: 521 - protein, 441 aa, synthetic construct
  REGION 1..441: note = Engineered antibody sequence
EVQLVESGGGLVQPGGSLRLSCAVSGIGLSSYYMQWVRQAPGKGLEWVGVIGSDGKTYYATWAKGRFTISRDNSKTTVYLQMNSLRAEDTAVYFCTRGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 522 - protein, 111 aa, synthetic construct; identical to SEQ ID NO: 362
  REGION 1..111: note = Engineered antibody sequence

SEQ ID NO: 523 - protein, 30 aa, synthetic construct; identical to SEQ ID NO: 363
  REGION 1..30: note = Engineered antibody sequence

SEQ ID NO: 524 - protein, 5 aa, synthetic construct
  REGION 1..5: note = Engineered antibody sequence
SYYMQ

SEQ ID NO: 525 - protein, 14 aa, synthetic construct
  REGION 1..14: note = Engineered antibody sequence
WVRQAPGKGLEWVG

SEQ ID NO: 526 - protein, 16 aa, synthetic construct
  REGION 1..16: note = Engineered antibody sequence
VIGSDGKTYYATWAKG

SEQ ID NO: 527 - protein, 32 aa, synthetic construct; identical to SEQ ID NO: 367
  REGION 1..32: note = Engineered antibody sequence

SEQ ID NO: 528 - skipped

SEQ ID NO: 529 - protein, 11 aa, synthetic construct
  REGION 1..11: note = Engineered antibody sequence
WGQGTLVTVSS

SEQ ID NO: 530 - protein, 330 aa, synthetic construct; identical to SEQ ID NO: 90
  REGION 1..330: note = Engineered antibody sequence

SEQ ID NO: 531 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcggcctcagtagctactacatgcaatgggtccgtcaggctccagggaaggggctggagtgggtcggagtcattggtagtgatggtaagacatactacgcgacctgggcgaaaggccgattcaccatctccagagacaattccaagaccacggtgtatcttcaaatgaacagcctgagagctgaggacactgctgtgtatttctgtaccagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacgcgagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 532 - DNA, 333 bp, synthetic construct; identical to SEQ ID NO: 372
  misc_feature 1..333: note = Engineered antibody sequence
  source 1..333: mol_type = other DNA

SEQ ID NO: 533 - DNA, 90 bp, synthetic construct; identical to SEQ ID NO: 373
  misc_feature 1..90: note = Engineered antibody sequence
  source 1..90: mol_type = other DNA

SEQ ID NO: 534 - DNA, 15 bp, synthetic construct
  misc_feature 1..15: note = Engineered antibody sequence
  source 1..15: mol_type = other DNA
agctactacatgcaa

SEQ ID NO: 535 - DNA, 42 bp, synthetic construct; identical to SEQ ID NO: 55
  misc_feature 1..42: note = Engineered antibody sequence
  source 1..42: mol_type = other DNA

SEQ ID NO: 536 - DNA, 48 bp, synthetic construct; identical to SEQ ID NO: 336
  misc_feature 1..48: note = Engineered antibody sequence
  source 1..48: mol_type = other DNA

SEQ ID NO: 537 - DNA, 96 bp, synthetic construct; identical to SEQ ID NO: 377
  misc_feature 1..96: note = Engineered antibody sequence
  source 1..96: mol_type = other DNA

SEQ ID NO: 538 - skipped

SEQ ID NO: 539 - DNA, 33 bp, synthetic construct; identical to SEQ ID NO: 59
  misc_feature 1..33: note = Engineered antibody sequence
  source 1..33: mol_type = other DNA

SEQ ID NO: 540 - DNA, 993 bp, synthetic construct; identical to SEQ ID NO: 100
  misc_feature 1..993: note = Engineered antibody sequence
  source 1..993: mol_type = other DNA

SEQ ID NO: 541 - protein, 219 aa, synthetic construct; identical to SEQ ID NO: 381
  REGION 1..219: note = Engineered antibody sequence

SEQ ID NO: 542 - protein, 113 aa, synthetic construct; identical to SEQ ID NO: 382
  REGION 1..113: note = Engineered antibody sequence

SEQ ID NO: 543 - protein, 22 aa, synthetic construct
  REGION 1..22: note = Engineered antibody sequence
QVLTQSPSSLSASVGDRVTINC

SEQ ID NO: 544 - protein, 13 aa, synthetic construct
  REGION 1..13: note = Engineered antibody sequence
QASQNVYNNNYLA

SEQ ID NO: 545 - protein, 15 aa, synthetic construct
  REGION 1..15: note = Engineered antibody sequence
WYQQKPGKVPKQLIY

SEQ ID NO: 546 - protein, 7 aa, synthetic construct
  REGION 1..7: note = Engineered antibody sequence
STSTLAS

SEQ ID NO: 547 - protein, 32 aa, synthetic construct; identical to SEQ ID NO: 67
  REGION 1..32: note = Engineered antibody sequence

SEQ ID NO: 548 - protein, 13 aa, synthetic construct
  REGION 1..13: note = Engineered antibody sequence
LGSYDCSRGDCFV

SEQ ID NO: 549 - protein, 11 aa, synthetic construct
  REGION 1..11: note = Engineered antibody sequence
FGGGTKVEIKR

SEQ ID NO: 550 - protein, 106 aa, synthetic construct; identical to SEQ ID NO: 30
  REGION 1..106: note = Engineered antibody sequence

SEQ ID NO: 551 - DNA, 660 bp, synthetic construct; identical to SEQ ID NO: 391
  misc_feature 1..660: note = Engineered antibody sequence
  source 1..660: mol_type = other DNA

SEQ ID NO: 552 - DNA, 339 bp, synthetic construct; identical to SEQ ID NO: 392
  misc_feature 1..339: note = Engineered antibody sequence
  source 1..339: mol_type = other DNA

SEQ ID NO: 553 - DNA, 66 bp, synthetic construct; identical to SEQ ID NO: 73
  misc_feature 1..66: note = Engineered antibody sequence
  source 1..66: mol_type = other DNA

SEQ ID NO: 554 - DNA, 39 bp, synthetic construct; identical to SEQ ID NO: 394
  misc_feature 1..39: note = Engineered antibody sequence
  source 1..39: mol_type = other DNA

SEQ ID NO: 555 - DNA, 45 bp, synthetic construct; identical to SEQ ID NO: 75
  misc_feature 1..45: note = Engineered antibody sequence
  source 1..45: mol_type = other DNA

SEQ ID NO: 556 - DNA, 21 bp, synthetic construct
  misc_feature 1..21: note = Engineered antibody sequence
  source 1..21: mol_type = other DNA
tctacatccactctggcatct

SEQ ID NO: 557 - DNA, 96 bp, synthetic construct; identical to SEQ ID NO: 77
  misc_feature 1..96: note = Engineered antibody sequence
  source 1..96: mol_type = other DNA

SEQ ID NO: 558 - DNA, 39 bp, synthetic construct; identical to SEQ ID NO: 398
  misc_feature 1..39: note = Engineered antibody sequence
  source 1..39: mol_type = other DNA

SEQ ID NO: 559 - DNA, 33 bp, synthetic construct; identical to SEQ ID NO: 79
  misc_feature 1..33: note = Engineered antibody sequence
  source 1..33: mol_type = other DNA

SEQ ID NO: 560 - DNA, 321 bp, synthetic construct; identical to SEQ ID NO: 40
  misc_feature 1..321: note = Engineered antibody sequence
  source 1..321: mol_type = other DNA

SEQ ID NO: 561 - protein, 37 aa, Homo sapiens
  REGION 1..37: note = C-term amidated
ACDTATCVTHRLAGLLSRSGGVVKNNFVPTNVGSKAF

SEQ ID NO: 562 - protein, 37 aa, Homo sapiens
  REGION 1..37: note = C-term amidated
ACNTATCVTHRLAGLLSRSGGMVKSNFVPTNVGSKAF

SEQ ID NO: 563 - protein, 106 aa, Homo sapiens; identical to SEQ ID NO: 30

SEQ ID NO: 564 - protein, 330 aa, synthetic construct; identical to SEQ ID NO: 10
  REGION 1..330: note = Engineered antibody sequence

SEQ ID NO: 565 - protein, 329 aa, synthetic construct
  REGION 1..329: note = Engineered antibody sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 566 - protein, 440 aa, synthetic construct
  REGION 1..440: note = Engineered antibody sequence
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 567 - DNA, 1323 bp, synthetic construct
  misc_feature 1..1323: note = Engineered antibody sequence
  source 1..1323: mol_type = other DNA
gaggtgcagcttgtggagtctgggggaggcttggtccagcctggggggtccctgagactctcctgtgcagtctctggaatcgacctcagtggctactacatgaactgggtccgtcaggctccagggaaggggctggagtgggtcggagtcattggtattaatggtgccacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacgcgagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

(19) United States
(12) Patent Application Publication
(10) Pub. No.: US 2026/0001942 A1
(43) Pub. Date: Jan. 1, 2026

(54) TREATMENT OF HEADACHE DISORDERS AND/OR PSYCHIATRIC SYMPTOMS USING ANTI-CGRP ANTIBODIES, AND COMPOSITIONS AND METHODS RELATED THERETO

(71) Applicant: H. LUNDBECK A/S, Valby (DK)

(72) Inventors: Bjarke EBERT, Valby (DK); Anders Janusz ETTRUP, Valby (DK); Xin Ying LEE, Valby (DK); Susanne AWAD, Valby (DK); Line Pickering BOSERUP, Valby (DK)

(21) Appl. No.: 19/243,807

(22) Filed: Jun. 20, 2025

Related U.S. Application Data
(60) Provisional application No. 63/662,745, filed on Jun. 21, 2024.

(51) Int. Cl. C07K 16/18; A61K 39/00; A61K 45/06; A61P 25/06; A61P 25/24

(52) U.S. Cl. CPC C07K 16/18; A61K 45/06; A61P 25/06; A61P 25/24; A61K 2039/505; A61K 2039/545; C07K 2317/52; C07K 2317/565; C07K 2317/567

(57) ABSTRACT

Methods for treating or preventing migraine and/or headache are provided comprising the administration of an anti-CGRP antagonist antibody to a migraine patient having one or more psychiatric symptoms such as depressive symptoms and/or suffering from a psychiatric disorder such as depression. Methods of treating or preventing one or more psychiatric symptoms such as depressive symptoms or a psychiatric disorder such as depression are also provided comprising the administration of an anti-CGRP antagonist antibody to a patient in need thereof. Pharmaceutical compositions related to such methods are also provided.

### CROSS-REFERENCE TO RELATED APPLICATIONS

This U.S. nonprovisional application claims priority to U.S. Provisional Appl. No. 63/662,745 filed on Jun. 21, 2024, the contents of which are incorporated by reference in their entirety.

### SEQUENCE LISTING DISCLOSURE

The contents of the electronic sequence listing (1143257o018001.xml; Size: 771,706 bytes; and Date of Creation: Jun. 18, 2025) is herein incorporated by reference in its entirety.

### BACKGROUND

### Field

This invention pertains to treatment methods using antibodies and fragments thereof (including Fab fragments) that specifically bind to human Calcitonin Gene Related Peptide (hereinafter “CGRP”).

### Description of Related Art

Calcitonin Gene Related Peptide (CGRP) is produced as a multifunctional neuropeptide of 37 amino acids in length. Two forms of CGRP, the CGRP-alpha and CGRP-beta forms, exist in humans and have similar activities. CGRP-alpha and CGRP-beta differ by three amino acids in humans, and are derived from different genes. CGRP is released from numerous tissues such as trigeminal nerves, which when activated release neuropeptides within the meninges, mediating neurogenic inflammation that is characterized by vasodilation, vessel leakage, and mast-cell degradation. Durham, P. L., New Eng. J. Med., 350 (11):1073-75 (2004). Biological effects of CGRP are mediated via the CGRP receptor (CGRP-R), which consists of a seven-transmembrane component, in conjunction with receptor-associated membrane protein (RAMP). CGRP-R further requires the activity of the receptor component protein (RCP), which is essential for an efficient coupling to adenylate cyclase through G proteins and the production of cAMP. Doods, H., Curr. Op. Invest. Drugs, 2(9):1261-68 (2001).

Migraines are neurovascular disorder affecting approximately 10% of the adult population in the U.S., and are typically accompanied by intense headaches. CGRP is believed to play a prominent role in the development of migraines. In fact, several companies, i.e., Amgen, Eli Lilly, Teva and Alder Biopharmaceuticals (recently acquired by Lundbeck A/S) have developed anti-CGRP and anti-CGRP-R antibodies for use in treating or preventing migraine headaches. The present assignee has previously filed patent applications related to anti-CGRP antibodies and uses thereof including published PCT Application WO/2012/162243 filed May 21, 2012 entitled “ANTI-CGRP COMPOSITIONS AND USE THEREOF”, published PCT Application WO/2012/162257 filed May 21, 2012, entitled “USE OF ANTI-CGRP ANTIBODIES AND ANTIBODY FRAGMENTS TO PREVENT OR INHIBIT PHOTOPHOBIA OR LIGHT AVERSION IN SUBJECTS IN NEED THEREOF, ESPECIALLY MIGRAINE SUFFERERS” published PCT Application WO/2012/162253, filed May 21, 2012, entitled “USE OF ANTI-CGRP OR ANTI-CGRP-R ANTIBODIES OR ANTIBODY FRAGMENTS TO TREAT OR PREVENT CHRONIC AND ACUTE FORMS OF DIARRHEA” and published PCT Application WO/2015/003122, filed Jul. 3, 2014, entitled “REGULATION OF GLUCOSE METABOLISM USING ANTI-CGRP ANTIBODIES” all of which applications are incorporated by reference in their entirety.

### BRIEF SUMMARY

In one aspect, the present disclosure provides methods for preventing or treating migraine or headache in a subject having one or more psychiatric symptoms.

The method may comprise administering an effective amount of an anti CGRP antibody or antigen-binding fragment thereof to the subject. The method may comprise administering to the subject an effective amount of an anti-CGRP antibody or antigen-binding fragment thereof comprising the 6 CDRs of Ab6 as disclosed in FIGS. 1-12. The method may comprise administering to the subject an effective amount of an anti-CGRP antibody or antigen-binding fragment thereof comprising: (A) a heavy chain variable domain (VH) comprising a CDRH1, a CDRH2, and a CDRH3; and (B) a light chain variable domain (VL) comprising a CDRL1, a CDRL2, and a CDRL3, wherein: (A) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and (B) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively. SEQ ID NO: 208 has the amino acid sequence GDI.

In some embodiments, the one or more psychiatric symptoms may comprise one or more depressive symptoms.

In certain embodiments, the one or more depressive symptoms may comprise or may be any one or more of the depressive symptoms defined by the Diagnostic and Statistical Manual of Mental Disorders, the 5th Edition, Text Revision (DSM-5-TR). In certain embodiments, the one or more depressive symptoms may comprise or may be any one or more of the following: (1) depressed mood (or irritable mood in children and adolescents); (2) diminished interest in pleasure; (3) weight loss when not dieting or weight gain (e.g., a change of more than 5% of body weight in a month), or decrease or increase in appetite nearly every day (or failure to make expected weight gain in children); (4) insomnia or hypersomnia; (5) psychomotor agitation or retardation; (6) fatigue or loss of energy; (7) feelings of worthlessness or excessive or inappropriate guilt; (8) diminished ability to think or concentrate, or indecisiveness; and (9) recurrent thoughts of death, recurrent suicidal ideation without a specific plan, or a suicide attempt or a specific plan for committing suicide. In certain embodiments, the one or more depressive symptoms may comprise or may be one or more selected from the group comprising feelings of sadness, tearfulness, emptiness or hopelessness, angry outbursts, irritability or frustration, loss of interest or pleasure, such as sex, hobbies or sports, sleep disturbances, including insomnia or sleeping too much, tiredness and lack of energy, reduced appetite and weight loss or increased cravings for food and weight gain, anxiety, agitation or restlessness, slowed thinking, speaking or body movements, feelings of worthlessness or guilt, fixating on past failures or self-blame, trouble thinking, concentrating, making decisions and remembering things, frequent or recurrent thoughts of death, suicidal thoughts, suicide attempts or suicide, unexplained physical problems, such as back pain or headaches.

In certain embodiments, the subject may: (I) (i) experiences two to four depressive symptoms: (ii) experiences one or more minor depressive episodes, optionally wherein the occurrence of the minor depressive episodes is recurrent; and/or (iii) has or is diagnosed with minor depressive disorder; or (II) (i) experiences five, six, seven, eight, or more depressive symptoms: (ii) experiences one or more major depressive episodes, optionally wherein the occurrence of the major depressive episodes is recurrent; and/or (iii) has or is diagnosed with major depressive disorder. The depressive symptoms may be selected from the following: (1) depressed mood most of the day, nearly every day, as indicated in the subjective report (e.g., feels sad, empty, hopeless) or in observation made by others (e.g., appears tearful) (In children and adolescents, can be irritable mood); (2) markedly diminished interest in pleasure in all, or almost all, activities most of the day and nearly every day (as indicated by either subjective account or observation); (3) significant weight loss when not dieting or weight gain (e.g., a change of more than 5% of body weight in a month), or decrease or increase in appetite nearly every day. (In children, consider failure to make expected weight gain.); (4) insomnia or hypersomnia nearly every day; (5) psychomotor agitation or retardation nearly every day (observable by others, not merely subjective feelings of restlessness or being slowed down); (6) fatigue or loss of energy nearly every day; (7) feelings of worthlessness or excessive or inappropriate guilt (which may be delusional) nearly every day (not merely self-reproach or guilt about being sick); (8) diminished ability to think or concentrate, or indecisiveness nearly every day (either by subjective account or as observed by others); and (9) recurrent thoughts of death (not just fear of dying), recurrent suicidal ideation without a specific plan, or a suicide attempt or a specific plan for committing suicide. In particular embodiments, the subject may experience at least (1) and/or (2).

In some embodiments, the subject may have or may be diagnosed with one or more of the following psychiatric disorders: depressive disorders, anxiety disorders, mixed anxiety-depressive disorder (MADD), bipolar and related disorders, schizophrenia spectrum or other psychotic disorders, substance-related and addictive disorders, medication-induced movement disorders and other adverse effects of medication, neurocognitive disorders, neurodevelopmental disorders, trauma- and stressor-related disorders, dissociative disorders, obsessive-compulsive and related disorders, personality disorders, somatic symptom and related disorders, feeding and eating disorders, elimination disorders, sleep-wake disorders, sexual dysfunctions, gender dysphoria, disruptive, impulse-control, and conduct disorders, paraphilic disorders, and other mental disorders, optionally wherein the psychiatric disorder is as defined by the DSM-5-TR.

In some embodiments, the subject may have or may be diagnosed with a depressive disorder or mixed anxiety-depressive disorder, optionally wherein the depressive disorder is selected from: major depressive disorder, minor depressive disorder, bipolar depression, mood dysregulation disorder, persistent depressive disorder, premenstrual dysphoric disorder, substance/medication-induced depressive disorder, depressive disorder due to another medical condition, another specified depressive disorder, or an unspecified depressive disorder, dysthymic disorder, emotional disorder (seasonal affective disorder and the like), recurrent depression, postpartum depression, stress disorder, major depressive disorder concomitant with psychosis (including delusive disorders and schizophrenia), manic or mixed mood episode, hypomanic mood episode, depression episode with atypical features, depression episode with melancholic features, depressive episodes with tonic features, depression episode after stroke, delirium, peripheral symptoms of dementia (mental symptoms or behavior abnormalities). In particular embodiments, the depressive disorder may comprise or may be major depressive disorder or minor depressive disorder.

In some embodiments, the subject may have or may be diagnosed with migraine, optionally as defined by the International Classification of Headache Disorders (ICHD-3).

In certain embodiments, the subject may have one or more of chronic migraine, episodic migraine, chronic/episodic migraine, migraines with or without aura, acute migraine or headache, hemiplegic migraines, cluster headaches, migrainous neuralgia, chronic headaches, tension headaches, general headaches, headaches due to an underlying structural problem in the head or neck, sinus headaches (such as for example associated with sinusitis), and allergy-induced headaches or migraines. In particular embodiments, the subject may have chronic migraine or episodic migraine.

In some embodiments, the subject may be administered the anti-CGRP antibody or antigen-binding fragment thereof during and/or on the onset of a depressive episode, optionally a major depressive episode or a minor depressive episode. In certain embodiments, the subject may have been diagnosed with a depressive disorder, an anxiety disorder, a mixed anxiety-depressive disorder (MADD), and/or a bipolar disorder. In certain embodiments, the administration may alleviate the severity and/or duration of the depressive episode and/or the frequency and/or severity of later depressive episodes.

In some embodiments, the subject may have a history of previous failure with one or more preventive migraine medications, optionally two or more preventive migraine medications, further optionally two to four preventive migraine medications.

In certain embodiments, the previous failure may be with one or more preventive migraine medications selected from: (i) antiepileptic drugs or anticonvulsants, optionally divalproex, valproate, topiramate, and carbamazepine; (ii) beta-blockers, optionally propranolol, metoprolol, timolol, atenolol, nadolol, nebivolol, and pindolol; (iii) triptans, optionally frovatriptan, naratriptan, and zolmitriptan; (iv) antidepressants (such as SSRI, SNRI, and TCA), optionally amitriptyline and venlafaxine; (v) calcium channel blocker, optionally flunarizine; (vi) angiotensin II receptor antagonists, optionally candesartan; (vii) ACE inhibitors, optionally lisinopril; (viii) α-agonists, optionally clonidine and guanfacine; (ix) antihistamines, optionally cyproheptadine; and (x) other medications approved for prevention of migraine.

In certain embodiments, the previous failure may be with one or more preventive migraine medications selected from: carbonic anhydrase inhibitors such as acetazolamide; antithrombotics such as coumadin and picotamide; antidepressants (such as TCA, SSRI, and SNRI) such as protriptyline, fluvoxamine, and fluoxetine; antiepileptic drugs such as gabapentin; beta-blockers such as bisoprolol; Ca2+ blockers such as nicardipine, nifedipine, nimodipine, and verapamil; and vascular smooth muscle relaxants such as cyclandelate;

In certain embodiments, the previous failure may be with one or more preventive migraine medications comprising one or more CGRP receptor antagonistic small molecules. In particular embodiments, the one or more CGRP receptor antagonistic small molecules may comprise one or more gepants, optionally atogepant (QULIPTA®) and/or rimegepant (NURTEC® ODT).

In certain embodiments, the previous failure may be with one or more preventive migraine medications comprising one or more anti-CGRP antibodies and/or anti-CGRP receptor antibodies. In particular embodiments, the one or more anti-CGRP antibodies may comprise fremanezumab (AJOVY®) and/or galcanezumab (EMGALITY®). In particular embodiments, the one or more anti-CGRP receptor antibodies may comprise erenumab (AIMOVIG®).

In some embodiments, the subject may be in need of immediate preventative treatment of migraine or headache. In some embodiments, the subject may have at least one headache and/or migraine symptom and/or the one or more psychiatric symptoms at the time of administration. In certain embodiments, the at least one migraine symptom may comprise one or more of pain, nausea, photophobia, phonophobia, or aura. In particular embodiments, the pain may be head pain.

In some embodiments, the subject may not be administered any acute migraine medication (other than the anti-CGRP antibody or antigen-binding fragment thereof) within a period of time before and after the administration of the anti-CGRP antibody or antigen-binding fragment thereof, such as within 15 minutes, within 30 minutes, within 1 hour, within 2 hours, within 3 hours, within 4 hours, within 5 hours, or within 6 hours before and after said administration.

In some embodiments, the subject has migraine and the method may reduce monthly migraine days (MMD) by at least about 3 days, at least about 3.5 days, at least about 4 days, at least about 4.5 days, optionally about 12 weeks or about 24 weeks after the administering.

In some embodiments, the subject has migraine and the method may reduce MMD by at least about 50%, at least about 55%, at least about 60%, at least about 65%, at least about 70%, at least about 75.

In some embodiments, the subject has migraine and the method may ameliorate migraine based on Patient Global Impression of Change (PGIC). In certain embodiments, the subject may report “minimally improved”, “much improved”, or “very much improved” as the PGIC. In particular embodiments, the subject may report “much improved” or “very much improved” as the PGIC.

In some embodiments, the subject has depression or has one or more depressive symptoms and the method may reduce the number of or ameliorates depressive symptoms. In some embodiments, the subject has depression or has one or more depressive symptoms and the method may prevent or treat depression (e.g., based on the DSM-5-TR criteria). In some embodiments, the subject has depression or has one or more depressive symptoms and the method may prevent or treat suicidal ideation by the subject.

In one aspect, the present disclosure provides methods for preventing or treating one or more psychiatric symptoms in a subject. The method may comprise administering to the subject an effective amount of an anti-CGRP antibody or antigen-binding fragment thereof. The method may comprise administering to the subject an effective amount of an anti-CGRP antibody or antigen-binding fragment thereof comprising the 6 CDRs of Ab 6 as disclosed in FIGS. 1-12. The method may comprise administering to the subject an effective amount of an anti-CGRP antibody or antigen-binding fragment thereof comprising: (A) a heavy chain variable domain (VH) comprising a CDRH1, a CDRH2, and a CDRH3; and (B) a light chain variable domain (VL) comprising a CDRL1, a CDRL2, and a CDRL3, wherein: (A) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and (B) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively. SEQ ID NO: 208 has the amino acid sequence GDI.

In some embodiments, the one or more psychiatric symptoms comprises one or more depressive symptoms.

In certain embodiments, the one or more depressive symptoms may comprise or may be any one or more of the depressive symptoms defined by the Diagnostic and Statistical Manual of Mental Disorders, the 5th Edition, Text Revision (DSM-5-TR). In certain embodiments, the one or more depressive symptoms may comprise or may be any one or more of the following: (1) depressed mood (or irritable mood in children and adolescents); (2) diminished interest in pleasure; (3) weight loss when not dieting or weight gain (e.g., a change of more than 5% of body weight in a month), or decrease or increase in appetite nearly every day (or failure to make expected weight gain in children); (4) insomnia or hypersomnia; (5) psychomotor agitation or retardation; (6) fatigue or loss of energy; (7) feelings of worthlessness or excessive or inappropriate guilt; (8) diminished ability to think or concentrate, or indecisiveness; and (9) recurrent thoughts of death, recurrent suicidal ideation without a specific plan, or a suicide attempt or a specific plan for committing suicide. In certain embodiments, the one or more depressive symptoms may comprise or may be any one or more selected from the group comprising feelings of sadness, tearfulness, emptiness or hopelessness, angry outbursts, irritability or frustration, loss of interest or pleasure, such as sex, hobbies or sports, sleep disturbances, including insomnia or sleeping too much, tiredness and lack of energy, reduced appetite and weight loss or increased cravings for food and weight gain, anxiety, agitation or restlessness, slowed thinking, speaking or body movements, feelings of worthlessness or guilt, fixating on past failures or self-blame, trouble thinking, concentrating, making decisions and remembering things, frequent or recurrent thoughts of death, suicidal thoughts, suicide attempts or suicide, unexplained physical problems, such as back pain or headaches.

In some embodiments, the subject may: (I) (i) experience two to four depressive symptoms: (ii) experience one or more minor depressive episodes, optionally wherein the occurrence of the minor depressive episodes is recurrent; and/or (iii) have or be diagnosed with minor depressive disorder; or (II) (i) experience five, six, seven, eight, or more depressive symptoms: (ii) experience one or more major depressive episodes, optionally wherein the occurrence of the major depressive episodes is recurrent; and/or (iii) have or be diagnosed with major depressive disorder. The depressive symptoms may be selected from the following: (1) depressed mood most of the day, nearly every day, as indicated in the subjective report (e.g., feels sad, empty, hopeless) or in observation made by others (e.g., appears tearful) (In children and adolescents, can be irritable mood); (2) markedly diminished interest in pleasure in all, or almost all, activities most of the day and nearly every day (as indicated by either subjective account or observation); (3) significant weight loss when not dieting or weight gain (e.g., a change of more than 5% of body weight in a month), or decrease or increase in appetite nearly every day. (In children, consider failure to make expected weight gain.); (4) insomnia or hypersomnia nearly every day; (5) psychomotor agitation or retardation nearly every day (observable by others, not merely subjective feelings of restlessness or being slowed down); (6) fatigue or loss of energy nearly every day; (7) feelings of worthlessness or excessive or inappropriate guilt (which may be delusional) nearly every day (not merely self-reproach or guilt about being sick); (8) diminished ability to think or concentrate, or indecisiveness nearly every day (either by subjective account or as observed by others); and (9) recurrent thoughts of death (not just fear of dying), recurrent suicidal ideation without a specific plan, or a suicide attempt or a specific plan for committing suicide. In particular embodiments, the subject may experience at least (1) and/or (2).

In some embodiments, the subject may have or may be diagnosed with one or more of the following psychiatric disorders: depressive disorders, anxiety disorders, mixed anxiety-depressive disorder (MADD), bipolar and related disorders, schizophrenia spectrum or other psychotic disorders, substance-related and addictive disorders, medication-induced movement disorders and other adverse effects of medication, neurocognitive disorders, neurodevelopmental disorders, trauma- and stressor-related disorders, dissociative disorders, obsessive-compulsive and related disorders, personality disorders, somatic symptom and related disorders, feeding and eating disorders, elimination disorders, sleep-wake disorders, sexual dysfunctions, gender dysphoria, disruptive, impulse-control, and conduct disorders, paraphilic disorders, and other mental disorders, optionally wherein the psychiatric disorder is as defined by the DSM-5-TR.

In some embodiments, the subject may have or may be diagnosed with a depressive disorder or mixed anxiety-depressive disorder, optionally wherein the depressive disorder is selected from: major depressive disorder, minor depressive disorder, bipolar depression, mood dysregulation disorder, persistent depressive disorder, premenstrual dysphoric disorder, substance/medication-induced depressive disorder, depressive disorder due to another medical condition, another specified depressive disorder, or an unspecified depressive disorder, dysthymic disorder, emotional disorder (seasonal affective disorder and the like), recurrent depression, postpartum depression, stress disorder, major depressive disorder concomitant with psychosis (including delusive disorders and schizophrenia), manic or mixed mood episode, hypomanic mood episode, depression episode with atypical features, depression episode with melancholic features, depressive episodes with tonic features, depression episode after stroke, delirium, peripheral symptoms of dementia (mental symptoms or behavior abnormalities). In particular embodiments, the subject may have or may be diagnosed with major depressive disorder or minor depressive disorder.

In some embodiments, the subject may not have migraine or headache. In some embodiments, the subject may not have or may not be diagnosed with migraine.

In some embodiments, the subject may have migraine or headache. In some embodiments, the subject may have or may be diagnosed with migraine, optionally as defined by the International Classification of Headache Disorders (ICHD-3). In certain embodiments, the subject may have one or more of: chronic migraine, episodic migraine, chronic/episodic migraine, migraine with or without aura, acute migraine or headache, hemiplegic migraines, cluster headaches, migrainous neuralgia, chronic headaches, tension headaches, general headaches, headaches due to an underlying structural problem in the head or neck, sinus headaches (such as for example associated with sinusitis), and allergy-induced headaches or migraines. In particular embodiments, the subject may have chronic migraine, episodic migraine, or chronic/episodic migraine.

In some embodiments, the subject may be administered the anti-CGRP antibody or antigen-binding fragment thereof during and/or on the onset of a depressive episode, optionally a major depressive episode or a minor depressive episode. In certain embodiments, the subject may have been diagnosed with a depressive disorder, an anxiety disorder, a mixed anxiety-depressive disorder (MADD), and/or a bipolar disorder. In certain embodiments, the administration may alleviate the severity and/or duration of the bout of depression and/or the frequency and/or severity of later depressive episodes.

In some embodiments, the method may reduce the number of or ameliorates depressive symptoms. In some embodiments, the method may prevent or treat depression (e.g., based on the DSM-5-TR criteria). In some embodiments, the method may prevent or treat suicidal ideation by the subject.

In some embodiments, the method may further comprise administering to the subject one or more additional medications. In certain embodiments, the one or more additional medications may comprise one or more antidepressants. In certain embodiments, the one or more additional medications may comprise one or more headache or migraine medications. In certain embodiments, the one or more additional medications may be administered to the subject together with the anti-CGRP antibody or antigen-binding fragment thereof. In certain embodiments, the one or more additional medications may be administered to the subject separately from the anti-CGRP antibody or antigen-binding fragment thereof, optionally prior to or after the administration of the anti-CGRP antibody or antigen-binding fragment thereof, further optionally within about 15 minutes, within about 30 minutes, within about 1 hour, within about 2 hours, within about 3 hours, within about 4 hours, within about 5 hours, or within about 6 hours before and/or after the administration of the anti-CGRP antibody or antigen-binding fragment thereof. In certain embodiments, the one or more additional medications and the anti-CGRP antibody or antigen-binding fragment thereof may be contained in the same pharmaceutical composition or in separate pharmaceutical compositions.

In some cases, the one or more antidepressants to be administered may comprise one or more selected from: (i) monoamine oxidase inhibitors (MAOIs), optionally selected from isocarboxazid, nialamide, phenelzine, tranylcypromine, iproniazide, iproclozide, safrazine, monoamine, moclobemide, toloxatone, minaprine, bifemelane, and selegiline; (ii) tricyclic antidepressants (TCAs), optionally selected from imipramine, imipramine oxide, amitriptyline, desipramine, nortriptyline, amoxapine, clomipramine, lofepramine, dosulepin, doxepin, trimipramine, protriptyline, opipramol, dibenzepin, butriptyline, amineptine, iprindole, melitracen, dimetacrine, and quinupramine; (iii) serotonin reuptake inhibitors (SSRIs), optionally selected from fluoxetine, fluvoxamine, paroxetine, sertraline, vilazodone, vortioxetine, citalopram, alaproclate, etoperidone, escitalopram, and imelidine; (iv) serotonin (5HT)-norepinephrine (NE) reuptake inhibitors (SNRIs), optionally selected from milnacipran, venlafaxine, sibutramine, levomilnacipran, duloxetine, desvenlafaxine, and tramadol; (v) tetracyclic antidepressants (TeCAs), optionally selected from maprotiline, mianserin, setiptiline, and mirtazapine; (vi) serotonin antagonist and reuptake inhibitors (SARIs), optionally selected from trazodone, etoperidone, lorpiprazole, mepiprazole, and nefazodone; (vii) N-methyl-D-aspartate (NMDA) antagonists, optionally bupropion; (viii) serotonin-dopamine activity modulators (SDAMs), optionally selected from brexpiprazole and aripiprazole; and (ix) other antidepressants, optionally selected from oxitriptan, tryptophan, nomifensine, viloxazine, oxaflozane, medifoxamine, tianeptine, pivagabine, reboxetine, gepirone, agomelatine, hyperici herba, esketamine, brexanolone, and dextromethorphan.

In some cases, the one or more headache or migraine medications to be administered may comprise one or more selected from: (i) an ergotamine or an ergot derivative, (ii) a triptan, optionally selected from sumatriptan, zolmitriptan, naratriptan, rizatriptan, eletriptan, almotriptan, and/or frovatriptan; (iii) a non-opioid analgesic, optionally selected from; (iv) an opioid/narcotic, optionally selected from oxycodone, tramadol, butorphanol, morphine, codeine, and/or hydrocodone; (v) acetaminophen or paracetamol; (vi) a nonsteroidal anti-inflammatory drug (NSAID), optionally selected from acetylsalicylic acid (aspirin), celecoxib, choline salicylate, diclofenac, diflunisal, etodolac, fenoprofen, flurbiprofen sodium, ibuprofen, indomethacin, ketoprofen, ketorolac tromethamine, meclofenamate, meloxicam, nabumetone, naproxen, oxaprozin, phenylbutazone, piroxicam, rofecoxib, salsalate, sulindac, tolmetin, valdecoxib, and/or zomepirac; (vii) a combination medication, optionally selected from: a combination of two or more drugs with analgesic effects (for example, paracetamol and codeine); a combination of an analgesic and an adjuvant (for example, paracetamol and caffeine); a combination which comprises at least one opioid (such as oxycodone, tramadol, butorphanol, morphine, codeine, hydrocodone, or any combination thereof); a combination which comprises at least one barbiturate such as butalbital; a combination which comprises at least caffeine; and/or a combination which comprises acetylsalicylic acid (aspirin), paracetamol, and caffeine (e.g., EXCEDRIN®, EXCEDRIN MIGRAINE®); (viii) CGRP receptor antagonistic small molecules, optionally gepants, further optionally ubrogepant (UBRELVY®), atogepant (QULIPTA®), rimegepant (NURTEC® ODT), zavegepant (ZAVZPRET™); and/or (ix) another anti-CGRP antibody and/or an anti-CGRP receptor antibody.

In some embodiments, (A) the amino acid sequence of the VH may have at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 202; and/or (B) the amino acid sequence of the VL may have at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 222.

In some embodiments, (A) the amino acid sequence of the VH may comprise or consist of SEQ ID NO: 202; and/or (B) the amino acid sequence of the VL may comprise or consist of SEQ ID NO: 222.

In some embodiments, the anti-CGRP antibody or antigen-binding fragment thereof may comprise a scFv fragment, a diabody, a minibody, a scFv-Fc, a Fab fragment, a Fab′ fragment, a Fab′-SH fragment, a F(ab′)2 fragment, a small modular immunopharmaceuticals (SMIPs), and/or a Fv fragment.

In some embodiments, the anti-CGRP antibody or antigen-binding fragment thereof may comprise: (A) a heavy chain polypeptide comprising (i) the VH and (ii) a heavy chain constant region (CH); and (B) a light chain polypeptide comprising (i) the VL and (ii) a light chain constant region (CL).

In certain embodiments, the anti-CGRP antibody or antigen-binding fragment thereof may comprise: an immunoglobulin molecule comprised of two said heavy chain polypeptides and two said light chain polypeptides interconnected by disulfide bonds, or a multimer of said immunoglobulin molecule. In particular embodiments, the anti-CGRP antibody or antigen-binding fragment thereof may comprise or may be an IgG, IgA, IgD, IgE, or IgM antibody, further optionally an IgG1, IgG2, IgG3, IgG4, IgA1, or IgA2 antibody.

In certain embodiments, (A) the amino acid sequence of the heavy chain polypeptide may comprise or consist of SEQ ID NO: 201 or SEQ ID NO: 566; and/or (B) the amino acid sequence of the light chain polypeptide may comprise or consist of SEQ ID NO: 221.

In some embodiments, the effective amount (of the anti-CGRP antibody or antigen-binding fragment thereof) may be about 100 mg or about 300 mg. In some embodiments, the effective amount may be about 100 to about 300 mg, about 100 to about 500 mg, about 50 to about 1000 mg, or about 50 to about 1500 mg. In some embodiments, the effective amount may be about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, about 1000 mg, about 1100 mg, about 1200 mg, about 1300 mg, about 1400 mg, or about 1500 mg.

In some embodiments, the anti-CGRP antibody or antigen-binding fragment thereof may be administered intravenously. In certain embodiments, the anti-CGRP antibody or antigen-binding fragment thereof may be infused over a period of approximately 30 min to 60 minutes.

In some embodiments, the method may further comprise intravenously administering an effective amount of the anti-CGRP antibody or antigen-binding fragment thereof about every 10-14 weeks. In certain embodiments, the method may further comprise intravenously administering an effective amount of the anti-CGRP antibody or antigen-binding fragment thereof about every 11-13 weeks. In particular embodiments, the method may further comprise intravenously administering an effective amount of the anti-CGRP antibody or antigen-binding fragment thereof about every 12 weeks or about every three months. In some cases, the total administration (or the number of dosing) may be at least twice. In certain cases, the total administration (or the number of dosing) may be at least three times, four times, five time, six times, seven times, eight times, or more. In certain embodiments, the effective amount (of the anti-CGRP antibody for each of the repeated doses) may be about 100 mg to about 300 mg. In particular embodiments, the effective amount (of the anti-CGRP antibody for each of the repeated doses) may be the about 100 mg or about 300 mg.

In some embodiments, the anti-CGRP antibody or antigen-binding fragment thereof may be comprised in a formulation comprising histidine (L-histidine and/or L-histidine monohydrate), sorbitol, polysorbate 80, and water. In certain embodiments, the formulation may comprise, per 1 mL volume, about 100 mg or about 300 mg anti-CGRP antibody, about 3.1 mg Histidine, about 40.5 mg Sorbitol, and about 0.15 mg Polysorbate 80, and may have a pH of about 5.8.

In some embodiments, the anti-CGRP antibody or antigen-binding fragment thereof may be expressed in or obtained by expression in Pichia pastoris.

In some embodiments, the anti-CGRP antibody or antigen-binding fragment thereof may be expressed in or obtained by expression in CHO cells.

The present disclosure also encompasses any of the anti-CGRP antibodies and antigen-binding fragments thereof disclosed herein, including but not limited to those comprising the 6 CDRs of Ab6, or a pharmaceutical composition containing such an anti-CGRP antibody or antigen-binding fragment thereof and a pharmaceutically acceptable carrier, for use in preventing or treating migraine or headache in a subject having one or more psychiatric symptoms. In some embodiments, the use may be according to any of the methods for preventing or treating migraine or headache in a subject having one or more psychiatric symptoms as described above and/or herein.

The present disclosure also encompasses any of the anti-CGRP antibodies and antigen-binding fragments thereof disclosed herein, including but not limited to those comprising the 6 CDRs of Ab6, or a pharmaceutical composition containing such an anti-CGRP antibody or antigen-binding fragment thereof and a pharmaceutically acceptable carrier, for use in preventing or treating one or more psychiatric symptoms in a subject. In some embodiments, the use may be according to any one of the methods for preventing or treating one or more psychiatric symptoms as described above and/or herein.

The present disclosure further encompasses use of any of the anti-CGRP antibodies and antigen-binding fragments thereof disclosed herein, including but not limited to those comprising the 6 CDRs of Ab6, or a pharmaceutical composition containing such an anti-CGRP antibody or antigen-binding fragment thereof and a pharmaceutically acceptable carrier, for the manufacture of a medicament for preventing or treating migraine or headache in a subject having one or more psychiatric symptoms. In some embodiments, the medicament may be used according to any one of the methods for preventing or treating migraine or headache in a subject having one or more psychiatric symptoms as described above and/or herein.

The present disclosure further encompasses use of any of the anti-CGRP antibodies and antigen-binding fragments thereof disclosed herein, including but not limited to those comprising the 6 CDRs of Ab6, or a pharmaceutical composition containing such an anti-CGRP antibody or antigen-binding fragment thereof and a pharmaceutically acceptable carrier, for the manufacture of a medicament for preventing or treating one or more psychiatric symptoms in a subject. In some embodiments, the medicament may be used according to any one of the methods for preventing or treating one or more psychiatric symptoms as described above and/or herein.

The present disclosure further encompasses use of any of the anti-CGRP antibodies and antigen-binding fragments thereof disclosed herein, including but not limited to those comprising the 6 CDRs of Ab6, or a pharmaceutical composition containing such an anti-CGRP antibody or antigen-binding fragment thereof and a pharmaceutically acceptable carrier, for preventing or treating migraine or headache in a subject having one or more psychiatric symptoms. The use may be according to any one of the methods for preventing or treating migraine or headache in a subject having one or more psychiatric symptoms as described above and/or herein.

The present disclosure further encompasses use of any of the anti-CGRP antibodies and antigen-binding fragments thereof disclosed herein, including but not limited to those comprising the 6 CDRs of Ab6, or a pharmaceutical composition containing such an anti-CGRP antibody or antigen-binding fragment thereof and a pharmaceutically acceptable carrier, for preventing or treating one or more psychiatric symptoms in a subject. The use may be used according to any of the methods for preventing or treating one or more psychiatric symptoms as described above and/or herein.

In another aspect, the present disclosure provides pharmaceutical compositions comprising: (I) an effective amount of an anti-CGRP antibody or antigen-binding fragment thereof; (II) one or more antidepressants; and (III) a pharmaceutically acceptable carrier. The anti-CGRP antibody or antigen-binding fragment thereof may comprise the 6 CDRs of Ab6 as disclosed in FIGS. 1-12. The method may comprise: (A) a heavy chain variable domain (VH) comprising a CDRH1, a CDRH2, and a CDRH3; and (B) a light chain variable domain (VL) comprising a CDRL1, a CDRL2, and a CDRL3, wherein: (A) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and (B) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively. SEQ ID NO: 208 has the amino acid sequence GDI

In some embodiments, the one or more antidepressants may comprise one or more selected from: (i) monoamine oxidase inhibitors (MAOIs), optionally selected from isocarboxazid, nialamide, phenelzine, tranylcypromine, iproniazide, iproclozide, safrazine, monoamine, moclobemide, toloxatone, minaprine, bifemelane, and selegiline; (ii) tricyclic antidepressants (TCAs), optionally selected from imipramine, imipramine oxide, amitriptyline, desipramine, nortriptyline, amoxapine, clomipramine, lofepramine, dosulepin, doxepin, trimipramine, protriptyline, opipramol, dibenzepin, butriptyline, amineptine, iprindole, melitracen, dimetacrine, and quinupramine; (iii) serotonin reuptake inhibitors (SSRIs), optionally selected from fluoxetine, fluvoxamine, paroxetine, sertraline, vilazodone, vortioxetine, citalopram, alaproclate, etoperidone, escitalopram, and imelidine; (iv) serotonin (5HT)-norepinephrine (NE) reuptake inhibitors (SNRIs), optionally selected from milnacipran, venlafaxine, sibutramine, levomilnacipran, duloxetine, desvenlafaxine, and tramadol; (v) tetracyclic antidepressants (TeCAs), optionally selected from maprotiline, mianserin, setiptiline, and mirtazapine; (vi) serotonin antagonist and reuptake inhibitors (SARIs), optionally selected from trazodone, etoperidone, lorpiprazole, mepiprazole, and nefazodone; (vii) N-methyl-D-aspartate (NMDA) antagonists, optionally bupropion; (viii) serotonin-dopamine activity modulators (SDAMs), optionally selected from brexpiprazole and aripiprazole; and (ix) other antidepressants, optionally selected from oxitriptan, tryptophan, nomifensine, viloxazine, oxaflozane, medifoxamine, tianeptine, pivagabine, reboxetine, gepirone, agomelatine, hyperici herba, esketamine, brexanolone, and dextromethorphan.

In some embodiments, (A) the amino acid sequence of the VH may have at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 202; and/or (B) the amino acid sequence of the VL may have at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 222.

In some embodiments, (A) the amino acid sequence of the VH may comprise or consist of SEQ ID NO: 202; and/or (B) the amino acid sequence of the VL may comprise or consist of SEQ ID NO: 222.

In some embodiments, the anti-CGRP antibody or antigen-binding fragment thereof may comprise a scFv fragment, a diabody, a minibody, a scFv-Fc, a Fab fragment, a Fab′ fragment, a Fab′-SH fragment, a F(ab′)2 fragment, a small modular immunopharmaceuticals (SMIPs), and/or a Fv fragment.

In some embodiments, the anti-CGRP antibody or antigen-binding fragment thereof may comprise: (A) a heavy chain polypeptide comprising (i) the VH and (ii) a heavy chain constant region (CH); and (B) a light chain polypeptide comprising (i) the VL and (ii) a light chain constant region (CL). In certain embodiments, the anti-CGRP antibody or antigen-binding fragment thereof may comprise: an immunoglobulin molecule comprised of two said heavy chain polypeptides and two said light chain polypeptides interconnected by disulfide bonds, or a multimer of said immunoglobulin molecule. In particular embodiments, the anti-CGRP antibody or antigen-binding fragment thereof may comprise an IgG, IgA, IgD, IgE, or IgM antibody, optionally an IgG1, IgG2, IgG3, IgG4, IgA1, or IgA2 antibody.

In some embodiments, (A) the amino acid sequence of the heavy chain polypeptide may comprise or consist of SEQ ID NO: 201 or SEQ ID NO: 566; and/or (B) the amino acid sequence of the light chain polypeptide may comprise or consist of SEQ ID NO: 221.

In some embodiments, the effective amount (of the anti-CGRP antibody or antigen-binding fragment thereof) may be about 100 mg or about 300 mg. In some embodiments, the effective amount (of the anti-CGRP antibody or antigen-binding fragment thereof) may be about 100 to about 300 mg, about 100 to about 500 mg, about 50 to about 1000 mg, or about 50 to about 1500 mg. In some embodiments, the effective amount (of the anti-CGRP antibody or antigen-binding fragment thereof) may be about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, about 1000 mg, about 1100 mg, about 1200 mg, about 1300 mg, about 1400 mg, or about 1500 mg.

The present disclosure also encompasses any of the pharmaceutical compositions disclosed above and/or herein for use in preventing or treating one or more psychiatric symptoms in a subject. In some embodiments, the use may be according to any one of the methods for preventing or treating one or more psychiatric symptoms as described above and/or herein.

The present disclosure further encompasses any of the pharmaceutical compositions disclosed above and/or herein for the manufacture of a medicament for preventing or treating one or more psychiatric symptoms in a subject. In some embodiments, the medicament may be used according to any one of the methods for preventing or treating one or more psychiatric symptoms as described above and/or herein.

The present disclosure further encompasses any of the pharmaceutical compositions disclosed above and/or herein for preventing or treating one or more psychiatric symptoms in a subject. In some embodiments, the use is according to any one of the methods for preventing or treating one or more psychiatric symptoms as described above and/or herein.

### BRIEF DESCRIPTION OF THE DRAWINGS

FIGS. 1A-1F provide the polypeptide sequences of the full-length heavy chain for antibodies Ab1-Ab14 with their framework regions (FR), complementarity determining regions (CDRs), and constant region sequences delimited.

FIGS. 2A-2D provide the polypeptide sequences of the full-length light chain for antibodies Ab1-Ab14 with their framework regions (FR), complementarity determining regions (CDRs), and constant region sequences delimited.

FIGS. 3A-3P provide exemplary polynucleotide sequences encoding the full-length heavy chain for antibodies Ab1-Ab14 with their framework regions (FR), complementarity determining regions (CDRs), and constant region coding sequences delimited.

FIGS. 4A-4I provide exemplary polynucleotide sequences encoding the full-length light chain for antibodies Ab1-Ab14 with their framework regions (FR), complementarity determining regions (CDRs), and constant region coding sequences delimited.

FIG. 5 provides the polypeptide sequence coordinates within the full-length heavy chain polypeptide sequences of antibodies Ab1-Ab14 of sequence features including the variable region and complementarity determining regions (CDRs), and the SEQ ID NO of each individual feature.

FIG. 6 provides the polypeptide sequence coordinates within the full-length heavy chain polypeptide sequences of antibodies Ab1-Ab14 of sequence features including the framework regions (FRs) and constant region, and the SEQ ID NO of each individual feature.

FIG. 7 provides the polypeptide sequence coordinates within the full-length light chain polypeptide sequences of antibodies Ab1-Ab14 of sequence features including the variable region and complementarity determining regions (CDRs), and the SEQ ID NO of each individual feature.

FIG. 8 provides the polypeptide sequence coordinates within the full-length light chain polypeptide sequences of antibodies Ab1-Ab14 of sequence features including the framework regions (FRs) and constant region, and the SEQ ID NO of each individual feature.

FIG. 9 provides the polynucleotide sequence coordinates within the exemplary polynucleotide sequences encoding the full-length heavy chain polypeptide sequences of antibodies Ab1-Ab14 of sequence features including the variable region and complementarity determining regions (CDRs), and the SEQ ID NO of each individual feature.

FIG. 10 provides the polynucleotide sequence coordinates within the exemplary polynucleotide sequences encoding the full-length heavy chain polypeptide sequences of antibodies Ab1-Ab14 of sequence features including the framework regions (FRs) and constant region, and the SEQ ID NO of each individual feature.

FIG. 11 provides the polynucleotide sequence coordinates within the exemplary polynucleotide sequences encoding the full-length light chain polypeptide sequences of antibodies Ab1-Ab14 of sequence features including the variable region and complementarity determining regions (CDRs), and the SEQ ID NO of each individual feature.

FIG. 12 provides the polynucleotide sequence coordinates within the exemplary polynucleotide sequences encoding the full-length light chain polypeptide sequences of antibodies Ab1-Ab14 of sequence features including the framework regions (FRs) and constant region, and the SEQ ID NO of each individual feature.

FIG. 13A provides exemplary results showing mean changes in MMD from baseline in eptinezumab (100 mg and 300 mg doses combined) and placebo groups for the total patient population, patient population with ≥1 psychiatric comorbidity (“Any psychiatric comorbidity), and patient population with depressive symptoms at baseline (“Depressive symptoms”). As shown in the graph (left), across populations, mean changes from baseline in MMDs were greater with eptinezumab than with placebo. As shown in the tables (right), the differences from placebo increased from Weeks 1-12 to 13-24 cross populations and were largest in the subgroup with depressive symptoms, followed by the subgroup with any psychiatric comorbidity.

FIG. 13B provides exemplary results showing mean changes MMD in eptinezumab (100 mg and 300 mg doses combined) and placebo groups for the patient population with ≥1 psychiatric comorbidity (“Any psychiatric comorbidity”). In the subgroup reporting ≥1 psychiatric comorbidity, there were fewer MMD with eptinezumab treatment than with placebo, with improvements in MMD frequency observed from Weeks 1-4 through Weeks 21-24.

FIG. 13C provides exemplary results showing mean changes from baseline in MMD in patients with depressive symptoms at baseline (“Depressive symptoms” subgroup) with previous treatment failure with amitriptyline who received epitunezumab (100 mg and 300 mg doses combined). Epitunezumab treatment reduced the mean MMD by 6.37 days in Weeks 1-12, by 7.54 days in Weeks 13-24, and by 7.32 days in Weeks 25-72.

FIG. 14 provides exemplary results showing ≥50% MRR, i.e., % of patients who showed ≥50% reduction in MMD, for the total patient population, patient population with ≥1 psychiatric comorbidity (“Any psychiatric comorbidity), and patient population with depressive symptoms at baseline (“Depressive symptoms”). As shown in the graph (left), across patient populations, ≥50% MRRs were numerically higher with eptinezumab than with placebo. As shown in the tables (right), the odds ratios of response with eptinezumab versus placebo were ≥4-fold across time points and patient populations. The odds ratios of response with eptinezumab versus placebo were largest in the population with depressive symptoms, followed by the population with any psychiatric comorbidity.

FIG. 15 provides exemplary results showing % PGIC responders, i.e., % of patients who reported “much improved” or “very much improved” in the PGIC test, at Weeks 12 and 24 for the total patient population, patient population with ≥1 psychiatric comorbidity (“Any psychiatric comorbidity), and patient population with depressive symptoms at baseline (“Depressive symptoms”). As shown in the graph (left), PGIC responder rates were generally consistent across populations, with eptinezumab having a numerically higher response than placebo. As shown in the tables (right), the odds ratios of response with eptinezumab versus placebo were ≥5-fold across time points and patient populations. The odds ratios of response with eptinezumab versus placebo were largest in the population with depressive symptoms, followed by the population with any psychiatric comorbidity, at Weeks 12 and 24, and were higher at Week 24 than Week 12 in both the population with depressive symptoms and the population with any psychiatric comorbidity.

### DETAILED DESCRIPTION

In the present invention SEQ ID NO 208 is disclosing the CDR 3 region on the heavy chain which consists of the amino acids GDI.

Several antibodies and small molecules which block the CGRP pathway have been clinically used for preventing and/or treating migraine.

Applicant surprisingly discovered that Applicant's anti-CGRP antibodies are capable of preventing/treating migraine in patients with psychiatric comorbidity including patients with depressive symptoms, which have been considered hard-to-treat patients. Furthermore, it was surprisingly discovered that the effects are particularly pronounced in migraine patients with psychiatric comorbidity, particularly in patients with depressive symptoms.

Applicant further surprisingly discovered that Applicant's anti-CGRP antibodies appear to reduce psychiatric symptoms and/or depression. Psychiatric symptoms such as depressive symptoms are among the adverse effects observed upon administration of CGRP blocking therapies (e.g., anti-CGRP antibodies and gepants), and such adverse effects often lead to patients' decision to discontinue the use of the therapies (see e.g., https://mdedge.com/migraine-icymi/article/254294/headache-migraine/safety-concems-cgrp-monoclonal-antibodies; and Ashina et al., Headache. 2023 January; 63(1):79-88.). Despite such observations by others, patients treated with Applicant's anti-CGRP antibody surprisingly experienced reduced psychiatric symptoms and/or depression.

Use of anti-CGRP antibodies for preventing or treating headache and/or migraine is described herein. Use of anti-CGRP antibodies for preventing or treating psychiatric symptoms such as depressive symptoms is also described herein. Additionally, pharmaceutical compositions comprising at least an anti-CGRP antibody, which may be used for precenting or treating psychiatric symptoms such as depressive symptoms are also provided.

### Definitions

It is to be understood that this invention is not limited to the particular methodology, protocols, cell lines, animal species or genera, and reagents described, as such may vary. It is also to be understood that the terminology used herein is for the purpose of describing particular embodiments, only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth.

As used herein, the term “about” or “approximately” when used in reference to a particular recited numerical value, means that the value may vary from the recited value by no more than 10%. For example, as used herein, the expression “about 100” includes 90 and 110 and all values in between (e.g., 91, 92, 93, 99, 99.1, 99.2, 99.3, 99.4, 100, 100.8, 100.9, 101, 106, 107, 108, 109, etc.).

It is understood that aspects and embodiments of the disclosure described herein include “comprising,” “consisting,” and “consisting essentially of” aspects and embodiments. Transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

By “comprising,” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g., poly-adenylation sequence, enhancer elements, other genes, linker domains, etc. The terms “including”, “includes”, “having”, “has”, “with”, or variants thereof used herein are intended to be inclusive in a manner similar to the term “comprising”.

By “consisting essentially of,” it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g., linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g., 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of,” it is meant the exclusion from the composition, method, or kit of any element, step, or ingredient not specified in the claim. For example, a polypeptide or polypeptide domain “consisting of” a recited sequence contains only the recited sequence.

All technical and scientific terms used herein have the same meaning as commonly understood to one of ordinary skill in the art to which this invention belongs unless clearly indicated otherwise.

As used herein, the term “reduction in migraine prevalence” refers to a reduction (e.g., a stated percentage reduction, such as 50%) in the likelihood of a patient having a migraine in the stated period, such as the 18 hour, 20 hour, 24 hour, 28 hour, or 30 hour period, preferably the 24 hour period, after a first dosage of an antibody, or on the first day following the day of antibody administration (i.e., on the first full day following the day on which the antibody administration is completed). It is to be understood that a given patient may or may not have a migraine during that period, as the reduction in likelihood may be observable over a large number of patients irrespective of the outcome for an individual patient.

As used herein, the term “diagnosed with” in relation to a given disease or condition in a subject or patient refers to a patient meeting the clinical criteria for the disease or condition, whether or not a formal diagnosis of that subject or patient was performed.

As used herein, the term “intravenously administering” refers to a mode of administration wherein a substance, e.g., an antibody, is introduced directly into the circulation of that patient, most typically into the venous circulation. The substance may be introduced in a carrier fluid, such as an aqueous solution, e.g., normal saline. The substance may be administered in a single formulation or in multiple formulations, as long as the administration is completed over a short period of time (e.g., within 1 day, preferably within 12 hours, more preferably within 6 hours, and most preferably within 1-2 hours).

As used herein, the term “immediate relief” is intended to mean a relief in headache or migraine symptoms in a patient, e.g., headache or migraine symptoms associated with an acute migraine or chronic/episodic migraine or another headache or migraine condition associated with frequent headache or migraine episodes, wherein said relief of symptoms is experienced rapidly or immediately after anti-CGRP antibody treatment, e.g., relief of one or more symptoms is experienced by the patient within a short time period post-infusion with Ab6, such as within minutes or a few hours, such as within 10 minutes, 20 minutes, 30 minutes, 60 minutes, 1 hour, 2 hours or 6 hours, up to e.g. a day.

As used herein, the term “immediate preventative treatment” is intended to mean prevention of headache or migraine symptoms in a patient, e.g., prevention of headache or migraine symptoms associated with an acute migraine or chronic/episodic migraine or another headache or migraine condition. In this context, “immediate preventative treatment” refers to the prophylactic treatment of a subject who is at risk of developing migraine or headache, resulting in a decrease in the probability that the subject will develop headache or migraine. Typically, due to a patient history of headache or migraine episodes, there is a high risk of a new headache or migraine episode in the patient. Typically, the prevention of symptoms is experienced rapidly or immediately after anti-CGRP antibody treatment, e.g., prevention of one or more symptoms is experienced by the patient within a short time period post-infusion with Ab6, such as within minutes or a few hours, such as within 10 minutes, 20 minutes, 30 minutes, 60 minutes, 1 hour, 2 hours or 6 hours, up to e.g. a day.

Calcitonin Gene Related Peptide (CGRP): As used herein, CGRP encompasses not only the following Homo sapiens CGRP-alpha and Homo sapiens CGRP-beta amino acid sequences available from American Peptides (Sunnyvale CA) and Bachem (Torrance, CA):
CGRP-alpha: ACDTATCVTHRLAGLLSRSGGVVKNNFVPTNVGSKAF-NH₂ (SEQ ID NO: 561), wherein the terminal phenylalanine is amidated;
CGRP-beta: ACNTATCVTHRLAGLLSRSGGMVKSNFVPTNVGSKAF-NH₂ (SEQ ID NO: 562), wherein the terminal phenylalanine is amidated; but also any membrane-bound forms of these CGRP amino acid sequences, as well as mutants (muteins), splice variants, isoforms, orthologs, homologues and variants of this sequence.

The term “antibody” is used herein in the broadest sense and encompasses various antibody or immunoglobulin structures and/or fragments thereof (preferably those fragments that exhibit the desired antigen-binding activity, which are referred to as “antigen-binding antibody fragments”).

An antibody may include a polypeptide chain-containing molecular structure with a specific shape that fits to and recognizes an epitope, where one or more non-covalent binding interactions stabilize the complex between the molecular structure and the epitope. An antibody comprises an antigen-binding region comprising at least one variable domain, typically (with some exceptions, e.g., in case of nanobody which only has one VH and no VL) a pair of a heavy chain variable domain (VH) and a light chain variable domain (VL).

Each VH and VL polypeptide is composed of three complementarity-determining regions (CDRs) and four framework regions (FRs), arranged from amino-terminus to carboxy-terminus in the following order: FR1, CDR1, FR2, CDR2, FR3, CDR3, FR4. The CDRs in a VH (a heavy chain) are designated “CDRH1,” “CDRH2,” and “CDRH3,” respectively, and the CDRs in a VL (or a light chain) are designated “CDRL1,” “CDRL2,” and “CDRL3.” Therefore, “a set of heavy chain CDRs” (or the like) of an antibody means the set or combination of the CDRH1, CDRH2, and CDRH3 of the antibody, “a set of light chain CDRs” (or the like) of an antibody means the set or combination of the CDRL1, CDRL2, and CDRL3 of the antibody, and “a set of heavy and light chain CDRs” (or the like) of an antibody means the set or combination of the CDRH1, CDRH2, CDRH3, CDRL1, CDRL2, and CDRL3 of the antibody. The FRs in a heavy chain are designated “FRH1,” “FRH2,” “FR3,” and “FRH4,” respectively, and the FRs in a light chain are designated “FRL1,” “FRL2,” “FRL3,” and “FRL4.”

The terms “intact antibody,” “full length antibody,” and “whole antibody” or the like are used herein interchangeably and refer to an antibody having a structure substantially similar to a native antibody.

The general structure of antibodies in vertebrates now is well understood (Edelman, G. M., Ann. N.Y. Acad. Sci., 190: 5 (1971)). Antibodies in vertebrates typically comprise two pairs (e.g., in case of IgG, IgE, IgD) of a light chain polypeptide of molecular weight approximately 23,000 Daltons (the “light chain”) and a heavy chain polypeptide of molecular weight 53,000-70,000 (the “heavy chain”), or multimers thereof (e.g., dimer of such two pairs in case of IgA, and pentamer of such two pairs in case of IgM). The four chains (in such two pairs) are joined by disulfide bonds in a “Y” configuration wherein the light chains bracket the heavy chains starting at the mouth of the “Y” configuration. The “branch” portion of the “Y” configuration is designated the F_{ab} region; the stem portion of the “Y” configuration is designated the Fc region. The amino acid sequence orientation runs from the N-terminal end at the top of the “Y” configuration to the C-terminal end at the bottom of each chain. The N-terminal end possesses a variable region having specificity for the antigen that elicited it, and is approximately 100 amino acids in length, there being slight variations between light and heavy chain and from antibody to antibody.

In intact antibodies, each heavy chain comprises a variable domain, such as a heavy chain variable domain (VH) (also referred to as heavy chain variable region), and a heavy chain constant region (CH). In case of an intact antibody, a CH comprises a heavy chain constant domain 1 (CH1), a hinge, a heavy chain constant domain 2 (CH2), and a heavy chain constant domain 3 (CH3), with some exceptions, e.g., a camel IgG2 or camel IgG3, in which a CH comprises a hinge, a CH2, and a CH3, or a shark antibody (e.g., IgNAR), in which a CH comprises a hinge, a CH1, a CH2, a CH3, a CH4, and a CH5. In an intact antibody, each light chain is comprised of: a light chain variable domain (VL); and a light chain constant domain (CL). Typically (with some exceptions such as nanobodies, camelid heavy chain antibodies, IgNARs, and the like), one VH and one VL may form an antigen-binding structure.

The variable region is linked in each chain to a constant region that extends the remaining length of the chain and that within a particular class of antibody does not vary with the specificity of the antibody (i.e., the antigen eliciting it).

There are five known major classes of heavy chain constant regions (CH) that determine the class of the immunoglobulin molecule (IgG, IgM, IgA, IgD, and IgE corresponding to γ, μ, α, δ, and ε (gamma, mu, alpha, delta, or epsilon) heavy chain constant regions). Several of these classes may be further divided into subclasses (isotypes), e.g., IgG1, IgG2, IgG3, IgG4, IgA1, and IgA2. The constant region or class/subclass determines subsequent effector function of the antibody, including activation of complement (Kabat, E. A., Structural Concepts in Immunology and Immunochemistry, 2nd Ed., p. 413-436, Holt, Rinehart, Winston (1976)), and other cellular responses (Andrews, D. W., et al., Clinical Immunobiology, pp 1-18, W. B. Sanders (1980); Kohl, S., et al., Immunology, 48: 187 (1983)); while the variable region determines the antigen with which it will react. Light chains are classified as either κ (kappa) or λ (lambda). Each heavy chain class can be prepared with either kappa or lambda light chain. The light and heavy chains are covalently bonded to each other, and the “tail” portions of the two heavy chains are bonded to each other by covalent disulfide linkages when the immunoglobulins are generated either by hybridomas or by B cells.

A “Fc region” is a C-terminal region of an immunoglobulin heavy chain that contains at least a portion of the constant region, including native sequence Fc regions and variant Fc regions. A human IgG heavy chain Fc region can extend from Asp221 to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, as described in Kabat et al., Sequences of Proteins of Immunological Interest, 5th Ed. Public Health Service, National Institutes of Health, Bethesda, Md., 1991.

The phrase “effector function” of an antibody refers to biological activities attributable to the Fc region of an antibody, which varies by antibody class or isotype. Exemplary effector functions include: complement (e.g., C1q) binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

There are two major CL isotypes, kappa (κ) and lambda (λ), and such CL domains are referred to herein as kappa CL domain (CLκ domain) and lambda CL domain (CLλ domain).

The expressions “complementarity determining region,” “hypervariable region,” or “CDR” refer to one or more of the hyper-variable or complementarity determining regions (CDRs) found in the variable regions of light or heavy chains of an antibody (See Kabat, E. A. et al., Sequences of Proteins of Immunological Interest, National Institutes of Health, Bethesda, Md., (1987)). These expressions include the hypervariable regions as defined by Kabat et al. (“Sequences of Proteins of Immunological Interest,” Kabat E., et al., US Dept. of Health and Human Services, 1983) or the hypervariable loops in 3-dimensional structures of antibodies (Chothia and Lesk, J Mol. Biol. 196 901-917 (1987)). The CDRs in each chain are held in close proximity by framework regions and, with the CDRs from the other chain, contribute to the formation of the antigen binding site. Within the CDRs there are select amino acids that have been described as the selectivity determining regions (SDRs) which represent the critical contact residues used by the CDR in the antibody-antigen interaction (Kashmiri, S., Methods, 36:25-34 (2005)).

In general, CDRs may be determined using any appropriate manner. For example, CDR sequences may be: determined based on an appropriate numbering system (e.g., Kabat numbering or IMGT numbering, or alternatively Chothia numbering, Martin numbering (also known as AbM numbering), Gelfand numbering, Honneger's numbering (also known as AHo's numbering), or Chemical Computing Group (CCG) numbering for variable regions; determined structurally (see e.g., IgBlast (https://www.ncbi.nlm.nih.gov/igblast/); Dondelinger et al., Front Immunol. 2018 Oct. 16; 9:2278; http://www.bioinf.org.uk/abs/info.html#cdrid; http://opig.stats.ox.ac.uk/webapps/newsabdab/sabpred/anarci/), e.g., by using x-ray crystallography to visualize specific epitope contact residues; or a combination thereof. In the present invention when specific antibody amino acid or nucleic acid residues of CDRs are referenced by number, unless otherwise specified, this generally refers to its position within a specified amino acid or nucleic acid sequence (i.e., particular sequence identifier) and/or in accordance with Kabat et al numbering.

The expressions “framework region” or “FR” refer to one or more of the framework regions within the variable regions of the light and heavy chains of an antibody (See Kabat, E. A. et al., Sequences of Proteins of Immunological Interest, National Institutes of Health, Bethesda, Md., (1987)). These expressions include those amino acid sequence regions interposed between the CDRs within the variable regions of the light and heavy chains of an antibody.

Unless otherwise specified, the numbering of amino acid residues in antibody constant regions and constant domains may be performed by the EU-index or EU numbering system, as described in Kabat et al., Sequences of Proteins of Immunological Interest, 5th Ed. Public Health Service, National Institutes of Health, Bethesda, Md. (1991). The EU numbering system is used in the present specification unless otherwise specified.

An “antigen-binding fragment of an antibody” or “antigen-binding antibody fragment” includes any naturally occurring, enzymatically obtainable, synthetic (e.g., domain orders are rearranged relative to naturally occurring antibody), or genetically engineered polypeptide or protein or glycoprotein that comprises an antigen-binding antibody domain (e.g., a VH domain, or VH and VL domains) specifically binds an antigen to form a complex. Exemplary antigen-binding antibody fragments include, but are not limited to: Fv; fragment antigen-binding (“Fab”) fragment; single-chain Fab (scFab); Fab′ fragment; Fab′ containing a free sulfhydryl group (‘Fab′-SH’); F(ab′)₂ fragment; F(ab′)₃ fragment; diabody; triabody; tetrabody; linear antibodies; single-chain antibody molecules (e.g. single-chain variable fragment (“scFv”), tandem scFv; scFv-Fc; scFv-CH (e.g., scFv-CH3, scFv-CH2, scFv-CH1, scFv-CH2/CH3, scFv-CH1/CH2, scFv-CH1/CH3, scFv-CH1/CH2/CH3, scFv-CH1/hinge/CH2/CH3, minibody, or the like); scFv-zipper; tandem scFv-Fc; tandem scFv-CH (e.g., tandem scFv-CH3, tandem scFv-CH2, tandem scFv-CH1, tandem scFv-CH2/CH3, tandem scFv-CH1/CH2, tandem scFv-CH1/CH3, tandem scFv-CH1/CH2/CH3, tandem scFv-CH1/hinge/CH2/CH3, or the like); tandem scFv-zipper; diabody-Fc; diabody-CH (e.g., diabody-CH3, diabody-CH2, diabody-CH1, diabody-CH2/CH3, diabody-CH1/CH2, diabody-CH1/CH3, diabody-CH1/CH2/CH3, diabody-CH1/hinge/CH2/CH3, or the like); diabody-zipper; single domain antibody (sdAb); nanobody or VHH (i.e., VH domain only and no VL); tandem sdAb or tandem nanobody; sdAb-Fc or nanobody-Fc; sdAb-CH or nanobody-CH (e.g., sdAb-CH3, sdAb-CH2, sdAb-CH1, sdAb-CH2/CH3, sdAb-CH1/CH2, sdAb-CH1/CH3, sdAb-CH1/CH2/CH3, sdAb-CH1/hinge/CH2/CH3, or the like); a heavy chain-only antibody (HCAb); camel Ig; immunoglobulin new antigen receptor (IgNAR); “half-antibody” variant of any of the foregoing (when the structure allows); and monospecific or multispecific antibodies formed from one or more of antibody fragments such as the foregoing. Further exemplary antigen-binding antibody fragments include, but are not limited to: minimal recognition units consisting of the amino acid residues that mimic the hypervariable region of an antibody (e.g., an isolated complementarity determining region (CDR) such as a CDR3 peptide), or a constrained FR3-CDR3-FR4 peptide; and small modular immunopharmaceuticals (SMIPs). In some embodiments, antibodies (e.g., multispecific antibodies) described herein comprise one or more Fabs, scFvs, scFabs, sdAbs, and/or IgGs.

For a review of certain antibody fragments, see, e.g., Hudson et al. Nat. Med. 9:129-134 (2003). For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “scFv,” “single-chain Fv,” or “single-chain variable fragment” refers to a fusion protein comprising at least one VH and at least one VL of an antibody, wherein the VH and the VL are contiguously linked and wherein the scFv retains the specificity of the antibody from which it is derived (the antibody from which the VH and the VL are derived). Unless specified, as used herein an scFv may have the VH and the VL in either order, e.g., with respect to the N-terminal and C-terminal ends of the polypeptide. For example, the VH and the VL may be linked via a linker, such as a synthetic and/or flexible polypeptide linker, and the scFv may be capable of being expressed as a single chain polypeptide. When a linker connects the VH and the VL, a scFv may comprise the structure of VL-linker-VH or VH-linker-VL, in a direction from the N-terminus to the C-terminus. When a linker connects the VH and the VL, the linker may be any appropriate linker such as but not limited to any of the linkers described herein. In some cases, the VH and the VL are additionally or alternatively connected by one or more disulfide bonds. In certain cases, the VH and/or the VL sequences may be modified (e.g., one or more amino acids may be substituted) to comprise a cysteine residue to allow for such a disulfide bond (e.g., see Weatherill et al., Protein Eng Des Sel. 2012 July; 25(7):321-9.). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

A “half-antibody” variant of a given antibody or antigen-binding antibody fragment as used herein refers to an antigen-binding antibody structure which comprises a symmetrical half or an essentially symmetrical half of the given antibody or antigen-binding antibody fragment. Therefore, when the given antibody or antigen-binding fragment is a homodimer (or an essentially homodimer), the half-antibody variant thereof comprises the corresponding monomer. For example, a half-antibody variant of an intact IgG comprises one (but not two) set of a heavy chain comprising a VH, a CH1, a hinge, a CH2, and a CH3 and a light chain comprising a VL and a CL. Similarly, a half-antibody variant of a scFv-Fc comprises one (but not two) polypeptide comprising a scFv, a hinge (or a linker), a CH2, and a CH3, and a half-antibody variant of a minibody comprises one (but not two) polypeptide comprising a scFv, a hinge (or a linker), and a CH3. Since a half-antibody variant of a scFv-Fc and a half-antibody variant of a minibody are both single-chain molecules, they may also be referred to as a single-chain variant of a scFv-Fc and a single-chain variant of a minibody, respectively. Further similarly, a half-antibody variant of a camel Ig (or camel IgG, such as camel IgG2 or camel IgG3) comprises one (but not two) polypeptide comprising a VH, a hinge (or a linker), a CH2, and a CH3, and a half-antibody variant of an IgNAR comprises one (but not two) polypeptide comprising a VH, a hinge (or a linker), a CH1, a CH2, a CH3, a CH4, and a CH5. Since a half-antibody variant of a camel Ig and a half-antibody variant of an IgNAR are both single-chain molecules, they may also be referred to as a single-chain variant of a camel Ig and a single-chain variant of an IgNAR, respectively.

A “humanized antibody” refers to an antibody comprising amino acid residues from non-human CDRs and amino acid residues from human FRs. In certain embodiments, a humanized antibody will comprise substantially all of at least one, and typically two, variable domains, in which all or substantially all of the (e.g., CDRs) correspond to those of a non-human antibody, and all or substantially all of the FRs correspond to those of a human antibody. A humanized antibody optionally may comprise at least a portion of an antibody constant region derived from a human antibody. A humanized form of an antibody, e.g., a non-human antibody, refers to an antibody that has undergone humanization.

Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward in practice. See, e.g., U.S. Pat. No. 6,187,287, incorporated fully herein by reference.

In addition to entire immunoglobulins (or their recombinant counterparts), immunoglobulin fragments comprising the epitope binding site (e.g., Fab′, F(ab′)₂, or other fragments) may be synthesized. “Fragment,” or minimal immunoglobulins may be designed utilizing recombinant immunoglobulin techniques. For instance “Fv” immunoglobulins for use in the present invention may be produced by synthesizing a fused variable light chain region and a variable heavy chain region. Combinations of antibodies are also of interest, e.g. diabodies, which comprise two distinct Fv specificities. In another embodiment of the invention, SMIPs (small molecule immunopharmaceuticals), camelbodies, nanobodies, and IgNAR are encompassed by immunoglobulin fragments.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

The term “polynucleotide” or “nucleic acid” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment described herein that is a polynucleotide encompasses both the double-stranded form and each of two complementary single-stranded forms known or predicted to make up the double-stranded form.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

A polynucleotide or polypeptide has a certain percent “sequence identity” to another polynucleotide or polypeptide, meaning that, when aligned, that percentage of bases or amino acids are the same when comparing the two sequences. The terms “identical” or “identity” when used in the context of two or more nucleic acids or polypeptide sequences, refer to the number or percentage of residues that are the same in a sequence of interest and a reference sequence. The percentage can be calculated by optimally aligning the sequence of interest to the reference sequence; comparing the two sequences over the entire length of the reference sequence; determining the number of positions at which the identical amino acid residue or nucleic acid base occurs in both sequences to yield the number of matched positions; dividing the number of matched positions by the total number of positions in the reference sequence adjusted by adding the number of gap positions introduced into the reference sequence in generating the alignment; and multiplying the result by 100 to yield the percentage of sequence identity. When comparing DNA and RNA, thymine (T) and uracil (U) can be considered equivalent. Sequence identity may be determined by using the stand-alone executable BLAST engine program for blasting two sequences (bl2seq), which can be retrieved from the National Center for Biotechnology Information (NCBI) ftp site or over the worldwide web at ncbi.nlm.nih.gov/BLAST/, using the default parameters (Tatusova and Madden, FEMS Microbiol Lett., 1999, 174, 247-250; which is incorporated herein by reference in its entirety).

“Conservative amino acid substitutions” are known in the art and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a β-branched side-chain substituted for another amino acid with a 0-branched side-chain (e.g., Ile, Thr, and Val), an amino acid with an aromatic side-chain substituted for another amino acid with an aromatic side chain (e.g., His, Phe, Trp, and Tyr), etc.

The term “native” or “wild-type” as used herein refers to a nucleotide sequence, e.g., gene, or gene product, e.g., RNA or protein, that is present in a wild-type cell, tissue, organ or organism. The term “variant” as used herein in relation to a polynucleotide or polypeptide refers to a mutant of a reference polynucleotide or polypeptide sequence, for example a native or wild-type polynucleotide or polypeptide sequence, i.e., having less than 100% sequence identity with the reference polynucleotide or polypeptide sequence. Put another way, a variant polypeptide or polynucleotide comprises at least one amino acid difference (e.g., amino acid substitution, amino acid insertion, and amino acid deletion) or at least one nucleic acid different (e.g., base substitution, base insertion, and base deletion), respectively, relative to a reference polypeptide or polynucleotide sequence, e.g., a native or wild-type, polypeptide or polynucleotide sequence. For example, a variant polypeptide or polynucleotide may be a polypeptide or polynucleotide having a sequence identity of 50% or more, 60% or more, or 70% or more to a full-length native or wild-type, polypeptide or polynucleotide sequence, e.g., an identity of 75% or 80% or more, such as 85%, 90%, or 95% or more, for example, 98% or 99% identity with the full-length native or wild-type, polypeptide or polynucleotide sequence. Variants may also include variant fragments of a reference, e.g., native, sequence sharing a sequence identity of 70% or more with a fragment of the reference, e.g., native, sequence, e.g., an identity of 75% or 80% or more, such as 85%, 90%, or 95% or more, for example, 98% or 99% identity to the native or wild-type sequence.

The term “substantial identity”, or “substantially identical,” when referring to a nucleic acid or fragment thereof, or an amino acid sequence or a fragment thereof, indicates that, when optimally aligned with appropriate insertions or deletions with another nucleic acid sequence or amino acid sequence, as the case may be, (or its complementary strand), there is sequence identity in at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80%, and optionally so on, as measured by any well-known algorithm of sequence identity, such as FASTA, BLAST or GAP.

The term “recombinant” generally refers to any protein, polypeptide, or cell expressing a gene of interest that is produced by genetic engineering methods. Therefore, the term “recombinant” as used with respect to a protein or polypeptide, means a protein or polypeptide produced by expression of a recombinant polynucleotide. “Recombinant,” as applied to a polynucleotide means that the polynucleotide is the product of various combinations of cloning, restriction or ligation steps, and other procedures that result in a construct that is distinct from a polynucleotide found in nature.

A polynucleotide sequence “corresponds” to a polypeptide sequence if translation of the polynucleotide sequence in accordance with the genetic code yields the polypeptide sequence (i.e., the polynucleotide sequence “encodes” the polypeptide sequence), one polynucleotide sequence “corresponds” to another polynucleotide sequence if the two sequences encode the same polypeptide sequence.

A “heterologous” region or domain of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A “coding sequence” is an in-frame sequence of codons that (in view of the genetic code) correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide. A polyadenylation signal and transcription termination sequence will usually be located 3′ to the coding sequence. A “promoter sequence” is a DNA regulatory region capable of binding RNA polymerase in a cell and initiating transcription of a downstream (3′ direction) coding sequence. Promoter sequences typically contain additional sites for binding of regulatory molecules (e.g., transcription factors) which affect the transcription of the coding sequence. A coding sequence is “under the control” of the promoter sequence or “operatively linked” to the promoter when RNA polymerase binds the promoter sequence in a cell and transcribes the coding sequence into mRNA, which is then in turn translated into the protein encoded by the coding sequence.

A “vectors” is a compound or a composition of matter which comprises an isolated nucleic acid and which can be used to introduce a foreign substance, such as DNA, RNA or protein, into an organism or host cell. Numerous vectors are known in the art including, but not limited to, linear polynucleotides, polynucleotides associated with ionic or amphiphilic compounds, plasmids, viruses, and virus-like particles (VLPs). Therefore, the term “vector” encompasses expression vectors, an autonomously replicating plasmid, a self-replicating RNA, and/or viral particles. A vector may be a DNA vector or an RNA vector. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like. Typical vectors include recombinant viruses (for polynucleotides) and liposomes (for polypeptides). A “DNA vector” may be a replicon, such as plasmid, phage or cosmid, to which another polynucleotide segment may be attached so as to bring about the replication of the attached segment.

An “expression vector” is a polynucleotide-based vector designed for gene expression in cells and contains regulatory sequences which will direct polypeptide synthesis by an appropriate host cell. In case of a DNA vector, this usually means a promoter to bind RNA polymerase and initiate transcription of mRNA, as well as ribosome binding sites and initiation signals to direct translation of the mRNA into a polypeptide(s). Incorporation of a polynucleotide sequence into an expression vector at the proper site and in correct reading frame, followed by transformation of an appropriate host cell by the vector, enables the production of a polypeptide encoded by said polynucleotide sequence. “Amplification” of polynucleotide sequences is the in vitro production of multiple copies of a particular nucleic acid sequence. The amplified sequence is usually in the form of DNA. A variety of techniques for carrying out such amplification are described in a review article by Van Brunt (1990, Bio/Technol., 8(4):291-294). Polymerase chain reaction or PCR is a prototype of nucleic acid amplification, and use of PCR herein should be considered exemplary of other suitable amplification techniques.

The term “recombinant cell” or “host cell” refers to cells into which an exogenous nucleic acid sequence has been introduced, including the progeny of such cells. Such cells include transformants and transformed cells, which include the primary transformed cell and progeny derived therefrom without regard to the number of passages.

A “pharmaceutical composition” refers to a chemical or biological composition suitable for administration to a mammal. Such compositions may be specifically formulated for administration via one or more of a number of routes, including but not limited to buccal, epicutaneous, epidural, inhalation, intraarterial, intracardial, intracerebroventricular, intradermal, intramuscular, intranasal, intraocular, intraperitoneal, intraspinal, intrathecal, intravenous, oral, parenteral, rectally via an enema or suppository, subcutaneous, subdermal, sublingual, transdermal, and transmucosal, preferably intravenous. In addition, administration can occur by means of injection, powder, liquid, gel, drops, or other means of administration. A “pharmaceutical excipient” or a “pharmaceutically acceptable excipient” is a carrier, usually a liquid, in which an active therapeutic agent is formulated. In one embodiment of the invention, the active therapeutic agent is a humanized antibody described herein, or one or more fragments thereof.

The excipient generally does not provide any pharmacological activity to the formulation, though it may provide chemical and/or biological stability, and release characteristics. Exemplary formulations can be found, for example, in Remington's Pharmaceutical Sciences, 19^{th} Ed., Grennaro, A., Ed., 1995 which is incorporated by reference. As used herein “pharmaceutically acceptable carrier” or “excipient” includes any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, or sublingual administration. Pharmaceutically acceptable carriers include sterile aqueous solutions or dispersions and sterile powders for the extemporaneous preparation of sterile injectable solutions or dispersions. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

An “effective amount” of an antibody disclosed herein or a composition (e.g., pharmaceutical composition), is at least the minimum amount required to achieve the desired therapeutic or prophylactic result, e.g., a measurable improvement or prevention of a particular disorder, e.g., cancer, preferably with minimal or no toxic or detrimental effects. An effective amount may vary according to inter alia disease state, age, sex, and weight of the patient, and the ability of the antibody (or antigen-binding fragment thereof) to elicit a desired response in the individual and, in some instances, by co-administering one or more additional therapeutic agents. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved. An “effective amount” may be ascertainable by one skilled in the art using known techniques (see, for example, Lloyd (1999) The Art, Science and Technology of Pharmaceutical Compounding).

The terms “individual,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

As used herein, the term “treat,” “treatment,” or “treating” generally refers to the clinical procedure for reducing or ameliorating the progression, severity, and/or duration of a disease, a disorder, or a condition, or for ameliorating one or more conditions or symptoms (preferably, one or more discernible ones) of a disease, a disorder, or a condition. The type of disease or condition to be treated may be, for example, but are not limited to, cancer and cancer-associated diseases and conditions. In specific embodiments, the effect of the “treatment” may be evaluated by the amelioration of at least one measurable physical parameter of a disease, resulting from the administration of one or more therapies (e.g., an anti-CD28 antibody or antigen-binding antibody fragment, a multispecific antibody having specificity to CD28). The parameter may be, for example, gene expression profiles, the mass of disease-affected tissues, inflammation-associated markers, cancer-associated markers, the number or frequency of disease-associated cells, tumor burden, the presence or absence of certain cytokines or chemokines or other disease-associated molecules, and may not necessarily be discernible by the patient. In other embodiments “treat”, “treatment,” or “treating” may result in the inhibition of the progression of a disease, either physically by, e.g., stabilization of a discernible symptom, physiologically by, e.g., stabilization of a physical parameter, or both. In other embodiments the terms “treat”, “treatment” and “treating” refer to the reduction or stabilization of diseased tissue (e.g., cancerous tissues, tumors) or cells (e.g., cancer cells). Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

As used herein, “delaying progression” of a disorder or disease means to defer, hinder, slow, retard, stabilize, and/or postpone development of the disease or disorder (e.g., a cell proliferative disorder, e.g., cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

As used herein, the terms “ameliorate” and “alleviate” refer to a reduction or diminishment in the severity a condition or any symptoms thereof.

The term “EC₅₀” refers to the “half maximal effective concentration”, which value measures the effectiveness of compound (e.g. an anti-CD28 bonding domain-comprising antibody) towards a biological or biochemical utility. This quantitative measure indicates the quantity or concentration required for a particular compound or antibody to elicit a given biological process to half of the maximal response.

“Cmax” refers to the maximum (or peak) concentration that an antibody or other compound achieves in tested area (e.g., in the serum or another compartment such as cerebrospinal fluid) after the drug has been administered. For example, serum Cmax may be measured from serum, e.g., prepared by collecting a blood sample, allowing it to clot and separating solid components by centrifugation or other means to yield serum (blood containing neither blood cells nor clotting factors), and then detecting the concentration of the analyte in the serum by ELISA or other means known in the art.

“AUC” refers to the area under the concentration-time curve which is expressed in units of mg/mL*hr (or equivalently mg*hr/ml) unless otherwise specified. “AUC₀₋ₜ” refers to the area under the concentration-time curve from time=0 to last quantifiable concentration. “AUC_{0-inf}” refers to the area under the concentration-time curve from time=0 extrapolated to infinity.

“Iₘₐₓ” refers to the maximal pharmacodynamic response elicited by an anti-CGRP antibody dosage, preferably a dosage of 350 mg or more, more typically at least 750 or 1000 mg, as compared to the response elicited by lower anti-CGRP antibody doses, e.g., wherein such response may be detected by the inhibition of vasodilation after topical application of capsaicin.

The term “opioid analgesic” herein refers to all drugs, natural or synthetic, with morphine-like actions. The synthetic and semi-synthetic opioid analgesics are derivatives of five chemical classes of compound: phenanthrenes; phenylheptylamines; phenylpiperidines; morphinans; and benzomorphans, all of which are within the scope of the term. Exemplary opioid analgesics include codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine or pharmaceutically acceptable salts thereof.

The term “NSAID” refers to a non-steroidal anti-inflammatory compound. NSAIDs are categorized by virtue of their ability to inhibit cyclooxygenase. Cyclooxygenase 1 and cyclooxygenase 2 are two major isoforms of cyclooxygenase and most standard NSAIDs are mixed inhibitors of the two isoforms. Most standard NSAIDs fall within one of the following five structural categories: (1) propionic acid derivatives, such as ibuprofen, naproxen, naprosyn, diclofenac, and ketoprofen; (2) acetic acid derivatives, such as tolmetin and slindac; (3) fenamic acid derivatives, such as mefenamic acid and meclofenamic acid; (4) biphenylcarboxylic acid derivatives, such as diflunisal and flufenisal; and (5) oxicams, such as piroxim, sudoxicam, and isoxicam. Another class of NSAID has been described which selectively inhibit cyclooxygenase 2. Cox-2 inhibitors have been described, e.g., in U.S. Pat. Nos. 5,616,601; 5,604,260; 5,593,994; 5,550,142; 5,536,752; 5,521,213; 5,475,995; 5,639,780; 5,604,253; 5,552,422; 5,510,368; 5,436,265; 5,409,944; and 5,130,311, all of which are hereby incorporated by reference. Certain exemplary COX-2 inhibitors include celecoxib (SC-58635), DUP-697, flosulide (CGP-28238), meloxicam, 6-methoxy-2 naphthylacetic acid (6-NINA), rofecoxib, MK-966, nabumetone (prodrug for 6-MNA), nimesulide, NS-398, SC-5766, SC-58215, T-614; or combinations thereof.

“Gepants” as used herein refers to a group of small-molecule, CGRP receptor antagonists. UBRELVY® (ubrogepant) is an oral tablet approved by the FDA for the acute treatment of migraine with or without aura in adults. QULIPTA® (atogepant) is an oral tablet approved by the FDA for the preventive treatment of episodic migraine in adults. NURTEC® ODT (rimegepant) is an oral tablet approved by the FDA for the acute treatment of migraine with or without aura in adults and for the prevention of episodic migraine in adults. ZAVZPRET™ (zavegepant) is a nasal spray approved by the FDA for the acute treatment of migraine with or without aura in adults.

### Migraine

Migraine as used herein may be or may comprise for example any of the migraine types as defined in the International Classification of Headache Disorders (ICHD-3) (see Headache Classification Committee of the International Headache Society (IHS), The International Classification of Headache Disorders, 3rd edition, Cephalalgia. 2018 January; 38(1):1-211, which is hereby incorporated by reference in its entirety).

According to the ICHD-3, a patient with migraine without aura may have the following: A) at least 5 attacks fulfilling criteria B-D; B) headache attacks lasting 4-72 hours (when untreated or unsuccessfully treated); C) headache has at least two of the following characteristics, 1) unilateral location, 2) pulsating quality, 3) moderate or severe pain intensity, and/or 4) aggravation by or causing avoidance of routine physical activity (e.g., walking or climbing); and D) during headache at least one of the following, 1) nausea and/or vomiting and/or 2) photophobia and phonophobia. The attacks may not be better accounted for by another ICHD-3 diagnosis.

According to the ICHD-3, a patient with migraine with aura may have the following: A) at least 2 attacks fulfilling criteria B and C; B) one or more of the following fully reversible aura symptoms, 1) visual, 2) sensory, 3) speech and/or language, 4) motor, 5) brainstem, 6) retinal; C) at least three of the following six characteristics, 1) at least one aura symptom spreads gradually over ≥5 minutes, 2) two or more aura symptoms occur in succession, 3) each individual aura symptom lasts 5-60 minutes, 4) at least one aura symptom is unilateral, 5) at least one aura symptom is positive, and/or 6) the aura is accompanies, or followed within 60 minutes, by headache. The attacks may not be better accounted for by another ICHD-3 diagnosis.

According to the ICHD-3, a patient with chronic migraine may have the following: A) headache (migraine-like or tension-type-like) occurring on ≥15 days/month for >3 months, and fulfilling criteria B and C; B) occurring in a patient who has had at least five attacks fulfilling criteria B)-D) for “migraine without aura” and/or criteria B) and C) for “migraine with aura”; and C) on ≥8 days/month for >3 months, fulfilling any of the following, 1) criteria C) and D) for “migraine without aura”, and 2) criteria B) and C) for “migraine with aura”, and 3) believed by the patient to be migraine at onset and relieved by a triptan or ergot derivative. The symptoms may not be better accounted for by another ICHD-3 diagnosis.

A patient with episodic migraine may have the following: A) headache (migraine-like or tension-type-like) on <15 days/month, and fulfilling criteria B and C; B) occurring in a patient who has had at least five attacks fulfilling criteria B)-D) for“migraine without aura” and/or criteria B) and C) for “migraine with aura”; and C) on ≥8 days/month for >3 months, fulfilling any of the following, 1) criteria C) and D) for “migraine without aura”, and 2) criteria B) and C) for “migraine with aura”, and 3) believed by the patient to be migraine at onset and relieved by a triptan or ergot derivative.

### Migraine Preventive Medications

Preventive therapy is a crucial to migraine management.

Medications classically used for migraine prevention include but not are not limited to: antiepileptic drugs or anticonvulsants such as divalproex, valproate, topiramate, and carbamazepine; beta-blockers such as propranolol, metoprolol, timolol, atenolol, nadolol, nebivolol, and pindolol; triptans such as frovatriptan, naratriptan, and zolmitriptan; antidepressants (such as SSRI, SNRI, and TCA) such as amitriptyline and venlafaxine; calcium channel blocker such as flunarizine; angiotensin II receptor antagonists such as candesartan; ACE inhibitors such as lisinopril; α-agonists such as clonidine and guanfacine; antihistamines such as cyproheptadine; and medications locally approved for prevention of migraine.

In some embodiments, a subject may have prior preventive treatment failures with one or more of such migraine preventive medications. In certain embodiments, a subject may have prior preventive treatment failures with two or more of such migraine preventive medications. In particular embodiments, a subject may have prior preventive treatment failures with 2, 3, or 4 of such migraine preventive medications.

Additional medications which have been used for migraine prevention include but not are not limited to: carbonic anhydrase inhibitors such as acetazolamide; antithrombotics such as coumadin and picotamide; antidepressants (such as TCA, SSRI, and SNRI) such as protriptyline, fluvoxamine, and fluoxetine; antiepileptic drugs such as gabapentin; beta-blockers such as bisoprolol; Ca2+ blockers such as nicardipine, nifedipine, nimodipine, and verapamil; and vascular smooth muscle relaxants such as cyclandelate.

In some embodiments, a subject may have prior preventive treatment failures with any one or more of the above-mentioned migraine preventive medications. In certain embodiments, a subject may have prior preventive treatment failures with two or more of the above-mentioned migraine preventive medications. In particular embodiments, a subject may have prior preventive treatment failures with 2, 3, or 4 of the above-mentioned migraine preventive medications.

Certain small molecules which antagonizes CGRP receptor have also been used as migraine preventive medications. Such small molecules include: atogepant (QULIPTA®) and rimegepant (NURTEC® ODT).

In some embodiments, a subject may have prior preventive treatment failures with one or more of gepants used for preventing migraine.

Antibodies which target the CGRP pathway are a new class of migraine preventive medications. Such antibodies include anti-CGRP antibodies and anti-CGRP receptor antibodies. Exemplary anti-CGRP antibodies may include: eptinezumab (VYEPTI®), fremanezumab (AJOVY®), and galcanezumab (EMGALITY®). Exemplary anti-CGRP receptor antibodies may include erenumab (AIMOVIG®).

In some embodiments, a subject may have prior preventive treatment failures with one or more of fremanezumab (AJOVY®), galcanezumab (EMGALITY®), and erenumab (AIMOVIG®).

### Acute Medications for Migraine

Migraine may also be treated acutely.

Medications classically used for acute or symptomatic treatment of migraine include but not are not limited to: ergotamine or ergot derivatives; triptans; non-opioid analgesics; acetaminophen or paracetamol; opioids/narcotics; nonsteroidal anti-inflammatory drugs (NSAIDs); and combination medications. Additional medications used for acute or symptomatic treatment of migraine include certain CGRP blockers. While some of the acute medications (e.g., triptans) are specifically for migraine, many acute medications (e.g., NSAIDS) may also be useful for non-migraine headaches.

In some embodiments, a subject may not be administered an acute migraine medication before (e.g., about 15 minutes, about 30 minutes, about 1, 2, 3, 4, 5, or 6 hours before) or after (e.g., about 15 minutes, about 30 minutes, about 1, 2, 3, 4, 5, or 6 hours after) administration of an anti-CGRP antibody.

In some embodiments, a subject may be administered an acute migraine medication in addition to an anti-CGRP antibody.

In certain embodiments, the acute migraine medication may be ergotamine or ergot derivatives.

In certain embodiments, the acute migraine medication may be triptans. In particular embodiments, the triptan may comprise sumatriptan, zolmitriptan, naratriptan, rizatriptan, eletriptan, almotriptan, and/or frovatriptan.

In certain embodiments, the acute migraine medication may be non-opioid analgesics. In certain embodiments, the acute migraine medication may be acetaminophen or paracetamol.

In certain embodiments, the acute migraine medication may be opioids/narcotics. In particular embodiments, the opioids may comprise sumatriptan, zolmitriptan, naratriptan, rizatriptan, eletriptan, almotriptan, and/or frovatriptan.

In certain embodiments, the acute migraine medication may be nonsteroidal anti-inflammatory drugs (NSAIDs). In particular embodiments, the NSAIDs may comprise acetylsalicylic acid (aspirin), celecoxib, choline salicylate, diclofenac, diflunisal, etodolac, fenoprofen, flurbiprofen sodium, ibuprofen, indomethacin, ketoprofen, ketorolac tromethamine, meclofenamate, meloxicam, nabumetone, naproxen, oxaprozin, phenylbutazone, piroxicam, rofecoxib, salsalate, sulindac, tolmetin, valdecoxib, and/or zomepirac.

In certain embodiments, the acute migraine medication may comprise combination medications. The combination may comprise any one or more of the acute migraine medication categories, e.g., a combination comprising an opioid, a combination comprising a barbiturate, a combination comprising an analgesic and an adjuvant, etc. In particular embodiments, the combination may comprise paracetamol and codeine. In particular embodiments, the combination may comprise acetylsalicylic acid (aspirin), paracetamol, and caffeine.

In certain embodiments, the acute migraine medication may be a CGRP blocker such as a gepant. In particular embodiments, the gepant may comprise ubrogepant (e.g., UBRELVY®), Rimegepant (e.g., NURTEC® ODT), and/or zavegepant (ZAVZPRET™).

### Psychiatric Disorders and Psychiatric Symptoms

A psychiatric disorder and/or a psychiatric symptom as used herein may be or may comprise for example any of the mental disorders or symptoms thereof as defined in the Diagnostic and Statistical Manual of Mental Disorders, the 5th Edition (DSM-5) (American Psychiatric Association. Diagnostic and Statistical Manual of Mental Disorders. 5th ed. Washington D.C.: 2013.), or the Diagnostic and Statistical Manual of Mental Disorders, the 5th Edition, Text Revision (DSM-5-TR) (American Psychiatric Association. Diagnostic and Statistical Manual of Mental Disorders, 5th ed., Text Revision. Washington D.C.: 2022.).

In some embodiments, a psychiatric disorder may comprise any one or more of the following: depressive disorders, anxiety disorders, mixed anxiety-depressive disorder (MADD), bipolar and related disorders, schizophrenia spectrum or other psychotic disorders, substance-related and addictive disorders, medication-induced movement disorders and other adverse effects of medication, neurocognitive disorders, neurodevelopmental disorders, trauma- and stressor-related disorders, dissociative disorders, obsessive-compulsive and related disorders, personality disorders, somatic symptom and related disorders, feeding and eating disorders, elimination disorders, sleep-wake disorders, sexual dysfunctions, gender dysphoria, disruptive, impulse-control, and conduct disorders, paraphilic disorders, and other mental disorders. In some embodiments, a psychiatric symptom may comprise one or more symptoms of such a psychiatric disorder.

In certain embodiments, a psychiatric disorder may comprise one or more of a depressive disorder, such as major depressive disorder, major depressive episode, minor depressive disorder, minor depressive episode, mood dysregulation disorder, persistent depressive disorder, premenstrual dysphoric disorder, substance/medication-induced depressive disorder, depressive disorder due to another medical condition, or another specified depressive disorder or an unspecified depressive disorder. In certain embodiments, a psychiatric symptom may comprise one or more symptoms of such a depressive disorder. In particular embodiments, a psychiatric disorder may comprise major depressive disorder, major depressive episode, minor depressive disorder, and/or minor depressive episode. In particular embodiments, a psychiatric symptom may comprise one or more symptoms of major depressive disorder, major depressive episode, minor depressive disorder, and/or minor depressive episode.

In certain embodiments, a psychiatric disorder may comprise an anxiety disorder, such as but not limited to: generalized anxiety disorder, substance/medication-induced anxiety disorder, anxiety disorder due to another medical condition, separation anxiety disorder, selective mutism, specific phobia, social anxiety disorder, panic disorder, panic attack, agoraphobia, other specified anxiety disorder, or unspecified anxiety disorder. In certain embodiments, a psychiatric symptom may comprise one or more symptoms of such an anxiety disorder.

In certain embodiments, a psychiatric disorder may comprise mixed anxiety-depressive disorder (MADD). A MADD patient may suffer from both anxiety and depressive symptoms of limited and equal intensity accompanied by at least some autonomic features.

In certain embodiments, a psychiatric disorder may comprise a bipolar disorder or a related disorder, such as but not limited to: bipolar I disorder, bipolar II disorder, substance/medication-induced bipolar and related disorder, bipolar and related disorder due to another medical condition, cyclothymic disorder, another specified bipolar and related disorder, unspecified bipolar or related disorder, or an unspecified mood disorder. In certain embodiments, a psychiatric symptom may comprise one or more symptoms of such a bipolar disorder.

In certain embodiments, a psychiatric symptom may comprise schizophrenia spectrum and/or another psychotic disorder, such as but not limited to; delusional disorder, brief psychotic disorder, schizophreniform disorder, schizophrenia, schizoaffective disorder, substance/medication-induced psychotic disorder, psychotic disorder due to another medical condition, catatonia associated with another mental disorder, catatonic disorder due to another medical condition, unspecified catatonia, other specified schizophrenia spectrum or other psychotic disorder, or unspecified schizophrenia spectrum or other psychotic disorder. In certain embodiments, a psychiatric symptom may comprise one or more symptoms of such a schizophrenia spectrum and/or another psychotic disorder.

### Depression and Depressive Symptoms

Depression, also referred to as depressive disorder, is a disorder characterized by a low mood or loss of pleasure or interest in activities for long periods of time. Depression may be triggered by social, psychological and/or biological factors.

Diagnosis of depression may be for example according to the Diagnostic and Statistical Manual of Mental Disorders, the 5th Edition (DSM-5) (American Psychiatric Association. Diagnostic and Statistical Manual of Mental Disorders. 5th ed. Washington D.C.: 2013.), or the Diagnostic and Statistical Manual of Mental Disorders, the 5th Edition, Text Revision (DSM-5-TR) (American Psychiatric Association. Diagnostic and Statistical Manual of Mental Disorders, 5th ed., Text Revision. Washington D.C.: 2022.).

In some embodiments, a subject may have major depressive disorder (including major depressive episode). In some embodiments, a subject may have minor depressive disorder (including minor depressive episode). In some embodiments, a subject may have mood dysregulation disorder. In some embodiments, a subject may have persistent depressive disorder. In some embodiments, a subject may have premenstrual dysphoric disorder. In some embodiments, a subject may have substance/medication-induced depressive disorder. In some embodiments, a subject may have depressive disorder due to another medical condition. In some embodiments, a subject may have other specified depressive disorder. In some embodiments, a subject may have unspecified depressive disorder.

In some embodiments, a subject may have a major depressive episode. According to DSM-5-TR, in a major depressive episode, a subject may present five or more of the following depressive symptoms (not including symptoms clearly attributable to another medical condition.) during the same 2-week period: (1) Depressed mood most of the day, nearly every day, as indicated in the subjective report (e.g., feels sad, empty, hopeless) or in observation made by others (e.g., appears tearful) (In children and adolescents, can be irritable mood); (2) Markedly diminished interest in pleasure in all, or almost all, activities most of the day and nearly every day (as indicated by either subjective account or observation); (3) Significant weight loss when not dieting or weight gain (e.g., a change of more than 5% of body weight in a month), or decrease or increase in appetite nearly every day. (In children, consider failure to make expected weight gain.); (4) Insomnia or hypersomnia nearly every day; (5) Psychomotor agitation or retardation nearly every day (observable by others, not merely subjective feelings of restlessness or being slowed down); (6) Fatigue or loss of energy nearly every day; (7) Feelings of worthlessness or excessive or inappropriate guilt (which may be delusional) nearly every day (not merely self-reproach or guilt about being sick); (8) Diminished ability to think or concentrate, or indecisiveness nearly every day (either by subjective account or as observed by others); and (9) Recurrent thoughts of death (not just fear of dying), recurrent suicidal ideation without a specific plan, or a suicide attempt or a specific plan for committing suicide. At least one of the five or more of the symptoms experienced may be either (1) depressed mood or (2) loss of interest or pleasure. The symptoms may cause clinically significant distress or impairment in social, occupational, or other important areas of functioning, and the episode may not be attributable to the physiological effects of a substance or another medical condition.

In certain embodiments, a subject may present five of the depressive symptoms (1)-(9). In certain embodiments, a subject may present six of the depressive symptoms (1)-(9). In certain embodiments, a subject may present seven of the depressive symptoms (1)-(9). In certain embodiments, a subject may present eight of the above depressive symptoms (1)-(9). In certain embodiments, a subject may present all of the above depressive symptoms (1)-(9).

In some embodiments, a subject may have a major depressive disorder. In certain embodiments, a subject may have a major depressive disorder with a single, major depressive episode. In certain embodiments, a subject may have a major depressive disorder with recurrent, major depressive episodes.

In certain embodiments, the severity of the major depressive disorder or major depressive episode may be mild. In a subject with mild, depressive disorder or episode, few (if any) symptoms in excess of those required to make the diagnosis may be present (for example but not limited to 5 or 6 of the above depressive symptoms (1)-(9)), the intensity of the symptoms may be distressing but manageable, and the symptoms may result in minor impairment in social or occupational functioning.

In certain embodiments, the severity of the major depressive disorder or major depressive episode may be severe. In a subject with severe, depressive disorder or episode, the number of symptoms may be substantially in excess of that required to make the diagnosis (for example but not limited to 8 or 9 of the above depressive symptoms (1)-(9)), the intensity of the symptoms may be seriously distressing and unmanageable, and the symptoms may markedly interfere with social and occupational functioning.

In certain embodiments, the severity of the major depressive disorder or major depressive episode may be moderate. In a subject with moderate, depressive disorder or episode, the number of symptoms, intensity of symptoms, and/or functional impairment are between those specified for “mild” and “severe.”

In some embodiments, a subject may have a minor depressive episode. In a minor depressive episode, a subject may present two to four of the above depressive symptoms (1)-(9) (not including symptoms clearly attributable to another medical condition.) during the same 2-week period. In certain embodiments, a subject may present two of the depressive symptoms (1)-(9). In certain embodiments, a subject may present three of the depressive symptoms (1)-(9). In certain embodiments, a subject may present four of the depressive symptoms (1)-(9).

In some embodiments, a subject may have a minor depressive disorder. In certain embodiments, a subject may have a minor depressive disorder with a single, minor depressive episode. In certain embodiments, a subject may have a minor depressive disorder with recurrent, minor depressive episodes.

In some embodiments, a subject may present at least one of the above-mentioned symptoms of (1)-(9). In certain embodiments, least one of the symptoms experienced may be either (1) depressed mood or (2) loss of interest or pleasure.

In some embodiments, a subject may have persistent depressive disorder. A subject having persistent depressive disorder may: (A) present depressed mood for most of the day, for more days than not, as indicated by either subjective account or observation by others, for at least 2 years (in children and adolescents, mood can be irritable and duration and may be at least 1 year); and (B) while depressed, show two or more of the following: (B-1) Poor appetite or overeating, (B-2) Insomnia or hypersomnia, (B-3) Low energy or fatigue, (B-4) Low self-esteem, (B-5) Poor concentration or difficulty making decisions, and (B-6) Feelings of hopelessness. The symptoms may cause clinically significant distress or impairment in social, occupational, or other important areas of functioning, and the episode may not be attributable to the physiological effects of a substance (e.g., a drug of abuse, a medication) or another medical condition (e.g., hypothyroidism). In certain embodiments, during the at least two years (at least 1 year for children and adolescents), the subject may have never been without the symptoms in Criteria A and B for more than 2 months at a time. In certain embodiments, in a subject having persistent depressive disorder, the criteria for a major depressive disorder may be continuously present for 2 years.

In some embodiments, a subject may have premenstrual dysphoric disorder. A subject having premenstrual dysphoric disorder may meet the following criteria (A)-(C) for most menstrual cycles that occurred in the preceding year: (A) in the majority of menstrual cycles, present at least five symptoms may be present in the final week before the onset of menses, start to improve within a few days after the onset of menses, and become minimal or absent in the week post menses; (B) present one or more of the following symptoms: (B-1) Marked affective lability (e.g., mood swings; feeling suddenly sad or tearful, or increased sensitivity to rejection), (B-2) Marked irritability or anger or increased interpersonal conflicts, (B-3) Marked depressed mood, feelings of hopelessness, or self-deprecating thoughts, and (B-4) Marked anxiety, tension, and/or feelings of being keyed up or on edge); and (C) additionally present one or more of the following symptoms to reach a total of five symptoms when combined with symptoms from Criterion (B): (C-1) Decreased interest in usual activities (e.g., work, school, friends, hobbies), (C-2) Subjective difficulty in concentration, (C-3) Lethargy, easy fatigability, or marked lack of energy, (C-4) Marked change in appetite; overeating; or specific food cravings, (C-5) Hypersomnia or insomnia, (C-6) A sense of being overwhelmed or out of control, and (C-7) Physical symptoms such as breast tenderness or swelling, joint or muscle pain, a sensation of “bloating,” or weight gain. The symptoms may cause clinically significant distress or interference with work, school, usual social activities, or relationships with others (e.g., avoidance of social activities; decreased productivity and efficiency at work, school, or home). The symptoms may not be attributable to the physiological effects of a substance (e.g., a drug of abuse, a medication, other treatment) or another medical condition (e.g., hyperthyroidism).

In some embodiments, a subject may have substance/medication-induced depressive disorder. In substance/medication-induced depressive disorder, (1) a prominent and persistent disturbance in mood that predominates in the clinical picture and is characterized by depressed mood or markedly diminished interest or pleasure in all, or almost all, activities may develop during or soon after substance intoxication or withdrawal or after exposure to or withdrawal from a medication, and (2) the involved substance/medication is capable of producing such symptoms. The substance and/or medication may include for example one or more of: alcohol; phencyclidine; other hallucinogen; inhalant; opioid; sedative, hypnotic, or anxiolytic; amphetamine-type substance (or other stimulant); cocaine; and/or other or unknown substance, and the disturbance in mood may not be better explained by a depressive disorder that is not substance/medication-induced.

In some embodiments, a subject may have depressive disorder due to another medical condition. In depressive disorder due to another medical condition, (A) a subject may present prominent and persistent disturbance in mood that predominates in the clinical picture and is characterized by depressed mood or markedly diminished interest or pleasure in all, or almost all, activities and (B) there is evidence from the history, physical examination, or laboratory findings that the disturbance is the direct pathophysiological consequence of another medical condition. Such another medical condition may include but not be limited to cerebrovascular accident, Huntington's disease, Parkinson's disease, traumatic brain injury, a neuroendocrine condition such as Cushing's syndrome or hypothyroidism, an autoimmune disorder such as systemic lupus erythematosus, deficiencies of certain vitamins (e.g., vitamin B12), multiple sclerosis, neoplasm affecting certain brain regions, and pancreatic cancer.

In some embodiments, a subject may have another specified depressive disorder, such as: recurrent brief depression; short-duration depressive episode; depressive episode with insufficient symptoms; or major depressive episode superimposed on schizophrenia, schizophreniform disorder, delusional disorder, or other specified and unspecified schizophrenia spectrum and other psychotic disorder.

In some embodiments, a subject may have one or more of the above-described depression or depressive disorders.

In some embodiments, in addition to depression (including any of the above-described depression or depressive disorders), a subject may further have one or more of substance-related disorders, panic disorder, generalized anxiety disorder, posttraumatic stress disorder, obsessive compulsive disorder, anorexia nervosa, bulimia nervosa, and borderline personality disorder.

### Antidepressants

Four major classes of antidepressants may be: monoamine oxidase inhibitors (MAOIs), tricyclic antidepressants (TCAs), serotonin reuptake inhibitors (SSRIs), and serotonin (5HT)-norepinephrine (NE) reuptake inhibitors (SNRIs). Further classes of antidepressants may include but are not limited to: tetracyclic antidepressants (TeCAs), serotonin antagonist and reuptake inhibitors (SARIs), N-methyl-D-aspartate (NMDA) antagonists, and serotonin-dopamine activity modulators (SDAMs).

Exemplary MAOIs may include but are not limited to: isocarboxazid, nialamide, phenelzine, tranylcypromine, iproniazide, iproclozide, safrazine, monoamine, moclobemide, toloxatone, minaprine, bifemelane, and selegiline. Exemplary SSRIs may include but are not limited to: fluoxetine, fluvoxamine, paroxetine, sertraline, vilazodone, vortioxetine, citalopram, alaproclate, etoperidone, escitalopram, and imelidine. Exemplary SNRIs may include but are not limited to: milnacipran, venlafaxine, sibutramine, levomilnacipran, duloxetine, desvenlafaxine, and tramadol. Exemplary TCAs may include but are not limited to: imipramine, imipramine oxide, amitriptyline, desipramine, nortriptyline, amoxapine, clomipramine, lofepramine, dosulepin, doxepin, trimipramine, protriptyline, opipramol, dibenzepin, butriptyline, amineptine, iprindole, melitracen, dimetacrine, and quinupramine. Exemplary TeCAs may include but are not limited to: maprotiline, mianserin, setiptiline, and mirtazapine. Exemplary SARIs may include and are not limited to: trazodone, etoperidone, lorpiprazole, mepiprazole, and nefazodone. Exemplary SDAMs may include and are not limited to: brexpiprazole and aripiprazole. Exemplary NMDA antagonists may include bupropion. Further exemplary antidepressants may include but not be limited to: oxitriptan, tryptophan, nomifensine, viloxazine, oxaflozane, medifoxamine, tianeptine, pivagabine, reboxetine, gepirone, agomelatine, hyperici herba, esketamine, brexanolone, and dextromethorphan.

In some embodiments, a subject may have been taking one or more antidepressants before or may be taking at the time of the administration of an anti-CGRP antibody according to the present disclosure. In some embodiments, a subject may be administered one or more antidepressants in combination with an anti-CGRP antibody according to the present disclosure. In certain embodiments, the one or more antidepressants and the anti-CGRP antibody may be administered separately. In certain embodiments, the one or more antidepressants and the anti-CGRP antibody may be administered simultaneously. In particular embodiments, the one or more antidepressants and the anti-CGRP antibody may be contained together in a pharmaceutical composition.

### Anti-CGRP Antibodies and Binding Fragments Thereof Having Binding Specificity for CGRP

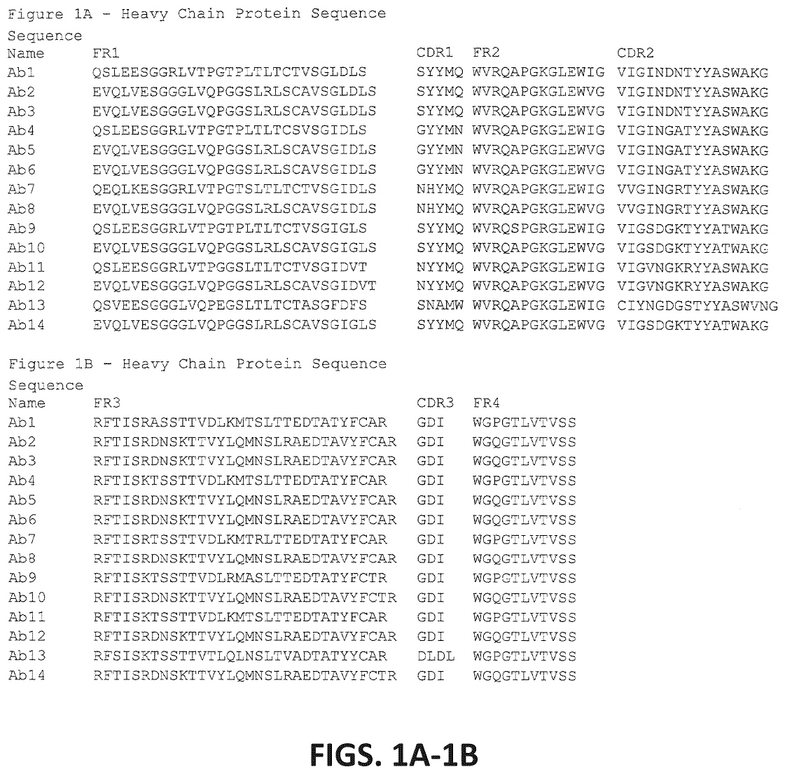

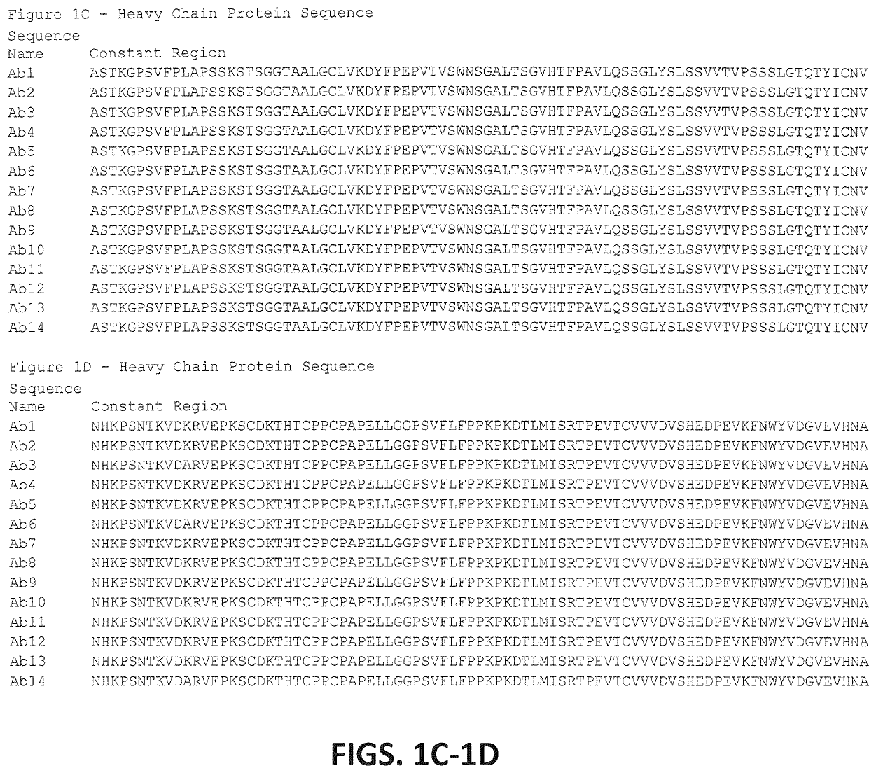

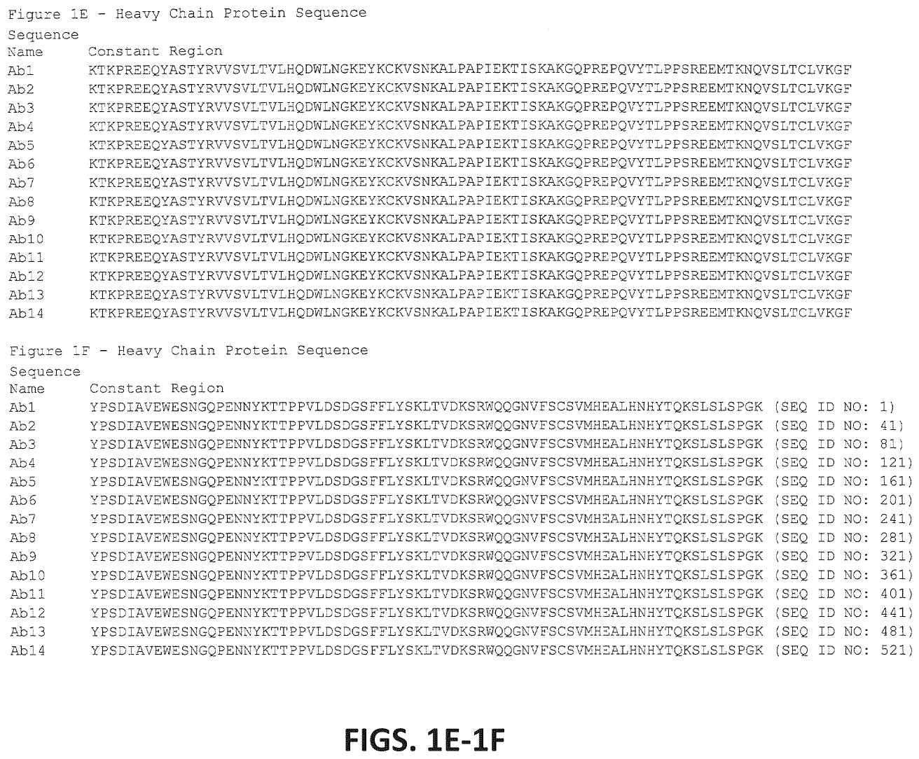

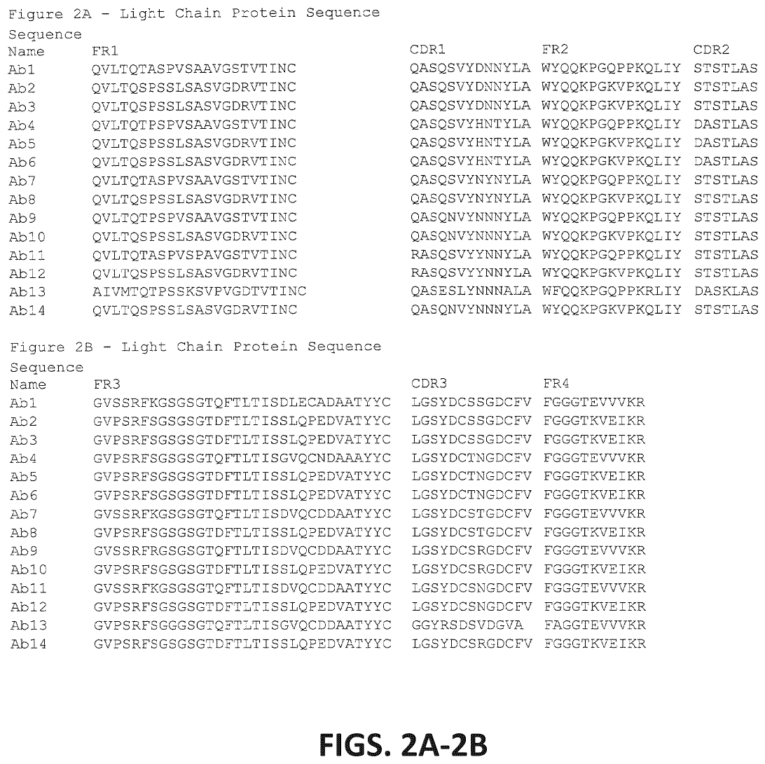

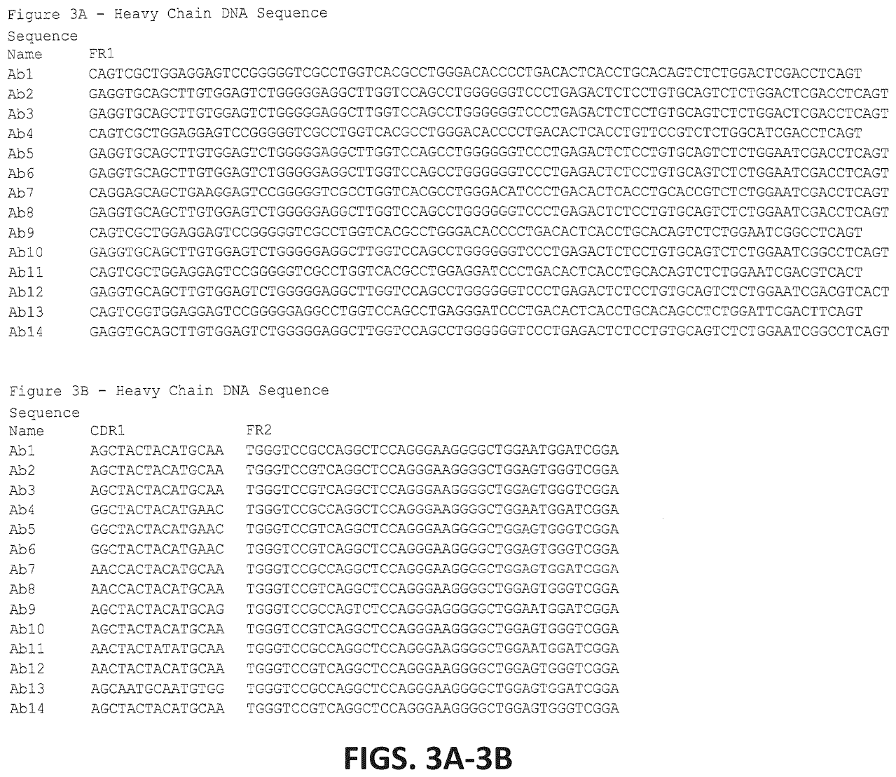

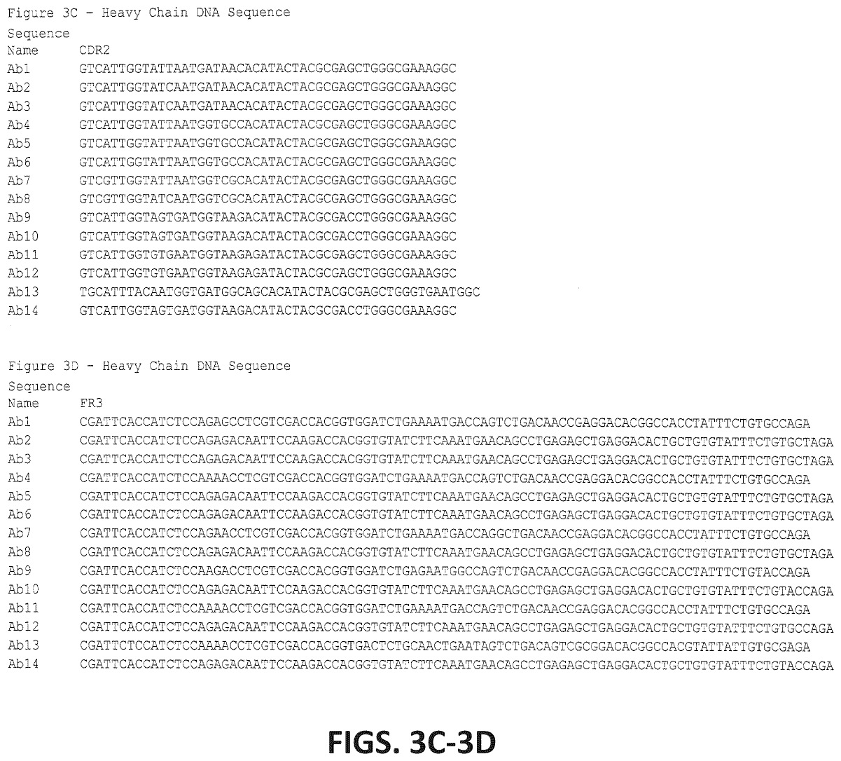

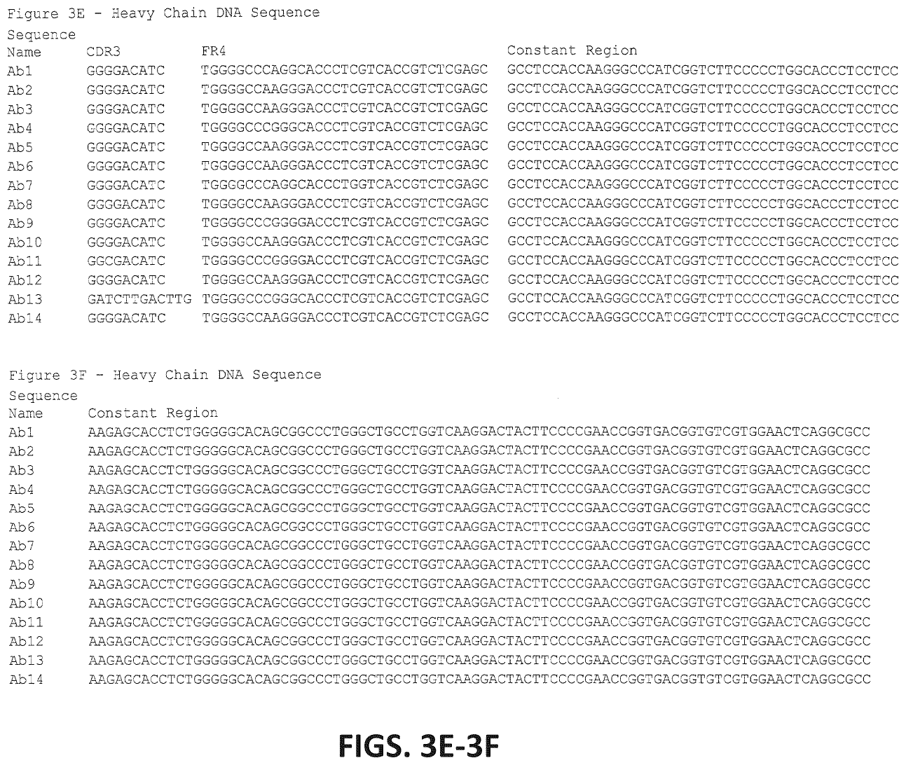

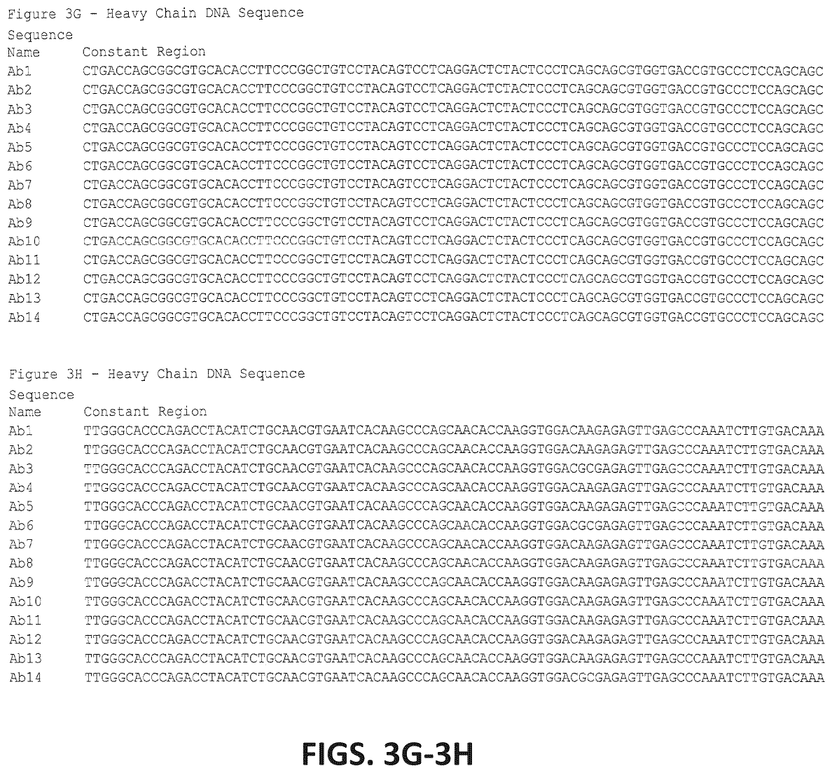

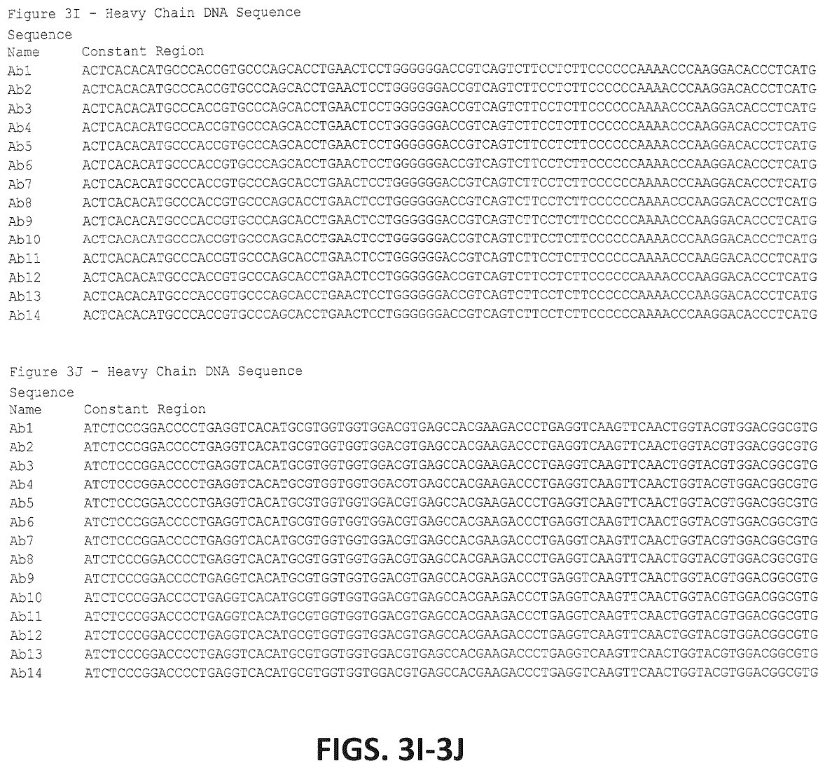

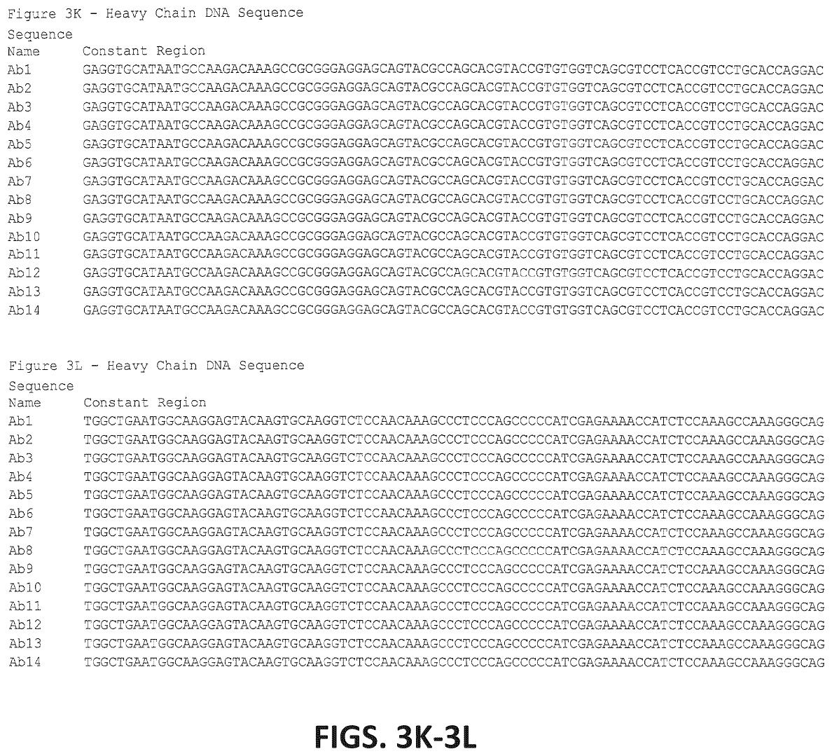

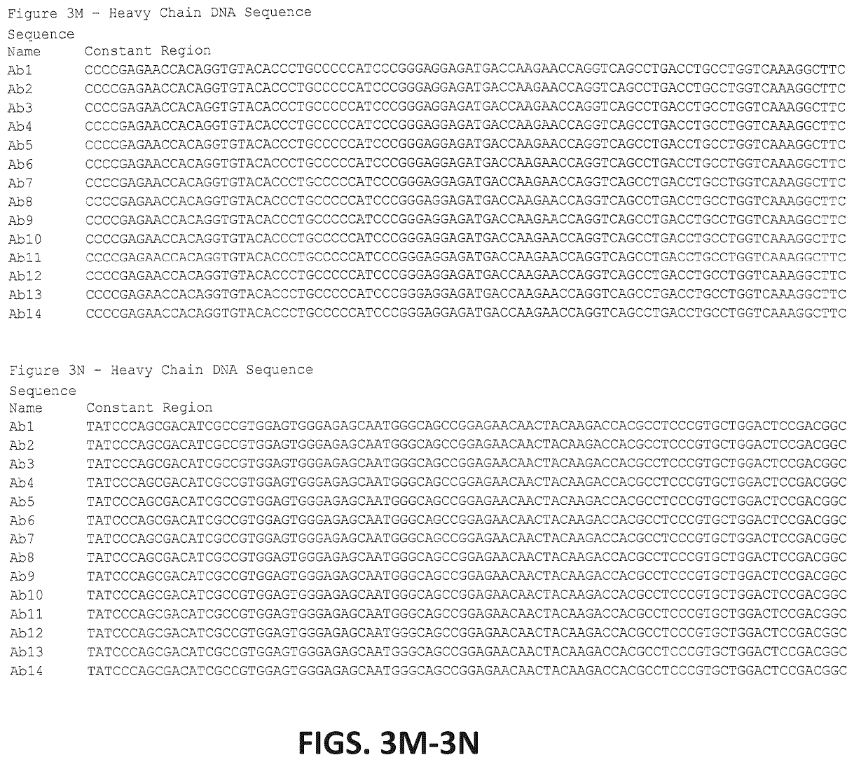

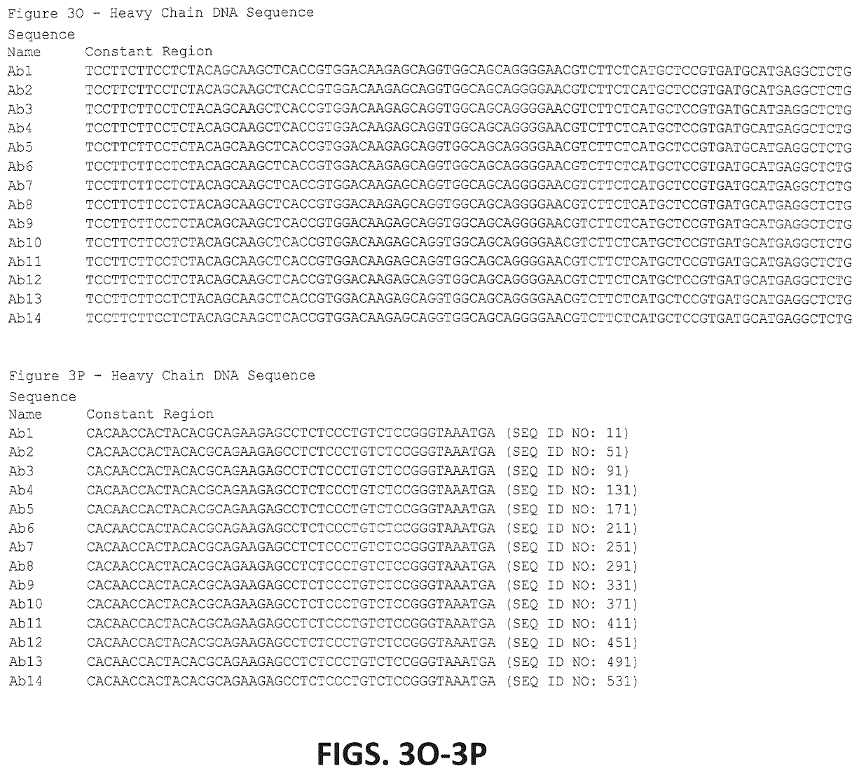

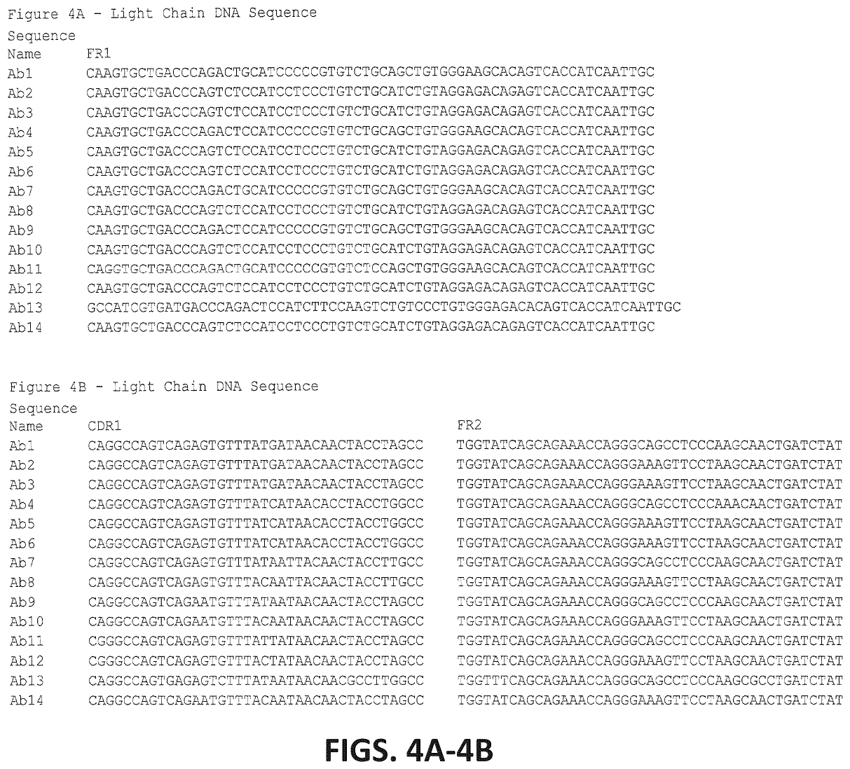

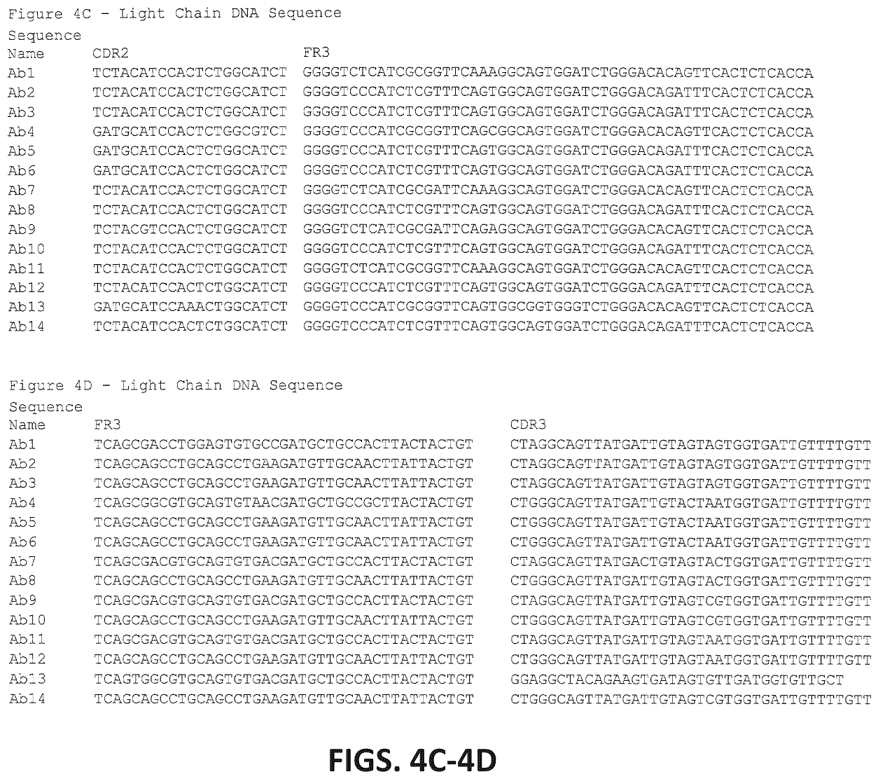

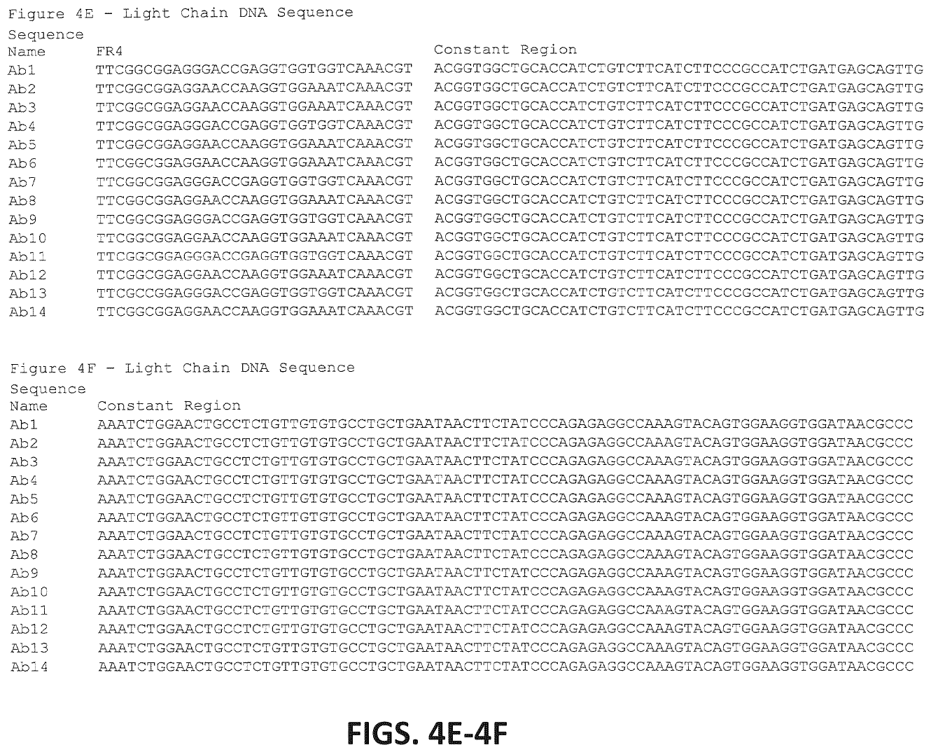

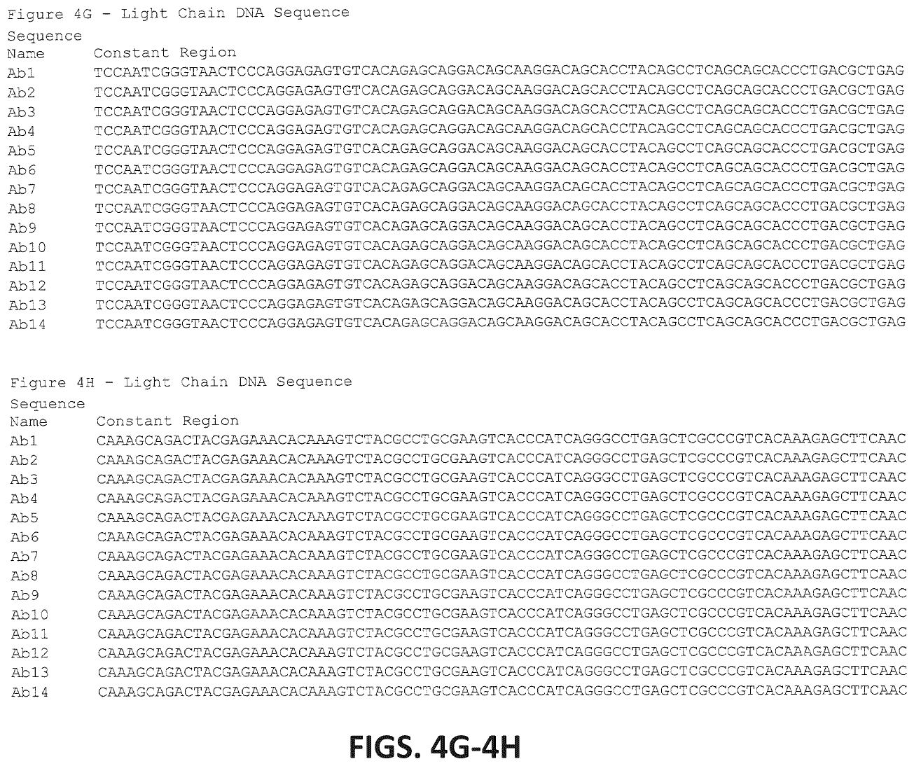

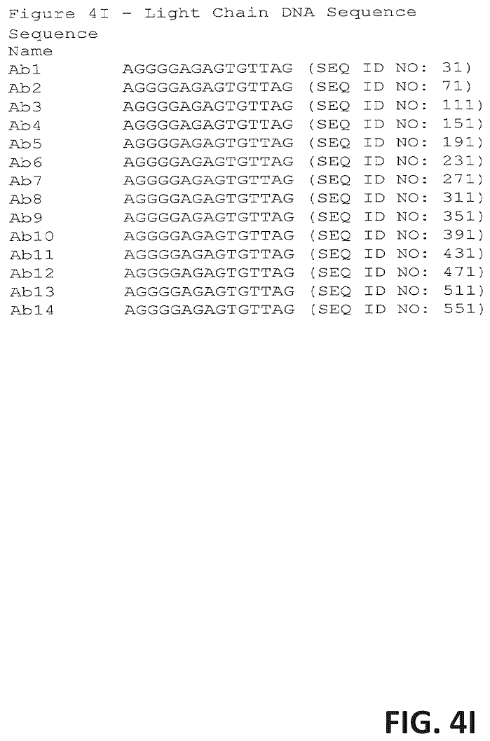

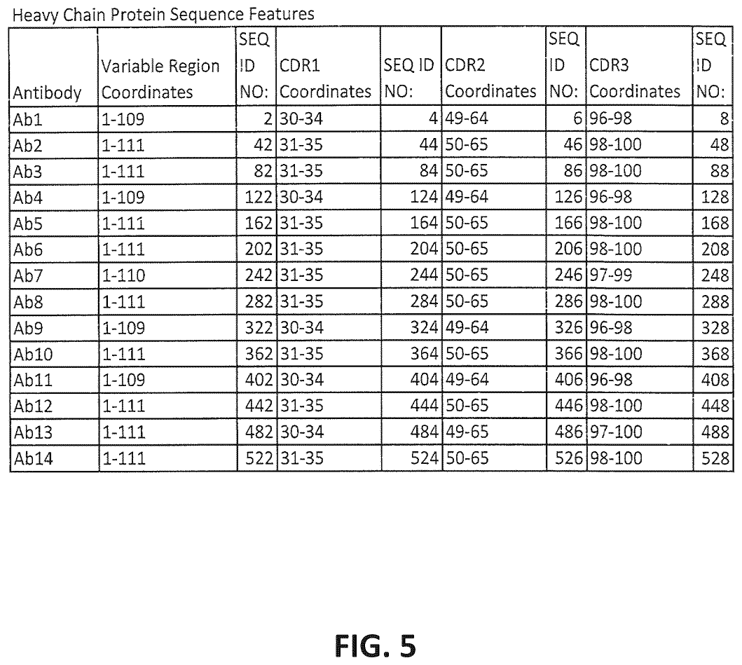

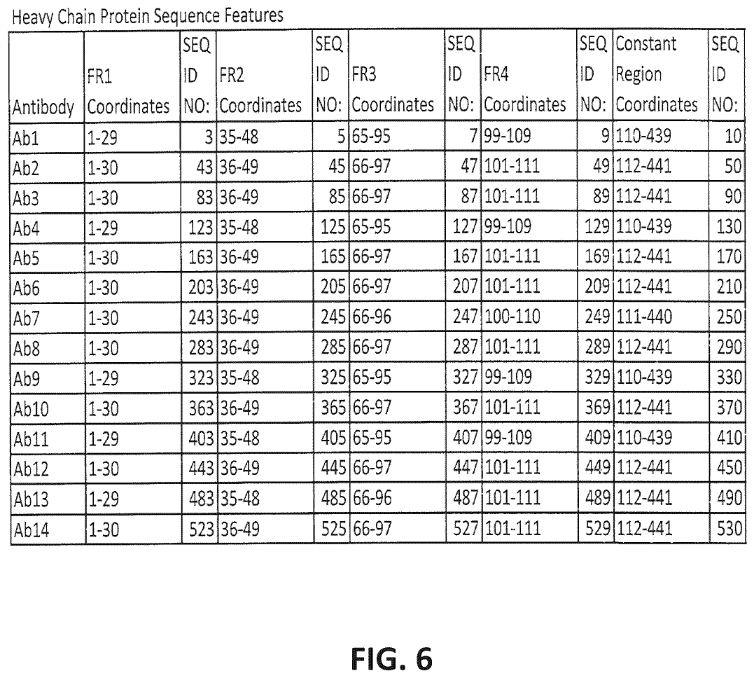

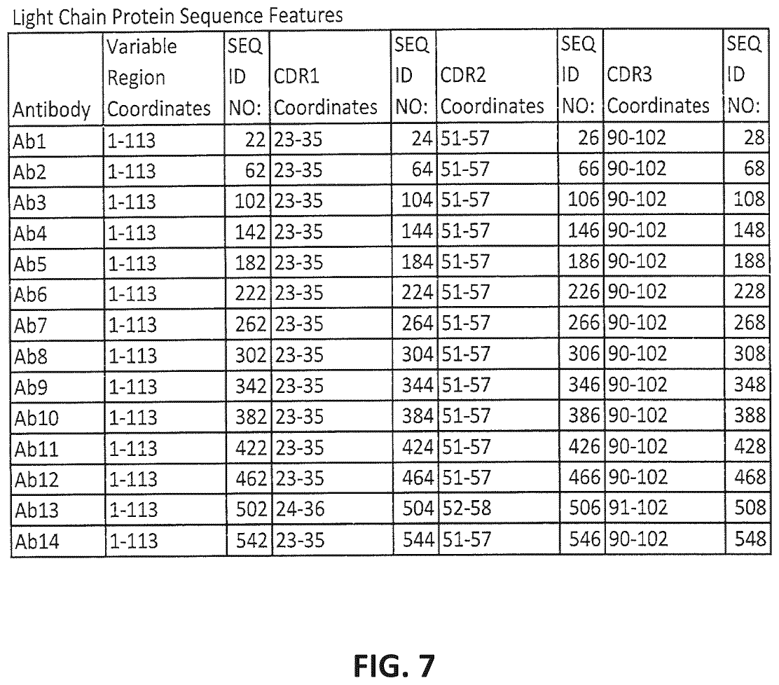

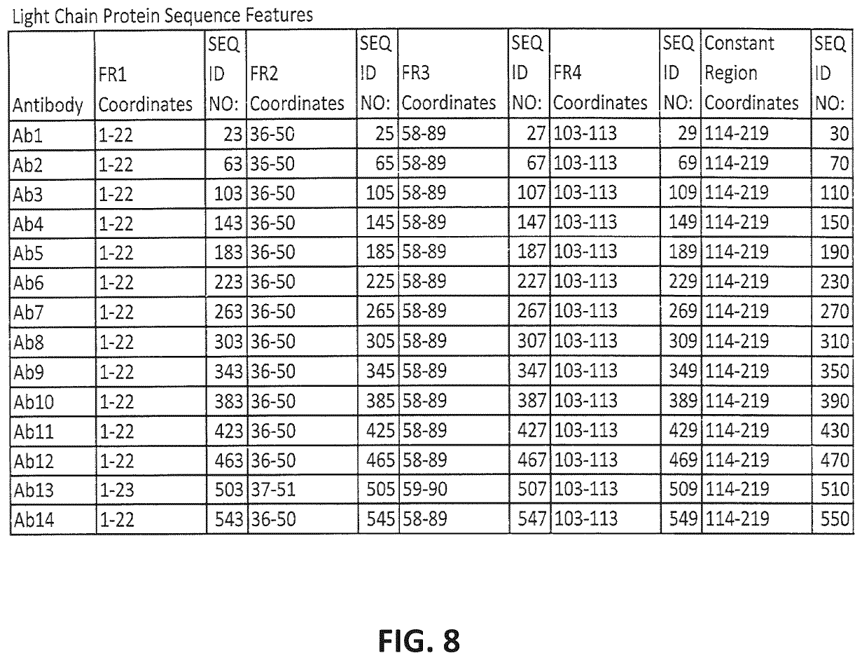

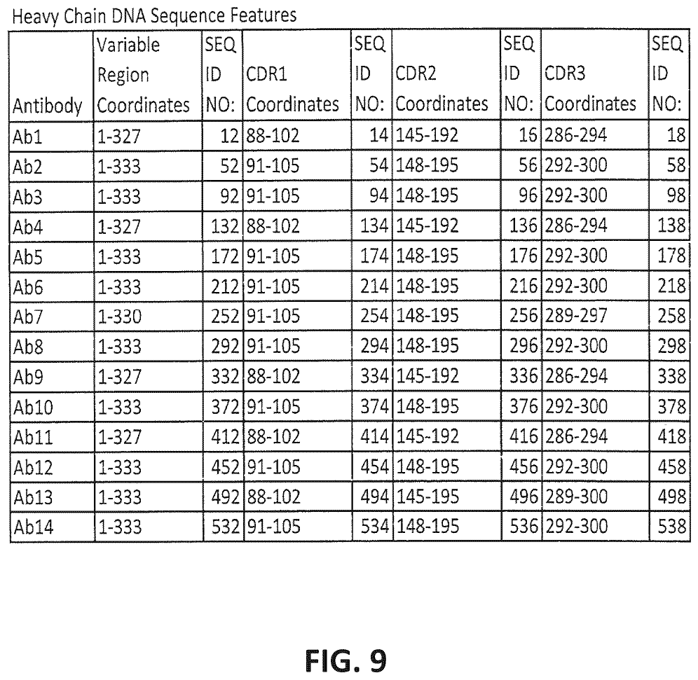

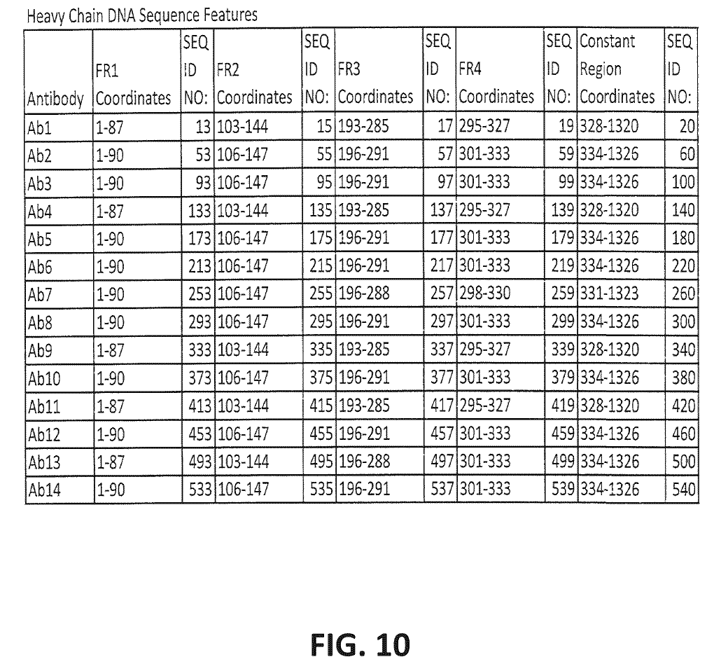

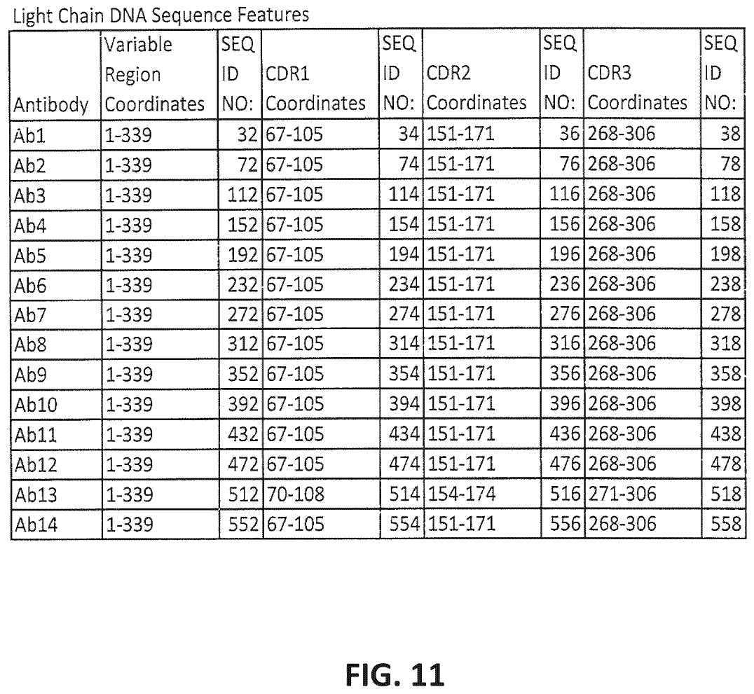

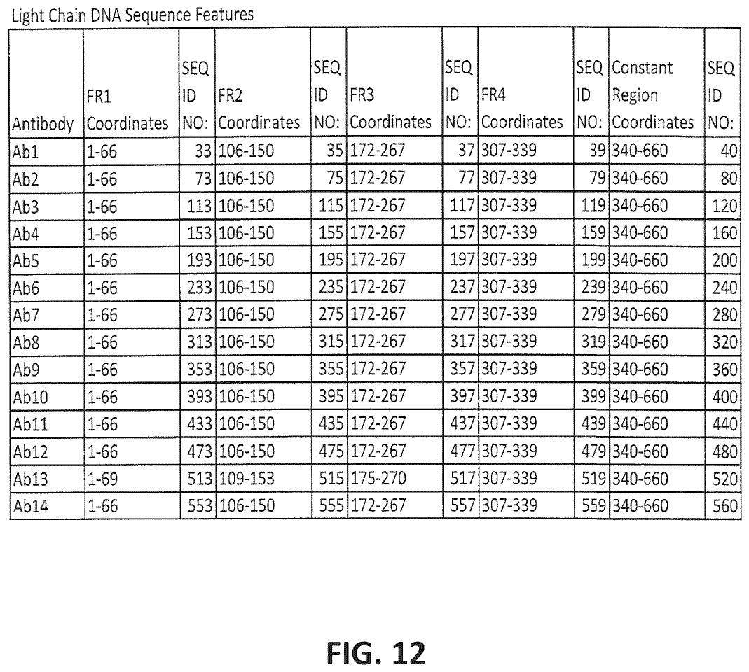

The invention specifically includes the use of specific anti-CGRP antibodies and antibody fragments referred to herein as Ab1-Ab14 which comprise or consist of the CDR, VL, VH, CL, CH polypeptides sequences identified in FIGS. 1A-12. The polypeptides comprised in an especially preferred anti-CGRP antibody, Ab6 is further described below.

### Antibody Ab6

In a preferred exemplary embodiment, the invention includes humanized antibodies having binding specificity to CGRP and possessing a variable light chain sequence comprising the sequence set forth below:

(SEQ ID NO: 222)
QVLTQSPSSLSASVGDRVTINCQASQSVYHNTYLAWYQQKPGKVPKQLI
YDASTLASGVPSRFSGSGSGTDFTLTISSLQPEDVATYYCLGSYDCTNG
DCFVFGGGTKVEIKR.

The invention also includes humanized antibodies having binding specificity to CGRP and possessing a light chain sequence comprising the sequence set forth below:

(SEQ ID NO: 221)
QVLTQSPSSLSASVGDRVTINCQASQSVYHNTYLAWYQQKPGKVPKQLI
YDASTLASGVPSRFSGSGSGTDFTLTISSLQPEDVATYYCLGSYDCTNG
DCFVFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

The invention further includes humanized antibodies having binding specificity to CGRP and possessing a variable heavy chain sequence comprising the sequence set forth below:

(SEQ ID NO: 202)
EVQLVESGGGLVQPGGSLRLSCAVSGIDLSGYYMNWVRQAPGKGLEWVG
VIGINGATYYASWAKGRFTISRDNSKTTVYLQMNSLRAEDTAVYFCARG
DIWGQGTLVTVSS.

The invention also includes humanized antibodies having binding specificity to CGRP and possessing a heavy chain sequence comprising the sequence set forth below:

(SEQ ID NO: 201)
EVQLVESGGGLVQPGGSLRLSCAVSGIDLSGYYMNWVRQAPGKGLEWVG
VIGINGATYYASWAKGRFTISRDNSKTTVYLQMNSLRAEDTAVYFCARG
DIWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNV
NHKPSNTKVDARVEPKSCDKTHTCPPCPAPELLGGPSVFLFPPKPKDTL
MISRTPEVTCVVVDVSHEDPEVKFNWYVDGVEVHNAKTKPREEQYASTY
RVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK.

Alternatively, the heavy chain of Ab6 may lack the C-terminal lysine of SEQ ID NO: 201, i.e., a heavy chain sequence comprising the sequence set forth below:

(SEQ ID NO: 566)
EVQLVESGGGLVQPGGSLRLSCAVSGIDLSGYYMNWVRQAPGKGLEWVG
VIGINGATYYASWAKGRFTISRDNSKTTVYLQMNSLRAEDTAVYFCARG
DIWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNV
NHKPSNTKVDARVEPKSCDKTHTCPPCPAPELLGGPSVFLFPPKPKDTL
MISRTPEVTCVVVDVSHEDPEVKFNWYVDGVEVHNAKTKPREEQYASTY
RVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG.

The invention further contemplates antibodies comprising one or more of the polypeptide sequences of SEQ ID NO: 224; SEQ ID NO: 226; and SEQ ID NO: 228 which correspond to the complementarity-determining regions (CDRs, or hypervariable regions) of the variable light chain sequence of SEQ ID NO: 222 or the light chain sequence of SEQ ID NO: 221, and/or one or more of the polypeptide sequences of SEQ ID NO: 204; SEQ ID NO: 206; and SEQ ID NO: 208 which correspond to the complementarity-determining regions (CDRs, or hypervariable regions) of the variable heavy chain sequence of SEQ ID NO: 202 or the heavy chain sequence of SEQ ID NO: 201 or SEQ ID NO: 566, or combinations of these polypeptide sequences. SEQ ID NO: 208 has the amino acid sequence GDI. In another embodiment of the invention, the antibodies of the invention or fragments thereof comprise, or alternatively consist of, combinations of one or more of the CDRs, the variable heavy and variable light chain sequences, and the heavy and light chain sequences set forth above, including all of them.

The invention also contemplates fragments of the antibody having binding specificity to CGRP. In one embodiment of the invention, antibody fragments of the invention comprise, or alternatively consist of, the polypeptide sequence of SEQ ID NO: 222 or SEQ ID NO: 221. In another embodiment of the invention, antibody fragments of the invention comprise, or alternatively consist of, the polypeptide sequence of SEQ ID NO: 202 or SEQ ID NO: 201 or SEQ ID NO: 566.

In a further embodiment of the invention, fragments of the antibody having binding specificity to CGRP comprise, or alternatively consist of, one or more of the polypeptide sequences of SEQ ID NO: 224; SEQ ID NO: 226; and SEQ ID NO: 228 which correspond to the complementarity-determining regions (CDRs, or hypervariable regions) of the variable light chain sequence of SEQ ID NO: 222 or the light chain sequence of SEQ ID NO: 221.

In a further embodiment of the invention, fragments of the antibody having binding specificity to CGRP comprise, or alternatively consist of, one or more of the polypeptide sequences of SEQ ID NO: 204; SEQ ID NO: 206; and SEQ ID NO: 208 which correspond to the complementarity-determining regions (CDRs, or hypervariable regions) of the variable heavy chain sequence of SEQ ID NO: 202 or the heavy chain sequence of SEQ ID NO: 201 or SEQ ID NO: 566. SEQ ID NO 208 has the amino acids GDI.

The invention also contemplates antibody fragments which include one or more of the antibody fragments described herein. In one embodiment of the invention, fragments of the antibodies having binding specificity to CGRP comprise, or alternatively consist of, one, two, three or more, including all of the following antibody fragments: the variable light chain region of SEQ ID NO: 222; the variable heavy chain region of SEQ ID NO: 202; the complementarity-determining regions (SEQ ID NO: 224; SEQ ID NO: 226; and SEQ ID NO: 228) of the variable light chain region of SEQ ID NO: 222; and the complementarity-determining regions (SEQ ID NO: 204; SEQ ID NO: 206; and SEQ ID NO: 208) of the variable heavy chain region of SEQ ID NO: 202. SEQ ID NO: 208 has the has the amino acids GDI.

In a particularly preferred embodiment of the invention, the humanized anti-CGRP antibody is Ab6, comprising, or alternatively consisting of, SEQ ID NO: 221 and SEQ ID NO: 201 or SEQ ID NO: 566, and having at least one of the biological activities set forth herein.

In a further particularly preferred embodiment of the invention, antibody fragments comprise, or alternatively consist of, Fab (fragment antigen binding) fragments having binding specificity for CGRP. With respect to antibody Ab6, the Fab fragment includes the variable light chain sequence of SEQ ID NO: 222 and the variable heavy chain sequence of SEQ ID NO: 202. This embodiment of the invention further contemplates additions, deletions, and variants of SEQ ID NO: 222 and/or SEQ ID NO: 202 in said Fab while retaining binding specificity for CGRP.

In another particularly preferred embodiment of the invention, said anti-CGRP antibody may comprise the antibody expression product isolated from recombinant cells which express nucleic acid sequences encoding the variable light chain polypeptide of SEQ ID NO: 222 and the variable heavy chain polypeptide of SEQ ID NO: 202, which polypeptides optionally are respectively linked to human light and heavy constant region polypeptides, e.g., human IgG1, IgG2, IgG3 or IgG4 constant regions, which constant regions optionally may be modified to alter glycosylation or proteolysis, wherein said recombinant cells optionally comprise yeast or mammalian cells, e.g., Pichia pastoris or CHO cells.

In another particularly preferred embodiment of the invention, said anti-CGRP antibody may comprise the antibody expression product isolated from recombinant cells which express nucleic acid sequences encoding the light chain of SEQ ID NO: 221 and the heavy chain polypeptide of SEQ ID NO: 201 or SEQ ID NO: 566, wherein said recombinant cells optionally comprise yeast or mammalian cells, e.g., Pichia pastoris or CHO cells, wherein the constant regions thereof optionally may be modified to alter glycosylation or proteolysis or other effector functions.

In another particularly preferred embodiment of the invention, any of the aforementioned anti-CGRP antibodies or antibody fragments may be optionally comprised in a formulation as disclosed herein, e.g., comprising histidine (L-histidine), sorbitol, polysorbate 80, such as, per 1 mL volume, about 100 mg anti-CGRP antibody, about 3.1 mg L-Histidine, about 40.5 mg Sorbitol, and about 0.15 mg Polysorbate 80, having a pH of about 5.8.

In one embodiment of the invention described herein (infra), Fab fragments may be produced by enzymatic digestion (e.g., papain) of Ab6. In another embodiment of the invention, anti-CGRP antibodies such as Ab6 or Fab fragments thereof may be produced via expression in mammalian cells such as CHO, NSO or HEK 293 cells, fungal, insect, or microbial systems such as yeast cells (for example diploid yeast such as diploid Pichia) and other yeast strains. Suitable Pichia species include, but are not limited to, Pichia pastoris.

In another embodiment, antibody fragments may be present in one or more of the following non-limiting forms: Fab, Fab′, F(ab′)₂, Fv and single chain Fv antibody forms. In a preferred embodiment, the anti-CGRP antibodies described herein further comprises the kappa constant light chain sequence comprising the sequence set forth below:

(SEQ ID NO: 563)
TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC.

In another preferred embodiment, the anti-CGRP antibodies described herein further comprises the gamma-1 constant heavy chain polypeptide sequence comprising the sequence set forth below or the same sequence lacking the carboxy terminal lysine residue (SEQ ID NO: 564 and SEQ ID NO: 565, respectively):

(SEQ ID NO: 564)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCVVV
DVSHEDPEVKFNWYVDGVEVHNAKTKPREEQYASTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.
(SEQ ID NO: 565)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCVVV
DVSHEDPEVKFNWYVDGVEVHNAKTKPREEQYASTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPG.

For clarity, any antibody disclosed herein is intended to include any variant of the disclosed constant region variant sequences, e.g., Ab6 may comprise the constant region of SEQ ID NO: 564 containing the C-terminal lysine or may comprise the constant region of SEQ ID NO: 565 lacking the C-terminal lysine. Thus, every disclosure herein of the heavy chain of SEQ ID NO: 201 also includes a variant lacking the C-terminal lysine residue thereof, i.e., having the heavy chain variable region sequence of Ab6 (SEQ ID NO: 202) and the constant region sequence of SEQ ID NO: 565. For example, the sequence encoding an antibody comprising a C-terminal lysine in the heavy chain may, when expressed in cell lines such as CHO cells, produce an antibody lacking said C-terminal lysine due to proteolysis, or a mixture of heavy chains containing or lacking said C-terminal lysine.

In another embodiment, the invention contemplates use of an isolated anti-CGRP antibody comprising a V_{H} polypeptide sequence selected from: SEQ ID NO: 2, SEQ ID NO: 42, SEQ ID NO: 82, SEQ ID NO: 122, SEQ ID NO: 162, SEQ ID NO: 202, SEQ ID NO: 242, SEQ ID NO: 282, SEQ ID NO: 322, SEQ ID NO: 362, SEQ ID NO: 402, SEQ ID NO: 442, SEQ ID NO: 482, or SEQ ID NO: 522, or a variant thereof; and further comprising a V_{L} polypeptide sequence selected from: SEQ ID NO: 22, SEQ ID NO: 62, SEQ ID NO: 102, SEQ ID NO: 142, SEQ ID NO: 182, SEQ ID NO: 222, SEQ ID NO: 262, SEQ ID NO: 302, SEQ ID NO: 342, SEQ ID NO: 382, SEQ ID NO: 422, SEQ ID NO: 462, SEQ ID NO: 502, or SEQ ID NO: 542, or a variant thereof, wherein one or more of the framework residues (FR residues) in said V_{H} or V_{L} polypeptide has been substituted with another amino acid residue resulting in an anti-CGRP antibody that specifically binds CGRP. The invention contemplates humanized and chimeric forms of these antibodies. The chimeric antibodies may include an Fc derived from IgG1, IgG2, IgG3, or IgG4 constant regions.

In one embodiment of the invention, the antibodies or V_{H} or V_{L} polypeptides originate or are selected from one or more rabbit B cell populations prior to initiation of the humanization process referenced herein.

In another embodiment of the invention, the anti-CGRP antibodies and fragments thereof do not have binding specificity for CGRP-R In a further embodiment of the invention, the anti-CGRP antibodies and fragments thereof inhibit the association of CGRP with CGRP-R. In another embodiment of the invention, the anti-CGRP antibodies and fragments thereof inhibit the association of CGRP with CGRP-R and/or additional proteins and/or multimers thereof, and/or antagonizes the biological effects thereof.

As stated herein, antibodies and fragments thereof may be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

### Modifications of Antibodies

The one or more anti-human CGRP antibodies or use thereof according to the disclosure may be glycosylated or non-glycosylated. In some embodiments, the anti-human CGRP antibodies are aglycosylated or if glycosylated are only mannosylated; that contain an Fc region that has been modified to alter effector function, half-life, proteolysis, and/or glycosylation; are human, humanized, single chain or chimeric; and are a humanized antibody derived from a rabbit (parent) anti-human CGRP antibody. An exemplary mutation which impairs glycosylation comprises the mutation of the Asn residue at position 297 of an IgG heavy chain constant region such as IgG1 to another amino acid, such as Ala as described in U.S. Pat. No. 5,624,821, which is incorporated by reference in its entirety.

Immunoglobulins and fragments thereof may be modified post-translationally, e.g. to add effector moieties such as chemical linkers, detectable moieties, such as fluorescent dyes, enzymes, toxins, substrates, bioluminescent materials, radioactive materials, chemiluminescent moieties and the like, or specific binding moieties, such as streptavidin, avidin, or biotin, and the like may be utilized in the methods and compositions of the present invention. Examples of additional effector molecules are provided infra.

Antibodies or fragments thereof may also be chemically modified to provide additional advantages such as increased solubility, stability and circulating time (in vivo half-life) of the polypeptide, or decreased immunogenicity (See U.S. Pat. No. 4,179,337). The chemical moieties for derivatization may be selected from water soluble polymers such as polyethylene glycol, ethylene glycol/propylene glycol copolymers, carboxymethylcellulose, dextran, polyvinyl alcohol and the like. The antibodies and fragments thereof may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

There are a number of attachment methods available to those skilled in the art, See e.g., EP 0 401 384, herein incorporated by reference (coupling PEG to G-CSF), See also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. For example, polyethylene glycol can be linked to polypeptides via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) or to more than one type of amino acid residue (e.g., lysine, histidine, aspartic acid, glutamic acid, cysteine and combinations thereof).

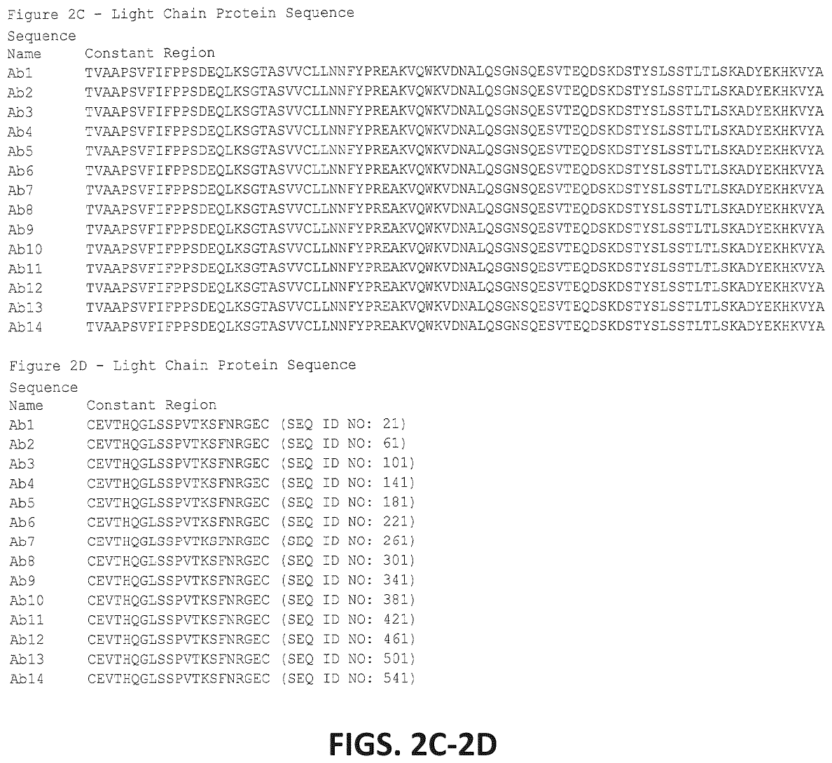

Alternatively, antibodies or fragments thereof may have increased in vivo half-lives via fusion with albumin (including but not limited to recombinant human serum albumin or fragments or variants thereof (See, e.g., U.S. Pat. No. 5,876,969, issued Mar. 2, 1999, EP Patent 0 413 622, and U.S. Pat. No. 5,766,883, issued Jun. 16, 1998, herein incorporated by reference in their entirety)) or other circulating blood proteins such as transferrin or ferritin. In a preferred embodiment, polypeptides and/or antibodies of the present invention (including fragments or variants thereof) are fused with the mature form of human serum albumin (i.e., amino acids 1-585 of human serum albumin as shown in FIGS. 1 and 2 of EP Patent 0 322 094) which is herein incorporated by reference in its entirety. Polynucleotides encoding fusion proteins of the invention are also encompassed by the invention.

Regarding detectable moieties, further exemplary enzymes include, but are not limited to, horseradish peroxidase, acetylcholinesterase, alkaline phosphatase, beta-galactosidase and luciferase. Further exemplary fluorescent materials include, but are not limited to, rhodamine, fluorescein, fluorescein isothiocyanate, umbelliferone, dichlorotriazinylamine, phycoerythrin and dansyl chloride. Further exemplary chemiluminescent moieties include, but are not limited to, luminol. Further exemplary bioluminescent materials include, but are not limited to, luciferin and aequorin. Further exemplary radioactive materials include, but are not limited to, Iodine 125 (¹²¹I), Carbon 14 (¹⁴C), Sulfur 35 (³⁵S), Tritium (³H) and Phosphorus 32 (³²P).

Regarding functional moieties, exemplary cytotoxic agents include, but are not limited to, methotrexate, aminopterin, 6-mercaptopurine, 6-thioguanine, cytarabine, 5-fluorouracil decarbazine; alkylating agents such as mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin (formerly daunomycin), doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimitotic agents such as the vinca alkaloids, vincristine and vinblastine. Other cytotoxic agents include paclitaxel (Taxol), ricin, pseudomonas exotoxin, gemcitabine, cytochalasin B, gramicidin D, ethidium bromide, emetine, etoposide, tenoposide, colchicine, dihydroxy anthracin dione, 1-dehydrotestosterone, glucocorticoids, procaine, tetracaine, lidocaine, propranolol, puromycin, procarbazine, hydroxyurea, asparaginase, corticosteroids, mytotane (O,P′-(DDD)), interferons, and mixtures of these cytotoxic agents.

Further cytotoxic agents include, but are not limited to, chemotherapeutic agents such as carboplatin, cisplatin, paclitaxel, gemcitabine, calicheamicin, doxorubicin, 5-fluorouracil, mitomycin C, actinomycin D, cyclophosphamide, vincristine and bleomycin. Toxic enzymes from plants and bacteria such as ricin, diphtheria toxin and Pseudomonas toxin may be conjugated to the humanized or chimeric antibodies, or binding fragments thereof, to generate cell-type-specific-killing reagents (Youle, et al., Proc. Nat'l Acad. Sci. USA 77:5483 (1980); Gilliland, et al., Proc. Nat'l Acad. Sci. USA 77:4539 (1980); Krolick, et al., Proc. Nat'l Acad. Sci. USA 77:5419 (1980)).

Other cytotoxic agents include cytotoxic ribonucleases as described by Goldenberg in U.S. Pat. No. 6,653,104. Embodiments of the invention also relate to radioimmunoconjugates where a radionuclide that emits alpha or beta particles is stably coupled to the antibody, or binding fragments thereof, with or without the use of a complex-forming agent. Such radionuclides include beta-emitters such as Phosphorus-32 (³²P), Scandium-47 (⁴⁷Sc), Copper-67 (⁶⁷Cu), Gallium-67 (⁶⁷Ga), Yttrium-88 (⁸⁸Y), Yttrium-90 (⁹⁰Y), Iodine-125 (¹²⁵I), Iodine-131 (¹³¹I), Samarium-153 (¹⁵³Sm), Lutetium-177 (¹⁷⁷Lu), Rhenium-186 (¹⁸⁶Re) or Rhenium-188 (¹⁸⁸Re), and alpha-emitters such as Astatine-211 (²¹¹At), Lead-212 (²¹²Pb), Bismuth-212 (²¹²Bi) or -213 (²¹³Bi) or Actinium-225 (²²⁵Ac).

Methods are known in the art for conjugating an antibody or binding fragment thereof to a detectable moiety and the like, such as for example those methods described by Hunter et al, Nature 144:945 (1962); David et al, Biochemistry 13:1014 (1974); Pain et al, J. Immunol. Meth. 40:219 (1981); and Nygren, J., Histochem. and Cytochem. 30:407 (1982).

Embodiments described herein further include variants and equivalents that are substantially homologous to the antibodies, antibody fragments, diabodies, SMIPs, camelbodies, nanobodies, IgNAR, polypeptides, variable regions and CDRs set forth herein. These may contain, e.g., conservative substitution mutations, (i.e., the substitution of one or more amino acids by similar amino acids). For example, conservative substitution refers to the substitution of an amino acid with another within the same general class, e.g., one acidic amino acid with another acidic amino acid, one basic amino acid with another basic amino acid, or one neutral amino acid by another neutral amino acid. What is intended by a conservative amino acid substitution is well known in the art.

In another embodiment, the invention contemplates polypeptide sequences having at least 90% or greater sequence homology to any one or more of the polypeptide sequences of antibody fragments, variable regions and CDRs set forth herein. More preferably, the invention contemplates polypeptide sequences having at least 95% or greater sequence homology, even more preferably at least 98% or greater sequence homology, and still more preferably at least 99% or greater sequence homology to any one or more of the polypeptide sequences of antibody fragments, variable regions and CDRs set forth herein. Methods for determining homology between nucleic acid and amino acid sequences are well known to those of ordinary skill in the art.

In another embodiment, the invention further contemplates the above-recited polypeptide homologs of the antibody fragments, variable regions and CDRs set forth herein further having anti-CGRP activity. Non-limiting examples of anti-CGRP activity are set forth herein.

The present invention also contemplates anti-CGRP antibodies comprising any of the polypeptide or polynucleotide sequences described herein substituted for any of the other polynucleotide sequences described herein. For example, without limitation thereto, the present invention contemplates antibodies comprising the combination of any of the variable light chain and variable heavy chain sequences described herein, and further contemplates antibodies resulting from substitution of any of the CDR sequences described herein for any of the other CDR sequences described herein.

### Additional Anti-CGRP Antibodies and Exemplary Embodiments

In another embodiment, the invention contemplates treatment methods using one or more anti-human CGRP antibodies or antibody fragments thereof which specifically bind to the same overlapping linear or conformational epitope(s) and/or competes for binding to the same overlapping linear or conformational epitope(s) on an intact human CGRP polypeptide or fragment thereof as an anti-human CGRP antibody selected from Ab6, Ab1, Ab2, Ab3, Ab4, Ab5, Ab7, Ab8, Ab9, Ab10, Ab11, Ab12, Ab13, or Ab14. In a preferred embodiment, the anti-human CGRP antibody or fragment thereof specifically binds to the same overlapping linear or conformational epitope(s) and/or competes for binding to the same overlapping linear or conformational epitope(s) on an intact human CGRP polypeptide or a fragment thereof as Ab6, Ab3, Ab13, or Ab14.

A preferred embodiment of the invention is directed to treatment methods using chimeric or humanized antibodies and fragments thereof (including Fab fragments) having binding specificity for CGRP and inhibiting biological activities mediated by the binding of CGRP to the CGRP receptor. In a particularly preferred embodiment of the invention, the chimeric or humanized anti-CGRP antibodies are selected from Ab6, Ab3, Ab13, or Ab14.

In another embodiment of the invention, the anti-human CGRP antibody used in the described treatment methods is an antibody which specifically binds to the same overlapping linear or conformational epitopes on an intact CGRP polypeptide or fragment thereof that is (are) specifically bound by Ab3, Ab6, Ab13, or Ab14 as ascertained by epitopic mapping using overlapping linear peptide fragments which span the full length of the native human CGRP polypeptide.

In another embodiment, the invention is also directed to treatment methods using an isolated anti-CGRP antibody or antibody fragment comprising one or more of the CDRs contained in the V_{H} polypeptide sequences selected from: 3, 13, 23, 33, 43, 53, 63, 73, 83, 93, 103, 113, 123, or 133, or a variant thereof, and/or one or more of the CDRs contained in the V_{L} polypeptide sequences selected from: 1, 11, 21, 31, 41, 51, 61, 71, 81, 91, 101, 111, 121, or 131, or a variant thereof.

In one embodiment of the invention, the anti-human CGRP antibody discussed in the two prior paragraphs comprises at least 2 complementarity determining regions (CDRs) in each the variable light and the variable heavy regions which are identical to those contained in an anti-human CGRP antibody selected from Ab6, Ab1, Ab2, Ab3, Ab4, Ab5, Ab7, Ab8, Ab9, Ab10, Ab11, Ab12, Ab13, or Ab14.

In a preferred embodiment, the anti-human CGRP antibody used in the described treatment methods comprises at least 2 complementarity determining regions (CDRs) in each the variable light and the variable heavy regions which are identical to those contained in Ab3 or Ab6. In another embodiment, all of the CDRs of the anti-human CGRP antibody discussed above are identical to the CDRs contained in an anti-human CGRP antibody selected from Ab6, Ab1, Ab2, Ab3, Ab4, Ab5, Ab7, Ab8, Ab9, Ab10, Ab11, Ab12, Ab13, or Ab14. In a preferred embodiment of the invention, all of the CDRs of the anti-human CGRP antibody discussed above are identical to the CDRs contained in an anti-human CGRP antibody selected from Ab6 or Ab3. In a preferred embodiment of the invention, all of the CDRs of the anti-human CGRP antibody are identical to the CDRs contained in Ab6.

The invention further contemplates one or more anti-human CGRP antibodies wherein the framework regions (FRs) in the variable light region and the variable heavy regions of said antibody respectively are human FRs which are unmodified or which have been modified by the substitution of one or more human FR residues in the variable light or heavy chain region with the corresponding FR residues of the parent rabbit antibody, and wherein said human FRs have been derived from human variable heavy and light chain antibody sequences which have been selected from a library of human germline antibody sequences based on their high level of homology to the corresponding rabbit variable heavy or light chain regions relative to other human germline antibody sequences contained in the library.

### Functions and Properties of Anti-CGRP Antibodies

The anti-CGRP activity of the anti-CGRP antibodies of the present invention, and fragments thereof having binding specificity to CGRP, may also be described by their strength of binding or their affinity for CGRP. In one embodiment of the invention, the anti-CGRP antibodies of the present invention, and fragments thereof having binding specificity to CGRP, bind to CGRP with a dissociation constant (K_{D}) of less than or equal to 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, or 10⁻¹³ M. Preferably, the anti-CGRP antibodies and fragments thereof bind CGRP with a dissociation constant of less than or equal to 10⁻¹¹ M, 5×10⁻¹² M, or 10⁻¹² M. In another embodiment of the invention, the anti-CGRP antibodies of the present invention, and fragments thereof having binding specificity to CGRP, bind to a linear or conformational CGRP epitope.

In another embodiment of the invention, the anti-CGRP activity of the anti-CGRP antibodies of the present invention, and fragments thereof having binding specificity to CGRP, bind to CGRP with an off-rate of less than or equal to 10⁻⁴ S⁻¹, 5×10⁻⁵ S⁻¹, 10⁻⁵ S⁻¹, 5×10⁻⁶ S⁻¹, 10⁻⁶ S⁻¹, 5×10⁻⁷ S⁻¹, or 10⁻⁷ S⁻¹.

In a further embodiment of the invention, the anti-CGRP activity of the anti-CGRP antibodies of the present invention, and fragments thereof having binding specificity to CGRP, exhibit anti-CGRP activity by preventing, ameliorating or reducing the symptoms of, or alternatively treating, diseases and disorders associated with CGRP. Non-limiting examples of diseases and disorders associated with CGRP are set forth herein and include headache and migraine disorders.

### Polynucleotides Encoding Anti-CGRP Antibody Polypeptides, Vectors, and Host Cells

Further disclosed herein are polynucleotides encoding any of the anti-CGRP antibodies disclosed herein, vectors comprising one or more polynucleotides encoding any of the anti-CGRP antibodies disclosed herein, and host cells comprising one or more polynucleotides encoding any of the anti-CGRP antibodies disclosed herein.

Polynucleotides as described herein may encode any of the anti-CGRP antibodies disclosed herein, including but not limited to those comprising the 6 CDRs of Ab1 through Ab14, such as Ab6.

In some embodiments, the VH and VL of the antibody (or in some cases the heavy chain and light chain) may be encoded by separate polynucleotides.

In some embodiments, the VH and VL of the antibody (or in some cases the heavy chain and light chain) may be encoded by the same polynucleotide.

In certain embodiments, the VH and VL of the antibody (or in some cases the heavy chain and light chain) may be encoded by separate strands of the same polynucleotide. In some cases, the VH and VL of the antibody (or in some cases the heavy chain and light chain) may be encoded under the same promoter (e.g., bidirectional). In some cases, the VH and VL of the antibody (or in some cases the heavy chain and light chain) may be encoded under separate promoters.

In certain embodiments, the VH and VL of the antibody (or in some cases the heavy chain and light chain) may be encoded by the same strand of the same polynucleotide. In some cases, the VH and VL of the antibody (or in some cases the heavy chain and light chain) may be encoded under the same promoter. In some cases, the VH and VL of the antibody (or in some cases the heavy chain and light chain) may be encoded under separate promoters.

As aforementioned the invention specifically includes the use of specific anti-CGRP antibodies and antibody fragments referred to herein as Ab1-Ab14 which comprise or consist of the CDR, VL, VH, CL, and CH polypeptides having the sequences identified in FIGS. 1A-12. The nucleic acid sequences encoding the foregoing VL, VH, CL, and CH polypeptides comprised in Ab1-Ab14 are also comprised in FIGS. 1A-12. The nucleic acid sequences which encode the CDR, VL, VH, CL, and CH polypeptides of an especially preferred anti-CGRP antibody, Ab6, are further described below. Antibody Ab6

The invention is further directed to polynucleotides encoding antibody polypeptides having binding specificity to CGRP. In one embodiment of the invention, polynucleotides of the invention comprise, or alternatively consist of, the following polynucleotide sequence encoding the variable light chain polypeptide sequence of SEQ ID NO: 222:

(SEQ ID NO: 232)
CAAGTGCTGacccagtctccatcctccctgtctgcatctgtaggagaca
gagtcaccatcAATtgcCAGGCCAGTCAGAGTGTTTATCATAACACCTA
CCTGGCCtggtatcagcagaaaccagggaaagttcctaagCAActgatc
tatGATGCATCCACTCTGGCATCTggggtcccatctcgtttcagtggca
gtggatctgggacagatttcactctcaccatcagcagcctgcagcctga
agatgttgcaacttattactgtCTGGGCAGTTATGATTGTACTAATGGT
GATTGTTTTGTTttcggcggaggaaccaaggtggaaatcaaacgt.

In one embodiment of the invention, polynucleotides of the invention comprise, or alternatively consist of, the following polynucleotide sequence encoding the light chain polypeptide sequence of SEQ ID NO: 221:

(SEQ ID NO: 231)
CAAGTGCTGacccagtctccatcctccctgtctgcatctgtaggagaca
gagtcaccatcAATtgcCAGGCCAGTCAGAGTGTTTATCATAACACCTA
CCTGGCCtggtatcagcagaaaccagggaaagttcctaagCAActgatc
tatGATGCATCCACTCTGGCATCTggggtcccatctcgtttcagtggca
gtggatctgggacagatttcactctcaccatcagcagcctgcagcctga
agatgttgcaacttattactgtCTGGGCAGTTATGATTGTACTAATGGT
GATTGTTTTGTTttcggcggaggaaccaaggtggaaatcaaacgtACGG
TGGCTGCACCATCTGTCTTCATCTTCCCGCCATCTGATGAGCAGTTGAA
ATCTGGAACTGCCTCTGTTGTGTGCCTGCTGAATAACTTCTATCCCAGA
GAGGCCAAAGTACAGTGGAAGGTGGATAACGCCCTCCAATCGGGTAACT
CCCAGGAGAGTGTCACAGAGCAGGACAGCAAGGACAGCACCTACAGCCT
CAGCAGCACCCTGACGCTGAGCAAAGCAGACTACGAGAAACACAAAGTC
TACGCCTGCGAAGTCACCCATCAGGGCCTGAGCTCGCCCGTCACAAAGA
GCTTCAACAGGGGAGAGTGTTAG.

In another embodiment of the invention, polynucleotides of the invention comprise, or alternatively consist of, the following polynucleotide sequence encoding the variable heavy chain polypeptide sequence of SEQ ID NO: 202:

(SEQ ID NO: 212)
gaggtgcagctTgtggagtctgggggaggcttggtccagcctggggggt
ccctgagactctcctgtgcaGTCtctggaATCGACCTCagtGGCTACTA
CATGAACtgggtccgtcaggctccagggaaggggctggagtgggtcGGA
GTCATTGGTATTAATGGTGCCACATACTACGCGAGCTGGGCGAAAGGCc
gattcaccatctccagagacaattccaagACCACGGTGtatcttcaaat
gaacagcctgagagctgaggacactgctgtgtatTTCtgtGCTAGAGGG
GACATCtggggccaagggaccctcgtcaccgtcTCGAGC.

In one embodiment of the invention, polynucleotides of the invention comprise, or alternatively consist of, the following polynucleotide sequence encoding the heavy chain polypeptide sequence of SEQ ID NO: 201:

(SEQ ID NO: 211)
[]gaggtgcagctTgtggagtctgggggaggcttggtccagcctggggggt
ccctgagactctcctgtgcaGTCtctggaATCGACCTCagtGGCTACTA
CATGAACtgggtccgtcaggctccagggaaggggctggagtgggtcGGA
GTCATTGGTATTAATGGTGCCACATACTACGCGAGCTGGGCGAAAGGCc
gattcaccatctccagagacaattccaagACCACGGTGtatcttcaaat
gaacagcctgagagctgaggacactgctgtgtatTTCtgtGCTAGAGGG
GACATCtggggccaagggaccctcgtcaccgtcTCGAGCGCCTCCACCA
AGGGCCCATCGGTCTTCCCCCTGGCAcCCTCCTCCaAGAGCACCTCTGG
GGGCACAGCGGCCCTGGGCTGCCTGGTCAAGGACTACTTCCCCGAACCG
GTGACGGTGTCGTGGAACTCAGGCGCCCTGACCAGCGGCGTGCACACCT
TCCCGGCTGTCCTACAGTCCTCAGGACTCTACTCCCTCAGCAGCGTGGT
GACCGTGCCCTCCAGCAGCTTGGGCACCCAGACCTACATCTGCAACGTG
AATCACAAGCCCAGCAACACCAAGGTGGACGCGAGAGTTGAGCCCAAAT
CTTGTGACAAAACTCACACATGCCCACCGTGCCCAGCACCTGAACTCCT
GGGGGGACCGTCAGTCTTCCTCTTCCCCCCAAAACCCAAGGACACCCTC
ATGaTCTCCCgGACCCCTGAGGTCACATGCGTGGTGGTGGACGTGAGCC
ACGAAGACCCTGAGGTCAAGTTCAACTGGTACGTGGACGGCGTGGAGGT
GCATAATGCCAAGACAAAGCCGCGGGAGGAGCAGTACGCCAGCACGTAC
CGTGTGGTCAGCGTCCTCACCGTCCTGCACCAGGACTGGCTGAATGGCA
AGGAGTACAAGTGCAAGGTCTCCAACAAAGCCCTCCCAGCCCCCATCGA
GAAAACCATCTCCAAAGCCAAAGGGCAGCCCCGAGAACCACAGGTGTAC
ACCCTGCCCCCATCCCGGGAGGAGATGACCAAGAACCAGGTCAGCCTGA
CCTGCCTGGTCAAAGGCTTCTATCCCAGCGACATCGCCGTGGAGTGGGA
GAGCAATGGGCAGCCGGAGAACAACTACAAGACCACGCCTCCCGTGCTG
GACTCCGACGGCTCCTTCTTCCTCTACAGCAAGCTCACCGTGGACAAGA
GCAGGTGGCAGCAGGGGAACGTCTTCTCATGCTCCGTGATGCATGAGGC
TCTGCACAACCACTACACGCAGAAGAGCCTCTCCCTGTCTCCGGGTAAA
TGA.

In one embodiment of the invention, polynucleotides of the invention comprise, or alternatively consist of, the following polynucleotide sequence encoding the heavy chain polypeptide sequence of SEQ ID NO: 566:

(SEQ ID NO: 567)
gaggtgcagctTgtggagtctgggggaggcttggtccagcctggggggt
ccctgagactctcctgtgcaGTCtctggaATCGACCTCagtGGCTACTA
CATGAACtgggtccgtcaggctccagggaaggggctggagtgggtcGGA
GTCATTGGTATTAATGGTGCCACATACTACGCGAGCTGGGCGAAAGGCc
gattcaccatctccagagacaattccaagACCACGGTGtatcttcaaat
gaacagcctgagagctgaggacactgctgtgtatTTCtgtGCTAGAGGG
GACATCtggggccaagggaccctcgtcaccgtcTCGAGCGCCTCCACCA
AGGGCCCATCGGTCTTCCCCCTGGCAcCCTCCTCCaAGAGCACCTCTGG
GGGCACAGCGGCCCTGGGCTGCCTGGTCAAGGACTACTTCCCCGAACCG
GTGACGGTGTCGTGGAACTCAGGCGCCCTGACCAGCGGCGTGCACACCT
TCCCGGCTGTCCTACAGTCCTCAGGACTCTACTCCCTCAGCAGCGTGGT
GACCGTGCCCTCCAGCAGCTTGGGCACCCAGACCTACATCTGCAACGTG
AATCACAAGCCCAGCAACACCAAGGTGGACGCGAGAGTTGAGCCCAAAT
CTTGTGACAAAACTCACACATGCCCACCGTGCCCAGCACCTGAACTCCT
GGGGGGACCGTCAGTCTTCCTCTTCCCCCCAAAACCCAAGGACACCCTC
ATGaTCTCCCgGACCCCTGAGGTCACATGCGTGGTGGTGGACGTGAGCC
ACGAAGACCCTGAGGTCAAGTTCAACTGGTACGTGGACGGCGTGGAGGT
GCATAATGCCAAGACAAAGCCGCGGGAGGAGCAGTACGCCAGCACGTAC
CGTGTGGTCAGCGTCCTCACCGTCCTGCACCAGGACTGGCTGAATGGCA
AGGAGTACAAGTGCAAGGTCTCCAACAAAGCCCTCCCAGCCCCCATCGA
GAAAACCATCTCCAAAGCCAAAGGGCAGCCCCGAGAACCACAGGTGTAC
ACCCTGCCCCCATCCCGGGAGGAGATGACCAAGAACCAGGTCAGCCTGA
CCTGCCTGGTCAAAGGCTTCTATCCCAGCGACATCGCCGTGGAGTGGGA
GAGCAATGGGCAGCCGGAGAACAACTACAAGACCACGCCTCCCGTGCTG
GACTCCGACGGCTCCTTCTTCCTCTACAGCAAGCTCACCGTGGACAAGA
GCAGGTGGCAGCAGGGGAACGTCTTCTCATGCTCCGTGATGCATGAGGC
TCTGCACAACCACTACACGCAGAAGAGCCTCTCCCTGTCTCCGGGTTGA.

In a further embodiment of the invention, polynucleotides encoding antibody fragments having binding specificity to CGRP comprise, or alternatively consist of, one or more of the polynucleotide sequences of SEQ ID NO: 234; SEQ ID NO: 236; and SEQ ID NO: 238 which correspond to polynucleotides encoding the complementarity-determining regions (CDRs, or hypervariable regions) of the light chain variable sequence of SEQ ID NO: 222 or the light chain sequence of SEQ ID NO: 221.

In a further embodiment of the invention, polynucleotides encoding antibody fragments having binding specificity to CGRP comprise, or alternatively consist of, one or more of the polynucleotide sequences of SEQ ID NO: 214; SEQ ID NO: 216; and SEQ ID NO: 218 which correspond to polynucleotides encoding the complementarity-determining regions (CDRs, or hypervariable regions) of the heavy chain variable sequence of SEQ ID NO: 202 or the heavy chain sequence of SEQ ID NO: 201 or SEQ ID NO: 566.

The invention also contemplates polynucleotide sequences including one or more of the polynucleotide sequences encoding antibody fragments described herein. In one embodiment of the invention, polynucleotides encoding antibody fragments having binding specificity to CGRP comprise, or alternatively consist of, one, two, three or more, including all of the following polynucleotides encoding antibody fragments: the polynucleotide SEQ ID NO: 232 encoding the light chain variable sequence of SEQ ID NO: 222; the polynucleotide SEQ ID NO: 231 encoding the light chain sequence of SEQ ID NO: 221; the polynucleotide SEQ ID NO: 212 encoding the heavy chain variable sequence of SEQ ID NO: 202; the polynucleotide SEQ ID NO: 211 encoding the heavy chain sequence of SEQ ID NO: 201; the polynucleotide SEQ ID NO: 567 encoding the heavy chain sequence of SEQ ID NO: 566; polynucleotides encoding the complementarity-determining regions (SEQ ID NO: 234; SEQ ID NO: 236; and SEQ ID NO: 238) of the light chain variable sequence of SEQ ID NO: 222 or the light chain sequence of SEQ ID NO: 221; and polynucleotides encoding the complementarity-determining regions (SEQ ID NO: 214; SEQ ID NO: 216; and SEQ ID NO: 218) of the heavy chain variable sequence of SEQ ID NO: 202 or the heavy chain sequence of SEQ ID NO: 201 or SEQ ID NO: 566.

In a preferred embodiment of the invention, polynucleotides of the invention comprise, or alternatively consist of, polynucleotides encoding Fab (fragment antigen binding) fragments having binding specificity for CGRP. With respect to antibody Ab6, the polynucleotides encoding the full length Ab6 antibody comprise, or alternatively consist of, the polynucleotide SEQ ID NO: 231 encoding the light chain sequence of SEQ ID NO: 221 and the polynucleotide SEQ ID NO: 211 encoding the heavy chain sequence of SEQ ID NO: 201 or the polynucleotide SEQ ID NO: 567 encoding the heavy chain sequence of SEQ ID NO: 566.

In one embodiment, the invention is directed to an isolated polynucleotide comprising a polynucleotide encoding an anti-CGRP V_{H} antibody amino acid sequence selected from SEQ ID NO: 2, SEQ ID NO: 42, SEQ ID NO: 82, SEQ ID NO: 122, SEQ ID NO: 162, SEQ ID NO: 202, SEQ ID NO: 242, SEQ ID NO: 282, SEQ ID NO: 322, SEQ ID NO: 362, SEQ ID NO: 402, SEQ ID NO: 442, SEQ ID NO: 482, or SEQ ID NO: 522 or encoding a variant thereof wherein at least one framework residue (FR residue) has been substituted with an amino acid present at the corresponding position in a rabbit anti-CGRP antibody V_{H} polypeptide or a conservative amino acid substitution.

In another embodiment, the invention is directed to an isolated polynucleotide comprising the polynucleotide sequence encoding an anti-CGRP V_{L} antibody amino acid sequence of SEQ ID NO: 22, SEQ ID NO: 62, SEQ ID NO: 102, SEQ ID NO: 142, SEQ ID NO: 182, SEQ ID NO: 222, SEQ ID NO: 262, SEQ ID NO: 302, SEQ ID NO: 342, SEQ ID NO: 382, SEQ ID NO: 422, SEQ ID NO: 462, SEQ ID NO: 502, or SEQ ID NO: 542, or encoding a variant thereof wherein at least one framework residue (FR residue) has been substituted with an amino acid present at the corresponding position in a rabbit anti-CGRP antibody V_{L} polypeptide or a conservative amino acid substitution.

In yet another embodiment, the invention is directed to one or more heterologous polynucleotides comprising a sequence encoding the polypeptides contained in SEQ ID NO: 22 and SEQ ID NO: 2; SEQ ID NO: 62 and SEQ ID NO: 42; SEQ ID NO: 102 and SEQ ID NO: 82; SEQ ID NO: 142 and SEQ ID NO: 122; SEQ ID NO: 182 and SEQ ID NO: 162; SEQ ID NO: 222 and SEQ ID NO: 202; SEQ ID NO: 262 and SEQ ID NO: 242; SEQ ID NO: 302 and SEQ ID NO: 282; SEQ ID NO: 342 and SEQ ID NO: 322; SEQ ID NO: 382 and SEQ ID NO: 362; SEQ ID NO: 422 and SEQ ID NO: 402; SEQ ID NO: 462 and SEQ ID NO: 442; SEQ ID NO: 502 and SEQ ID NO: 482; or SEQ ID NO: 542 and SEQ ID NO: 522.

In another embodiment, the invention is directed to an isolated polynucleotide that expresses a polypeptide containing at least one CDR polypeptide derived from an anti-CGRP antibody wherein said expressed polypeptide alone specifically binds CGRP or specifically binds CGRP when expressed in association with another polynucleotide sequence that expresses a polypeptide containing at least one CDR polypeptide derived from an anti-CGRP antibody wherein said at least one CDR is selected from those contained in the V_{L} or V_{H} polypeptides of SEQ ID NO: 22, SEQ ID NO: 2, SEQ ID NO: 62, SEQ ID NO: 42, SEQ ID NO: 102, SEQ ID NO: 82, SEQ ID NO: 142, SEQ ID NO: 122, SEQ ID NO: 182, SEQ ID NO: 162, SEQ ID NO: 222, SEQ ID NO: 202, SEQ ID NO: 262, SEQ ID NO: 242, SEQ ID NO: 302, SEQ ID NO: 282, SEQ ID NO: 342, SEQ ID NO: 322, SEQ ID NO: 382, SEQ ID NO: 362, SEQ ID NO: 422, SEQ ID NO: 402, SEQ ID NO: 462, SEQ ID NO: 442, SEQ ID NO: 502, SEQ ID NO: 482, SEQ ID NO: 542, or SEQ ID NO: 522.

Antibody coding sequences of interest include those encoded by native sequences, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues, etc). Variants also include fragments of the polypeptides disclosed herein, particularly biologically active fragments and/or fragments corresponding to functional domains. Techniques for in vitro mutagenesis of cloned genes are known. Also included in the subject invention are polypeptides that have been modified using ordinary molecular biological techniques so as to improve their resistance to proteolytic degradation or to optimize solubility properties or to render them more suitable as a therapeutic agent.

Vectors as described herein may comprise one or more polynucleotides which individually or in combination encode any of the anti-CGRP antibodies disclosed herein, including but not limited to those comprising the 6 CDRs of Ab1 through Ab14, such as Ab6.

In some embodiments, a polynucleotide encoding the VH (or in some cases the heavy chain) and a polynucleotide encoding the VL (or in some cases the light chain) may be contained in separate vectors.

In some embodiments, separate polynucleotides, one encoding the VH (or in some cases the heavy chain) and another encoding the VL (or in some cases the light chain), may be contained in the same vector.

In some embodiments, a polynucleotide encoding both a VH (or in some cases the heavy chain) and a VL (or in some cases the light chain) may be contained in a vector. In certain embodiments, the VH and VL of the antibody (or in some cases the heavy chain and light chain) may be encoded by separate strands of the same polynucleotide. In some cases, the VH and VL of the antibody (or in some cases the heavy chain and light chain) may be encoded under the same promoter (e.g., bidirectional) or separate promoters. In certain embodiments, the VH and VL of the antibody (or in some cases the heavy chain and light chain) may be encoded by the same strands of the same polynucleotide. In some cases, the VH and VL of the antibody (or in some cases the heavy chain and light chain) may be encoded under the same promoter or separate promoters.

In some embodiments, a vector may be for expressing the encoded anti-CGRP antibody in a host cell of interest, i.e., is an expression vector. In some embodiments, a host cell may be for expressing an anti-CGRP antibody encoded by one or more polynucleotides contained in the cell.

Therefore, these polynucleotides may be incorporated into an expression vector for expression in any appropriate cells, including but not limited to mammalian cells such as CHO, NSO, HEK-293, or fungal, insect, or microbial systems such as yeast cells such as the yeast Pichia. Suitable Pichia species include, but are not limited to, Pichia pastoris.

Expression Vectors, e.g., DNA vectors, typically contain elements that facilitate manipulation for the expression of a foreign protein within the target host cell, e.g., a yeast or mammalian cell such as Pichia pastoris or CHO cells. Conveniently, manipulation of sequences and production of DNA for transformation is first performed in a bacterial host, e.g. E. coli, and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, (b) complement auxotrophic deficiencies, or (c) supply critical nutrients not available from complex media. Exemplary vectors and methods for transformation of yeast are described, for example, in Burke, D., Dawson, D., & Steams, T. (2000). Methods in yeast genetics: a Cold Spring Harbor Laboratory course manual. Plainview, N.Y.: Cold Spring Harbor Laboratory Press.

Expression vectors for use in yeast or mammalian cells will generally further include yeast or mammalian specific sequences, including a selectable auxotrophic or drug marker for identifying transformed yeast strains or transformed mammalian cells. A drug marker may further be used to amplify copy number of the vector in the host cell.

The polypeptide coding sequence of interest may be operably linked to transcriptional and translational regulatory sequences that provide for expression of the polypeptide in host cells, e.g., Pichia pastoris or CHO cells. These vector components may include, but are not limited to, one or more of the following: an enhancer element, a promoter, and a transcription termination sequence. Sequences for the secretion of the polypeptide may also be included, e.g. a signal sequence, and the like. A yeast or mammalian origin of replication is optional, as expression vectors are often integrated into the host cell genome. In one embodiment of the invention, the polypeptide of interest is operably linked, or fused, to sequences providing for optimized secretion of the polypeptide from yeast diploid cells.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites or alternatively via a PCR/recombination method familiar to those skilled in the art (Gateway® Technology; Invitrogen, Carlsbad California). If such sites do not exist, the synthetic oligonucleotide adapters or linkers are used in accordance with conventional practice.

Promoters are untranslated sequences located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (that increase levels of transcription in response to absence of a repressor). Inducible promoters may initiate increased levels of transcription from DNA under their control in response to some change in culture conditions, e.g., the presence or absence of a nutrient or a change in temperature.

The promoter fragment may also serve as the site for homologous recombination and integration of the expression vector into the same site in the host genome; alternatively a selectable marker is used as the site for homologous recombination. Examples of suitable promoters from Pichia include the AOX1 and promoter (Cregg et al. (1989) Mol. Cell. Biol. 9:1316-1323); ICL1 promoter (Menendez et al. (2003) Yeast 20(13):1097-108); glyceraldehyde-3-phosphate dehydrogenase promoter (GAP) (Waterham et al. (1997) Gene 186(1):37-44); and FLD1 promoter (Shen et al. (1998) Gene 216(1):93-102). The GAP promoter is a strong constitutive promoter and the AOX and FLD1 promoters are inducible.

Other yeast promoters include ADH1, alcohol dehydrogenase II, GAL4, PHO3, PHO5, Pyk, and chimeric promoters derived therefrom. Additionally, non-yeast promoters may be used in the invention such as mammalian, insect, plant, reptile, amphibian, viral, and avian promoters. Most typically the promoter will comprise a mammalian promoter (potentially endogenous to the expressed genes) or will comprise a yeast or viral promoter that provides for efficient transcription in yeast systems.

Examples of mammalian promoters include cytomegalovirus (CMV) derived promoters, chicken 3-actin (CBM) derived promoters, adenomatous polyposis coli (APC) derived promoters, leucine-rich repeat containing G protein-coupled receptor 5 (LGR5) promoters, CAG promoter, Beta actin promoter, elongation factor-1 (EF1) promoter, early growth response 1 (EGR-1) promoter, eukaryotic initiation factor 4A (EIF4A1) promoter, simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter, among others. Combinations of two or more of the foregoing promoters may also be used. Further, inducible promoters may be used. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The polypeptides of interest may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the polypeptide coding sequence that is inserted into the vector. The heterologous signal sequence selected preferably is one that is recognized and processed through one of the standard pathways available within the host cell. The S. cerevisiae alpha factor pre-pro signal has proven effective in the secretion of a variety of recombinant proteins from P. pastoris. Other yeast signal sequences include the alpha mating factor signal sequence, the invertase signal sequence, and signal sequences derived from other secreted yeast polypeptides. Additionally, these signal peptide sequences may be engineered to provide for enhanced secretion in diploid yeast expression systems. Secretion signals for use in mammalian as well as yeast cells include mammalian signal sequences, which may be heterologous to the protein being secreted, or may be a native sequence for the protein being secreted. Signal sequences include pre-peptide sequences, and in some instances may include propeptide sequences. Many such signal sequences are known in the art, including the signal sequences found on immunoglobulin chains, e.g., K28 preprotoxin sequence, PHA-E, FACE, human MCP-1, human serum albumin signal sequences, human Ig heavy chain, human Ig light chain, and the like. For example, see Hashimoto et. al. Protein Eng 11(2) 75 (1998); and Kobayashi et. al. Therapeutic Apheresis 2(4) 257 (1998).

Transcription may be increased by inserting a transcriptional activator sequence into the vector. These activators are cis-acting elements of DNA, usually about from 10 to 300 bp, which act on a promoter to increase its transcription. Transcriptional enhancers are relatively orientation and position independent, having been found 5′ and 3′ to the transcription unit, within an intron, as well as within the coding sequence itself. The enhancer may be spliced into the expression vector at a position 5′ or 3′ to the coding sequence, but is preferably located at a site 5′ from the promoter.

Expression vectors used in eukaryotic host cells may also contain sequences necessary for the termination of transcription and for stabilizing the mRNA. Such sequences are commonly available from 3′ to the translation termination codon, in untranslated regions of eukaryotic or viral DNAs or cDNAs. These regions contain nucleotide segments transcribed as polyadenylated fragments in the untranslated portion of the mRNA.

Construction of suitable vectors containing one or more of the above-listed components employs standard ligation techniques or PCR/recombination methods. Isolated plasmids or DNA fragments are cleaved, tailored, and re-ligated in the form desired to generate the plasmids required or via recombination methods. For analysis to confirm correct sequences in plasmids constructed, the ligation mixtures are used to transform host cells, and successful transformants selected by antibiotic resistance (e.g. ampicillin or Zeocin) where appropriate. Plasmids from the transformants are prepared, analyzed by restriction endonuclease digestion and/or sequenced.

As an alternative to restriction and ligation of fragments, recombination methods based on att sites and recombination enzymes may be used to insert DNA sequences into a vector. Such methods are described, for example, by Landy (1989) Ann. Rev. Biochem. 58:913-949; and are known to those of skill in the art. Such methods utilize intermolecular DNA recombination that is mediated by a mixture of lambda and E. coli-encoded recombination proteins. Recombination occurs between specific attachment (att) sites on the interacting DNA molecules. For a description of att sites see Weisberg and Landy (1983) Site-Specific Recombination in Phage Lambda, in Lambda II, Weisberg, ed. (Cold Spring Harbor, NY:Cold Spring Harbor Press), pp. 211-250. The DNA segments flanking the recombination sites are switched, such that after recombination, the att sites are hybrid sequences comprised of sequences donated by each parental vector. The recombination can occur between DNAs of any topology.

Att sites may be introduced into a sequence of interest by ligating the sequence of interest into an appropriate vector; generating a PCR product containing aft B sites through the use of specific primers; generating a cDNA library cloned into an appropriate vector containing att sites; and the like.

Folding, as used herein, refers to the three-dimensional structure of polypeptides and proteins, where interactions between amino acid residues act to stabilize the structure. Proper folding is typically the arrangement of a polypeptide that results in optimal biological activity, and in the case of antibodies can conveniently be monitored by assays for activity, e.g. antigen binding.

The expression host may be further modified by the introduction of sequences encoding one or more enzymes that enhance folding and disulfide bond formation, i.e. foldases, chaperonins, etc. Such sequences may be constitutively or inducibly expressed in the yeast host cell, using vectors, markers, etc. as known in the art. Preferably the sequences, including transcriptional regulatory elements sufficient for the desired pattern of expression, are stably integrated in the yeast genome through a targeted methodology.

For example, the eukaryotic PDI is not only an efficient catalyst of protein cysteine oxidation and disulfide bond isomerization, but also exhibits chaperone activity. Co-expression of PDI can facilitate the production of active proteins having multiple disulfide bonds. Also of interest is the expression of BIP (immunoglobulin heavy chain binding protein); cyclophilin; and the like. In one embodiment of the invention, each of the haploid parental strains expresses a distinct folding enzyme, e.g. one strain may express BIP, and the other strain may express PDI or combinations thereof.

In some embodiments, the cell may be or may comprise a non-mammalian cell, optionally bacterial, yeast, fungal, protozoa, plant, or insect, bacterial cell(s). In some embodiments, the cell may be or may comprise a mammalian cell, optionally human, non-human primate, monkey, rabbit, rodent, hamster, rat, or mouse cell(s). In particular embodiments of the invention, the host cell may comprise an yeast cell, optionally belonging to the genus Pichia. In particular embodiments, the host cell may comprise a Chinese Hamster Ovary (CHO) cell, a lymphoid cell (e.g., Y0, NS0, Sp20 cell), or a HEK cell (e.g., HEK293 cell).

### Methods of Producing Antibodies and Fragments Thereof

In one aspect, the present invention contemplates methods for producing anti-CGRP antibodies and fragments thereof. Any appropriate methods may be used to produce an anti-CGRP antibody described herein.

In some embodiments, the antibody may be produced by culturing a host cell comprising one or more polynucleotides encoding an anti-CGRP antibody of interest to allow for expression of the anti-CGRP antibody. The host cells may be for example produced by introducing the one or more polynucleotides encoding an anti-CGRP antibody of interest into a host cell type of interest. A vector such as an expression vector containing such one or more polynucleotides may be used for introducing the polynucleotides in the cell. The expressed anti-CGRP may be harvested using any appropriate manner, including but not limited to collecting supernatant of the cell culture (if the antibody is secreted) and/or lysing the host cell (e.g., for antibodies remaining in the cell) and purifying the desired antibody (e.g., using protein A purification and/or appropriate chromatography).

In one embodiment of the invention described herein (infra), Fab fragments may be produced by enzymatic digestion (e.g., papain) of Ab6 (or another anti-CGRP antibody described herein) following expression of the full-length polynucleotides in a suitable host.

In some embodiments, anti-CGRP antibodies such as Ab6 (or another anti-CGRP antibody described herein) or Fab fragments thereof may be produced via expression of Ab6 polynucleotides in any appropriate cells, including but not limited to mammalian cells such as CHO, NSO or HEK 293 cells, or fungal, insect, or microbial systems such as yeast cells (for example diploid yeast such as diploid Pichia) and other yeast strains. Suitable Pichia species include, but are not limited to, Pichia pastoris.

The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammals, chicken, other avians, etc., are considered to be “antibodies.” A preferred source for producing antibodies useful as starting material (e.g., the source/origin of the CDRs) according to the invention is rabbits. Numerous antibody coding sequences have been described; and others may be raised by methods well-known in the art. Examples thereof include chimeric antibodies, human antibodies and other non-human mammalian antibodies, humanized antibodies, single chain antibodies (such as scFvs), camelbodies, nanobodies, IgNAR (single-chain antibodies derived from sharks), small-modular immunopharmaceuticals (SMIPs), and antibody fragments such as Fabs, Fab′, F(ab′)₂ and the like. See Streltsov V A, et al., Structure of a shark IgNAR antibody variable domain and modeling of an early-developmental isotype, Protein Sci. 2005 November; 14(11):2901-9. Epub 2005 Sep. 30; Greenberg A S, et al., A new antigen receptor gene family that undergoes rearrangement and extensive somatic diversification in sharks, Nature. 1995 Mar. 9; 374(6518):168-73; Nuttall S D, et al., Isolation of the new antigen receptor from wobbegong sharks, and use as a scaffold for the display of protein loop libraries, Mol Immunol. 2001 August; 38(4):313-26; Hamers-Casterman C, et al., Naturally occurring antibodies devoid of light chains, Nature. 1993 Jun. 3; 363(6428):446-8; Gill D S, et al., Biopharmaceutical drug discovery using novel protein scaffolds, Curr Opin Biotechnol. 2006 December; 17(6):653-8. Epub 2006 Oct. 19.

For example, antibodies or antigen binding fragments may be produced by genetic engineering. In this technique, as with other methods, antibody-producing cells are sensitized to the desired antigen or immunogen. The messenger RNA isolated from antibody producing cells is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host cell. When antibody gene synthesis is induced in the transfected host, the heavy and light chain proteins self-assemble to produce active antibodies that can be detected by screening with the antigen or immunogen.

Methods for producing antibodies and fragments thereof secreted from polyploidal, preferably diploid or tetraploid strains of mating competent yeast are taught, for example, in U.S. patent application publication no. US 2009/0022659 to Olson et al., and in U.S. Pat. No. 7,935,340 to Garcia-Martinez et al., the disclosures of each of which are herein incorporated by reference in their entireties. Methods for producing antibodies and fragments thereof in mammalian cells, e.g., CHO cells are further well known in the art.

Other methods of producing antibodies are also well known to those of ordinary skill in the art. For example, methods of producing chimeric antibodies are now well known in the art (See, for example, U.S. Pat. No. 4,816,567 to Cabilly et al.; Morrison et al., P.N.A.S. USA, 81:8651-55 (1984); Neuberger, M. S. et al., Nature. 314:268-270 (1985); Boulianne, G. L. et al., Nature, 312:643-46 (1984), the disclosures of each of which are herein incorporated by reference in their entireties).

Likewise, other methods of producing humanized antibodies are now well known in the art (See, for example, U.S. Pat. Nos. 5,530,101, 5,585,089, 5,693,762, and 6,180,370 to Queen et al; U.S. Pat. Nos. 5,225,539 and 6,548,640 to Winter; U.S. Pat. Nos. 6,054,297, 6,407,213 and 6,639,055 to Carter et al; U.S. Pat. No. 6,632,927 to Adair; Jones, P. T. et al, Nature, 321:522-525 (1986); Reichmann, L., et al, Nature. 332:323-327 (1988); Verhoeyen, M, et al, Science, 239:1534-36 (1988), the disclosures of each of which are herein incorporated by reference in their entireties).

Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward in practice. See, e.g., U.S. Pat. No. 6,187,287, incorporated fully herein by reference. Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Chimeric antibodies may be made by recombinant means by combining the VH and VL obtained from antibody producing cells of one species with the constant light and heavy chain regions from another. Typically chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated herein by reference in its entirety). It is further contemplated that the human constant regions of chimeric antibodies of the invention may be selected from IgG1, IgG2, IgG3, and IgG4 constant regions.

### Administration (Dose, Frequency, Route, Etc)

In one embodiment of the invention, the anti-CGRP antibodies described herein, or CGRP binding fragments thereof, as well as combinations of said antibodies or antibody fragments, are administered to a subject at a concentration of between about 0.1 and 100.0 mg/kg of body weight of recipient subject.

In a preferred embodiment of the invention, the anti-CGRP antibodies described herein, or CGRP binding fragments thereof, as well as combinations of said antibodies or antibody fragments, are administered to a subject at a concentration of about 0.4 mg/kg of body weight of recipient subject and/or at a dosage of about 50 mg to about 1500 mg. In some cases, a subject is administered about 50 to about 1000 mg, about 100 mg to about 500 mg, or about 100 mg to about 300 mg of an anti-CGRP antibody. In particular cases, a subject is administered about 100 or about 300 mg of an anti-CGRP antibody.

In a preferred embodiment of the invention, the anti-CGRP antibodies described herein, or CGRP binding fragments thereof, as well as combinations of said antibodies or antibody fragments, or pharmaceutical compositions described herein are administered to a recipient subject with a frequency of once every twenty-six weeks or six months or less, such as once every sixteen weeks or four months or less, once every eight weeks or two months or less, once every four weeks or monthly or less, once every two weeks or bimonthly or less, once every week or less, or once daily or less. In general the administration of sequential doses may vary by plus or minus a few days from the aforementioned schedule, e.g., administration every 3 months or every 12 weeks includes administration of a dose varying from the schedule day by plus or minus 1, 2, 3, 4, 5, 5, or 7 days.

Fab fragments may be administered every two weeks or less, every week or less, once daily or less, multiple times per day, and/or every few hours. In one embodiment of the invention, a patient receives Fab fragments of 0.1 mg/kg to 40 mg/kg per day given in divided doses of 1 to 6 times a day, or in a sustained release form, effective to obtain desired results.

It is to be understood that the concentration of the antibody or Fab administered to a given patient may be greater or lower than the exemplary administration concentrations set forth above.

A person of skill in the art would be able to determine an effective dosage and frequency of administration through routine experimentation, for example guided by the disclosure herein and the teachings in Goodman, L. S., Gilman, A., Brunton, L. L., Lazo, J. S., & Parker, K. L. (2006). Goodman & Gilman's the pharmacological basis of therapeutics. New York: McGraw-Hill; Howland, R. D., Mycek, M. J., Harvey, R. A., Champe, P. C., & Mycek, M. J. (2006). Pharmacology. Lippincott's illustrated reviews. Philadelphia: Lippincott Williams & Wilkins; and Golan, D. E. (2008). Principles of pharmacology: the pathophysiologic basis of drug therapy. Philadelphia, Pa., [etc.]: Lippincott Williams & Wilkins.

For administering an anti-CGRP antibody or a pharmaceutical composition according to the present disclosure to a subject in need thereof, any appropriate administration route may be used.

In some embodiments, an anti-CGRP antibody or a pharmaceutical composition may be administered parenterally. In certain embodiments, the administration may be by injection, such as but not limited to intravenous, intramuscular, subcutaneous, intradermal, intraparenchymal, intrathecal, intraarterial, intraarticular, intraosseous, or intraperitoneal administration, or by inhalation.

In some embodiments, an anti-CGRP antibody or a pharmaceutical composition may be administered locally. In certain embodiments, the administration may be to the eye, ear, nose (optionally intranasally), skin (optionally transdermally or epicutaneously), mucosa, skin, or vagina, or by inhalation.

In some embodiments, an anti-CGRP antibody or a pharmaceutical composition may be administered enterally. In certain embodiments, an anti-CGRP antibody or a pharmaceutical composition may be administered orally, sublingually, buccally, or rectally.

In particular embodiments, an anti-CGRP antibody or a pharmaceutical composition may be administered to a subject intravenously.

Administration of an anti-CGRP antibody or a pharmaceutical composition to a subject according to the present disclosure may start or occur while the subject is experiencing a symptom, and the administration may ameliorate or treat the already ongoing symptom in the subject.

In some embodiments, the administration may start or occur while the subject has a headache and/or a migraine attack (e.g., the symptom may be a head pain, photophobia, phonophobia, and/or nausea), and the administration may ameliorate or treat the headache or one or more symptoms of migraine.

In some embodiments, the administration may start or occur while the subject has one or more depressive symptoms or a depressive episode, and the administration may ameliorate or treat one or more of the depressive symptoms or the depressive episode. In certain embodiments, the administration may reduce the severity and/or duration of the symptom or episode that is already ongoing and/or reduce the severity, duration, and/or frequency of future depressive symptoms or episodes.

### Pharmaceutical Compositions

In some embodiments of the invention, the anti-CGRP antibodies described herein, or CGRP-binding fragments thereof, as well as combinations of said antibodies or antibody fragments and/or a combination of an anti-CGRP antibody and another medication, are administered to a subject in a pharmaceutical formulation or composition.

In some embodiments, one or more polynucleotides encoding an anti-CGRP antibody or antigen-binding fragment thereof as disclosed herein may be administered to a subject in a pharmaceutical formulation or composition.

In some embodiments, one or more vectors comprising one or more polynucleotides encoding an anti-CGRP antibody or antigen-binding fragment thereof as disclosed herein may be administered to a subject in a pharmaceutical formulation or composition.

In some embodiments, one or more host cells comprising one or more polynucleotides encoding an anti-CGRP antibody or antigen-binding fragment thereof as disclosed herein may be administered to a subject in a pharmaceutical formulation or composition.

A pharmaceutical formulation or composition may comprise such an active ingredient (one or more antibodies, one or more polynucleotides, one or more vectors, or one or more host cells) and a pharmaceutically acceptable carrier.

In some embodiments, a pharmaceutical formulation or composition may further comprise another active ingredient such as another medication.

In certain embodiments, said another active ingredient may comprise one or more antidepressants. Such a formulation or composition may be administered to a subject when the subject has one or more depressive symptoms or have a depressive disorder. In some cases, the antidepressants may be selected from any of the antidepressants described herein. In certain cases, the anti-CGRP antibody according to the present disclosure and the one or more antidepressants may provide a synergistic effect in reducing, preventing, or treating one or more depressive symptoms or depression.

In certain embodiments, said another active ingredient may comprise one or more headache or migraine medications. Such a formulation or composition may be administered to a subject when the subject has headache or migraine. In some cases, the headache or migraine medications may be selected from any of the headache or migraine medications described herein. In certain cases, the anti-CGRP antibody according to the present disclosure and the one or more headache or migraine medications may provide a synergistic effect in reducing, preventing, or treating headache or migraine.

Pharmaceutical compositions typically must be sterile and stable under the conditions of manufacture and storage. The invention contemplates that the pharmaceutical composition is present in lyophilized form. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol), and suitable mixtures thereof. The invention further contemplates the inclusion of a stabilizer in the pharmaceutical composition. The proper fluidity can be maintained, for example, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the alkaline polypeptide can be formulated in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

An exemplary composition comprises, consists essentially of, or consists of an anti-CGRP antibody or fragment thereof (e.g., Ab6), an excipient such as histidine, an isotonic agent such as sorbitol, and a surfactant such as polysorbate 80 in an aqueous solution. For example, the composition may comprise, consist essentially of, or consist of histidine (L-histidine), sorbitol, polysorbate 80, such as, per 1 mL volume, about 100 mg anti-CGRP antibody (e.g., Ab6), about 3.1 mg L-Histidine, about 40.5 mg Sorbitol, and about 0.15 mg Polysorbate 80, having a pH of about 5.8, or approximately that constitution, e.g., within 10% of those values, within 5% of those values, within 1% of those values, within 0.5% of those values, or within 0.1% of those values, and water. For example, the pH value may be within 10% of 5.8, i.e., between 5.22 and 6.38. The Ab6 antibody may comprise or consist of the variable light and heavy chain polypeptides of SEQ ID NO: 222 and SEQ ID NO: 202 respectively, or the light and heavy chain polypeptides of SEQ ID NO: 221 and SEQ ID NO: 201 respectively, or the light and heavy chain polypeptides of SEQ ID NO: 221 and SEQ ID NO: 566 respectively. The composition may be in the form of an aqueous solution, or a concentrate (e.g., lyophilized) which when reconstituted, e.g., by addition of water, yields the aforementioned constitution. An exemplary composition consists of, per mL, 100 mg of the light and heavy chain polypeptides of SEQ ID NO: 221 and SEQ ID NO: 201 respectively, about 3.1 mg L-Histidine, about 40.5 mg Sorbitol, and about 0.15 mg Polysorbate 80, and water Q.S, or approximately that constitution, e.g., within 10% of those quantities, within 5% of those quantities, within 1% of those quantities, within 0.5% of those quantities, or within 0.1% of those quantities. Another exemplary composition consists of, per mL, 100 mg of the light and heavy chain polypeptides of SEQ ID NO: 221 and SEQ ID NO: 566 respectively, about 3.1 mg L-Histidine, about 40.5 mg Sorbitol, and about 0.15 mg Polysorbate 80, and water Q.S, or approximately that constitution, e.g., within 10% of those quantities, within 5% of those quantities, within 1% of those quantities, within 0.5% of those quantities, or within 0.1% of those quantities. The composition may be suitable for intravenous or subcutaneous administration, preferably intravenous administration. For example, the composition may be suitable for mixing with an intravenous solution (such as 0.9% sodium chloride) at an amount of between about 100 mg and about 300 mg antibody added to 100 mL of intravenous solution. Preferably the composition may be shelf-stable for at least 1, 3, 6, 12, 18, or 24 months, e.g., showing formation of aggregates of no more than 5% or no more than 10% of the antibody or fragment after storage at room temperature or when refrigerated at 4° C. for the specified duration, or in an accelerated aging test that simulates storage for that duration.

For each of the recited embodiments, the compounds can be administered by a variety of dosage forms. Any biologically-acceptable dosage form known to persons of ordinary skill in the art, and combinations thereof, are contemplated. Examples of such dosage forms include, without limitation, reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal, preferably intravenous), infusions, and combinations thereof.

### Subjects/Patients

In one aspect, an anti-CGRP antibody or a pharmaceutical composition according to the present disclosure may be administered to a subject to prevent or treat headache or migraine.

In some embodiments, the subject may be a mammal, such as but not limited to a human, a non-human primate, a monkey, a horse, a cow, sheep, a goat, a pig, a dog, a cat, a rabbit, a rodent (mouse, rat, guinea pig, hamster), a rat, or a mouse.

In particular embodiments, the subject may be a human.

In some embodiments, the subject administered an anti-CGRP antibody or a pharmaceutical composition according to the present disclosure may have headache and/or migraine and may also have one or more psychiatric symptoms, such as one or more depressive symptoms.

In certain embodiments, the migraine may be episodic migraine. In certain embodiments, the migraine may be chronic migraine. In certain embodiments, the migraine may be migraine without aura. In certain embodiments, the migraine may be migraine with aura.

In certain embodiments, the subject may have a history of previous failure with one or more, optionally two or more, preventive migraine medications. In particular embodiments, the subject may have a history of previous failure with two to four preventive migraine medications.

In some cases, the previous failure may be with one or more of the following preventive migraine medications: (i) antiepileptic drugs or anticonvulsants; (ii) beta-blockers; (iii) triptans; (iv) antidepressants (such as SSRI, SNRI, and TCA); (v) calcium channel blocker; (vi) angiotensin II receptor antagonists; (vii) ACE inhibitors; (viii) α-agonists; (ix) antihistamines; and (x) other medications approved for prevention of migraine.

In certain cases, the previous failure may be with one or more of the following preventive migraine medications: (i) divalproex, valproate, topiramate, and carbamazepine; (ii) propranolol, metoprolol, timolol, atenolol, nadolol, nebivolol, and pindolol; (iii) frovatriptan, naratriptan, and zolmitriptan; (iv) amitriptyline and venlafaxine; (v) flunarizine; (vi) candesartan; (vii) lisinopril; (viii) clonidine and guanfacine; and (ix) cyproheptadine.

In particular cases, the previous failure may be with one or more of the following preventive migraine medications: beta-blockers, such as propranolol or metoprolol; anticonvulsants such as topiramate, valproate, or divalproex; tricyclic antidepressants such as amitriptyline; calcium channel blocker such as flunarizine; angiotensin II receptor antagonist such as candesartan; and other medications approved for prevention of migraine.

In another aspect, an anti-CGRP antibody or a pharmaceutical composition according to the present disclosure may be administered to a subject to prevent or treat one or more psychiatric symptoms (e.g., depressive symptoms) or psychiatric disorder (e.g., depressive disorder).

In some embodiments, the subject administered an anti-CGRP antibody or a pharmaceutical composition according to the present disclosure may have one or more psychiatric symptoms, such as one or more depressive symptoms. In some embodiments, such a subject may have and/or be diagnosed with a psychiatric disorder, such as a depressive disorder.

In certain embodiments, a subject may have and/or be diagnosed with a major depressive disorder. In certain embodiments, a subject may have experienced one or more major depressive episodes, for example within a year, 2, 3, 4, 5, 6, 7, 8, 9, or 10 years before administration of the anti-CGRP antibody or pharmaceutical composition according to the present disclosure.

In certain embodiments, a subject may have and/or be diagnosed with a minor depressive disorder. In certain embodiments, a subject may have experienced one or more minor depressive episodes, for example within a year, 2, 3, 4, 5, 6, 7, 8, 9, or 10 years before administration of the anti-CGRP antibody or pharmaceutical composition according to the present disclosure.

In certain embodiments, a subject may have one or more of the depressive symptoms (1)-(9) described herein, i.e., (1) depressed mood (or irritable mood in children and adolescents); (2) diminished interest in pleasure; (3) weight loss when not dieting or weight gain (e.g., a change of more than 5% of body weight in a month), or decrease or increase in appetite nearly every day (or failure to make expected weight gain in children); (4) insomnia or hypersomnia; (5) psychomotor agitation or retardation; (6) fatigue or loss of energy; (7) feelings of worthlessness or excessive or inappropriate guilt; (8) diminished ability to think or concentrate, or indecisiveness; and (9) recurrent thoughts of death, recurrent suicidal ideation without a specific plan, or a suicide attempt or a specific plan for committing suicide.

In certain embodiments, a subject may have at least one or more of (1) depressed mood and (2) loss of interest or pleasure.

In certain embodiments, a subject may have suicidal ideation.

In certain embodiments, a subject may present five or more of the depressive symptoms (1)-(9). In particular embodiments, a subject may present five of the depressive symptoms (1)-(9). In particular embodiments, a subject may present six of the depressive symptoms (1)-(9). In particular embodiments, a subject may present seven of the depressive symptoms (1)-(9). In particular embodiments, a subject may present eight of the above depressive symptoms (1)-(9). In particular embodiments, a subject may present all of the above depressive symptoms (1)-(9).

In certain embodiments, a subject may present two to four of the depressive symptoms (1)-(9). In particular embodiments, a subject may present two of the depressive symptoms (1)-(9). In particular embodiments, a subject may present three of the depressive symptoms (1)-(9). In particular embodiments, a subject may present four of the depressive symptoms (1)-(9).

In certain embodiments, the subject may further have headache and/or migraine.

In certain embodiments, the subject may not have headache and/or migraine.

In certain embodiments, a subject may have a history of previous failure with one or more antidepressants. In some cases, the antidepressants may be any of those described herein. In certain cases, the antidepressants may be selected from tricyclic antidepressants (TCAs), monoamine oxidase inhibitors (MAOIs), serotonin reuptake inhibitors (SSRIs), serotonin (5HT)-norepinephrine (NE) reuptake inhibitors (SNRIs), tetracyclic antidepressants (TeCAs), serotonin antagonist and reuptake inhibitors (SARIs), N-methyl-D-aspartate (NMDA) antagonists, serotonin-dopamine activity modulators (SDAMs), and other antidepressants. In particular cases, the antidepressants may be a TCA, such as imipramine, imipramine oxide, amitriptyline, desipramine, nortriptyline, amoxapine, clomipramine, lofepramine, dosulepin, doxepin, trimipramine, protriptyline, opipramol, dibenzepin, butriptyline, amineptine, iprindole, melitracen, dimetacrine, or quinupramine. In particular embodiments, a subject may have a history of previous failure with a TCA, such as amitriptyline.

### Readouts

The effects of the treatment according to the present disclosure may be evaluated using any appropriate parameters.

In some embodiments, the effect on headache frequency may be evaluated based on changes in the monthly headache days (MHD). As used herein, the term “monthly headache days (MHD)” (also referred to as “headache days per month”) refers to the number of days per month on which a patient has a headache, i.e., at any time during that day, the patient has symptoms that meet the clinical definition of a headache. The number of headache days per month may be determined by recording each day whether or not a headache occurred. In certain embodiments, a method according to the present disclosure may reduce MHD by at least 3, 4, or 5 days. In certain embodiments, a method according to the present disclosure may reduce MHD by at least 7 days. In certain embodiments, a method according to the present disclosure may reduce MHD by at least about 30%, at least about 40%, or at least about 50%.

In some embodiments, the effect on pain levels may be evaluated using the 4-point verbal rating scale (VRS-4) (see “The International Classification of Headache Disorders, 3rd edition”, Cephalalgia, 2018, Vol. 38(1) 1-211, at pg. 210 (“intensity of pain”)). As used herein, the terms “4-point scale” or “4-point pain scale” or “VRS” or “VRS-4” refer to the 4-point verbal rating scale (VRS) used to measure pain (VRS-4) (see “The International Classification of Headache Disorders, 3rd edition”, Cephalalgia, 2018, Vol. 38(1) 1-211, at pg. 210 (“intensity of pain”)). In the VRS-4, the patient is asked to rate the pain verbally on a 4-point scale (between 0 and 3), with 3 being severe, 2 being moderate, 1 being mild, and 0 being no pain. It may also be scored on a verbal rating scale expressed in terms of its functional consequence: 0, no pain; 1, mild pain, does not interfere with usual activities; 2, moderate pain, inhibits but does not wholly prevent usual activities; 3, severe pain, prevents all activities. In certain embodiments, a method according to the present disclosure may reduce the severity of pain in a subject by at least 1 point. In certain embodiments, a method according to the present disclosure may reduce the severity of pain in a subject by at least 2 points. In certain embodiments, a method according to the present disclosure may reduce the severity of pain in a subject by at least 3 points. In certain embodiments, a method according to the present disclosure may reduce the severity of pain in a subject by 4 points.

In some embodiments, the effect on migraine in a subject may be evaluated based on changes in the monthly migraine days (MMD). As used herein, the term “monthly migraine days (MMD)” (also referred to as “migraine days per month”) refers to the number of days per month on which a patient has a migraine attack, i.e., at any time during that day, the patient has symptoms that meet the clinical definition of migraine. The number of migraine days per month may be determined by recording each day whether or not a migraine occurred. A migraine attack may be defined to be an attack that meets the migraine definition as outlined in the third edition of the International Classification of Headache Disorders (ICHD-3) (see Headache Classification Committee of the International Headache Society (IHS), The International Classification of Headache Disorders, 3rd edition, Cephalalgia. 2018 January; 38(1):1-211, which is hereby incorporated by reference in its entirety). In certain embodiments, a method according to the present disclosure may reduce MMD by at least 3, 4, or 5 days. In certain embodiments, a method according to the present disclosure may reduce MMD by at least 7 days. In certain embodiments, a method according to the present disclosure may reduce MMD by at least about 30%, at least about 40%, or at least about 50%.

In some embodiments, the effect on migraine in a subject population may be evaluated based on ≥50% migraine responder rates (MRRs). ≥50% MRR is the proportion (such as %) of a subject population who experienced 50% or more reduction in MMD.

In some embodiments, the effect on migraine in a subject population may be evaluated based on the number of acute headache or migraine medications per month taken by the subject (monthly medication days). In certain embodiments, a method according to the present disclosure may reduce monthly medication days by at least 3, 4, or 5 days. In certain embodiments, a method according to the present disclosure may reduce monthly medication days by at least 7 days. In certain embodiments, a method according to the present disclosure may reduce monthly medication days by at least about 30%, at least about 40%, or at least about 50%.

In some embodiments, the effect on migraine may be evaluated based on the patient global impression of change (PGIC) associated with migraine. PGIC is a parameter comprising a single question concerning the patient's own impression of the overall change (improvement or worsening since the start of the study, i.e., baseline) in the disease status evaluated on a 7-point Likert scale of very much improved, much improved, minimally improved, no change, minimally worse, much worse, and very much worse. In certain embodiments, treatment may be considered effective when the PGIC is “minimally improved”, “much improved”, or “very much improved”. In particular embodiments, treatment may be considered effective when the PGIC is “much improved” or “very much improved”.

In some embodiments, the effect on migraine may be evaluated using the 6-item Headache Impact Test (HIT-6). HIT-6 is a well well-known tool for assessing the impact of headache. It uses six questions and helps patients describe and communicate the way patients feel and what patients cannot do because of headaches. Patient answers each of the six questions using: “never”, “rarely”, “sometimes”, “very often”, or “always”. The responses are summed to produce a total HIT-6 score that ranges from 36 to 78, where a higher score indicates a greater impact of headache on the daily life of the respondent. The headache severity may be categorized as little or no impact (HIT-6 score of 49 or less), some impact (HIT-6 score of 50-55), substantial impact (HIT-6 score of 56-59), and severe impact (HIT-6 score of 60-78). In certain embodiments, an improvement in the HIT-6 score may mean a reduction of the score by at least 2.3 (units). In certain embodiments, an improvement in the HIT-6 score may mean a reduction of the score by at least 3. In certain embodiments, an improvement in the HIT-6 score may mean a reduction of the score by at least 5. In certain embodiments, an improvement in the HIT-6 score may mean ≥1 categorical change in the headache severity category (e.g., change from substantial impact to some impact).

In some embodiments, the effect on migraine may be evaluated based on the Migraine-Specific Quality of Life questionnaire (MSQ). MSQ is one of the most commonly used disease-specific tools for assessing the impact of migraine on quality of life (QOL), developed by Glaxo Wellcome Inc. MSQ is a 14-item (in case of Version 2.1) measure of health-related QOL evaluating impacts attributed to migraine over the past 4 weeks and comprises 3 domains: (i) Role Function-Restrictive (RFR); (it) Role Function-Preventive (RFP); and (iii) Emotional Function (EF). The impact may be categorized as normal (0-2), mild (3-5), moderate (6-8), and severe (9-12). In certain embodiments, a method according to the present disclosure may improve MSQ by at least 2 points (e.g., 12 to 10, 8 to 6 etc). In certain embodiments, a method according to the present disclosure may improve MSQ by at least 3 points (e.g., 12 to 9 etc). In certain embodiments, a method according to the present disclosure may improve MSQ by at least 1 category (e.g., sever to moderate, moderate to mild, or mild to normal). In certain embodiments, a method according to the present disclosure may improve MSQ by at least 2 categories (e.g., sever to mild, or moderate to normal). In certain embodiments, a method according to the present disclosure may improve MSQ by 3 categories (e.g., sever to normal).

Evaluation of effects of the treatment may be for example based on the comparison of a parameter of interest at baseline (e.g., before treatment, day 0 before administration, etc) and at one or more time points of interest after the treatment (e.g., at least after one administration).

In some cases, the one or more timepoints of interest after the treatment may be about 3-90 weeks after the first treatment, about 3 weeks after the first treatment, about 6 weeks after the first treatment, about 9 weeks after the first treatment, about 12 weeks after the first treatment, about 15 weeks after the first treatment, about 18 weeks after the first treatment, about 21 weeks after the first treatment, about 24 weeks after the first treatment, about 27 weeks after the first treatment, about 30 weeks after the first treatment, about 33 weeks after the first treatment, about 36 weeks after the first treatment, about 39 weeks after the first treatment, about 42 weeks after the first treatment, about 45 weeks after the first treatment, about 48 weeks after the first treatment, about 51 weeks after the first treatment, about 54 weeks after the first treatment, about 57 weeks after the first treatment, about 60 weeks after the first treatment, about 63 weeks after the first treatment, about 66 weeks after the first treatment, about 69 weeks after the first treatment, about 72 weeks after the first treatment, about 75 weeks after the first treatment, about 78 weeks after the first treatment, about 81 weeks after the first treatment, about 84 weeks after the first treatment, about 87 weeks after the first treatment, about 90 weeks after the first treatment, or more weeks after the treatment.

In some cases, the one or more timepoints of interest after the treatment may be about 1-24 months after the first treatment, about 1 month after the first treatment, about 2 months after the first treatment, about 3 months after the first treatment, about 4 months after the first treatment, about 5 months after the first treatment, about 6 months after the first treatment, about 7 months after the first treatment, about 8 months after the first treatment, about 9 months after the first treatment, about 10 months after the first treatment, about 11 months after the first treatment, about 12 months after the first treatment, about 13 months after the first treatment, about 14 months after the first treatment, about 15 months after the first treatment, about 16 months after the first treatment, about 17 months after the first treatment, about 18 months after the first treatment, about 19 months after the first treatment, about 20 months after the first treatment, about 21 months after the first treatment, about 22 months after the first treatment, about 23 months after the first treatment, about 24 months after the first treatment, or more months from the first treatment.

In some cases, the one or more time points of interest after the treatment may be about 1-6 years after the first treatment, about 1 year after the first treatment, about 2 years after the first treatment, about 3 years after the first treatment, about 4 years after the first treatment, about 5 years after the first treatment, about 6 years after the first treatment, or more years after the first treatment.

In some embodiments, the comparison may be made relative to “the baseline number of migraine days” or “the baseline number of headache days”, which means the number of migraine days or headache days, respectively, exhibited by a subject in a specified time period, e.g., prior to treatment. For example, the baseline number of migraine days may be determined over a period of one month, or longer, e.g., by recording each day whether or not a migraine occurred.

In certain embodiments, the comparison may be made relative to “the baseline MMD”, i.e., the MMD value prior to the start of the treatment.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above.

These and other changes can be made to the invention in light of the above detailed description. In general, in the following claims, the terms used should not be construed to limit the invention to the specific embodiments disclosed in the specification and the claims. Accordingly, the invention is not limited by the disclosure, but instead the scope of the invention is to be determined entirely by the following claims.

The invention may be practiced in ways other than those particularly described in the foregoing description and examples. Numerous modifications and variations of the invention are possible in light of the above teachings and, therefore, are within the scope of the appended claims.

Certain CGRP antibody polynucleotides and polypeptides are disclosed in the sequence listing accompanying this patent application filing, and the disclosure of said sequence listing is herein incorporated by reference in its entirety.

The entire disclosure of each document cited (including patents, patent applications, journal articles, abstracts, manuals, books, or other disclosures) in the Background of the Invention, Detailed Description, and Examples is herein incorporated by reference in their entireties.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Additional Exemplary Embodiments

S1. Use of an anti-CGRP antibody or antigen-binding fragment thereof for the manufacture of a medicament for preventing or treating migraine or headache in a subject having one or more psychiatric symptoms, wherein said anti-CGRP antibody or antigen-binding fragment thereof comprises:
(A) a heavy chain variable domain (VH) comprising a CDRH1, a CDRH2, and a CDRH3; and
(B) a light chain variable domain (VL) comprising a CDRL1, a CDRL2, and a CDRL3, wherein:
(A) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and
   the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively.
   S2. The use of embodiment S1, wherein the one or more psychiatric symptoms comprise one or more depressive symptoms.
   S3. The use of embodiment S2, wherein the one or more depressive symptoms:
(I) comprise or are any one or more of the depressive symptoms defined by the Diagnostic and Statistical Manual of Mental Disorders, the 5th Edition, Text Revision (DSM-5-TR);
(II) comprise or are any one or more of the following:
   (1) depressed mood (or irritable mood in children and adolescents);
   (2) diminished interest in pleasure;
   (3) weight loss when not dieting or weight gain (e.g., a change of more than 5% of body weight in a month), or decrease or increase in appetite nearly every day (or failure to make expected weight gain in children);
   (4) insomnia or hypersomnia;
   (5) psychomotor agitation or retardation;
   (6) fatigue or loss of energy;
   (7) feelings of worthlessness or excessive or inappropriate guilt;
   (8) diminished ability to think or concentrate, or indecisiveness; and
   (9) recurrent thoughts of death, recurrent suicidal ideation without a specific plan, or a suicide attempt or a specific plan for committing suicide; and/or
(III) are one or more selected from the group comprising feelings of sadness, tearfulness, emptiness or hopelessness, angry outbursts, irritability or frustration, loss of interest or pleasure, such as sex, hobbies or sports, sleep disturbances, including insomnia or sleeping too much, tiredness and lack of energy, reduced appetite and weight loss or increased cravings for food and weight gain, anxiety, agitation or restlessness, slowed thinking, speaking or body movements, feelings of worthlessness or guilt, fixating on past failures or self-blame, trouble thinking, concentrating, making decisions and remembering things, frequent or recurrent thoughts of death, suicidal thoughts, suicide attempts or suicide, unexplained physical problems, such as back pain or headaches.
   S4. The use of any one of embodiments S1-S3, wherein the subject:
(I) (i) experiences two to four depressive symptoms:
(ii) experiences one or more minor depressive episodes, optionally wherein the occurrence of the minor depressive episodes is recurrent; and/or
(iii) has or is diagnosed with minor depressive disorder; or
(II) (i) experiences five, six, seven, eight, or more depressive symptoms:
(ii) experiences one or more major depressive episodes, optionally wherein the occurrence of the major depressive episodes is recurrent; and/or
(iii) has or is diagnosed with major depressive disorder,
   wherein the depressive symptoms are selected from the following:
(1) depressed mood most of the day, nearly every day, as indicated in the subjective report (e.g., feels sad, empty, hopeless) or in observation made by others (e.g., appears tearful) (In children and adolescents, can be irritable mood);
(2) markedly diminished interest in pleasure in all, or almost all, activities most of the day and nearly every day (as indicated by either subjective account or observation);
(3) significant weight loss when not dieting or weight gain (e.g., a change of more than 5% of body weight in a month), or decrease or increase in appetite nearly every day. (In children, consider failure to make expected weight gain.);
(4) insomnia or hypersomnia nearly every day;
(5) psychomotor agitation or retardation nearly every day (observable by others, not merely subjective feelings of restlessness or being slowed down);
(6) fatigue or loss of energy nearly every day;
(7) feelings of worthlessness or excessive or inappropriate guilt (which may be delusional) nearly every day (not merely self-reproach or guilt about being sick);
(8) diminished ability to think or concentrate, or indecisiveness nearly every day (either by subjective account or as observed by others); and
(9) recurrent thoughts of death (not just fear of dying), recurrent suicidal ideation without a specific plan, or a suicide attempt or a specific plan for committing suicide,
   optionally wherein the subject experiences at least (1) and/or (2).
   S5. The use of any one of embodiments S1-S4, wherein the subject has or is diagnosed with one or more of the following psychiatric disorders: depressive disorders, anxiety disorders, mixed anxiety-depressive disorder (MADD), bipolar and related disorders, schizophrenia spectrum or other psychotic disorders, substance-related and addictive disorders, medication-induced movement disorders and other adverse effects of medication, neurocognitive disorders, neurodevelopmental disorders, trauma- and stressor-related disorders, dissociative disorders, obsessive-compulsive and related disorders, personality disorders, somatic symptom and related disorders, feeding and eating disorders, elimination disorders, sleep-wake disorders, sexual dysfunctions, gender dysphoria, disruptive, impulse-control, and conduct disorders, paraphilic disorders, and other mental disorders, optionally wherein the psychiatric disorder is as defined by the DSM-5-TR.
   S6. The use of any one of embodiments S1-S5, wherein the subject has or is diagnosed with a depressive disorder or mixed anxiety-depressive disorder, optionally wherein the depressive disorder is selected from: major depressive disorder, minor depressive disorder, bipolar depression, mood dysregulation disorder, persistent depressive disorder, premenstrual dysphoric disorder, substance/medication-induced depressive disorder, depressive disorder due to another medical condition, another specified depressive disorder, or an unspecified depressive disorder, dysthymic disorder, emotional disorder (seasonal affective disorder and the like), recurrent depression, postpartum depression, stress disorder, major depressive disorder concomitant with psychosis (including delusive disorders and schizophrenia), manic or mixed mood episode, hypomanic mood episode, depression episode with atypical features, depression episode with melancholic features, depressive episodes with tonic features, depression episode after stroke, delirium, peripheral symptoms of dementia (mental symptoms or behavior abnormalities).
   S7. The use of any one of embodiments S1-S6, wherein:
(i) the subject has or is diagnosed with migraine, optionally as defined by the International Classification of Headache Disorders (ICHD-3); and/or
(ii) the subject has one or more of chronic migraine, episodic migraine, chronic/episodic migraine, migraines with or without aura, acute migraine or headache, hemiplegic migraines, cluster headaches, migrainous neuralgia, chronic headaches, tension headaches, general headaches, headaches due to an underlying structural problem in the head or neck, sinus headaches (such as for example associated with sinusitis), and allergy-induced headaches or migraines.
   S8. The use of any one of embodiments S1-S7, wherein the subject has a history of previous failure with one or more preventive migraine medications, optionally two or more preventive migraine medications, further optionally two to four preventive migraine medications.
   S9. The use of embodiment S8, wherein the previous failure is with one or more preventive migraine medications selected from:
(I) (i) antiepileptic drugs or anticonvulsants, optionally divalproex, valproate, topiramate, and carbamazepine; (ii) beta-blockers, optionally propranolol, metoprolol, timolol, atenolol, nadolol, nebivolol, and pindolol; (iii) triptans, optionally frovatriptan, naratriptan, and zolmitriptan; (iv) antidepressants (such as SSRI, SNRI, and TCA), optionally amitriptyline and venlafaxine; (v) calcium channel blocker, optionally flunarizine; (vi) angiotensin II receptor antagonists, optionally candesartan; (vii) ACE inhibitors, optionally lisinopril; (viii) α-agonists, optionally clonidine and guanfacine; (ix) antihistamines, optionally cyproheptadine; and (x) other medications approved for prevention of migraine;
(II) carbonic anhydrase inhibitors such as acetazolamide; antithrombotics such as coumadin and picotamide; antidepressants (such as TCA, SSRI, and SNRI) such as protriptyline, fluvoxamine, and fluoxetine; antiepileptic drugs such as gabapentin; beta-blockers such as bisoprolol; Ca2+ blockers such as nicardipine, nifedipine, nimodipine, and verapamil; and vascular smooth muscle relaxants such as cyclandelate;
(III) CGRP receptor antagonistic small molecules, optionally gepants, further optionally atogepant (QULIPTA®) and/or rimegepant (NURTEC® ODT); and/or (IV) anti-CGRP antibodies, optionally fremanezumab (AJOVY®) and/or galcanezumab (EMGALITY®), and/or anti-CGRP receptor antibodies erenumab (AIMIVOG®).
   S10. The use of any one of embodiments S1-S9, wherein:
(i) the subject is in need of immediate preventative treatment of migraine or headache; and/or
(ii) the subject exhibits at least one headache and/or migraine symptom and/or the one or more psychiatric symptoms at the time of administration, optionally wherein said at least one headache and/or migraine symptom comprises one or more of pain, nausea, photophobia, phonophobia, or aura, further optionally wherein said pain is head pain.
   S11. The use of any one of embodiments S1-S10, wherein the subject is not administered any acute migraine medication (other than the anti-CGRP antibody or antigen-binding fragment thereof) within a period of time before and after the administration of the anti-CGRP antibody or antigen-binding fragment thereof, such as within 15 minutes, within 30 minutes, within 1 hour, within 2 hours, within 3 hours, within 4 hours, within 5 hours, or within 6 hours before and after said administration.
   S12. The use of any one of embodiments S1-S11, wherein:
(I) the subject has migraine and the medicament:
   (i) reduces monthly migraine days (MMD) by at least about 3 days, at least about 3.5 days, at least about 4 days, at least about 4.5 days, optionally about 12 weeks or about 24 weeks after the administering;
   (ii) reduces MMD by at least about 50%, at least about 55%, at least about 60%, at least about 65%, at least about 70%, at least about 75; and/or
   (iii) ameliorates migraine based on Patient Global Impression of Change (PGIC); and/or
(II) the subject has depression or has one or more depressive symptoms and the medicament:
   (i) reduces the number of or ameliorates depressive symptoms;
   (ii) prevents or treats depression; and/or
   (ii) prevents or treats suicidal ideation by the subject.
      S13. Use of an anti-CGRP antibody or antigen-binding fragment thereof for the manufacture of a medicament for preventing or treating one or more psychiatric symptoms in a subject, the anti-CGRP antibody or antigen-binding fragment thereof comprising:
(A) a heavy chain variable domain (VH) comprising a CDRH1, a CDRH2, and a CDRH3; and
(B) a light chain variable domain (VL) comprising a CDRL1, a CDRL2, and a CDRL3,
   wherein:
(A) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and
(B) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively.
   S14. The use of embodiment S13, wherein the one or more psychiatric symptoms comprises one or more depressive symptoms.
   S15. The use of embodiment S14, wherein the one or more depressive symptoms:
(I) comprise or are any one or more of the depressive symptoms defined by the Diagnostic and Statistical Manual of Mental Disorders, the 5th Edition, Text Revision (DSM-5-TR);
(II) comprise or are any one or more of the following:
   (1) depressed mood (or irritable mood in children and adolescents);
   (2) diminished interest in pleasure;
   (3) weight loss when not dieting or weight gain (e.g., a change of more than 5% of body weight in a month), or decrease or increase in appetite nearly every day (or failure to make expected weight gain in children);
   (4) insomnia or hypersomnia;
   (5) psychomotor agitation or retardation;
   (6) fatigue or loss of energy;
   (7) feelings of worthlessness or excessive or inappropriate guilt;
   (8) diminished ability to think or concentrate, or indecisiveness; and
   (9) recurrent thoughts of death, recurrent suicidal ideation without a specific plan, or a suicide attempt or a specific plan for committing suicide; and/or
(III) are one or more selected from the group comprising feelings of sadness, tearfulness, emptiness or hopelessness, angry outbursts, irritability or frustration, loss of interest or pleasure, such as sex, hobbies or sports, sleep disturbances, including insomnia or sleeping too much, tiredness and lack of energy, reduced appetite and weight loss or increased cravings for food and weight gain, anxiety, agitation or restlessness, slowed thinking, speaking or body movements, feelings of worthlessness or guilt, fixating on past failures or self-blame, trouble thinking, concentrating, making decisions and remembering things, frequent or recurrent thoughts of death, suicidal thoughts, suicide attempts or suicide, unexplained physical problems, such as back pain or headaches.
   S16. The use of any one of embodiments S13-S15, wherein the subject:
(I) (i) experiences two to four depressive symptoms:
(ii) experiences one or more minor depressive episodes, optionally wherein the occurrence of the minor depressive episodes is recurrent; and/or
(iii) has or is diagnosed with minor depressive disorder; or
(II) (i) experiences five, six, seven, eight, or more depressive symptoms:
(ii) experiences one or more major depressive episodes, optionally wherein the occurrence of the major depressive episodes is recurrent; and/or
(iii) has or is diagnosed with major depressive disorder,
   wherein the depressive symptoms are selected from the following:
(1) depressed mood most of the day, nearly every day, as indicated in the subjective report (e.g., feels sad, empty, hopeless) or in observation made by others (e.g., appears tearful) (In children and adolescents, can be irritable mood);
(2) markedly diminished interest in pleasure in all, or almost all, activities most of the day and nearly every day (as indicated by either subjective account or observation);
(3) significant weight loss when not dieting or weight gain (e.g., a change of more than 5% of body weight in a month), or decrease or increase in appetite nearly every day. (In children, consider failure to make expected weight gain.);
(4) insomnia or hypersomnia nearly every day;
(5) psychomotor agitation or retardation nearly every day (observable by others, not merely subjective feelings of restlessness or being slowed down);
(6) fatigue or loss of energy nearly every day;
(7) feelings of worthlessness or excessive or inappropriate guilt (which may be delusional) nearly every day (not merely self-reproach or guilt about being sick);
(8) diminished ability to think or concentrate, or indecisiveness nearly every day (either by subjective account or as observed by others); and
(9) recurrent thoughts of death (not just fear of dying), recurrent suicidal ideation without a specific plan, or a suicide attempt or a specific plan for committing suicide,
   optionally wherein the subject experiences at least (1) and/or (2).
   S17. The use of any one of embodiments S13-S16, wherein the subject has or is diagnosed with one or more of the following psychiatric disorders: depressive disorders, anxiety disorders, mixed anxiety-depressive disorder (MADD), bipolar and related disorders, schizophrenia spectrum or other psychotic disorders, substance-related and addictive disorders, medication-induced movement disorders and other adverse effects of medication, neurocognitive disorders, neurodevelopmental disorders, trauma- and stressor-related disorders, dissociative disorders, obsessive-compulsive and related disorders, personality disorders, somatic symptom and related disorders, feeding and eating disorders, elimination disorders, sleep-wake disorders, sexual dysfunctions, gender dysphoria, disruptive, impulse-control, and conduct disorders, paraphilic disorders, and other mental disorders, optionally wherein the psychiatric disorder is as defined by the DSM-5-TR.
   S18. The use of any one of embodiments S13-S17, wherein the subject has or is diagnosed with a depressive disorder or mixed anxiety-depressive disorder, optionally wherein the depressive disorder is selected from: major depressive disorder, minor depressive disorder, bipolar depression, mood dysregulation disorder, persistent depressive disorder, premenstrual dysphoric disorder, substance/medication-induced depressive disorder, depressive disorder due to another medical condition, another specified depressive disorder, or an unspecified depressive disorder, dysthymic disorder, emotional disorder (seasonal affective disorder and the like), recurrent depression, postpartum depression, stress disorder, major depressive disorder concomitant with psychosis (including delusive disorders and schizophrenia), manic or mixed mood episode, hypomanic mood episode, depression episode with atypical features, depression episode with melancholic features, depressive episodes with tonic features, depression episode after stroke, delirium, peripheral symptoms of dementia (mental symptoms or behavior abnormalities).
   S19. The use of any one of embodiments S13-S18, wherein the subject:
(i) does not have migraine or headache; and/or
(ii) does not have or is not diagnosed with migraine.
   S20. The use of any one of embodiments S13-S18, wherein the subject
(i) has migraine or headache; and/or
(ii) has or is diagnosed with migraine, optionally as defined by the International Classification of Headache Disorders (ICHD-3),
   optionally wherein the subject has one or more of: chronic migraine, episodic migraine, chronic/episodic migraine, migraine with or without aura, acute migraine or headache, hemiplegic migraines, cluster headaches, migrainous neuralgia, chronic headaches, tension headaches, general headaches, headaches due to an underlying structural problem in the head or neck, sinus headaches (such as for example associated with sinusitis), and allergy-induced headaches or migraines.
   S21. The use of any one of embodiments S13-S20, wherein the medicament:
(i) reduces the number of or ameliorates depressive symptoms;
(ii) prevents or treats depression; and/or
(ii) prevents or treats suicidal ideation by the subject.
   S22. The use of any one of embodiments S1-S21, wherein the medicament is to be used with or further comprises one or more additional medications, optionally wherein the one or more additional medications:
(I) comprise one or more antidepressants;
(II) comprise one or more headache or migraine medications;
(III) are administered to the subject:
   (i) together with the anti-CGRP antibody or antigen-binding fragment thereof; or
   (ii) separately from the anti-CGRP antibody or antigen-binding fragment thereof, optionally prior to or after the administration of the anti-CGRP antibody or antigen-binding fragment thereof, further optionally within about 15 minutes, within about 30 minutes, within about 1 hour, within about 2 hours, within about 3 hours, within about 4 hours, within about 5 hours, or within about 6 hours before and/or after the administration of the anti-CGRP antibody or antigen-binding fragment thereof; and/or
(IV) and the anti-CGRP antibody or antigen-binding fragment thereof are contained in a same pharmaceutical composition or in separate pharmaceutical compositions.
   S23. The use of embodiment S22, wherein the one or more antidepressants comprise one or more selected from:
(i) monoamine oxidase inhibitors (MAOIs), optionally selected from isocarboxazid, nialamide, phenelzine, tranylcypromine, iproniazide, iproclozide, safrazine, monoamine, moclobemide, toloxatone, minaprine, bifemelane, and selegiline;
(ii) tricyclic antidepressants (TCAs), optionally selected from imipramine, imipramine oxide, amitriptyline, desipramine, nortriptyline, amoxapine, clomipramine, lofepramine, dosulepin, doxepin, trimipramine, protriptyline, opipramol, dibenzepin, butriptyline, amineptine, iprindole, melitracen, dimetacrine, and quinupramine;
(iii) serotonin reuptake inhibitors (SSRIs), optionally selected from fluoxetine, fluvoxamine, paroxetine, sertraline, vilazodone, vortioxetine, citalopram, alaproclate, etoperidone, escitalopram, and imelidine;
(iv) serotonin (5HT)-norepinephrine (NE) reuptake inhibitors (SNRIs), optionally selected from milnacipran, venlafaxine, sibutramine, levomilnacipran, duloxetine, desvenlafaxine, and tramadol;
(v) tetracyclic antidepressants (TeCAs), optionally selected from maprotiline, mianserin, setiptiline, and mirtazapine;
(vi) serotonin antagonist and reuptake inhibitors (SARIs), optionally selected from trazodone, etoperidone, lorpiprazole, mepiprazole, and nefazodone; (vii) N-methyl-D-aspartate (NMDA) antagonists, optionally bupropion;
(viii) serotonin-dopamine activity modulators (SDAMs), optionally selected from brexpiprazole and aripiprazole; and
(ix) other antidepressants, optionally selected from oxitriptan, tryptophan, nomifensine, viloxazine, oxaflozane, medifoxamine, tianeptine, pivagabine, reboxetine, gepirone, agomelatine, hyperici herba, esketamine, brexanolone, and dextromethorphan.
   S24. The use of embodiment S22 or S23, wherein the one or more headache or migraine medications comprise one or more selected from:
(i) an ergotamine or an ergot derivative,
(ii) a triptan, optionally selected from sumatriptan, zolmitriptan, naratriptan, rizatriptan, eletriptan, almotriptan, and/or frovatriptan;
(iii) a non-opioid analgesic, optionally selected from;
(iv) an opioid/narcotic, optionally selected from oxycodone, tramadol, butorphanol, morphine, codeine, and/or hydrocodone;
(v) acetaminophen or paracetamol;
(vi) a nonsteroidal anti-inflammatory drug (NSAID), optionally selected from acetylsalicylic acid (aspirin), celecoxib, choline salicylate, diclofenac, diflunisal, etodolac, fenoprofen, flurbiprofen sodium, ibuprofen, indomethacin, ketoprofen, ketorolac tromethamine, meclofenamate, meloxicam, nabumetone, naproxen, oxaprozin, phenylbutazone, piroxicam, rofecoxib, salsalate, sulindac, tolmetin, valdecoxib, and/or zomepirac;
(vii) a combination medication, optionally selected from: a combination of two or more drugs with analgesic effects (for example, paracetamol and codeine); a combination of an analgesic and an adjuvant (for example, paracetamol and caffeine); a combination which comprises at least one opioid (such as oxycodone, tramadol, butorphanol, morphine, codeine, hydrocodone, or any combination thereof); a combination which comprises at least one barbiturate such as butalbital; a combination which comprises at least caffeine; and/or a combination which comprises acetylsalicylic acid (aspirin), paracetamol, and caffeine (e.g., EXCEDRIN®, EXCEDRIN MIGRAINE®);
(viii) CGRP receptor antagonistic small molecules, optionally gepants, further optionally ubrogepant (UBRELVY®), atogepant (QULIPTA®), rimegepant (NURTEC® ODT), zavegepant (ZAVZPRET™); and/or
(ix) another anti-CGRP antibody and/or an anti-CGRP receptor antibody.
   S25. The use of any one of embodiments S1-S24, wherein:
(A) the amino acid sequence of the VH has at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 202; and/or
(B) the amino acid sequence of the VL has at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 222.
   S26. The use of any one of embodiments S1-S25, wherein:
(A) the amino acid sequence of the VH comprises or consists of SEQ ID NO: 202; and/or
(B) the amino acid sequence of the VL comprises or consists of SEQ ID NO: 222.
   S27. The use of any one of embodiments S1-S26, wherein the anti-CGRP antibody or antigen-binding fragment thereof comprises a scFv fragment, a diabody, a minibody, a scFv-Fc, a Fab fragment, a Fab′ fragment, a Fab′-SH fragment, a F(ab′)2 fragment, a small modular immunopharmaceuticals (SMIPs), and/or a Fv fragment.
   S28. The use of any one of embodiments S1-S26, wherein the anti-CGRP antibody or antigen-binding fragment thereof comprises:
(A) a heavy chain polypeptide comprising (i) the VH and (ii) a heavy chain constant region (CH); and
(B) a light chain polypeptide comprising (i) the VL and (ii) a light chain constant region (CL),
   optionally comprising: an immunoglobulin molecule comprised of two said heavy chain polypeptides and two said light chain polypeptides interconnected by disulfide bonds, or a multimer of said immunoglobulin molecule; optionally an IgG, IgA, IgD, IgE, or IgM antibody, further optionally an IgG1, IgG2, IgG3, IgG4, IgA1, or IgA2 antibody.
   S29. The use of embodiment S28, wherein:
(A) the amino acid sequence of the heavy chain polypeptide comprises or consists of SEQ ID NO: 201 or SEQ ID NO: 566; and/or
(B) the amino acid sequence of the light chain polypeptide comprises or consists of SEQ ID NO: 221.
   S30. The use of any one of embodiments S1-S29, wherein the medicament comprises an effective amount of the anti-CGRP antibody or antigen-binding fragment thereof, optionally wherein the effective amount is:
(i) about 100 mg or about 300 mg;
(ii) about 100 to about 300 mg, about 100 to about 500 mg, about 50 to about 1000 mg, or about 50 to about 1500 mg; or
(iii) about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, about 1000 mg, about 1100 mg, about 1200 mg, about 1300 mg, about 1400 mg, or about 1500 mg.
   S31. The use of any one of embodiments S1-S30, wherein the medicament is for intravenous administration, optionally for infusion over a period of approximately 30 min to 60 minutes.
   S32. The use of any one of embodiments S1-S31, wherein the medicament is for intravenous administration for about every 10-14 weeks, preferably about every 11-13 weeks, more preferably about every 12 weeks or about every three months, optionally wherein the total administration is at least twice, three times, four times, five time, six times, seven times, eight times, or more.
   S33. The use of embodiment S32, wherein the medicament comprises an effective amount of the anti-CGRP antibody or antigen-binding fragment thereof, optionally wherein the effective amount is about 100 mg to about 300 mg, optionally about 100 mg or about 300 mg.
   S34. The use of any one of embodiments S1-S33, wherein the medicament is comprised in a formulation comprising histidine (L-histidine and/or L-histidine monohydrate), sorbitol, polysorbate 80, and water, optionally wherein the formulation comprises, per 1 mL volume, about 100 mg or about 300 mg anti-CGRP antibody, about 3.1 mg Histidine, about 40.5 mg Sorbitol, and about 0.15 mg Polysorbate 80, and has a pH of about 5.8.
   S35. The use of any one of embodiments S1-S34, wherein said anti-CGRP antibody or antigen-binding fragment thereof is expressed in or obtained by expression in Pichia pastoris. S36. The use of any of any one of embodiments S1-S34, wherein said anti-CGRP antibody or antigen-binding fragment thereof is expressed in or obtained by expression in CHO cells.

### Further Exemplary Embodiments

E1. An anti-CGRP antibody or antigen-binding fragment thereof for use in preventing or treating migraine or headache in a patient having depression or having depressive symptoms, the anti-CGRP antibody or antigen-binding fragment thereof comprising:
(A) a heavy chain variable domain (VH) comprising a CDRH1, a CDRH2, and a CDRH3; and
(B) a light chain variable domain (VL) comprising a CDRL1, a CDRL2, and a CDRL3,
   wherein:
(A) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and
(B) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively.
   224, 226, and 228, respectively.
   E2. The anti-CGRP antibody or antigen-binding fragment thereof for use of embodiment E1, wherein the one or more psychiatric symptoms comprise one or more depressive symptoms.
   E3. The anti-CGRP antibody or antigen-binding fragment thereof for use of embodiment E2, wherein the one or more depressive symptoms:
(I) comprise or are any one or more of the depressive symptoms defined by the Diagnostic and Statistical Manual of Mental Disorders, the 5th Edition, Text Revision (DSM-5-TR);
(II) comprise or are any one or more of the following:
   (1) depressed mood (or irritable mood in children and adolescents);
   (2) diminished interest in pleasure;
   (3) weight loss when not dieting or weight gain (e.g., a change of more than 5% of body weight in a month), or decrease or increase in appetite nearly every day (or failure to make expected weight gain in children);
   (4) insomnia or hypersomnia;
   (5) psychomotor agitation or retardation;
   (6) fatigue or loss of energy;
   (7) feelings of worthlessness or excessive or inappropriate guilt;
   (8) diminished ability to think or concentrate, or indecisiveness; and
   (9) recurrent thoughts of death, recurrent suicidal ideation without a specific plan, or a suicide attempt or a specific plan for committing suicide; and/or
(III) are one or more selected from the group comprising feelings of sadness, tearfulness, emptiness or hopelessness, angry outbursts, irritability or frustration, loss of interest or pleasure, such as sex, hobbies or sports, sleep disturbances, including insomnia or sleeping too much, tiredness and lack of energy, reduced appetite and weight loss or increased cravings for food and weight gain, anxiety, agitation or restlessness, slowed thinking, speaking or body movements, feelings of worthlessness or guilt, fixating on past failures or self-blame, trouble thinking, concentrating, making decisions and remembering things, frequent or recurrent thoughts of death, suicidal thoughts, suicide attempts or suicide, unexplained physical problems, such as back pain or headaches.
   E4. The anti-CGRP antibody or antigen-binding fragment thereof for use of any one of embodiments E1-E3, wherein the subject:
(I) (i) experiences two to four depressive symptoms:
(ii) experiences one or more minor depressive episodes, optionally wherein the occurrence of the minor depressive episodes is recurrent; and/or
(iii) has or is diagnosed with minor depressive disorder; or
(II) (i) experiences five, six, seven, eight, or more depressive symptoms:
(ii) experiences one or more major depressive episodes, optionally wherein the occurrence of the major depressive episodes is recurrent; and/or
(iii) has or is diagnosed with major depressive disorder,
   wherein the depressive symptoms are selected from the following:
(1) depressed mood most of the day, nearly every day, as indicated in the subjective report (e.g., feels sad, empty, hopeless) or in observation made by others (e.g., appears tearful) (In children and adolescents, can be irritable mood);
(2) markedly diminished interest in pleasure in all, or almost all, activities most of the day and nearly every day (as indicated by either subjective account or observation);
(3) significant weight loss when not dieting or weight gain (e.g., a change of more than 5% of body weight in a month), or decrease or increase in appetite nearly every day. (In children, consider failure to make expected weight gain.);
(4) insomnia or hypersomnia nearly every day;
(5) psychomotor agitation or retardation nearly every day (observable by others, not merely subjective feelings of restlessness or being slowed down);
(6) fatigue or loss of energy nearly every day;
(7) feelings of worthlessness or excessive or inappropriate guilt (which may be delusional) nearly every day (not merely self-reproach or guilt about being sick);
(8) diminished ability to think or concentrate, or indecisiveness nearly every day (either by subjective account or as observed by others); and
(9) recurrent thoughts of death (not just fear of dying), recurrent suicidal ideation without a specific plan, or a suicide attempt or a specific plan for committing suicide,
   optionally wherein the subject experiences at least (1) and/or (2).
   E5. The anti-CGRP antibody or antigen-binding fragment thereof for use of any one of embodiments E1-E4, wherein the subject has or is diagnosed with one or more of the following psychiatric disorders: depressive disorders, anxiety disorders, mixed anxiety-depressive disorder (MADD), bipolar and related disorders, schizophrenia spectrum or other psychotic disorders, substance-related and addictive disorders, medication-induced movement disorders and other adverse effects of medication, neurocognitive disorders, neurodevelopmental disorders, trauma- and stressor-related disorders, dissociative disorders, obsessive-compulsive and related disorders, personality disorders, somatic symptom and related disorders, feeding and eating disorders, elimination disorders, sleep-wake disorders, sexual dysfunctions, gender dysphoria, disruptive, impulse-control, and conduct disorders, paraphilic disorders, and other mental disorders, optionally wherein the psychiatric disorder is as defined by the DSM-5-TR.
   E6. The anti-CGRP antibody or antigen-binding fragment thereof for use of any one of embodiments E1-5, wherein the subject has or is diagnosed with a depressive disorder or mixed anxiety-depressive disorder, optionally wherein the depressive disorder is selected from: major depressive disorder, minor depressive disorder, bipolar depression, mood dysregulation disorder, persistent depressive disorder, premenstrual dysphoric disorder, substance/medication-induced depressive disorder, depressive disorder due to another medical condition, another specified depressive disorder, or an unspecified depressive disorder, dysthymic disorder, emotional disorder (seasonal affective disorder and the like), recurrent depression, postpartum depression, stress disorder, major depressive disorder concomitant with psychosis (including delusive disorders and schizophrenia), manic or mixed mood episode, hypomanic mood episode, depression episode with atypical features, depression episode with melancholic features, depressive episodes with tonic features, depression episode after stroke, delirium, peripheral symptoms of dementia (mental symptoms or behavior abnormalities).
   E7. The anti-CGRP antibody or antigen-binding fragment thereof for use of any one of embodiments E1-E6, wherein:
(i) the subject has or is diagnosed with migraine, optionally as defined by the International Classification of Headache Disorders (ICHD-3); and/or
(ii) the subject has one or more of chronic migraine, episodic migraine, chronic/episodic migraine, migraines with or without aura, acute migraine or headache, hemiplegic migraines, cluster headaches, migrainous neuralgia, chronic headaches, tension headaches, general headaches, headaches due to an underlying structural problem in the head or neck, sinus headaches (such as for example associated with sinusitis), and allergy-induced headaches or migraines.
   E8. The anti-CGRP antibody or antigen-binding fragment thereof for use of any one of embodiments E1-E7, wherein the subject has a history of previous failure with one or more preventive migraine medications, optionally two or more preventive migraine medications, further optionally two to four preventive migraine medications.
   E9. The anti-CGRP antibody or antigen-binding fragment thereof for use of embodiment E8, wherein the previous failure is with one or more preventive migraine medications selected from:
(I) (i) antiepileptic drugs or anticonvulsants, optionally divalproex, valproate, topiramate, and carbamazepine; (ii) beta-blockers, optionally propranolol, metoprolol, timolol, atenolol, nadolol, nebivolol, and pindolol; (iii) triptans, optionally frovatriptan, naratriptan, and zolmitriptan; (iv) antidepressants (such as SSRI, SNRI, and TCA), optionally amitriptyline and venlafaxine; (v) calcium channel blocker, optionally flunarizine; (vi) angiotensin II receptor antagonists, optionally candesartan; (vii) ACE inhibitors, optionally lisinopril; (viii) α-agonists, optionally clonidine and guanfacine; (ix) antihistamines, optionally cyproheptadine; and (x) other medications approved for prevention of migraine;
(II) carbonic anhydrase inhibitors such as acetazolamide; antithrombotics such as coumadin and picotamide; antidepressants (such as TCA, SSRI, and SNRI) such as protriptyline, fluvoxamine, and fluoxetine; antiepileptic drugs such as gabapentin; beta-blockers such as bisoprolol; Ca2+ blockers such as nicardipine, nifedipine, nimodipine, and verapamil; and vascular smooth muscle relaxants such as cyclandelate;
(III) CGRP receptor antagonistic small molecules, optionally gepants, further optionally atogepant (QULIPTA®) and/or rimegepant (NURTEC® ODT); and/or
(IV) anti-CGRP antibodies, optionally fremanezumab (AJOVY®) and/or galcanezumab (EMGALITY®), and/or anti-CGRP receptor antibodies erenumab (AIMIVOG®).
   E10. The anti-CGRP antibody or antigen-binding fragment thereof for use of any one of embodiments E1-E9, wherein:
(i) the subject is in need of immediate preventative treatment of migraine or headache; and/or
(ii) the subject exhibits at least one headache and/or migraine symptom and/or the one or more psychiatric symptoms at the time of administration, optionally wherein
   said at least one headache and/or migraine symptom comprises one or more of pain, nausea, photophobia, phonophobia, or aura, further optionally wherein said pain is head pain.
   E11. The anti-CGRP antibody or antigen-binding fragment thereof for use of any one of embodiments E1-E10, wherein the subject is not administered any acute migraine medication (other than the anti-CGRP antibody or antigen-binding fragment thereof) within a period of time before and after the administration of the anti-CGRP antibody or antigen-binding fragment thereof, such as within 15 minutes, within 30 minutes, within 1 hour, within 2 hours, within 3 hours, within 4 hours, within 5 hours, or within 6 hours before and after said administration.
   E12. The anti-CGRP antibody or antigen-binding fragment thereof for use of any one of embodiments E1-E11, wherein:
(I) the subject has migraine and the use:
   (i) reduces monthly migraine days (MMD) by at least about 3 days, at least about 3.5 days, at least about 4 days, at least about 4.5 days, optionally about 12 weeks or about 24 weeks after the administering;
   (ii) reduces MMD by at least about 50%, at least about 55%, at least about 60%, at least about 65%, at least about 70%, at least about 75; and/or
   (iii) ameliorates migraine based on Patient Global Impression of Change (PGIC); and/or
(II) the subject has depression or has one or more depressive symptoms and the use:
   (i) reduces the number of or ameliorates depressive symptoms;
   (ii) prevents or treats depression; and/or
   (ii) prevents or treats suicidal ideation by the subject.
      E13. An anti-CGRP antibody or antigen-binding fragment thereof for use in preventing or treating one or more psychiatric symptoms in a subject, the use comprising administering to the subject an effective amount of an anti-CGRP antibody or antigen-binding fragment thereof comprising:
(A) a heavy chain variable domain (VH) comprising a CDRH1, a CDRH2, and a CDRH3; and
(B) a light chain variable domain (VL) comprising a CDRL1, a CDRL2, and a CDRL3,
   wherein:
(A) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and
(B) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively.
   E14. The anti-CGRP antibody or antigen-binding fragment thereof for use of embodiment E13, wherein the one or more psychiatric symptoms comprises one or more depressive symptoms.
   E15. The anti-CGRP antibody or antigen-binding fragment thereof for use of embodiment E14, wherein the one or more depressive symptoms:
(I) comprise or are any one or more of the depressive symptoms defined by the Diagnostic and Statistical Manual of Mental Disorders, the 5th Edition, Text Revision (DSM-5-TR);
(II) comprise or are any one or more of the following:
   (1) depressed mood (or irritable mood in children and adolescents);
   (2) diminished interest in pleasure;
   (3) weight loss when not dieting or weight gain (e.g., a change of more than 5% of body weight in a month), or decrease or increase in appetite nearly every day (or failure to make expected weight gain in children);
   (4) insomnia or hypersomnia;
   (5) psychomotor agitation or retardation;
   (6) fatigue or loss of energy;
   (7) feelings of worthlessness or excessive or inappropriate guilt;
   (8) diminished ability to think or concentrate, or indecisiveness; and
   (9) recurrent thoughts of death, recurrent suicidal ideation without a specific plan, or a suicide attempt or a specific plan for committing suicide; and/or
(III) are one or more selected from the group comprising feelings of sadness, tearfulness, emptiness or hopelessness, angry outbursts, irritability or frustration, loss of interest or pleasure, such as sex, hobbies or sports, sleep disturbances, including insomnia or sleeping too much, tiredness and lack of energy, reduced appetite and weight loss or increased cravings for food and weight gain, anxiety, agitation or restlessness, slowed thinking, speaking or body movements, feelings of worthlessness or guilt, fixating on past failures or self-blame, trouble thinking, concentrating, making decisions and remembering things, frequent or recurrent thoughts of death, suicidal thoughts, suicide attempts or suicide, unexplained physical problems, such as back pain or headaches.
   E16. The anti-CGRP antibody or antigen-binding fragment thereof for use of any one of embodiments E13-E15, wherein the subject:
(I) (i) experiences two to four depressive symptoms:
(ii) experiences one or more minor depressive episodes, optionally wherein the occurrence of the minor depressive episodes is recurrent; and/or
(iii) has or is diagnosed with minor depressive disorder; or
(II) (i) experiences five, six, seven, eight, or more depressive symptoms:
(ii) experiences one or more major depressive episodes, optionally wherein the occurrence of the major depressive episodes is recurrent; and/or
(iii) has or is diagnosed with major depressive disorder,
   wherein the depressive symptoms are selected from the following:
(1) depressed mood most of the day, nearly every day, as indicated in the subjective report (e.g., feels sad, empty, hopeless) or in observation made by others (e.g., appears tearful) (In children and adolescents, can be irritable mood);
(2) markedly diminished interest in pleasure in all, or almost all, activities most of the day and nearly every day (as indicated by either subjective account or observation);
(3) significant weight loss when not dieting or weight gain (e.g., a change of more than 5% of body weight in a month), or decrease or increase in appetite nearly every day. (In children, consider failure to make expected weight gain.);
(4) insomnia or hypersomnia nearly every day;
(5) psychomotor agitation or retardation nearly every day (observable by others, not merely subjective feelings of restlessness or being slowed down);
(6) fatigue or loss of energy nearly every day;
(7) feelings of worthlessness or excessive or inappropriate guilt (which may be delusional) nearly every day (not merely self-reproach or guilt about being sick);
(8) diminished ability to think or concentrate, or indecisiveness nearly every day (either by subjective account or as observed by others); and
(9) recurrent thoughts of death (not just fear of dying), recurrent suicidal ideation without a specific plan, or a suicide attempt or a specific plan for committing suicide,
   optionally wherein the subject experiences at least (1) and/or (2).
   E17. The anti-CGRP antibody or antigen-binding fragment thereof for use of any one of embodiments E13-E16, wherein the subject has or is diagnosed with one or more of the following psychiatric disorders: depressive disorders, anxiety disorders, mixed anxiety-depressive disorder (MADD), bipolar and related disorders, schizophrenia spectrum or other psychotic disorders, substance-related and addictive disorders, medication-induced movement disorders and other adverse effects of medication, neurocognitive disorders, neurodevelopmental disorders, trauma- and stressor-related disorders, dissociative disorders, obsessive-compulsive and related disorders, personality disorders, somatic symptom and related disorders, feeding and eating disorders, elimination disorders, sleep-wake disorders, sexual dysfunctions, gender dysphoria, disruptive, impulse-control, and conduct disorders, paraphilic disorders, and other mental disorders, optionally wherein the psychiatric disorder is as defined by the DSM-5-TR.
   E18. The anti-CGRP antibody or antigen-binding fragment thereof for use of any one of embodiments E13-E17, wherein the subject has or is diagnosed with a depressive disorder or mixed anxiety-depressive disorder, optionally wherein the depressive disorder is selected from: major depressive disorder, minor depressive disorder, bipolar depression, mood dysregulation disorder, persistent depressive disorder, premenstrual dysphoric disorder, substance/medication-induced depressive disorder, depressive disorder due to another medical condition, another specified depressive disorder, or an unspecified depressive disorder, dysthymic disorder, emotional disorder (seasonal affective disorder and the like), recurrent depression, postpartum depression, stress disorder, major depressive disorder concomitant with psychosis (including delusive disorders and schizophrenia), manic or mixed mood episode, hypomanic mood episode, depression episode with atypical features, depression episode with melancholic features, depressive episodes with tonic features, depression episode after stroke, delirium, peripheral symptoms of dementia (mental symptoms or behavior abnormalities).
   E19. The anti-CGRP antibody or antigen-binding fragment thereof for use of any one of embodiments E13-E18, wherein the subject:
(i) does not have migraine or headache; and/or
(ii) does not have or is not diagnosed with migraine.
   E20. The anti-CGRP antibody or antigen-binding fragment thereof for use of any one of embodiments E13-E18, wherein the subject:
(i) has migraine or headache; and/or
(ii) has or is diagnosed with migraine, optionally as defined by the International Classification of Headache Disorders (ICHD-3),
   optionally wherein the subject has one or more of: chronic migraine, episodic migraine, chronic/episodic migraine, migraine with or without aura, acute migraine or headache, hemiplegic migraines, cluster headaches, migrainous neuralgia, chronic headaches, tension headaches, general headaches, headaches due to an underlying structural problem in the head or neck, sinus headaches (such as for example associated with sinusitis), and allergy-induced headaches or migraines.
   E21. The anti-CGRP antibody or antigen-binding fragment thereof for use of any one of embodiments E13-E20, wherein the use:
(i) reduces the number of or ameliorates depressive symptoms;
(ii) prevents or treats depression; and/or
(ii) prevents or treats suicidal ideation by the subject.
   E22. The anti-CGRP antibody or antigen-binding fragment thereof for use of any one of embodiments E1-E21, wherein the use further comprises administering to the subject one or more additional medications, optionally wherein the one or more additional medications:
(I) comprise one or more antidepressants;
(II) comprise one or more headache or migraine medications;
(III) are administered to the subject:
   (i) together with the anti-CGRP antibody or antigen-binding fragment thereof; or
   (ii) separately from the anti-CGRP antibody or antigen-binding fragment thereof, optionally prior to or after the administration of the anti-CGRP antibody or antigen-binding fragment thereof, further optionally within about 15 minutes, within about 30 minutes, within about 1 hour, within about 2 hours, within about 3 hours, within about 4 hours, within about 5 hours, or within about 6 hours before and/or after the administration of the anti-CGRP antibody or antigen-binding fragment thereof; and/or
(IV) and the anti-CGRP antibody or antigen-binding fragment thereof are contained in a same pharmaceutical composition or in separate pharmaceutical compositions.
   E23. The anti-CGRP antibody or antigen-binding fragment thereof for use of embodiment E22, wherein the one or more antidepressants comprise one or more selected from:
(i) monoamine oxidase inhibitors (MAOIs), optionally selected from isocarboxazid, nialamide, phenelzine, tranylcypromine, iproniazide, iproclozide, safrazine, monoamine, moclobemide, toloxatone, minaprine, bifemelane, and selegiline;
(ii) tricyclic antidepressants (TCAs), optionally selected from imipramine, imipramine oxide, amitriptyline, desipramine, nortriptyline, amoxapine, clomipramine, lofepramine, dosulepin, doxepin, trimipramine, protriptyline, opipramol, dibenzepin, butriptyline, amineptine, iprindole, melitracen, dimetacrine, and quinupramine;
(iii) serotonin reuptake inhibitors (SSRIs), optionally selected from fluoxetine, fluvoxamine, paroxetine, sertraline, vilazodone, vortioxetine, citalopram, alaproclate, etoperidone, escitalopram, and imelidine;
(iv) serotonin (5HT)-norepinephrine (NE) reuptake inhibitors (SNRIs), optionally selected from milnacipran, venlafaxine, sibutramine, levomilnacipran, duloxetine, desvenlafaxine, and tramadol;
(v) tetracyclic antidepressants (TeCAs), optionally selected from maprotiline, mianserin, setiptiline, and mirtazapine;
(vi) serotonin antagonist and reuptake inhibitors (SARIs), optionally selected from trazodone, etoperidone, lorpiprazole, mepiprazole, and nefazodone; (vii) N-methyl-D-aspartate (NMDA) antagonists, optionally bupropion;
(viii) serotonin-dopamine activity modulators (SDAMs), optionally selected from brexpiprazole and aripiprazole; and
(ix) other antidepressants, optionally selected from oxitriptan, tryptophan, nomifensine, viloxazine, oxaflozane, medifoxamine, tianeptine, pivagabine, reboxetine, gepirone, agomelatine, hyperici herba, esketamine, brexanolone, and dextromethorphan.
   E24. The anti-CGRP antibody or antigen-binding fragment thereof for use of embodiment E22 or E23, wherein the one or more headache or migraine medications comprise one or more selected from:
(i) an ergotamine or an ergot derivative,
(ii) a triptan, optionally selected from sumatriptan, zolmitriptan, naratriptan, rizatriptan, eletriptan, almotriptan, and/or frovatriptan;
(iii) a non-opioid analgesic, optionally selected from;
(iv) an opioid/narcotic, optionally selected from oxycodone, tramadol, butorphanol, morphine, codeine, and/or hydrocodone;
(v) acetaminophen or paracetamol;
(vi) a nonsteroidal anti-inflammatory drug (NSAID), optionally selected from acetylsalicylic acid (aspirin), celecoxib, choline salicylate, diclofenac, diflunisal, etodolac, fenoprofen, flurbiprofen sodium, ibuprofen, indomethacin, ketoprofen, ketorolac tromethamine, meclofenamate, meloxicam, nabumetone, naproxen, oxaprozin, phenylbutazone, piroxicam, rofecoxib, salsalate, sulindac, tolmetin, valdecoxib, and/or zomepirac;
(vii) a combination medication, optionally selected from: a combination of two or more drugs with analgesic effects (for example, paracetamol and codeine); a combination of an analgesic and an adjuvant (for example, paracetamol and caffeine); a combination which comprises at least one opioid (such as oxycodone, tramadol, butorphanol, morphine, codeine, hydrocodone, or any combination thereof); a combination which comprises at least one barbiturate such as butalbital; a combination which comprises at least caffeine; and/or a combination which comprises acetylsalicylic acid (aspirin), paracetamol, and caffeine (e.g., EXCEDRIN®, EXCEDRIN MIGRAINE®);
(viii) CGRP receptor antagonistic small molecules, optionally gepants, further optionally ubrogepant (UBRELVY®), atogepant (QULIPTA®), rimegepant (NURTEC® ODT), zavegepant (ZAVZPRET™); and/or
(ix) another anti-CGRP antibody and/or an anti-CGRP receptor antibody.
   E25. The anti-CGRP antibody or antigen-binding fragment thereof for use of any one of embodiments E1-E24, wherein:
(A) the amino acid sequence of the VH has at least about 100%, at least about 99%, at least about 98%, at least about 97%, at least about 96%, at least about 95%, at least about 94%, at least about 9304 at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 202; and/or
(B) the amino acid sequence of the VL has at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 222.
   E26. The anti-CGRP antibody or antigen-binding fragment thereof for use of any one of embodiments E1-E25, wherein:
(A) the amino acid sequence of the VH comprises or consists of SEQ ID NO: 202; and/or
(B) the amino acid sequence of the VL comprises or consists of SEQ ID NO: 222.
   E27. The anti-CGRP antibody or antigen-binding fragment thereof for use of any one of embodiments E1-E26, wherein the anti-CGRP antibody or antigen-binding fragment thereof comprises a scFv fragment, a diabody, a minibody, a scFv-Fc, a Fab fragment, a Fab′ fragment, a Fab′-SH fragment, a F(ab′)2 fragment, a small modular immunopharmaceuticals (SMIPs), and/or a Fv fragment.
   E28. The anti-CGRP antibody or antigen-binding fragment thereof for use of any one of embodiments E1-E26, wherein the anti-CGRP antibody or antigen-binding fragment thereof comprises:
(A) a heavy chain polypeptide comprising (i) the VH and (ii) a heavy chain constant region (CH); and
(B) a light chain polypeptide comprising (i) the VL and (ii) a light chain constant region (CL),
   optionally comprising: an immunoglobulin molecule comprised of two said heavy chain polypeptides and two said light chain polypeptides interconnected by disulfide bonds, or a multimer of said immunoglobulin molecule; optionally an IgG, IgA, IgD, IgE, or IgM antibody, further optionally an IgG1, IgG2, IgG3, IgG4, IgA1, or IgA2 antibody.
   E29. The anti-CGRP antibody or antigen-binding fragment thereof for use of embodiment E28, wherein:
(A) the amino acid sequence of the heavy chain polypeptide comprises or consists of SEQ ID NO: 201 or SEQ ID NO: 566; and/or
(B) the amino acid sequence of the light chain polypeptide comprises or consists of SEQ ID NO: 221.
   E30. The anti-CGRP antibody or antigen-binding fragment thereof for use of any one of embodiments E1-E29, wherein the effective amount is:
(i) about 100 mg or about 300 mg;
(ii) about 100 to about 300 mg, about 100 to about 500 mg, about 50 to about 1000 mg, or about 50 to about 1500 mg; or
(iii) about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, about 1000 mg, about 1100 mg, about 1200 mg, about 1300 mg, about 1400 mg, or about 1500 mg.
   E31. The anti-CGRP antibody or antigen-binding fragment thereof for use of any one of embodiments E1-E30, wherein the anti-CGRP antibody or antigen-binding fragment thereof is administered intravenously, optionally infused over a period of approximately 30 min to 60 minutes.
   E32. The anti-CGRP antibody or antigen-binding fragment thereof for use of any one of embodiments E1-E31, wherein the use further comprises intravenously administering an effective amount of the anti-CGRP antibody or antigen-binding fragment thereof every 10⁻¹⁴ weeks, preferably every 11-13 weeks, more preferably every 12 weeks or about every three months, optionally wherein the total administration is at least twice, three times, four times, five time, six times, seven times, eight times, or more.
   E3. The anti-CGRP antibody or antigen-binding fragment thereof for use of embodiment E32, wherein said effective amount is about 100 mg to about 300 mg, optionally about 100 mg or about 300.
   E34. The anti-CGRP antibody or antigen-binding fragment thereof for use of any one of embodiments E1-E33, wherein said anti-CGRP antibody or antigen-binding fragment thereof is comprised in a formulation comprising histidine (L-histidine and/or L-histidine monohydrate), sorbitol, polysorbate 80, and water, optionally wherein the formulation comprises, per 1 mL volume, about 100 mg or about 300 mg anti-CGRP antibody, 3.1 mg L-Histidine, 40.5 mg Sorbitol, and 0.15 mg Polysorbate 80, and has a pH of about 5.8.
   E35. The anti-CGRP antibody or antigen-binding fragment thereof for use of any of one of embodiments E1-E34, wherein said anti-CGRP antibody or antigen-binding fragment thereof is expressed in or obtained by expression in Pichia pastoris.
   E36. The anti-CGRP antibody or antigen-binding fragment thereof for use of any of one of embodiments E1-E34, wherein said anti-CGRP antibody or antigen-binding fragment thereof is expressed in or obtained by expression in CHO cells.

### Further Embodiments of the Invention (Embodiments 1-54 Below)

Embodiment 1. A method for preventing or treating migraine or headache in a subject having one or more psychiatric symptoms, the method comprising administering to the subject an effective amount of an anti-CGRP antibody or antigen-binding fragment thereof comprising:
(A) a heavy chain variable domain (VH) comprising a CDRH1, a CDRH2, and a CDRH3; and
(B) a light chain variable domain (VL) comprising a CDRL1, a CDRL2, and a CDRL3,
   wherein:
(B) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and
(C) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively.
   Embodiment 2. The method of embodiment 1, wherein the one or more psychiatric symptoms comprise one or more depressive symptoms.
   Embodiment 3. The method of embodiment 2, wherein the one or more depressive symptoms:
(I) comprise or are any one or more of the depressive symptoms defined by the Diagnostic and Statistical Manual of Mental Disorders, the 5th Edition, Text Revision (DSM-5-TR);
(II) comprise or are any one or more of the following:
   (1) depressed mood (or irritable mood in children and adolescents);
   (2) diminished interest in pleasure;
   (3) weight loss when not dieting or weight gain (e.g., a change of more than 5% of body weight in a month), or decrease or increase in appetite nearly every day (or failure to make expected weight gain in children);
   (4) insomnia or hypersomnia;
   (5) psychomotor agitation or retardation;
   (6) fatigue or loss of energy;
   (7) feelings of worthlessness or excessive or inappropriate guilt;
   (8) diminished ability to think or concentrate, or indecisiveness; and
   (9) recurrent thoughts of death, recurrent suicidal ideation without a specific plan, or a suicide attempt or a specific plan for committing suicide; and/or
(III) are one or more selected from the group comprising feelings of sadness, tearfulness, emptiness or hopelessness, angry outbursts, irritability or frustration, loss of interest or pleasure, such as sex, hobbies or sports, sleep disturbances, including insomnia or sleeping too much, tiredness and lack of energy, reduced appetite and weight loss or increased cravings for food and weight gain, anxiety, agitation or restlessness, slowed thinking, speaking or body movements, feelings of worthlessness or guilt, fixating on past failures or self-blame, trouble thinking, concentrating, making decisions and remembering things, frequent or recurrent thoughts of death, suicidal thoughts, suicide attempts or suicide, unexplained physical problems, such as back pain or headaches.
   Embodiment 4. The method of any one of embodiments 1-3, wherein the subject:
(I) (i) experiences two to four depressive symptoms:
(ii) experiences one or more minor depressive episodes, optionally wherein the occurrence of the minor depressive episodes is recurrent; and/or
(iii) has or is diagnosed with minor depressive disorder; or
(II) (i) experiences five, six, seven, eight, or more depressive symptoms:
(ii) experiences one or more major depressive episodes, optionally wherein the occurrence of the major depressive episodes is recurrent; and/or
(iii) has or is diagnosed with major depressive disorder,
   wherein the depressive symptoms are selected from the following:
(1) depressed mood most of the day, nearly every day, as indicated in the subjective report (e.g., feels sad, empty, hopeless) or in observation made by others (e.g., appears tearful) (In children and adolescents, can be irritable mood);
(2) markedly diminished interest in pleasure in all, or almost all, activities most of the day and nearly every day (as indicated by either subjective account or observation);
(3) significant weight loss when not dieting or weight gain (e.g., a change of more than 5% of body weight in a month), or decrease or increase in appetite nearly every day. (In children, consider failure to make expected weight gain.);
(4) insomnia or hypersomnia nearly every day;
(5) psychomotor agitation or retardation nearly every day (observable by others, not merely subjective feelings of restlessness or being slowed down);
(6) fatigue or loss of energy nearly every day;
(7) feelings of worthlessness or excessive or inappropriate guilt (which may be delusional) nearly every day (not merely self-reproach or guilt about being sick);
(8) diminished ability to think or concentrate, or indecisiveness nearly every day (either by subjective account or as observed by others); and
(9) recurrent thoughts of death (not just fear of dying), recurrent suicidal ideation without a specific plan, or a suicide attempt or a specific plan for committing suicide,
   optionally wherein the subject experiences at least (1) and/or (2).
   Embodiment 5. The method of any one of embodiments 1-4, wherein the subject has or is diagnosed with one or more of the following psychiatric disorders: depressive disorders, anxiety disorders, mixed anxiety-depressive disorder (MADD), bipolar and related disorders, schizophrenia spectrum or other psychotic disorders, substance-related and addictive disorders, medication-induced movement disorders and other adverse effects of medication, neurocognitive disorders, neurodevelopmental disorders, trauma- and stressor-related disorders, dissociative disorders, obsessive-compulsive and related disorders, personality disorders, somatic symptom and related disorders, feeding and eating disorders, elimination disorders, sleep-wake disorders, sexual dysfunctions, gender dysphoria, disruptive, impulse-control, and conduct disorders, paraphilic disorders, and other mental disorders, optionally wherein the psychiatric disorder is as defined by the DSM-5-TR.
   Embodiment 6. The method of any one of embodiments 1-5, wherein the subject has or is diagnosed with a depressive disorder or mixed anxiety-depressive disorder, optionally wherein the depressive disorder is selected from: major depressive disorder, minor depressive disorder, bipolar depression, mood dysregulation disorder, persistent depressive disorder, premenstrual dysphoric disorder, substance/medication-induced depressive disorder, depressive disorder due to another medical condition, another specified depressive disorder, or an unspecified depressive disorder, dysthymic disorder, emotional disorder (seasonal affective disorder and the like), recurrent depression, postpartum depression, stress disorder, major depressive disorder concomitant with psychosis (including delusive disorders and schizophrenia), manic or mixed mood episode, hypomanic mood episode, depression episode with atypical features, depression episode with melancholic features, depressive episodes with tonic features, depression episode after stroke, delirium, peripheral symptoms of dementia (mental symptoms or behavior abnormalities).
   Embodiment 7. The method of any one of embodiments 1-6, wherein:
(i) the subject has or is diagnosed with migraine, optionally as defined by the International Classification of Headache Disorders (ICHD-3); and/or
(ii) the subject has one or more of chronic migraine, episodic migraine, chronic/episodic migraine, migraines with or without aura, acute migraine or headache, hemiplegic migraines, cluster headaches, migrainous neuralgia, chronic headaches, tension headaches, general headaches, headaches due to an underlying structural problem in the head or neck, sinus headaches (such as for example associated with sinusitis), and allergy-induced headaches or migraines.
   Embodiment 8. The method of any one of embodiments 1-7, wherein the subject is administered the anti-CGRP antibody or antigen-binding fragment thereof during and/or on the onset of a depressive episode, optionally a major depressive episode or a minor depressive episode, optionally wherein the subject has been diagnosed with a depressive disorder, an anxiety disorder, a mixed anxiety-depressive disorder (MADD), and/or a bipolar disorder, and the administration alleviates the severity and/or duration of the depressive episode and/or the frequency and/or severity of later depressive episodes.
   Embodiment 9. The method of any one of embodiments 1-8, wherein the subject has a history of previous failure with one or more preventive migraine medications, optionally two or more preventive migraine medications, further optionally two to four preventive migraine medications.
   Embodiment 10. The method of embodiment 9, wherein the previous failure is with one or more preventive migraine medications selected from:
(I) (i) antiepileptic drugs or anticonvulsants, optionally divalproex, valproate, topiramate, and carbamazepine; (ii) beta-blockers, optionally propranolol, metoprolol, timolol, atenolol, nadolol, nebivolol, and pindolol; (iii) triptans, optionally frovatriptan, naratriptan, and zolmitriptan; (iv) antidepressants (such as SSRI, SNRI, and TCA), optionally amitriptyline and venlafaxine; (v) calcium channel blocker, optionally flunarizine; (vi) angiotensin II receptor antagonists, optionally candesartan; (vii) ACE inhibitors, optionally lisinopril; (viii) α-agonists, optionally clonidine and guanfacine; (ix) antihistamines, optionally cyproheptadine; and (x) other medications approved for prevention of migraine;
(II) carbonic anhydrase inhibitors such as acetazolamide; antithrombotics such as coumadin and picotamide; antidepressants (such as TCA, SSRI, and SNRI) such as protriptyline, fluvoxamine, and fluoxetine; antiepileptic drugs such as gabapentin; beta-blockers such as bisoprolol; Ca2+ blockers such as nicardipine, nifedipine, nimodipine, and verapamil; and vascular smooth muscle relaxants such as cyclandelate;
(III) CGRP receptor antagonistic small molecules, optionally gepants, further optionally atogepant (QULIPTA®) and/or rimegepant (NURTEC® ODT); and/or
(IV) anti-CGRP antibodies, optionally fremanezumab (AJOVY®) and/or galcanezumab (EMGALITY®), and/or anti-CGRP receptor antibodies erenumab (AIMOVIG®).
   Embodiment 11. The method of any one of embodiments 1-10, wherein:
(i) the subject is in need of immediate preventative treatment of migraine or headache; and/or
(ii) the subject exhibits at least one headache and/or migraine symptom and/or the one or more psychiatric symptoms at the time of administration, optionally wherein said at least one headache and/or migraine symptom comprises one or more of pain, nausea, photophobia, phonophobia, or aura, further optionally wherein said pain is head pain.
   Embodiment 12. The method of any one of embodiments 1-11, wherein the subject is not administered any acute migraine medication (other than the anti-CGRP antibody or antigen-binding fragment thereof) within a period of time before and after the administration of the anti-CGRP antibody or antigen-binding fragment thereof, such as within 15 minutes, within 30 minutes, within 1 hour, within 2 hours, within 3 hours, within 4 hours, within 5 hours, or within 6 hours before and after said administration.
   Embodiment 13. The method of any one of embodiments 1-12, wherein:
(I) the subject has migraine and the method:
   (i) reduces monthly migraine days (MMD) by at least about 3 days, at least about 3.5 days, at least about 4 days, at least about 4.5 days, optionally about 12 weeks or about 24 weeks after the administering;
   (ii) reduces MMD by at least about 50%, at least about 55%, at least about 60%, at least about 65%, at least about 70%, at least about 75; and/or
   (iii) ameliorates migraine based on Patient Global Impression of Change (PGIC); and/or
(II) the subject has depression or has one or more depressive symptoms and the method:
   (i) reduces the number of or ameliorates depressive symptoms;
   (ii) prevents or treats depression; and/or
   (ii) prevents or treats suicidal ideation by the subject.
      Embodiment 14. A method for preventing or treating one or more psychiatric symptoms in a subject, the method comprising administering to the subject an effective amount of an anti-CGRP antibody or antigen-binding fragment thereof comprising:
(A) a heavy chain variable domain (VH) comprising a CDRH1, a CDRH2, and a CDRH3; and
(B) a light chain variable domain (VL) comprising a CDRL1, a CDRL2, and a CDRL3,
   wherein:
(C) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and
(D) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively.
   Embodiment 15. The method of embodiment 14, wherein the one or more psychiatric symptoms comprise one or more depressive symptoms.
   Embodiment 16. The method of embodiment 15, wherein the one or more depressive symptoms:
(I) comprise or are any one or more of the depressive symptoms defined by the Diagnostic and Statistical Manual of Mental Disorders, the 5th Edition, Text Revision (DSM-5-TR);
(II) comprise or are any one or more of the following:
   (1) depressed mood (or irritable mood in children and adolescents);
   (2) diminished interest in pleasure;
   (3) weight loss when not dieting or weight gain (e.g., a change of more than 5% of body weight in a month), or decrease or increase in appetite nearly every day (or failure to make expected weight gain in children);
   (4) insomnia or hypersomnia;
   (5) psychomotor agitation or retardation;
   (6) fatigue or loss of energy;
   (7) feelings of worthlessness or excessive or inappropriate guilt;
   (8) diminished ability to think or concentrate, or indecisiveness; and
   (9) recurrent thoughts of death, recurrent suicidal ideation without a specific plan, or a suicide attempt or a specific plan for committing suicide; and/or
(III) are one or more selected from the group comprising feelings of sadness, tearfulness, emptiness or hopelessness, angry outbursts, irritability or frustration, loss of interest or pleasure, such as sex, hobbies or sports, sleep disturbances, including insomnia or sleeping too much, tiredness and lack of energy, reduced appetite and weight loss or increased cravings for food and weight gain, anxiety, agitation or restlessness, slowed thinking, speaking or body movements, feelings of worthlessness or guilt, fixating on past failures or self-blame, trouble thinking, concentrating, making decisions and remembering things, frequent or recurrent thoughts of death, suicidal thoughts, suicide attempts or suicide, unexplained physical problems, such as back pain or headaches.
   Embodiment 17. The method of any one of embodiments 14-16, wherein the subject:
(I) (i) experiences two to four depressive symptoms:
(ii) experiences one or more minor depressive episodes, optionally wherein the occurrence of the minor depressive episodes is recurrent; and/or
(iii) has or is diagnosed with minor depressive disorder; or
(II) (i) experiences five, six, seven, eight, or more depressive symptoms:
(ii) experiences one or more major depressive episodes, optionally wherein the occurrence of the major depressive episodes is recurrent; and/or
(iii) has or is diagnosed with major depressive disorder,
   wherein the depressive symptoms are selected from the following:
(1) depressed mood most of the day, nearly every day, as indicated in the subjective report (e.g., feels sad, empty, hopeless) or in observation made by others (e.g., appears tearful) (In children and adolescents, can be irritable mood);
(2) markedly diminished interest in pleasure in all, or almost all, activities most of the day and nearly every day (as indicated by either subjective account or observation);
(3) significant weight loss when not dieting or weight gain (e.g., a change of more than 5% of body weight in a month), or decrease or increase in appetite nearly every day. (In children, consider failure to make expected weight gain.);
(4) insomnia or hypersomnia nearly every day;
(5) psychomotor agitation or retardation nearly every day (observable by others, not merely subjective feelings of restlessness or being slowed down);
(6) fatigue or loss of energy nearly every day;
(7) feelings of worthlessness or excessive or inappropriate guilt (which may be delusional) nearly every day (not merely self-reproach or guilt about being sick);
(8) diminished ability to think or concentrate, or indecisiveness nearly every day (either by subjective account or as observed by others); and
(9) recurrent thoughts of death (not just fear of dying), recurrent suicidal ideation without a specific plan, or a suicide attempt or a specific plan for committing suicide,
   optionally wherein the subject experiences at least (1) and/or (2).
   Embodiment 18. The method of any one of embodiments 14-17, wherein the subject has or is diagnosed with one or more of the following psychiatric disorders: depressive disorders, anxiety disorders, mixed anxiety-depressive disorder (MADD), bipolar and related disorders, schizophrenia spectrum or other psychotic disorders, substance-related and addictive disorders, medication-induced movement disorders and other adverse effects of medication, neurocognitive disorders, neurodevelopmental disorders, trauma- and stressor-related disorders, dissociative disorders, obsessive-compulsive and related disorders, personality disorders, somatic symptom and related disorders, feeding and eating disorders, elimination disorders, sleep-wake disorders, sexual dysfunctions, gender dysphoria, disruptive, impulse-control, and conduct disorders, paraphilic disorders, and other mental disorders, optionally wherein the psychiatric disorder is as defined by the DSM-5-TR.
   Embodiment 19. The method of any one of embodiments 14-18, wherein the subject has or is diagnosed with a depressive disorder or mixed anxiety-depressive disorder, optionally wherein the depressive disorder is selected from: major depressive disorder, minor depressive disorder, bipolar depression, mood dysregulation disorder, persistent depressive disorder, premenstrual dysphoric disorder, substance/medication-induced depressive disorder, depressive disorder due to another medical condition, another specified depressive disorder, or an unspecified depressive disorder, dysthymic disorder, emotional disorder (seasonal affective disorder and the like), recurrent depression, postpartum depression, stress disorder, major depressive disorder concomitant with psychosis (including delusive disorders and schizophrenia), manic or mixed mood episode, hypomanic mood episode, depression episode with atypical features, depression episode with melancholic features, depressive episodes with tonic features, depression episode after stroke, delirium, peripheral symptoms of dementia (mental symptoms or behavior abnormalities).
   Embodiment 20. The method of any one of embodiments 14-19, wherein the subject:
(i) does not have migraine or headache; and/or
(ii) does not have or is not diagnosed with migraine.
   Embodiment 21. The method of any one of embodiments 14-19, wherein the subject
(i) has migraine or headache; and/or
(ii) has or is diagnosed with migraine, optionally as defined by the International Classification of Headache Disorders (ICHD-3),
   optionally wherein the subject has one or more of: chronic migraine, episodic migraine, chronic/episodic migraine, migraine with or without aura, acute migraine or headache, hemiplegic migraines, cluster headaches, migrainous neuralgia, chronic headaches, tension headaches, general headaches, headaches due to an underlying structural problem in the head or neck, sinus headaches (such as for example associated with sinusitis), and allergy-induced headaches or migraines.
   Embodiment 22. The method of any one of embodiments 14-20, wherein the subject is administered the anti-CGRP antibody or antigen-binding fragment thereof during and/or on the onset of a depressive episode, optionally a major depressive episode or a minor depressive episode, optionally wherein the subject has been diagnosed with a depressive disorder, an anxiety disorder, a mixed anxiety-depressive disorder (MADD), and/or a bipolar disorder, and the administration alleviates the severity and/or duration of the bout of depression and/or the frequency and/or severity of later depressive episodes.
   Embodiment 23. The method of any one of embodiments 14-22, which
(i) reduces the number of or ameliorates depressive symptoms;
(ii) prevents or treats depression; and/or
(ii) prevents or treats suicidal ideation by the subject.
   Embodiment 24. The method of any one of embodiments 1-23, further comprising administering to the subject one or more additional medications, optionally wherein the one or more additional medications:
(I) comprise one or more antidepressants;
(II) comprise one or more headache or migraine medications;
(III) are administered to the subject:
   (i) together with the anti-CGRP antibody or antigen-binding fragment thereof; or
   (ii) separately from the anti-CGRP antibody or antigen-binding fragment thereof, optionally prior to or after the administration of the anti-CGRP antibody or antigen-binding fragment thereof, further optionally within about 15 minutes, within about 30 minutes, within about 1 hour, within about 2 hours, within about 3 hours, within about 4 hours, within about 5 hours, or within about 6 hours before and/or after the administration of the anti-CGRP antibody or antigen-binding fragment thereof; and/or
(IV) and the anti-CGRP antibody or antigen-binding fragment thereof are contained in a same pharmaceutical composition or in separate pharmaceutical compositions.
   Embodiment 25. The method of embodiment 24, wherein the one or more antidepressants comprise one or more selected from:
(i) monoamine oxidase inhibitors (MAOIs), optionally selected from isocarboxazid, nialamide, phenelzine, tranylcypromine, iproniazide, iproclozide, safrazine, monoamine, moclobemide, toloxatone, minaprine, bifemelane, and selegiline;
(ii) tricyclic antidepressants (TCAs), optionally selected from imipramine, imipramine oxide, amitriptyline, desipramine, nortriptyline, amoxapine, clomipramine, lofepramine, dosulepin, doxepin, trimipramine, protriptyline, opipramol, dibenzepin, butriptyline, amineptine, iprindole, melitracen, dimetacrine, and quinupramine;
(iii) serotonin reuptake inhibitors (SSRIs), optionally selected from fluoxetine, fluvoxamine, paroxetine, sertraline, vilazodone, vortioxetine, citalopram, alaproclate, etoperidone, escitalopram, and imelidine;
(iv) serotonin (5HT)-norepinephrine (NE) reuptake inhibitors (SNRIs), optionally selected from milnacipran, venlafaxine, sibutramine, levomilnacipran, duloxetine, desvenlafaxine, and tramadol;
(v) tetracyclic antidepressants (TeCAs), optionally selected from maprotiline, mianserin, setiptiline, and mirtazapine;
(vi) serotonin antagonist and reuptake inhibitors (SARIs), optionally selected from trazodone, etoperidone, lorpiprazole, mepiprazole, and nefazodone;
(vii) N-methyl-D-aspartate (NMDA) antagonists, optionally bupropion;
(viii) serotonin-dopamine activity modulators (SDAMs), optionally selected from brexpiprazole and aripiprazole; and
(ix) other antidepressants, optionally selected from oxitriptan, tryptophan, nomifensine, viloxazine, oxaflozane, medifoxamine, tianeptine, pivagabine, reboxetine, gepirone, agomelatine, hyperici herba, esketamine, brexanolone, and dextromethorphan.
   Embodiment 26. The method of embodiment 24 or 25, wherein the one or more headache or migraine medications comprise one or more selected from:
(i) an ergotamine or an ergot derivative,
(ii) a triptan, optionally selected from sumatriptan, zolmitriptan, naratriptan, rizatriptan, eletriptan, almotriptan, and/or frovatriptan;
(iii) a non-opioid analgesic, optionally selected from;
(iv) an opioid/narcotic, optionally selected from oxycodone, tramadol, butorphanol, morphine, codeine, and/or hydrocodone;
(v) acetaminophen or paracetamol;
(vi) a nonsteroidal anti-inflammatory drug (NSAID), optionally selected from acetylsalicylic acid (aspirin), celecoxib, choline salicylate, diclofenac, diflunisal, etodolac, fenoprofen, flurbiprofen sodium, ibuprofen, indomethacin, ketoprofen, ketorolac tromethamine, meclofenamate, meloxicam, nabumetone, naproxen, oxaprozin, phenylbutazone, piroxicam, rofecoxib, salsalate, sulindac, tolmetin, valdecoxib, and/or zomepirac;
(vii) a combination medication, optionally selected from: a combination of two or more drugs with analgesic effects (for example, paracetamol and codeine); a combination of an analgesic and an adjuvant (for example, paracetamol and caffeine); a combination which comprises at least one opioid (such as oxycodone, tramadol, butorphanol, morphine, codeine, hydrocodone, or any combination thereof); a combination which comprises at least one barbiturate such as butalbital; a combination which comprises at least caffeine; and/or a combination which comprises acetylsalicylic acid (aspirin), paracetamol, and caffeine (e.g., EXCEDRIN®, EXCEDRIN MIGRAINE®);
(viii) CGRP receptor antagonistic small molecules, optionally gepants, further optionally ubrogepant (UBRELVY®), atogepant (QULIPTA®), rimegepant (NURTEC® ODT), zavegepant (ZAVZPRET™); and/or
(ix) another anti-CGRP antibody and/or an anti-CGRP receptor antibody.
   Embodiment 27. The method of any one of embodiments 1-26, wherein:
(A) the amino acid sequence of the VH has at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 202; and/or
(B) the amino acid sequence of the VL has at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 222.
   Embodiment 28. The method of any one of embodiments 1-27, wherein:
(A) the amino acid sequence of the VH comprises or consists of SEQ ID NO: 202; and/or
(B) the amino acid sequence of the VL comprises or consists of SEQ ID NO: 222.
   Embodiment 29. The method of any one of embodiments 1-28, wherein the anti-CGRP antibody or antigen-binding fragment thereof comprises a scFv fragment, a diabody, a minibody, a scFv-Fc, a Fab fragment, a Fab′ fragment, a Fab′-SH fragment, a F(ab′)2 fragment, a small modular immunopharmaceuticals (SMIPs), and/or a Fv fragment.
   Embodiment 30. The method of any one of embodiments 1-28, wherein the anti-CGRP antibody or antigen-binding fragment thereof comprises:
(A) a heavy chain polypeptide comprising (i) the VH and (ii) a heavy chain constant region (CH); and
(B) a light chain polypeptide comprising (i) the VL and (ii) a light chain constant region (CL), optionally comprising: an immunoglobulin molecule comprised of two said heavy chain polypeptides and two said light chain polypeptides interconnected by disulfide bonds, or a multimer of said immunoglobulin molecule; optionally an IgG, IgA, IgD, IgE, or IgM antibody, further optionally an IgG1, IgG2, IgG3, IgG4, IgA1, or IgA2 antibody.
   Embodiment 31. The method of embodiment 30, wherein:
(A) the amino acid sequence of the heavy chain polypeptide comprises or consists of SEQ ID NO: 201 or SEQ ID NO: 566; and/or
(B) the amino acid sequence of the light chain polypeptide comprises or consists of SEQ ID NO: 221.
   Embodiment 32. The method of any one of embodiments 1-31, wherein the effective amount is:
(i) about 100 mg or about 300 mg;
(ii) about 100 to about 300 mg, about 100 to about 500 mg, about 50 to about 1000 mg, or about 50 to about 1500 mg; or
(iii) about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, about 1000 mg, about 1100 mg, about 1200 mg, about 1300 mg, about 1400 mg, or about 1500 mg.
   Embodiment 33. The method of any one of embodiments 1-32, wherein the anti-CGRP antibody or antigen-binding fragment thereof is administered intravenously, optionally infused over a period of approximately 30 min to 60 minutes.
   Embodiment 34. The method of any one of embodiments 1-33, further comprising intravenously administering an effective amount of the anti-CGRP antibody or antigen-binding fragment thereof about every 10⁻¹⁴ weeks, preferably about every 11-13 weeks, more preferably about every 12 weeks or about every three months, optionally wherein the total administration is at least twice, three times, four times, five time, six times, seven times, eight times, or more.
   Embodiment 35. The method of embodiment 34, wherein said effective amount is about 100 mg to about 300 mg, optionally about 100 mg or about 300 mg.
   Embodiment 36. The method of any one of embodiments 1-35, wherein said anti-CGRP antibody or antigen-binding fragment thereof is comprised in a formulation comprising histidine (L-histidine and/or L-histidine monohydrate), sorbitol, polysorbate 80, and water, optionally wherein the formulation: comprises, per 1 mL volume, about 100 mg or about 300 mg anti-CGRP antibody, about 3.1 mg Histidine, about 40.5 mg Sorbitol, and about 0.15 mg Polysorbate 80, and has a pH of about 5.8.
   Embodiment 37. The method of any one of embodiments 1-36, wherein said anti-CGRP antibody or antigen-binding fragment thereof is expressed in or obtained by expression in Pichia pastoris.
   Embodiment 38. The method of any of any one of embodiments 1-36, wherein said anti-CGRP antibody or antigen-binding fragment thereof is expressed in or obtained by expression in CHO cells.
   Embodiment 39. An anti-CGRP antibody or antigen-binding fragment thereof or a pharmaceutical composition containing, for use in preventing or treating migraine or headache in a subject having one or more psychiatric symptoms, wherein the anti-CGRP antibody or antigen-binding fragment comprises:
(A) a heavy chain variable domain (VH) comprising a CDRH1, a CDRH2, and a CDRH3; and
(B) a light chain variable domain (VL) comprising a CDRL1, a CDRL2, and a CDRL3,
   wherein:
(A) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and
(B) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively,
   optionally wherein the use is according to the method of any one of embodiments 1-13 or embodiments 24-38.
   Embodiment 40. An anti-CGRP antibody or antigen-binding fragment thereof or a pharmaceutical composition containing, for use in preventing or treating one or more psychiatric symptoms in a subject, wherein the anti-CGRP antibody or antigen-binding fragment comprises:
(A) a heavy chain variable domain (VH) comprising a CDRH1, a CDRH2, and a CDRH3; and
(B) a light chain variable domain (VL) comprising a CDRL1, a CDRL2, and a CDRL3,
   wherein:
(A) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and
(B) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively,
   optionally wherein the use is according to the method of any one of embodiments 14-38.
   Embodiment 41. Use of anti-CGRP antibody or antigen-binding fragment thereof or a pharmaceutical composition containing, for the manufacture of a medicament for preventing or treating migraine or headache in a subject having one or more psychiatric symptoms, optionally wherein the medicament is used according to the method of any one of embodiments 1-13 and 24-38.
   Embodiment 42. Use of anti-CGRP antibody or antigen-binding fragment thereof or a pharmaceutical composition containing, for the manufacture of a medicament for preventing or treating one or more psychiatric symptoms in a subject, optionally wherein the medicament is used according to the method of any one of embodiments 14-38.
   Embodiment 43. Use of anti-CGRP antibody or antigen-binding fragment thereof or a pharmaceutical composition containing, for preventing or treating migraine or headache in a subject having one or more psychiatric symptoms, optionally wherein the use is according to the method of any one of embodiments 1-13 or 24-38.
   Embodiment 44. Use of anti-CGRP antibody or antigen-binding fragment thereof or a pharmaceutical composition containing, for preventing or treating one or more psychiatric symptoms in a subject, optionally wherein the use is according to the method of any one of embodiments 14-38.
   4 Embodiment 5. A pharmaceutical composition comprising:
(I) an effective amount of an anti-CGRP antibody or antigen-binding fragment thereof comprising:
   (A) a heavy chain variable domain (VH) comprising a CDRH1, a CDRH2, and a CDRH3; and
   (B) a light chain variable domain (VL) comprising a CDRL1, a CDRL2, and a CDRL3,
   wherein:
   (C) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and
   (D) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively:
(II) one or more antidepressants; and
(III) a pharmaceutically acceptable carrier.
   Embodiment 46. The pharmaceutical composition of embodiment 45, wherein the one or more antidepressants comprise one or more selected from:
(i) monoamine oxidase inhibitors (MAOIs), optionally selected from isocarboxazid, nialamide, phenelzine, tranylcypromine, iproniazide, iproclozide, safrazine, monoamine, moclobemide, toloxatone, minaprine, bifemelane, and selegiline;
(ii) tricyclic antidepressants (TCAs), optionally selected from imipramine, imipramine oxide, amitriptyline, desipramine, nortriptyline, amoxapine, clomipramine, lofepramine, dosulepin, doxepin, trimipramine, protriptyline, opipramol, dibenzepin, butriptyline, amineptine, iprindole, melitracen, dimetacrine, and quinupramine;
(iii) serotonin reuptake inhibitors (SSRIs), optionally selected from fluoxetine, fluvoxamine, paroxetine, sertraline, vilazodone, vortioxetine, citalopram, alaproclate, etoperidone, escitalopram, and imelidine;
(iv) serotonin (5HT)-norepinephrine (NE) reuptake inhibitors (SNRIs), optionally selected from milnacipran, venlafaxine, sibutramine, levomilnacipran, duloxetine, desvenlafaxine, and tramadol;
(v) tetracyclic antidepressants (TeCAs), optionally selected from maprotiline, mianserin, setiptiline, and mirtazapine;
(vi) serotonin antagonist and reuptake inhibitors (SARIs), optionally selected from trazodone, etoperidone, lorpiprazole, mepiprazole, and nefazodone;
(vii) N-methyl-D-aspartate (NMDA) antagonists, optionally bupropion;
(viii) serotonin-dopamine activity modulators (SDAMs), optionally selected from brexpiprazole and aripiprazole; and
(ix) other antidepressants, optionally selected from oxitriptan, tryptophan, nomifensine, viloxazine, oxaflozane, medifoxamine, tianeptine, pivagabine, reboxetine, gepirone, agomelatine, hyperici herba, esketamine, brexanolone, and dextromethorphan.
   Embodiment 47. The pharmaceutical composition of embodiment 45 or 46, wherein:
(A) the amino acid sequence of the VH has at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 202; and/or
(B) the amino acid sequence of the VL has at least about 100%, at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at about 85%, at least about 84%, at least about 83%, at least about 82%, at least about 80% sequence identity to SEQ ID NO: 222.
   Embodiment 48. The pharmaceutical composition of any one of embodiments 45-47, wherein:
(A) the amino acid sequence of the VH comprises or consists of SEQ ID NO: 202; and/or
(B) the amino acid sequence of the VL comprises or consists of SEQ ID NO: 222.
   Embodiment 49. The pharmaceutical composition of any one of embodiments 45-48, wherein the anti-CGRP antibody or antigen-binding fragment thereof comprises a scFv fragment, a diabody, a minibody, a scFv-Fc, a Fab fragment, a Fab′ fragment, a Fab′-SH fragment, a F(ab′)2 fragment, a small modular immunopharmaceuticals (SMIPs), and/or a Fv fragment.
   Embodiment 50. The pharmaceutical composition of any one of embodiments 45-48, wherein the anti-CGRP antibody or antigen-binding fragment thereof comprises:
(A) a heavy chain polypeptide comprising (i) the VH and (ii) a heavy chain constant region (CH); and
(B) a light chain polypeptide comprising (i) the VL and (ii) a light chain constant region (CL), optionally comprising: an immunoglobulin molecule comprised of two said heavy chain polypeptides and two said light chain polypeptides interconnected by disulfide bonds, or a multimer of said immunoglobulin molecule; optionally an IgG, IgA, IgD, IgE, or IgM antibody, further optionally an IgG1, IgG2, IgG3, IgG4, IgA1, or IgA2 antibody.
   Embodiment 51. The pharmaceutical composition of embodiment 50, wherein:
(A) the amino acid sequence of the heavy chain polypeptide comprises or consists of SEQ ID NO: 201 or SEQ ID NO: 566; and/or
(B) the amino acid sequence of the light chain polypeptide comprises or consists of SEQ ID NO: 221.
   Embodiment 52. The pharmaceutical composition of any one of embodiments 45-51, wherein the effective amount is:
(i) about 100 mg or about 300 mg;
(ii) about 100 to about 300 mg, about 100 to about 500 mg, about 50 to about 1000 mg, or about 50 to about 1500 mg; or
(iii) about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, about 1000 mg, about 1100 mg, about 1200 mg, about 1300 mg, about 1400 mg, or about 1500 mg.
   Embodiment 53. The pharmaceutical composition of any one of embodiments 45-52 for use in preventing or treating one or more psychiatric symptoms in a subject, optionally wherein the use is according to the method of any one of embodiments 14-38.
   Embodiment 54. Use of the pharmaceutical composition of any one of embodiments 45-52 for the manufacture of a medicament for preventing or treating one or more psychiatric symptoms in a subject, optionally wherein the medicament is used according to the method of any one of embodiments 14-38.
   Embodiment 55. Use of the pharmaceutical composition of any one of embodiments 45-52 for preventing or treating one or more psychiatric symptoms in a subject, optionally wherein the use is according to the method of any one of embodiments 14-38.

### EXAMPLES

The following examples are provided in order to illustrate the invention, but are not to be construed as limiting the scope of the claims in any way.

### Example 1

Preparation of Antibodies that Bind CGRP

The preparation of exemplary anti-CGRP antibodies Ab1-Ab14 having the sequences in FIGS. 1A-12 is disclosed in commonly owned PCT Application WO/2012/162243, published on Nov. 29, 2012, the contents of which are incorporated by reference herein. This application exemplifies synthesis of these antibodies in Pichia pastoris cells. The present Applicant further contemplates synthesis of anti-CGRP antibodies Ab1-Ab14, and Ab6 in particular in CHO cells.

### Example 2

The DELIVER study evaluated the efficacy and safety of eptinezumab (“Ab6” disclosed herein) versus placebo for migraine prevention in adults with episodic or chronic migraine and history of 2-4 preventive migraine treatment failures (ClinicalTrials.gov: NCT04418765) (Ashina M, et al. Lancet Neurol. 2022; 21(7):597-607. Ashina M, et al. J Headache Pain. 2023; 24(1):155).

DELIVER comprised a 24-week double-blind placebo-controlled period followed by a 48-week dose-blinded extension period. Patients were first randomized to receive treatment with eptinezumab 100 mg IV (n=299), 300 mg IV (n=294), or placebo IV (n=298) on Day 0 and on Week 12 during the placebo-controlled period (i.e., 2 doses). Patients who received placebo IV during the placebo-controlled period were then randomized to receive treatment with eptinezumab 100 mg IV (n=145) or 300 mg IV (n=148) on Week 24 and every 12 weeks during the 48-week extension period (i.e., 4 doses). Patients who received eptinezumab 100 mg IV or 300 mg IV during the placebo-controlled period continued to receive eptinezumab 100 mg IV (n=288) or 300 mg IV (n=284), respectively, on Week 24 and every 12 weeks during the 48-week extension period (i.e., additional 4 doses). IV stands for intravenous administration.

### Inclusion Criteria:

The participant has a diagnosis of migraine, with a history of chronic or episodic migraines of at least 12 months prior to the Screening Visit
The participant has a migraine onset of ≤50 years of age.
The participant has ≥4 migraine days per month for each month within the past 3 months prior to the Screening Visit.
The participant has demonstrated compliance with the Headache eDiary by entry of data for at least 24 of the 28 days following the Screening Visit.
The participant fulfils the following criteria for chronic migraine (CM) or episodic migraine (EM) in prospectively collected information in the eDiary during the screening period:
For participants with CM: Migraine occurring on ≥8 days and headache occurring on >14 days
For participants with EM: Migraine occurring on ≥4 days and headache occurring on ≤14 days
The participant has documented evidence of treatment failure (must be supported by medical record or by physician's confirmation specific to each treatment) in the past 10 years of 2-4 different migraine preventive medications.
The participant has a history of either previous or active use of triptans for migraine.

### Exclusion Criteria:

The participant has experienced failure on a previous treatment targeting the calcitonin gene-related peptide (CGRP) pathway.
The participant has a treatment failure on valproate/divalproex or botulinum toxin A/B and the treatment is not the latest preventive medication prior to study inclusion. The medication is regarded as the latest if the medication start date is after the start date of the other preventive medications and the medication stop date is after the stop date of the other preventive medications.
The participant has confounding and clinically significant pain syndromes, (for example, fibromyalgia, chronic low back pain, complex regional pain syndrome).
The participant has a diagnosis of acute or active temporomandibular disorder.
The participant has a history or diagnosis of chronic tension-type headache, hypnic headache, cluster headache, hemicrania continua, new daily persistent headache, or unusual migraine subtypes such as hemiplegic migraine (sporadic and familial), ophthalmoplegic migraine, and migraine with neurological accompaniments that are not typical of migraine aura (diplopia, altered consciousness, or long duration).
The participant has a psychiatric condition that is uncontrolled and/or untreated for a minimum of 6 months prior to the Screening Visit. Participants with a lifetime history of psychosis and/or mania in the last 5 years prior to the Screening Visit are excluded.
The participant has a history of clinically significant cardiovascular disease or vascular ischemia or thromboembolic events (for example, cerebrovascular accident, deep vein thrombosis, or pulmonary embolism).

Analysis populations included the total population (“Total population”), the subgroup of patients reporting one or more psychiatric comorbidities at baseline (“Any psychiatric comorbidity”), and the subset reporting depressive symptoms at baseline (“Depressive symptoms”). Psychiatric comorbidities (including depressive symptoms) were self-reported by patients at screening and not cross-validated with medical records. Table 1 summarizes the number of patients who reported depressive disorders or symptoms at baseline in the “Depressive symptoms” subgroup who received 100 mg eptinezumab, 300 mg eptinezumab, or placebo.

TABLE 1
Depression and depressive symptoms reported at baseline
in the “depressive symptom” subgroup.

| | Eptinezumab | Eptinezumab | |
|---|---|---|---|
| | 100 mg | 300 mg | Placebo |
| Anxiety-depressive disorders | 4 | 4 | 3 |
| Recurrent depressive disorders | 1 | 1 | 1 |
| Depressive disorders | 5 | 6 | 3 |
| Depressive symptoms | 3 | 1 | 1 |
| Mild depressive disorder | 4 | 3 | 4 |
| Moderate depressive disorders | 0 | 3 | 0 |
| Total | 17 | 18 | 12 |

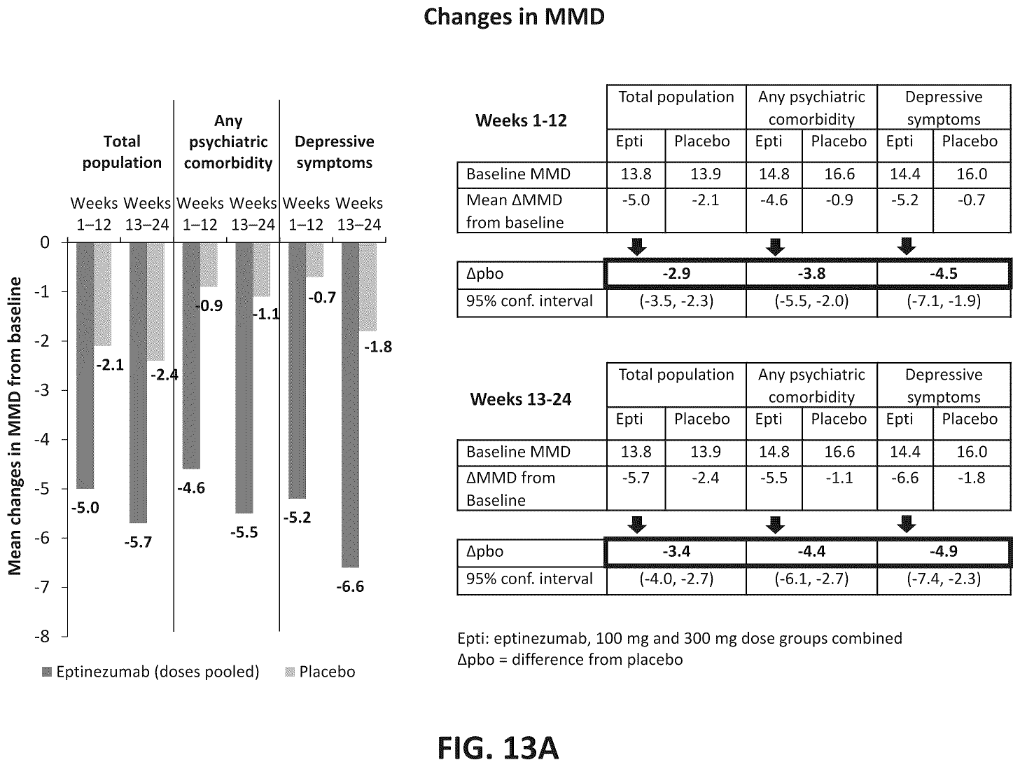

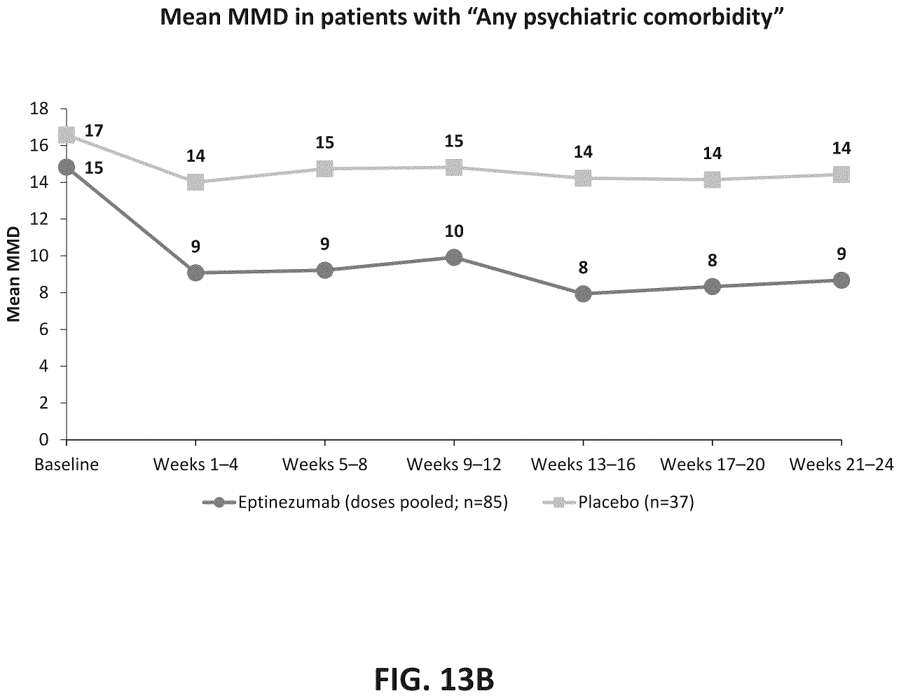

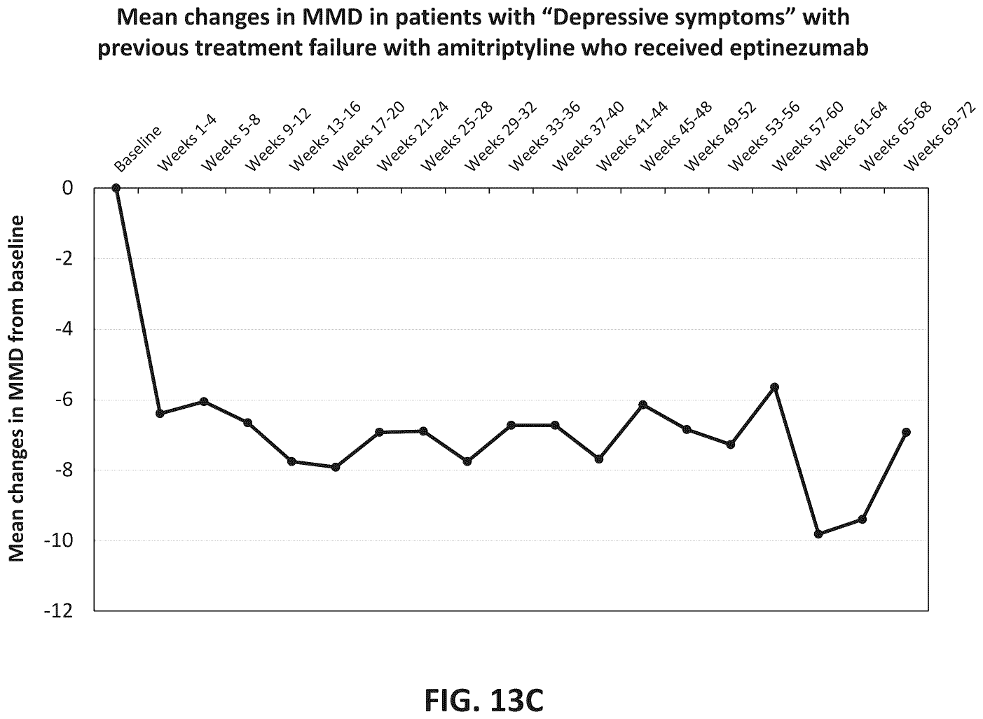

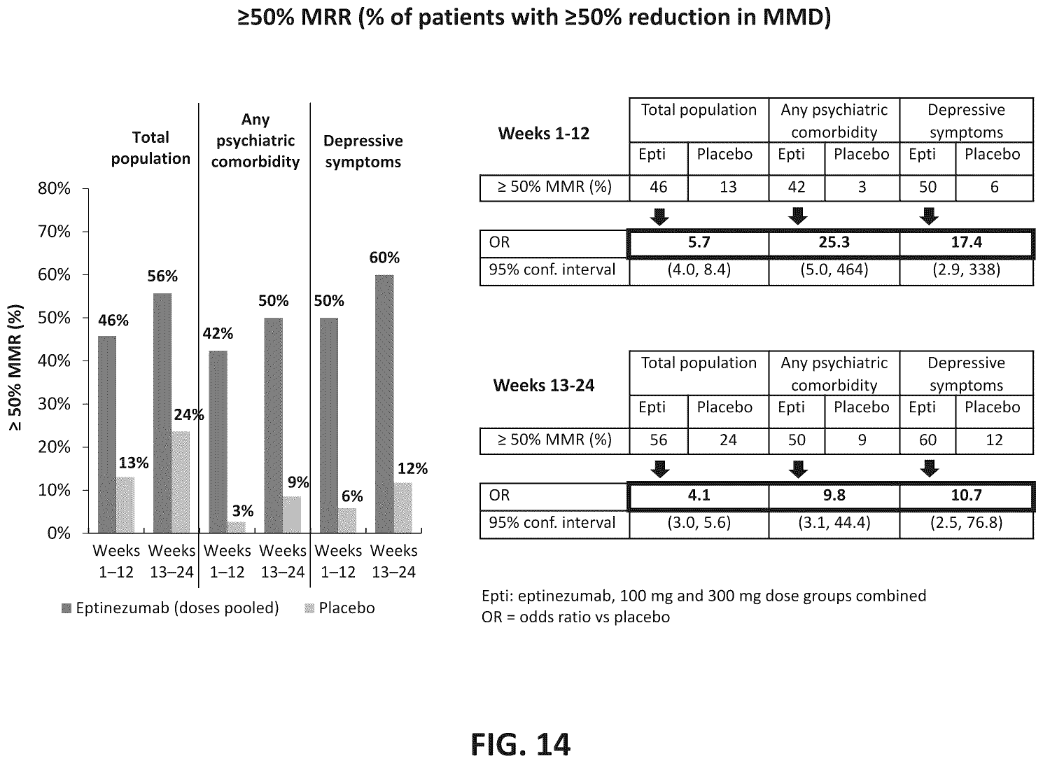

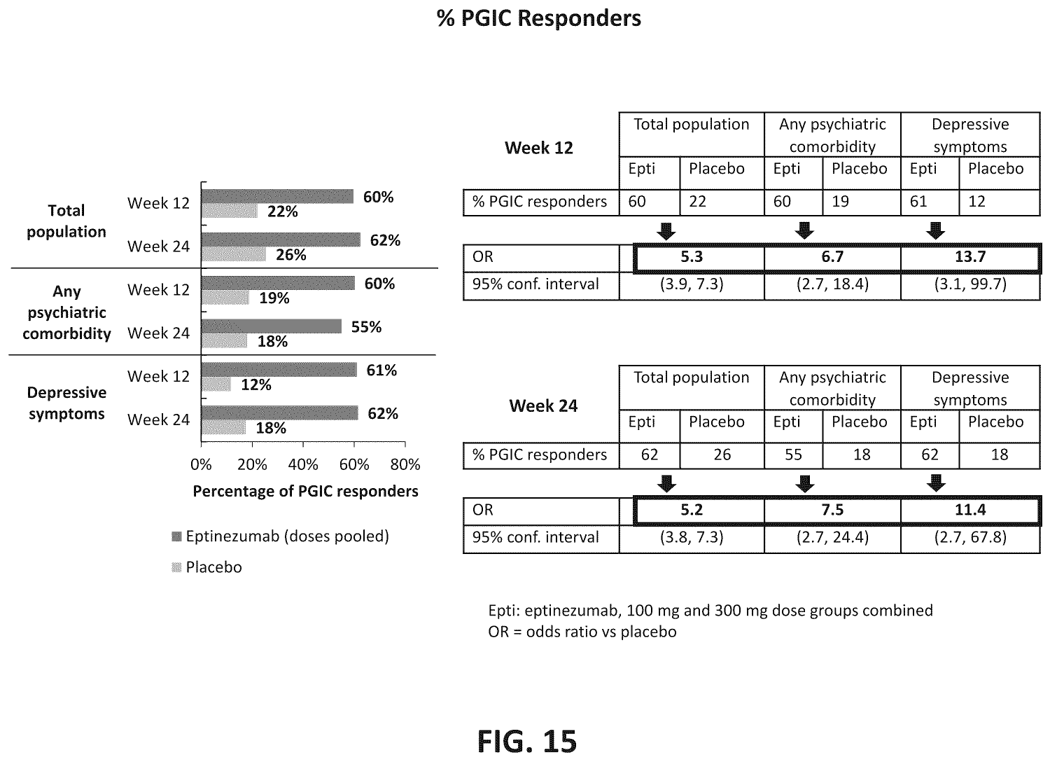

Outcomes included changes from baseline in monthly migraine days (MMD), ≥50% migraine responder rates (MRR), and Patient Global Impression of Change (PGIC) responder rates (percent of patients reporting being much or very much improved) for the “Total population”, “Any psychiatric comorbidity”), and “Depressive symptoms” groups. Results are shown in FIGS. 13A, 13B, 14, and 15.

There were 25 patients with previous treatment failure with amitriptyline (an antidepressant used as a migraine preventive medication) within the “Depressive symptoms” subgroup who received eptinezumab. The average MMD at baseline in these patients was 14.9 days. Changes from baseline in MMD in these patients are shown in FIG. 13C. The average change from baseline in mean MMD was −6.37 days in Weeks 1-12 and −7.54 days in Weeks 13-24.

Eptinezumab doses were pooled for analysis. As a post hoc analysis, no P-values are reported, and all results are descriptive.

Adverse events were also recorded. Psychiatric disorders/symptoms reported as serious and non-serious adverse events in the total patient population during the placebo-controlled period and the extension period are summarized in Tables 2 and 3, respectively.

TABLE 2
Serious adverse events (psychiatric disorders)

| Placebo-controlled period | Placebo | 100 mg | 300 mg | Placebo | | 100 mg | 300 mg |
|---|---|---|---|---|---|---|---|
| Extension period | NA | NA | NA | 100 mg | 300 mg | 100 mg | 300 mg |
| Analyzed population | 298 | 299 | 294 | 145 | 148 | 288 | 284 |
| Suicidal ideation | 1 | 0 | 0 | 0 | 0 | 0 | 0 |

TABLE 3
Non-serious adverse events (psychiatric disorders)

| Placebo-controlled period | Placebo | 100 mg | 300 mg | Placebo | | 100 mg | 300 mg |
|---|---|---|---|---|---|---|---|
| Extension period | NA | NA | NA | 100 mg | 300 mg | 100 mg | 300 mg |
| Analyzed population | 298 | 299 | 294 | 145 | 148 | 288 | 284 |
| Depressive symptom | 3 | 0 | 1 | 1 | 0 | 1 | 0 |
| Insomnia | 0 | 2 | 3 | 0 | 1 | 4 | 3 |
| Anxiety | 2 | 1 | 1 | 0 | 1 | 3 | 2 |
| Depression | 1 | 1 | 0 | 2 | 0 | 2 | 0 |

What is claimed is:

1. A method for preventing or treating migraine or headache in a subject having one or more psychiatric symptoms, the method comprising administering to the subject an effective amount of an anti-CGRP antibody or antigen-binding fragment thereof comprising:
(A) a heavy chain variable domain (VH) comprising a CDRH1, a CDRH2, and a CDRH3; and
(B) a light chain variable domain (VL) comprising a CDRL1, a CDRL2, and a CDRL3,
wherein:
(A) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and
(B) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively.

2. The method of claim 1, wherein the one or more psychiatric symptoms comprise one or more depressive symptoms.

3. The method of claim 2, wherein the one or more depressive symptoms:
(I) comprise or are any one or more of the depressive symptoms defined by the Diagnostic and Statistical Manual of Mental Disorders, the 5th Edition, Text Revision (DSM-5-TR);
(II) comprise or are any one or more of the following:
(1) depressed mood (or irritable mood in children and adolescents);
(2) diminished interest in pleasure;
(3) weight loss when not dieting or weight gain (e.g., a change of more than 5% of body weight in a month), or decrease or increase in appetite nearly every day (or failure to make expected weight gain in children);
(4) insomnia or hypersomnia;
(5) psychomotor agitation or retardation;
(6) fatigue or loss of energy;
(7) feelings of worthlessness or excessive or inappropriate guilt;
(8) diminished ability to think or concentrate, or indecisiveness; and
(9) recurrent thoughts of death, recurrent suicidal ideation without a specific plan, or a suicide attempt or a specific plan for committing suicide; and/or
(III) are one or more selected from the group comprising feelings of sadness, tearfulness, emptiness or hopelessness, angry outbursts, irritability or frustration, loss of interest or pleasure, such as sex, hobbies or sports, sleep disturbances, including insomnia or sleeping too much, tiredness and lack of energy, reduced appetite and weight loss or increased cravings for food and weight gain, anxiety, agitation or restlessness, slowed thinking, speaking or body movements, feelings of worthlessness or guilt, fixating on past failures or self-blame, trouble thinking, concentrating, making decisions and remembering things, frequent or recurrent thoughts of death, suicidal thoughts, suicide attempts or suicide, unexplained physical problems, such as back pain or headaches.

4. The method of claim 1, wherein the subject:
(I) (i) experiences two to four depressive symptoms:
(ii) experiences one or more minor depressive episodes, optionally wherein the occurrence of the minor depressive episodes is recurrent; and/or
(iii) has or is diagnosed with minor depressive disorder; or
(II) (i) experiences five, six, seven, eight, or more depressive symptoms:
(ii) experiences one or more major depressive episodes, optionally wherein the occurrence of the major depressive episodes is recurrent; and/or
(iii) has or is diagnosed with major depressive disorder,
wherein the depressive symptoms are selected from the following:
(1) depressed mood most of the day, nearly every day, as indicated in the subjective report (e.g., feels sad, empty, hopeless) or in observation made by others (e.g., appears tearful) (In children and adolescents, can be irritable mood);
(2) markedly diminished interest in pleasure in all, or almost all, activities most of the day and nearly every day (as indicated by either subjective account or observation);
(3) significant weight loss when not dieting or weight gain (e.g., a change of more than 5% of body weight in a month), or decrease or increase in appetite nearly every day. (In children, this may include failure to make expected weight gain.);
(4) insomnia or hypersomnia nearly every day;
(5) psychomotor agitation or retardation nearly every day (observable by others, not merely subjective feelings of restlessness or being slowed down);
(6) fatigue or loss of energy nearly every day;
(7) feelings of worthlessness or excessive or inappropriate guilt (which may be delusional) nearly every day (not merely self-reproach or guilt about being sick);
(8) diminished ability to think or concentrate, or indecisiveness nearly every day (either by subjective account or as observed by others); and
(9) recurrent thoughts of death (not just fear of dying), recurrent suicidal ideation without a specific plan, or a suicide attempt or a specific plan for committing suicide,
optionally wherein the subject experiences at least (1) and/or (2).

5. The method of claim 1, wherein the subject has or is diagnosed with one or more of the following psychiatric disorders: depressive disorders, anxiety disorders, mixed anxiety-depressive disorder (MADD), bipolar and related disorders, schizophrenia spectrum or other psychotic disorders, substance-related and addictive disorders, medication-induced movement disorders and other adverse effects of medication, neurocognitive disorders, neurodevelopmental disorders, trauma- and stressor-related disorders, dissociative disorders, obsessive-compulsive and related disorders, personality disorders, somatic symptom and related disorders, feeding and eating disorders, elimination disorders, sleep-wake disorders, sexual dysfunctions, gender dysphoria, disruptive, impulse-control, and conduct disorders, paraphilic disorders, and other mental disorders, optionally wherein the psychiatric disorder is as defined by the DSM-5-TR.

6. The method of claim 1, wherein the subject has or is diagnosed with a depressive disorder or mixed anxiety-depressive disorder, optionally wherein the depressive disorder is selected from: major depressive disorder, minor depressive disorder, bipolar depression, mood dysregulation disorder, persistent depressive disorder, premenstrual dysphoric disorder, substance/medication-induced depressive disorder, depressive disorder due to another medical condition, another specified depressive disorder, or an unspecified depressive disorder, dysthymic disorder, emotional disorder (seasonal affective disorder and the like), recurrent depression, postpartum depression, stress disorder, major depressive disorder concomitant with psychosis (including delusive disorders and schizophrenia), manic or mixed mood episode, hypomanic mood episode, depression episode with atypical features, depression episode with melancholic features, depressive episodes with tonic features, depression episode after stroke, delirium, peripheral symptoms of dementia (mental symptoms or behavior abnormalities).

7. The method of claim 1, wherein:
(i) the subject has or is diagnosed with migraine, optionally as defined by the International Classification of Headache Disorders (ICHD-3); and/or
(ii) the subject has one or more of chronic migraine, episodic migraine, chronic/episodic migraine, migraines with or without aura, acute migraine or headache, hemiplegic migraines, cluster headaches, migrainous neuralgia, chronic headaches, tension headaches, general headaches, headaches due to an underlying structural problem in the head or neck, sinus headaches (such as for example associated with sinusitis), and allergy-induced headaches or migraines.

8. The method of claim 1, wherein the subject is administered the anti-CGRP antibody or antigen-binding fragment thereof during and/or on the onset of a depressive episode, optionally a major depressive episode or a minor depressive episode, optionally wherein the subject has been diagnosed with a depressive disorder, an anxiety disorder, a mixed anxiety-depressive disorder (MADD), and/or a bipolar disorder, and the administration alleviates the severity and/or duration of the depressive episode and/or the frequency and/or severity of later depressive episodes.

9. The method of claim 1, wherein the subject has a history of previous failure with one or more preventive migraine medications, optionally two or more preventive migraine medications, further optionally two to four preventive migraine medications.

10. The method of claim 9, wherein the previous failure is with one or more preventive migraine medications selected from:
(I) (i) antiepileptic drugs or anticonvulsants, optionally divalproex, valproate, topiramate, and carbamazepine; (ii) beta-blockers, optionally propranolol, metoprolol, timolol, atenolol, nadolol, nebivolol, and pindolol; (iii) triptans, optionally frovatriptan, naratriptan, and zolmitriptan; (iv) antidepressants (such as SSRI, SNRI, and TCA), optionally amitriptyline and venlafaxine; (v) calcium channel blocker, optionally flunarizine; (vi) angiotensin II receptor antagonists, optionally candesartan; (vii) ACE inhibitors, optionally lisinopril; (viii) α-agonists, optionally clonidine and guanfacine; (ix) antihistamines, optionally cyproheptadine; and (x) other medications approved for prevention of migraine;
(II) carbonic anhydrase inhibitors such as acetazolamide; antithrombotics such as coumadin and picotamide; antidepressants (such as TCA, SSRI, and SNRI) such as protriptyline, fluvoxamine, and fluoxetine; antiepileptic drugs such as gabapentin; beta-blockers such as bisoprolol; Ca2+ blockers such as nicardipine, nifedipine, nimodipine, and verapamil; and vascular smooth muscle relaxants such as cyclandelate;
(III) CGRP receptor antagonistic small molecules, optionally gepants, further optionally atogepant (QULIPTA®) and/or rimegepant (NURTEC® ODT); and/or
(IV) anti-CGRP antibodies, optionally fremanezumab (AJOVY®) and/or galcanezumab (EMGALITY®), and/or anti-CGRP receptor antibodies erenumab (AIMOVIG®).

11. The method of claim 1, wherein:
(i) the subject is in need of immediate preventative treatment of migraine or headache; and/or
(ii) the subject exhibits at least one headache and/or migraine symptom and/or the one or more psychiatric symptoms at the time of administration, optionally wherein said at least one headache and/or migraine symptom comprises one or more of pain, nausea, photophobia, phonophobia, or aura, further optionally wherein said pain is head pain.

12. The method of claim 1, wherein the subject is not administered any acute migraine medication (other than the anti-CGRP antibody or antigen-binding fragment thereof) within a period of time before and after the administration of the anti-CGRP antibody or antigen-binding fragment thereof, such as within 15 minutes, within 30 minutes, within 1 hour, within 2 hours, within 3 hours, within 4 hours, within 5 hours, or within 6 hours before and after said administration.

13. The method of claim 1, wherein:
(I) the subject has migraine and the method:
(i) reduces monthly migraine days (MMD) by at least about 3 days, at least about 3.5 days, at least about 4 days, at least about 4.5 days, optionally about 12 weeks or about 24 weeks after the administering;
(ii) reduces MMD by at least about 50%, at least about 55%, at least about 60%, at least about 65%, at least about 70%, at least about 75; and/or
(iii) ameliorates migraine based on Patient Global Impression of Change (PGIC); and/or
(II) the subject has depression or has one or more depressive symptoms and the method:
(i) reduces the number of or ameliorates depressive symptoms;
(ii) prevents or treats depression; and/or
(ii) prevents or treats suicidal ideation by the subject.

14. A method for preventing or treating one or more psychiatric symptoms in a subject, the method comprising administering to the subject an effective amount of an anti-CGRP antibody or antigen-binding fragment thereof comprising:
(A) a heavy chain variable domain (VH) comprising a CDRH1, a CDRH2, and a CDRH3; and
(B) a light chain variable domain (VL) comprising a CDRL1, a CDRL2, and a CDRL3,
wherein:
(A) the amino acid sequences of the CDRH1, the CDRH2, and the CDRH3 comprise the heavy chain CDR 1, 2, and 3 sequences, respectively, contained in SEQ ID NO: 202, or comprise SEQ ID NOS: 204, 206, and 208, respectively: and
(B) the amino acid sequences of the CDRL1, the CDRL2, and the CDRL3 comprise the light chain CDR 1, 2, and 3 sequences, respectively, contained in SEQ ID NO: 222, or comprise SEQ ID NOS: 224, 226, and 228, respectively.

15. The method of claim 14, wherein the one or more psychiatric symptoms comprise one or more depressive symptoms.

16. The method of claim 15, wherein the one or more depressive symptoms:
(I) comprise or are any one or more of the depressive symptoms defined by the Diagnostic and Statistical Manual of Mental Disorders, the 5th Edition, Text Revision (DSM-5-TR);
(II) comprise or are any one or more of the following:
(1) depressed mood (or irritable mood in children and adolescents);
(2) diminished interest in pleasure;
(3) weight loss when not dieting or weight gain (e.g., a change of more than 5% of body weight in a month), or decrease or increase in appetite nearly every day (or failure to make expected weight gain in children);
(4) insomnia or hypersomnia;
(5) psychomotor agitation or retardation;
(6) fatigue or loss of energy;
(7) feelings of worthlessness or excessive or inappropriate guilt;
(8) diminished ability to think or concentrate, or indecisiveness; and
(9) recurrent thoughts of death, recurrent suicidal ideation without a specific plan, or a suicide attempt or a specific plan for committing suicide; and/or
(III) are one or more selected from the group comprising feelings of sadness, tearfulness, emptiness or hopelessness, angry outbursts, irritability or frustration, loss of interest or pleasure, such as sex, hobbies or sports, sleep disturbances, including insomnia or sleeping too much, tiredness and lack of energy, reduced appetite and weight loss or increased cravings for food and weight gain, anxiety, agitation or restlessness, slowed thinking, speaking or body movements, feelings of worthlessness or guilt, fixating on past failures or self-blame, trouble thinking, concentrating, making decisions and remembering things, frequent or recurrent thoughts of death, suicidal thoughts, suicide attempts or suicide, unexplained physical problems, such as back pain or headaches.

17. The method of claim 14, wherein the subject:
(I) (i) experiences two to four depressive symptoms:
(ii) experiences one or more minor depressive episodes, optionally wherein the occurrence of the minor depressive episodes is recurrent; and/or
(iii) has or is diagnosed with minor depressive disorder; or
(II) (i) experiences five, six, seven, eight, or more depressive symptoms:
(ii) experiences one or more major depressive episodes, optionally wherein the occurrence of the major depressive episodes is recurrent; and/or
(iii) has or is diagnosed with major depressive disorder,
wherein the depressive symptoms are selected from the following:
(1) depressed mood most of the day, nearly every day, as indicated in the subjective report (e.g., feels sad, empty, hopeless) or in observation made by others (e.g., appears tearful) (In children and adolescents, can be irritable mood);
(2) markedly diminished interest in pleasure in all, or almost all, activities most of the day and nearly every day (as indicated by either subjective account or observation);
(3) significant weight loss when not dieting or weight gain (e.g., a change of more than 5% of body weight in a month), or decrease or increase in appetite nearly every day. (In children, consider failure to make expected weight gain.);
(4) insomnia or hypersomnia nearly every day;
(5) psychomotor agitation or retardation nearly every day (observable by others, not merely subjective feelings of restlessness or being slowed down);
(6) fatigue or loss of energy nearly every day;
(7) feelings of worthlessness or excessive or inappropriate guilt (which may be delusional) nearly every day (not merely self-reproach or guilt about being sick);
(8) diminished ability to think or concentrate, or indecisiveness nearly every day (either by subjective account or as observed by others); and
(9) recurrent thoughts of death (not just fear of dying), recurrent suicidal ideation without a specific plan, or a suicide attempt or a specific plan for committing suicide,
optionally wherein the subject experiences at least (1) and/or (2).

18. The method of claim 14, wherein the subject has or is diagnosed with one or more of the following psychiatric disorders: depressive disorders, anxiety disorders, mixed anxiety-depressive disorder (MADD), bipolar and related disorders, schizophrenia spectrum or other psychotic disorders, substance-related and addictive disorders, medication-induced movement disorders and other adverse effects of medication, neurocognitive disorders, neurodevelopmental disorders, trauma- and stressor-related disorders, dissociative disorders, obsessive-compulsive and related disorders, personality disorders, somatic symptom and related disorders, feeding and eating disorders, elimination disorders, sleep-wake disorders, sexual dysfunctions, gender dysphoria, disruptive, impulse-control, and conduct disorders, paraphilic disorders, and other mental disorders, optionally wherein the psychiatric disorder is as defined by the DSM-5-TR.

19. The method of claim 14, wherein the subject has or is diagnosed with a depressive disorder or mixed anxiety-depressive disorder, optionally wherein the depressive disorder is selected from: major depressive disorder, minor depressive disorder, bipolar depression, mood dysregulation disorder, persistent depressive disorder, premenstrual dysphoric disorder, substance/medication-induced depressive disorder, depressive disorder due to another medical condition, another specified depressive disorder, or an unspecified depressive disorder, dysthymic disorder, emotional disorder (seasonal affective disorder and the like), recurrent depression, postpartum depression, stress disorder, major depressive disorder concomitant with psychosis (including delusive disorders and schizophrenia), manic or mixed mood episode, hypomanic mood episode, depression episode with atypical features, depression episode with melancholic features, depressive episodes with tonic features, depression episode after stroke, delirium, peripheral symptoms of dementia (mental symptoms or behavior abnormalities).

20. The method of claim 14, wherein the subject:
(i) does not have migraine or headache; and/or
(ii) does not have or is not diagnosed with migraine.

21. The method of claim 14, wherein the subject
(i) has migraine or headache; and/or
(ii) has or is diagnosed with migraine, optionally as defined by the International Classification of Headache Disorders (ICHD-3),
optionally wherein the subject has one or more of: chronic migraine, episodic migraine, chronic/episodic migraine, migraine with or without aura, acute migraine or headache, hemiplegic migraines, cluster headaches, migrainous neuralgia, chronic headaches, tension headaches, general headaches, headaches due to an underlying structural problem in the head or neck, sinus headaches (such as for example associated with sinusitis), and allergy-induced headaches or migraines.

22. The method of claim 14, wherein the subject is administered the anti-CGRP antibody or antigen-binding fragment thereof during and/or on the onset of a depressive episode, optionally a major depressive episode or a minor depressive episode, optionally wherein the subject has been diagnosed with a depressive disorder, an anxiety disorder, a mixed anxiety-depressive disorder (MADD), and/or a bipolar disorder, and the administration alleviates the severity and/or duration of the bout of depression and/or the frequency and/or severity of later depressive episodes.

23. The method of claim 14, which
(i) reduces the number of or ameliorates depressive symptoms;
(ii) prevents or treats depression; and/or
(ii) prevents or treats suicidal ideation by the subject.

24. The method of claim 14, wherein the subject is further administered one or more drugs used for treating and/or preventing said one or more psychiatric symptoms, optionally one or more antidepressants, further optionally wherein the administration of the anti-CGRP antibody or antigen-binding fragment thereof and said one or more drugs elicits an additive or synergistic effect on treating and/or preventing said one or more psychiatric symptoms in the subject.

25. The method of claim 24, wherein the one or more antidepressants are selected from:
(i) monoamine oxidase inhibitors (MAOIs), optionally selected from isocarboxazid, nialamide, phenelzine, tranylcypromine, iproniazide, iproclozide, safrazine, monoamine, moclobemide, toloxatone, minaprine, bifemelane, and selegiline;
(ii) tricyclic antidepressants (TCAs), optionally selected from imipramine, imipramine oxide, amitriptyline, desipramine, nortriptyline, amoxapine, clomipramine, lofepramine, dosulepin, doxepin, trimipramine, protriptyline, opipramol, dibenzepin, butriptyline, amineptine, iprindole, melitracen, dimetacrine, and quinupramine;
(iii) serotonin reuptake inhibitors (SSRIs), optionally selected from fluoxetine, fluvoxamine, paroxetine, sertraline, vilazodone, vortioxetine, citalopram, alaproclate, etoperidone, escitalopram, and imelidine;
(iv) serotonin (5HT)-norepinephrine (NE) reuptake inhibitors (SNRIs), optionally selected from milnacipran, venlafaxine, sibutramine, levomilnacipran, duloxetine, desvenlafaxine, and tramadol;
(v) tetracyclic antidepressants (TeCAs), optionally selected from maprotiline, mianserin, setiptiline, and mirtazapine;
(vi) serotonin antagonist and reuptake inhibitors (SARIs), optionally selected from trazodone, etoperidone, lorpiprazole, mepiprazole, and nefazodone;
(vii) N-methyl-D-aspartate (NMDA) antagonists, optionally bupropion;
(viii) serotonin-dopamine activity modulators (SDAMs), optionally selected from brexpiprazole and aripiprazole; and
(ix) other antidepressants, optionally selected from oxitriptan, tryptophan, nomifensine, viloxazine, oxaflozane, medifoxamine, tianeptine, pivagabine, reboxetine, gepirone, agomelatine, hyperici herba, esketamine, brexanolone, and dextromethorphan.
